(19) United States
(12) Patent Application Publication
(10) Pub. No.: US 2025/0186656 A1
(43) Pub. Date: Jun. 12, 2025

(54) SHAPE MORPHING HYDROGEL ACTUATORS AND CONSTRUCTS

(71) Applicant: Case Western Reserve University, Cleveland, OH (US)

(72) Inventors: Eben Alsberg, Chicago, IL (US); Aixiang Ding, Chicago, IL (US); Oju Jeon, Broadview Heights, OH (US)

(21) Appl. No.: 18/842,116

(22) PCT Filed: Feb. 28, 2023
(86) PCT No.: PCT/US2023/014129

§ 371 Date: Aug. 28, 2024

Related U.S. Application Data
(60) Provisional application No. 63/314,852, filed on Feb. 28, 2022.

(51) Int. Cl. A61L 27/52; A61L 27/16; A61L 27/22; A61L 27/38; A61L 27/58; B33Y 10/00; B33Y 30/00

(52) U.S. Cl. CPC A61L 27/52; A61L 27/16; A61L 27/222; A61L 27/3834; A61L 27/58; B33Y 10/00; B33Y 30/00

(57) ABSTRACT

A construct includes a biocompatible polymer-based shape morphing hydrogel or cell condensate that is configured to undergo multiple, reversible, and/or controllable different shape transformations over time via either pre-programmed design or user-controlled environmental condition alterations, wherein the hydrogel is cytocompatible and, upon degradation, produces substantially non-toxic products.

### RELATED APPLICATION

This application claims priority from U.S. Provisional Application No. 63/314,852, filed Feb. 28, 2022, the subject matter of which is incorporated herein by reference in its entirety.

### GOVERNMENT FUNDING

This invention was made with government support under AR066193 and AR069569 awarded by The National Institutes of Health. The government has certain rights in the invention.

### BACKGROUND

Continuous changes in tissue and organ architecture, such as rearrangement/reorganization, remodeling, and morphogenesis, occur throughout development. The resulting shapes of tissues and relative positions of different cell types, extracellular matrix molecules and soluble factors ultimately contribute to their functionality. For this reason, biomaterials and tissue engineering strategies that possess characteristics enabling morphological changes that biomimic these natural processes may enhance our ability to form complex tissues that structurally resemble native ones. However, the current state of tissue engineering, which holds great potential for replacement of damaged or lost tissues/organs, generally relies on geometrically static materials that are unable to recapitulate the aforementioned critical dynamic behaviors. Given the significance of the dynamic nature of tissues and organs during their formation to achieve their mature structure, there has recently been great interest in incorporating dynamic shape changing capabilities into materials. To make the materials intrinsically maneuverable and programmable, hydrogel actuators that can change their shape have been engineered for multiple applications in biomedical engineering. In contrast to geometrically static materials, hydrogel actuators respond to external stimuli by changing their shape. The capacity to temporally manipulate the actuator spatial structure and composition positioning may be valuable in guiding development-inspired morphogenetic processes during organoid formation and engineering of tissues.

Although hydrogel actuators exhibit great potential in tissue engineering, drawbacks of the current systems heavily restrict their practical applications. For example, non-biocompatible and/or cytotoxic materials and techniques are often used for hydrogel fabrication and/or harsh conditions, such as low pH, high temperature, and toxic chemicals and solvents, are needed to activate shape responses. Hydrogel actuators designed for tissue engineering applications aimed at replicating aspects of tissue morphogenesis should meet important criteria such as: a) mechanical integrity that allows repeatable shape manipulation and high resistance to complex cell-culture environments and long-time incubation; b) cyto- and bio-compatible stimulation that empowers their spatiotemporal tunability under biological conditions; c) a simple fabrication method that enables reproducible shape change response outcomes. Some cell-laden hydrogel actuators (CHAs) fabricated by using biocompatible materials such as polyethyleneglycol (PEG), collagen, and derivatives of hyaluronic acid and alginate have been designed to undergo deformations without compromising cell viability. However, they typically present limitations such as single-stage shape change (e.g., unidirectional bending/folding) and lack of controllability and reversibility over the shape manipulation. No work to date has been reported cytocompatible CHAs that enable complex multiple and reversible shape transformations in a programmed and/or “on-demand” controllable manner for biomimicry of native tissue morphogenesis.

### SUMMARY

Embodiments described herein relate to shape morphing hydrogel and/or cell condensate actuators or constructs that includes a biocompatible and/or cytocompatible polymer-based shape morphing hydrogel and/or cell condensate, which is configured to undergo multiple, reversible, and/or controllable different shape transformations over time via either pre-programmed design or user-controlled environmental condition alterations. Shape-morphing hydrogels bear promising prospects as soft actuators and for robotics. However, they are mostly restricted to applications in the abiotic domain due to the harsh physicochemical conditions typically necessary to induce shape morphing. We developed a biocompatible polymer-based shape morphing hydrogel and cell condensates using biocompatible polymers and cell condensates that permits encapsulation and maintenance of living cells and implements programmed and controlled actuations with multiple shape changes. The biocompatible polymer-based shape morphing hydrogel and cell condensates enable defined self-folding and/or user-regulated, on-demand-folding into specific 3D architectures under physiological conditions, with the capability to partially bioemulate complex developmental processes, such as branching morphogenesis. The biocompatible polymer-based shape morphing hydrogel and cell condensates can be utilized as platforms for promoting new complex tissue formation and regenerative medicine applications.

In some embodiments, a construct includes a biocompatible and/cytocompatible polymer-based shape morphing hydrogel that is configured to undergo one or more multiple, reversible, controllable and/or different shape transformations over time via either pre-programmed design or user-controlled environmental condition alterations, wherein the hydrogel is cytocompatible and, upon degradation, produces substantially non-toxic products.

In some embodiments, the shape morphing hydrogel includes at least one layer, wherein the swelling and/or degradation rate of the at least one layer actuates the shape transformations.

In some embodiments, the construct includes a first layer that includes a first hydrogel forming biocompatible polymer macromer and a second layer that includes a second hydrogel forming biocompatible polymer macromer different than the first hydrogel forming biocompatible polymer macromer.

In other embodiments, the construct includes multiple layers having similar or different swelling ratios.

In some embodiments, at least one of the layers includes hydrogel forming acrylated and/or methacrylated polymer macromers that are optionally oxidized. The acrylated and/or methacrylated polymer macromers can be reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable.

In some embodiments, the acrylated and/or methacrylated polymer macromers are acrylated and/or methacrylated polysaccharides that are optionally oxidized.

In some embodiments, at least one of the layers includes an acrylated and/or methacrylated alginate that is optionally oxidized and/or at least one of the layers includes an acrylated and/or methacrylated gelatin.

In some embodiments, at least one of the layers includes a first oxidized and acrylated and/or methacrylated natural polymer macromer and another layer includes a second oxidized and acrylated and/or methacrylated natural polymer macromer. The oxidation and/or acrylation and/or methacrylation of the second natural polymer macromer can be different from the oxidation and/or acrylation and/or methacrylation of the second polymer macromer.

In some embodiments, the shape morphing hydrogel can exhibit a repeatable and reversible shape change based on exogenous stimulation. The exogenous stimulation can include, for example, at least one of chemical, biochemical, irradiation, magnetic, biological, electric, ultrasound/sound, mechanical or a change in pH or temperature.

In some embodiments, the shape morphing hydrogel is ionically cross-linkable and the shape transformation is actuated by increasing or decreasing the concentration of ionic cross-linker in the shape morphing hydrogel.

In other embodiments, the shape morphing hydrogel is self-morphing and/or user regulated on-demand morphing into three dimensional architectures under physiological or non-physiological conditions.

In some embodiments, the construct can include a plurality of cells dispersed in the hydrogel. For example, at least a portion of the construct can have a cell density up to 1×10¹⁰ cells/ml. The plurality of cells can include progenitor cells, undifferentiated cells, differentiated cells, and/or cancer cells. In one example, the plurality of cells can include mesenchymal stem cells.

In some embodiments, the construct includes a plurality of layers of hydrogel forming polymer macromers. At least two of the layers can have different macromer concentration, acrylation and/or methacrylation, oxidation, thickness, and/or cell density.

In some embodiments, at least two layers are covalently linked at adjoining portions.

In some embodiments, the construct can include at least three layers, wherein a middle layer is covalently linked to adjoining portions of two outer layers.

In some embodiments, the construct includes the biocompatible, polymer-derived shape morphing hydrogel and a plurality of cells dispersed in at least a portion of the construct, wherein the plurality of cells has a cell density up to 1×10¹⁰ cells/ml.

In some embodiments, the shape morphing hydrogel is self-morphing and/or user regulated on-demand morphing into three dimensional architectures under physiological conditions.

Other embodiments described herein relates to a method of forming a construct. The method includes adhering a first layer that includes a first hydrogel forming natural polymer macromer to a second layer that includes a second hydrogel forming polymer macromer having a different swelling ratio and/or degradation rate than the first hydrogel forming natural polymer macromer. The different swelling ratio and/or degradation rate allows the hydrogel to undergo multiple, reversible, controllable and/or different shape transformations The hydrogel is cytocompatible and, upon degradation, produces substantially non-toxic products.

In some embodiments, at least three layers of hydrogel forming natural polymer macromer are adhered. At least two of layers can have different compositions and a different swelling ratio and/or degradation rate.

In some embodiments, the method can further include adhering a third layer to the first and second layer such that the second layer is sandwiched between the first layer and the third layer. The third layer includes a third hydrogel forming polymer macromer.

In some embodiments, the at least one of the first layer, the second layer, and/or third layer can have different swelling ratios.

In some embodiments, at least one of the layers includes hydrogel forming acrylated and/or methacrylated polymer macromers that are optionally oxidized. The acrylated and/or methacrylated polymer macromers can be reversibly and ionically crosslinkable. The acrylated and/or methacrylated natural polymer macromers can also be photocrosslinkable, ionically crosslinkable, pH crosslinkable, physically crosslinkable, dual crosslinkable, and/or thermally crosslinkable.

In some embodiments, the acrylated and/or methacrylated polymer macromers are acrylated and/or methacrylated polysaccharides that are optionally oxidized.

In some embodiments, at least one of the layers includes an acrylated and/or methacrylated alginate that is optionally oxidized and/or at least one of the layers includes an acrylated and/or methacrylated gelatin.

In other embodiments, at least one layer includes a first oxidized and acrylated and/or methacrylated natural polymer macromer and another layer includes a second oxidized and acrylated and/or methacrylated natural polymer macromer. The oxidation and/or acrylation and/or methacrylation of the second natural polymer macromer can be different from the oxidation and/or acrylation and/or methacrylation of the second polymer macromer.

In some embodiments, the shape morphing hydrogel can exhibit a repeatable and reversible shape change based on exogenous stimulation. The exogenous stimulation can include, for example, at least one of chemical, biochemical, irradiation, magnetic, biological, electric, ultrasound/sound, mechanical or a change in pH or temperature.

In some embodiments, the shape morphing hydrogel is ionically cross-linkable and the shape transformation is actuated by increasing or decreasing the concentration of ionic cross-linker in the shape morphing hydrogel.

In other embodiments, the shape morphing hydrogel is self-morphing and/or user regulated on-demand morphing into three dimensional architectures under physiological or non-physiological conditions.

In some embodiments, the method can include dispersing a plurality of cells in at least a portion of the construct. For example, at least a portion of the construct can have a cell density up to 1×10¹⁰ cells/ml. The plurality of cells can include progenitor cells, undifferentiated cells, differentiated cells, and/or cancer cells. In one example, the plurality of cells can include mesenchymal stem cells.

In some embodiments, the construct includes a plurality of layers of hydrogel forming polymer macromers. At least two of the layers can have different macromer concentration, acrylation and/or methacrylation, oxidation, thickness, and/or cell density.

In some embodiments, at least two layers are covalently linked at adjoining portions.

In some embodiments, the construct can include at least three layers, wherein a middle layer is covalently linked to adjoining portions of two outer layers.

In some embodiments, the construct includes the biocompatible, polymer-derived shape morphing hydrogel and a plurality of cells dispersed in at least a portion of the construct, wherein the plurality of cells has a cell density up to 1×10¹⁰ cells/ml.

In some embodiments, the shape morphing hydrogel is self-morphing and/or user regulated on-demand morphing into three dimensional architectures under physiological conditions.

In some embodiments, the shape morphing hydrogel biomimics tissue developmental processes. For example, the tissue developmental process includes at least one of lung or kidney branching morphogenesis or budding processes.

In some embodiments, the method includes printing the first hydrogel forming polymer macromer into a self-healing, shear thinning, crosslinkable, biocompatible hydrogel support medium. The printed first hydrogel forming polymer macromer can form the first layer having a defined shape. A second hydrogel forming polymer macromer can be printed into the support medium such that the second hydrogel forming polymer macromer forms the second layer with a defined shape. At least a portion of the second layer can adjoin at least a portion of the first layer. The hydrogel support medium can maintain the defined shape of the first layer and the second layer during printing and optionally culturing.

In some embodiments, at least one of the first hydrogel forming natural polymer macromer or the second hydrogel forming natural polymer macromer includes a plurality of cells.

In some embodiments, the method further includes culturing the printed first layer and the printed second layer to form a flow-resistant or free-standing cell condensation structure with a defined shape.

Other embodiments described herein relate to a construct that includes a biocompatible polymer-based shape morphing hydrogel that is configured to undergo multiple, reversible, controllable and/or different shape transformations over time via either pre-programmed design or user-controlled environmental condition alterations. The shape morphing hydrogel includes at least one gradient in polymer concentration, polymer type, polymer swelling, polymer degradation and/or polymer cross-linking density that extends through at least one portion of the shape morphing hydrogel and allows the shape morphing hydrogel to undergo the multiple, reversible, controllable and/or different shape transformations. The hydrogel is cytocompatible and, upon degradation, produces substantially non-toxic product.

In some embodiments, the at least one gradient is provided by layers, regions, or portions of the hydrogel having differing polymer concentration, polymer type, polymer swelling, polymer degradation and/or polymer cross-linking density.

In some embodiments, the hydrogel includes one or more acrylated and/or methacrylated polymer macromers that are optionally oxidized. The acrylated and/or methacrylated polymer macromers are reversibly and ionically crosslinkable.

In some embodiments, the acrylated and/or methacrylated polymer macromers are photocrosslinkable, ionically crosslinkable, physically crosslinkable, pH crosslinkable, dual crosslinkable, and/or thermally crosslinkable.

In some embodiments, the acrylated and/or methacrylated polymer macromers include acrylated and/or methacrylated polysaccharides that are optionally oxidized. For example, the hydrogel includes a mixture of acrylated and/or methacrylated alginate that is optionally oxidized and an acrylated and/or methacrylated gelatin.

In other embodiments, the shape morphing hydrogel exhibits a repeatable and reversible shape change based on exogenous stimulation. For example, the exogenous stimulation can include at least one of chemical, biochemical, irradiation, magnetic, biological, electric, ultrasound/sound, mechanical or a change in pH or temperature.

In some embodiments, the shape morphing hydrogel is self-morphing and/or user regulated on-demand morphing into three dimensional architectures under physiological or non-physiological conditions.

In other embodiments, the construct further includes a plurality of cells dispersed in the hydrogel. At least a portion of the construct can have a cell density up to 1×10¹⁰ cells/ml. The plurality of cells can include progenitor cells, undifferentiated cells, differentiated cells, and/or cancer cells. For example, the plurality cells can include mesenchymal stem cells.

In some embodiments, the shape morphing hydrogel can include a single biocompatible polymer or copolymer.

In some embodiments, the at least one gradient can include a gradient of polymer cross-linking density that extends through at least one portion of the shape morphing hydrogel and allows the shape morphing hydrogel to undergo one or more multiple, reversible, controllable and/or different shape transformations.

In some embodiments, the shape morphing hydrogel includes a photocrosslinkable hydrogel forming polymer and a photo-absorber and a photoinitiator dispersed within the hydrogel.

In other embodiments, the shape morphing hydrogel includes multiple gradients in polymer concentration, polymer type, polymer swelling, polymer degradation and/or polymer cross-linking density that extend through portions of the shape morphing hydrogel and allows the shape morphing hydrogel to undergo multiple, reversible, controllable and/or different shape transformations.

In some embodiments, the construct includes the biocompatible, polymer-derived shape morphing hydrogel and a plurality of cells dispersed in at least a portion of the construct. The plurality of cells can have a cell density up to 1×10¹⁰ cells/ml.

Other embodiments described herein relate to a method of forming a construct as described herein. The method includes providing at least one gradient in polymer concentration, polymer type, polymer swelling, polymer degradation and/or polymer cross-linking density in a biocompatible polymer-based hydrogel. The at least one gradient can extend through at least one portion of hydrogel and allows the hydrogel to undergo multiple, reversible, controllable and/or different shape transformations.

In some embodiments, the at least one gradient is provided by layers, regions, or portions of the hydrogel having differing polymer concentration, polymer type, polymer swelling, polymer degradation and/or polymer cross-linking density.

In some embodiments, the shape morphing hydrogel exhibits a repeatable and reversible shape change based on exogenous stimulation. The exogenous stimulation can include at least one of at least one of chemical, biochemical, irradiation, magnetic, biological, electric, ultrasound/sound, mechanical or a change in pH or temperature.

In some embodiments, the shape morphing hydrogel is self-morphing and/or user regulated on-demand morphing into three dimensional architectures under physiological or non-physiological conditions.

In some embodiments, the shape morphing hydrogel biomimics tissue developmental processes. The tissue developmental process can include at least one of lung or kidney branching morphogenesis or budding processes.

In some embodiments, the hydrogel is cytocompatible and, upon degradation, produces substantially non-toxic products.

In other embodiments, the method further includes providing a plurality of cells in at least one layer of the hydrogel. The cells can be provided in at least a portion of the hydrogel at a cell density of, for example, up to 1×10⁹ cells/ml. The plurality of cells can include progenitor cells, undifferentiated cells, differentiated cells, and/or cancer cells. For example, the plurality cells can include mesenchymal stem cells.

In some embodiments, the hydrogel includes a single biocompatible polymer or copolymer.

In some embodiments, the at least one gradient includes a gradient polymer cross-linking that extends through at least one portion of the shape morphing hydrogel and allows the shape morphing hydrogel to undergo multiple, reversible, controllable and/or different shape transformations.

In other embodiments, the shape morphing hydrogel includes a photocrosslinkable hydrogel forming polymer and a photo-absorber and a photoinitiator dispersed within the hydrogel.

In some embodiments, the method includes forming multiple gradients in polymer concentration, polymer type, polymer swelling, polymer degradation and/or polymer cross-linking that extend through portions of the shape morphing hydrogel and allow the shape morphing hydrogel to undergo multiple and reversible different shape transformations.

Other embodiments relate to a method of forming a construct. The method includes printing a bioink comprising a plurality of cells into a hydrogel support medium. The hydrogel support medium can include at least one gradient in polymer concentration, polymer type, polymer swelling and/or polymer cross-linking, wherein the at least one gradient extends through at least one portion of hydrogel and allows the hydrogel to undergo multiple and reversible different shape transformations.

In some embodiments, the method further includes culturing the printed plurality of cells to form a tissue construct, wherein the support medium maintains the defined shape of the printed bioink during culturing.

Other embodiments described herein relate to a method of forming a construct. The method includes providing a biocompatible polymer-based hydrogel that includes at least one gradient in polymer concentration, polymer type, polymer swelling and/or polymer cross-linking, wherein the at least one gradient extends through at least one portion of hydrogel and allows the hydrogel to undergo multiple and reversible different shape transformations, and seeding and culturing a layer of cells on a surface of the hydrogel.

In some embodiments, the hydrogel is firmly adhered on a surface of a substrate, such as a glass plate, by covalent bonding. The glass plate can include a surface that is modified with at least one molecule that facilitates binding of the hydrogel to the glass plate.

Still other embodiments relate to a construct that includes shape morphing cell condensate that is configured to undergo one or multiple, reversible, controllable and/or different shape transformations over time via either pre-programmed design or user-controlled environmental condition alterations.

In some embodiments, the cell contractile forces or exogenous stimulation allows the construct to undergo controllable different shape transformations over time.

In some embodiments, the cell to cell interactions, cell to extracellular matrix interactions, cell to aptamer interactions, and/or condensation of the cells of the condensate allow the construct to undergo controllable different shape transformations over time.

In some embodiments, the construct further includes a biocompatible polymer-based shape morphing layer that is conjugated to the cell condensate. The biocompatible polymer-based shape morphing layer includes at least one gradient in polymer concentration, polymer type, polymer swelling, polymer degradation and/or polymer cross-linking density that extends through at least one portion of the preformed biocompatible polymer-based shape morphing layer shape.

In some embodiments, the construct includes a preformed biocompatible polymer-based shape morphing layer that includes at least one gradient in polymer concentration, polymer type, polymer swelling, polymer degradation and/or polymer cross-linking density that extends through at least one portion of the preformed biocompatible polymer-based shape morphing layer shape; and a photocurable and degradable cell-supporting microgel (MG) layer that is printed with cells. The MG layer can maintain the shape of the printed cells upon printing. The degradation of the MG layer and/or differential swelling and/or degradation of the preformed biocompatible polymer-based shape morphing layer during culture in tissue-specific formation conditions allows the construct to undergo controllable different shape transformations over time.

In some embodiments, the preformed hydrogel layer includes hydrogel forming acrylated and/or methacrylated polymer macromers that are optionally oxidized. The acrylated and/or methacrylated polymer macromers can reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable.

In some embodiments, the acrylated and/or methacrylated polymer macromers include acrylated and/or methacrylated polysaccharides that are optionally oxidized.

In some embodiments, the photocurable and degradable cell-supporting microgel includes an acrylated and/or methacrylated alginate that is optionally oxidized.

In some embodiments, the preformed layer includes a mixture of an acrylated and/or methacrylated alginate that is optionally oxidized and acrylated and/or methacrylated gelatin.

In some embodiments, the printed photocurable and degradable cell-supporting microgel (MG) layer includes a plurality of printed cells. The printed cells can include progenitor cells, undifferentiated cells, differentiated cells, and/or cancer cells. For example, the plurality cells can include mesenchymal stem cells.

In some embodiments, the shape morphing hydrogel layer includes a single biocompatible polymer or copolymer.

In other embodiments, the preformed biocompatible polymer-based shape morphing layer includes a gradient of polymer cross-linking density through the thickness of the layer that allows the shape morphing hydrogel to undergo one or more multiple, reversible, and/or controllable different shape transformations.

In some embodiments, preformed biocompatible polymer-based shape morphing layer includes a photocrosslinkable hydrogel forming polymer and a photo absorber and a photoinitiator dispersed within the hydrogel.

In some embodiments, the preformed biocompatible polymer-based shape morphing layer includes one or multiple gradients in polymer concentration, polymer type, polymer swelling, polymer degradation and/or polymer cross-linking density that extend through portions of the preformed biocompatible polymer-based shape morphing layer.

In some embodiments, the preformed biocompatible polymer-based shape morphing layer includes a plurality of cells. The cells can include progenitor cells, undifferentiated cells, differentiated cells, and/or cancer cells. For example, the plurality of cells can include mesenchymal stem cells.

Other embodiments described herein relate to a method of forming a layered construct. The method includes providing a preformed biocompatible polymer-based shape morphing layer that includes at least one gradient in polymer concentration, polymer type, polymer swelling, polymer degradation and/or polymer cross-linking density that extends through at least one portion of the preformed biocompatible polymer-based shape morphing layer shape. A printed photocurable and degradable cell-supporting microgel (MG) layer that is configured to allow printing of cells inside MG layer and maintains shape initially upon printing is applied over at least a portion of the preformed biocompatible polymer-based shape morphing layer. Cells are then printed within the MG layer. The degradation of the MG layer and differential swelling and/or degradation of the preformed biocompatible polymer-based shape morphing layer during culture in specific tissue-specific formation conditions allows the construct to undergo controllable different shape transformations over time.

In some embodiments, the preformed hydrogel layer includes a hydrogel forming acrylated and/or methacrylated polymer macromers that are optionally oxidized. The acrylated and/or methacrylated polymer macromers can be reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable. For example, the acrylated and/or methacrylated polymer macromers include acrylated and/or methacrylated polysaccharides that are optionally oxidized.

In some embodiments, the photocurable and degradable cell-supporting microgel can include an acrylated and/or methacrylated alginate that is optionally oxidized.

In other embodiments, the preformed layer can include a mixture of an acrylated and/or methacrylated alginate that is optionally oxidized and acrylated and/or methacrylated gelatin.

In some embodiments, the printed cells can include progenitor cells, undifferentiated cells, differentiated cells, and/or cancer cells. For example, the printed cells can include mesenchymal stem cells.

In some embodiments, the preformed biocompatible polymer-based shape morphing layer includes a single biocompatible polymer or copolymer.

In other embodiments, the preformed biocompatible polymer-based shape morphing layer includes a gradient of polymer cross-linking density through the thickness of the layer that allows the shape morphing hydrogel to undergo one or more multiple, reversible, controllable and/or different shape transformations.

In some embodiments, the preformed biocompatible polymer-based shape morphing layer includes a photocrosslinkable hydrogel forming polymer, a photo-absorber, and a photoinitiator dispersed within the hydrogel.

In other embodiments, the preformed biocompatible polymer-based shape morphing layer includes one or multiple gradients in polymer concentration, polymer type, polymer swelling, polymer degradation and/or polymer cross-linking density that extend through portions of the preformed biocompatible polymer-based shape morphing layer.

In some embodiments, the preformed biocompatible polymer-based shape morphing layer includes a plurality of cells. The cells can include progenitor cells, undifferentiated cells, differentiated cells, and/or cancer cells.

In some embodiments, the method further includes crosslinking the MG layer printed with the cell to enhance the mechanical stability of the MG layer.

In other embodiments, the method includes culturing the layered construct in a culture medium. The culture medium can include a cell differentiation medium.

Other embodiments described herein relate to a composition that includes a plurality of polymer macromer nanoparticle and/or microparticle hydrogels (MGs) and optionally a plurality of cells. The composition is configurable into a stable 3D hydrogel (bio)construct in the absence/presence of cells and is configured to be crosslinkable to form a more robust hydrogel construct.

In some embodiments, the hydrogel construct includes at least one of an anisotropic property in crosslinking density, internal strain, and/or micro/macro-pores distribution.

In some embodiments, the MGs comprise jammed heterogenous natural or synthetic polymer macromer hydrogels. The MGs can include a photoinitiator (PI) and UV absorber.

In some embodiments, the composition is printed into 3D hydrogel (bio)constructs that are programmably reshaped into a defined shape.

In other embodiments, the composition is extrudable or printable into a defined shape.

In some embodiments, the composition is capable of being crosslinked to form a flow-resistant structure with the defined shape and with a gradient in crosslinking density that extends through at least one portion of the hydrogel. The gradient in crosslinking density can allow the 3D hydrogel (bio)construct to undergo one or multiple, reversible, controllable and/or different shape transformations.

In some embodiments, the composition is cytocompatible and, upon degradation, produces a substantially non-toxic product.

In some embodiments, the viscosity of the MGs can decrease with increased shear and/or strain on the MGs and recover after removal of the increased shear and/or strain. The increased shear and/or strain can be associated with extruding or printing the composition, and the viscosity of the composition can recover after extruding or printing the composition to provide the 3D hydrogel (bio)construct with the defined shape.

In some embodiments, the composition can include a plurality of cells. The cells can include progenitor cells, undifferentiated cells and/or differentiated cells. For example, the cells can include mesenchymal stem cells.

In some embodiments, the MGs can have a flake morphology with an average diameter of about 10 μm to about 70 μm.

Other embodiments described herein relate to a method of forming a shape-morphing construct. The method includes providing a plurality of polymer macromer nanoparticle and/or microparticle hydrogels (MGs) and optionally a plurality of cells dispersed with MGs. The MGs and optional cells are then printed into a 3D hydrogel (bio)construct having a defined shape. The 3D hydrogel (bio)construct is crosslinked to further stabilize the 3D hydrogel (bio)construct.

In some embodiments, the 3D hydrogel (bio)construct includes at least one of an anisotropic property in crosslinking density, internal strain, and/or micro/macro-pores distribution.

In some embodiments, the MGs can include a photoinitiator (PI) and UV absorber.

In some embodiments, the gradient in crosslinking density allows the 3D hydrogel (bio)construct to undergo one or multiple, reversible, controllable and/or different shape transformations.

In some embodiments, the shape-morphing construct is cytocompatible and, upon degradation, produces substantially non-toxic product.

In some embodiments, the viscosity of the MGs decreases with increased shear and/or strain on the MGs and recovers after removal of the increased shear and/or strain. The increased shear and/or strain can be associated with printing the MGs and the viscosity of the MGs recovering after printing the MGs to provide the 3D hydrogel (bio)construct with the defined shape.

In some embodiments, the cells can include progenitor cells, undifferentiated cells and/or differentiated cells. For example, the cells can include mesenchymal stem cells.

In some embodiments, the MGs can have a flake morphology with an average diameter of about 10 μm to about 70 μm.

Still other embodiments relate to a composition for forming a shape morphing cell-laden construct. The composition can include a plurality of cells and optionally at least one polymer macromer. The composition can be configurable into a stable 3D bioconstruct having an initial shape, wherein cell contractile forces of the cells of the 3D (bio)construct allows the 3D bioconstruct to undergo one or multiple, reversible, controllable and/or different shape transformations over time.

In some embodiments, the cell contractile forces are associated with at least one of cell to cell interactions, cell to extracellular matrix interactions, cell to aptamer interactions, and/or cell condensation.

In some embodiments, the composition includes a photoinitiator (PI).

In some embodiments, the composition can be printed into 3D hydrogel (bio)constructs that are programmably reshaped into a defined shape.

In other embodiments, the composition is extrudable or printable into a defined shape.

In some embodiments, the composition is capable of being crosslinked to form a flow-resistant structure with the defined shape.

In some embodiments, the shape morphing cell-laden construct can be cytocompatible and, upon degradation, producing substantially non-toxic product.

In some embodiments, the viscosity of the composition decreases with increased shear and/or strain on the composition and recovers after removal of the increased shear and/or strain. The increased shear and/or strain can be associated with extruding or printing the composition and the viscosity of the composition can recover after extruding or printing the composition to provide the 3D hydrogel construct with the defined shape.

In some embodiments, the cells can include progenitor cells, undifferentiated cells and/or differentiated cells. For example, the cells can include mesenchymal stem cells.

Other embodiments described herein relate to a construct that includes at least one degradable cell hydrogel layer or cell condensate layer whose initial shape is maintained by a support, wherein cell contractile forces of the cells of the construct allows the construct to undergo one or multiple, reversible, controllable, and/or different shape transformations over time.

In some embodiments, the cell contractile forces are associated with at least one of cell to cell interactions, cell to extracellular matrix interactions, cell to aptamer interactions, and/or cell condensation.

In some embodiments, the support includes hydrogel and/or microgel in the hydrogel layer.

In some embodiments, the support is external to the cell condensate.

In some embodiments, the at least one degradable cell hydrogel layer or cell condensate layer includes a mixture of oxidized and methacrylated alginate (OMA), methacrylated gelatin, uncrosslinked gelatin microspheres, and plurality of cells as well as optionally a photoinitiator (PI).

In some embodiments, the construct is capable of being crosslinked to form a flow-resistant structure with the defined shape.

In some embodiments, the construct is cytocompatible and, upon degradation, producing substantially non-toxic product.

In some embodiments, the cells can include progenitor cells, undifferentiated cells and/or differentiated cells. For example, the cells can include mesenchymal stem cells.

In some embodiments, the construct can include a hydrogel layer conjugated to the at least one degradable cell hydrogel layer or cell condensate layer. The hydrogel layer can be a non-swelling and/or swelling hydrogel layer.

In some embodiments, condensation of the cells in the cell hydrogel layer or cell condensate layer and optionally degradation of the hydrogel layer during culture allows the construct to undergo one or multiple, reversible, controllable and/or different shape transformations over time.

In some embodiments, the hydrogel layer includes a hydrogel forming acrylated and/or methacrylated polymer macromers that are optionally oxidized. The acrylated and/or methacrylated polymer macromers can be reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable.

In some embodiments, the acrylated and/or methacrylated polymer macromers include at least one of an acrylated and/or methacrylated alginate that is optionally oxidized and/or acrylated and/or methacrylated gelatin.

In some embodiments, the construct includes a first degradable cell laden hydrogel layer, and a second degradable cell laden hydrogel layer overlying the first degradable cell laden hydrogel layer. The first degradable cell laden hydrogel layer and second degradable cell laden hydrogel layer can differ in at least one of amount of cells, cell types, or cell adhesive properties.

Other embodiments relate to a construct that includes a biocompatible polymer-based hydrogel. The hydrogel includes a first portion and a second portion separated by an intermediate portion. The first portion and second portion include a plurality cells encapsulated by hydrogel and the intermediate portion being devoid of cells. The construct is configured to undergo one or multiple, reversible, controllable, and/or different shape transformations over time via cell interactions between cells in the first portion and the second portion and/or cell to extracellular matrix interaction of cells in the first portion and/or second portion.

Still other embodiments relate to a layered construct that includes a photocurable cell-supporting microgel (MG). The MG includes a first cell condensate layer and second cell condensate layer overlying the first cell condensate layer. The second cell condensate layer is different than the first cell condensate layer. The MG is configured to allow printing of cells inside MG layer and maintains shape initially upon printing. The cell condensation in the first layer and/or second layer during culture allows the construct to undergo one or multiple, reversible, controllable, and/or different shape transformations over time.

Other embodiments relate to a layered construct that includes a first degradable cell laden hydrogel layer, and a second degradable cell laden hydrogel layer overlying the first degradable cell laden hydrogel layer. The first degradable cell laden hydrogel layer and second degradable cell laden hydrogel layer differ in at least one of amount of cells, cell types, or cell adhesive properties, and wherein cell to cell interactions, cell to extracellular matrix interactions, cell to aptamer interactions, and/or condensation of the cells of the construct allows the construct to undergo one or multiple, reversible, controllable, and/or different shape transformations over time.

Other embodiments described herein relate to a construct that includes a biocompatible polymer-based hydrogel layer. The hydrogel layer includes a first portion and a second portion separated by an intermediate portion. The first portion and second portion include a plurality cells encapsulated by hydrogel of the layer. The intermediate portion is devoid of cells. The construct is configured to undergo one or multiple, reversible, controllable and/or different shape transformations over time by degradation of the intermediate portion relative to the first portion and second portion and/or cell contractile forces

Still other embodiments relate to a layered construct that includes a first biocompatible and/or cytocompatible hydrogel layer that is non-degradable or slowly degrades, and a second biocompatible and/or cytocompatible aptamer hydrogel layer overlying the first hydrogel layer wherein aptamer interactions result in layer degradation or expansion which allows the construct to undergo one or multiple, reversible, controllable and/or different shape transformations over time.

Other embodiments relate to a bio-kirigami construct that includes a preformed hydrogel frame; and a photocurable and degradable cell-supporting hydrogel member that includes a plurality of cell inside a hydrogel of the member. The member maintains shape initially upon fabrication. Swelling of the hydrogel frame and/or cell support hydrogel member, degradation of the hydrogel member and/or condensation of the cells during culture allows the construct to undergo one or multiple, reversible, controllable, and/or different shape transformations over time.

Other embodiments relate to a construct that includes a biocompatible polymer-based hydrogel. The hydrogel includes portions having at least one of differing stiffness, thickness, and/or degradation rates. The hydrogel includes a plurality cells encapsulated by hydrogel. The construct is configured to undergo one or multiple, reversible, controllable and/or different shape transformations over time based on cell mediated contractile forces within the portions of the hydrogel.

### BRIEF DESCRIPTION OF THE DRAWINGS

FIGS. 1(A-H3) illustrate (A) Schematic of the five-phase transitions of a trilayer hydrogel bar. (B) Sandwiching method to fabricate a trilayer hydrogel. (C) Linkers between OMA and GelMA chains at the layer interface. (D-F) The bending degree of the three trilayers as a function of time. (G) Cell viability in the constructs of the three groups after shape evolution: (1) O10M20A/GelMA/O10M30A, (2) O10M20A/GelMA/O10M45A, and (3) O10M30A/GelMA/O10M45A. (H) Cell-laden four-arm trilayer gripper and the programmable deformations: (H1) schematic illustrating the entire process of shape evolution, (H2) top view photomicrograph of a synthesized four-arm gripper, (H3) photographs of four-arm gripper shape changes over time. Scale bar indicates 0.5 cm. The two OMA layers were co-crosslinked with methacryloxyethyl thiocarbamoyl rhodamine B (0.005%) for visualization. Data are presented as mean±standard deviation (±SD), N=3.

FIGS. 2(A-C3) illustrate cell-laden “smart” trilayer hydrogel fabrication and the programmed deformation. (A) Overlapping parallel-strip patterns on both surfaces of a GelMA hydrogel: (a1) schematic of the sample design, (a2) photographs of top view and section view of a prepared sample, (a3) schematic illustrating the entire process of the construct shape changes over time, (a4) photographs of the construct actual shape changes over time, (a5) the speculated mechanism for the formation of an intermediate phase. (B) Parallel-strip patterns orthogonal to each other on both surfaces of a GelMA hydrogel: (b1) schematic of the sample design, (b2) top view and section view photographs of a prepared sample, (b3) schematic illustrating the entire process of the construct shape changes over time, (b4) photographs of the construct actual shape changes over time. All samples were cultured in GM at 37° C., and GM was replaced with PBS before taking images for clarity. Insets in a4 and b4 on the top left corner show the constructs from the top view, and insets on the top right corner of some images (a4, 5 min; b4, 1 h and 8 h) show the constructs from the side view; (C) Biomimicry of branching morphogenesis: (c1) schematic of branching morphogenesis of lung, step i: formation of a nascent bud, steps ii and iii: cleft formation and terminal bifurcation; (c2) photomicrograph of a typical cell-laden discrete trilayer hydrogel bar designed to undergo branching morphogenesis; (c3) photomicrographs of the 4D hydrogel system mimicking the process of lung branching morphogenesis by the discrete trilayer cultured in GM at 37° C. Images were taken after replacing the GM with PBS for clarity. The two OMA layers were co-crosslinked with methacryloxyethyl thiocarbamoyl rhodamine B (0.005%) for visualization. Scale bars indicate 4 mm.

FIGS. 3(A-E3) illustrate (A) schematic of proposed “on-demand” reversible deformations of a bilayer derived from a trilayer by switching between exposure to Ca²⁺ and EDTA solutions. (B) Cyclic reversible bending of O10M30A/GelMA bilayers due to alternating incubation in EDTA and Ca²⁺ solutions. Inset image shows the reversible bending of the hydrogel bar under alternating stimulations (transparent and red layers are the OMA layer and the GelMA layer, respectively). (C) Shape manipulation of a cell-laden bilayer by alternating Ca²⁺ and EDTA stimulation at 37° C. (D) Photomicrographs of the reversible 3D structure transitions of a quasi-four-petal flower by sequential treatment with Ca²⁺ (50 mM, 10 min) and EDTA (5 mM, 20 min) solutions. (E) (e1) Higher magnification images showing the cells in the GelMA layer and photomicrographs of live/dead stained cells (e2) prior to and (e3) after Ca²⁺ and EDTA treatment. Photos were taken after replacing the GM with PBS for clarity. Data are presented as mean±SD, N=3.

FIGS. 4(A-D) illustrate (A) Programmable shape changes of trilayer CHA (O10M20A/GelMA/O10M45A) with encapsulated cells undergoing chondrogenesis and biochemical quantification of DNA content and GAG/DNA at each time point. *p<0.05 compared to groups D1, D2, and Ctrl2; (B) Programmable shape changes of trilayer CHA with encapsulated cells undergoing osteogenesis and biochemical quantification of DNA content, ALP/DNA and calcium/DNA at each time point. ^{*,#,&}p<0.05 compared to all groups with a different symbol or lacking a symbol. Ctrl1 stands for GelMA only hydrogel bar cultured in chondrogenic media at D21 (in (a)) or osteogenic media at D28 (in (b)), Ctrl2 stands for trilayer hydrogel bar cultured in growth media at D21 (in (a)) and D28 (in (b)); (c) Shape changes of GelMA/O10M45A bilayer derived from the O10M20A/GelMA/O10M45A trilayer during 3-week culture in chondrogenic media: pre-programmed shape morphing (group 1), shape inversion at W1 by Ca²⁺ stimulation and subsequent culture to D21 (group 2), shape recovery at W2 by EDTA and subsequent culture to D21 (group 3); (D) DNA content and GAG/DNA ratios of all conditions at D21. Ctrl1 stands for GelMA only hydrogel bar cultured in chondrogenic media at D21. Ctrl2 stands for bilayer (GelMA/O10M45A) hydrogel bar cultured in growth media at D21. *p<0.05 compared to all other groups. GRP1, GRP2, GRP3 stand for group 1, group 2, and group 3, respectively. Black scale bars in the hydrogel photographs indicate 2 mm. Data are presented as mean±SD, N=3. Statistical tests were performed using one-way ANOVA.

FIGS. 5(A-D) illustrate a schematic illustration for patterning OMAs onto the surface of a GelMA hydrogel: (A) single GelMA surface patterning of OMA; (B) patterning OMA onto a quartz surface; formation of (C) parallel strips and (D) orthogonal strips on dual surfaces of GelMA hydrogel.

FIG. 6 illustrates a pictorial depiction of the definition of bending angle measurement for the programmable shape change of trilayer hydrogel bars. The bending angle is positive when the trilayer bends toward the slower-swelling OMA side (purple layer), and it becomes negative when the bending direction changes after the degradation of the outer faster-swelling OMA layer (red layer).

FIGS. 7(A-C) illustrate representative photomicrographs showing the actual shape changes of the trilayer constructs overtime: (A) O10M20A/GelMA/O10M30A, (B) O10M20A/GelMA/O10M45A, and (C) O10M30A/GelMA/O10M45A. Methacryloxyethyl thiocarbamoyl rhodamine B (RhB, 0.005%) was crosslinked within the OMA layer in red font to aid in visualization. The dotted outlines indicate the shape of the hydrogel strips. Scale bar: 5 mm.

FIGS. 8(A-C) illustrate representative photomicrographs of live/dead stained NIH3T3 cells encapsulated in the hydrogels after the five-phase transitions: (A) O10M20A/GelMA/O10M30A, (B) O10M20A/GelMA/O10M45A, and (C) O10M30A/GelMA/O10M45A.

FIGS. 9(A-B) illustrate (A) Swelling ratios of single layer OMA hydrogels and GelMA hydrogel in DMEM-LG at 4° C. after 10 hours, *p<0.05 compared to all other groups (one-way ANOVA); (B) Photographs of hydrogels after equilibrated swelling in DMEM-LG: (1) O10M45A, (2) O10M30A, (3) O10M20A, and (4) GelMA. Data are presented as mean±SD, N=3.

FIGS. 10(A-B) illustrate photomicrographs of (A) top and (B) side views of a discrete trilayer hydrogel bar.

FIG. 11 is a pictorial depiction of the definition of bending angle measurement for the “on-demand” shape changes of bilayer hydrogel bars. The bending angle is positive when the bilayer bends toward the GelMA side (green layer), and it becomes negative after changing its bending direction toward the OMA side (purple layer).

FIGS. 12(A-B) are (A) Schematic illustration and (B) the actual pornographic images of the reversible shape switching of a 4-arm bilayer gripper upon alternating Ca²⁺/EDTA treatment. The blue (in (A))/transparent (in (B)) and red layers are the OMA and GelMA layers, respectively.

FIG. 13 illustrates the application of the 4-arm gripper for transferring an aluminum ball (0.2 g). Scale bars indicate 2 cm.

FIG. 14 illustrates photomicrographs of live/dead stained NIH3T3 cells in bilayer hydrogel bars after treatment with Ca²⁺ (50 mM) or EDTA (10 mM) for 10 or 30 min, and subsequent 24 h culture in NIH3T3 GM.

FIGS. 15(A-B) illustrate OMA “X” patterned cell-laden GelMA layer folded into a “quasi-four-petal” flower: (A) photomicrographs of a sample and (B) a schematic showing the folding process.

FIGS. 16(A-C) illustrate swelling ratios of (A) O10M20A, (B) O10M30A, and (C) O10M45A hydrogels at different concentrations after 10 hours in DMEM-LG at 4° C., *p<0.05 compared to all other groups (one-way ANOVA). Data are presented as mean±SD, N=3.

FIG. 17 illustrates the impact of OMA concentrations (6%, 8%, or 10% in both OMA layers) on the bending angles of the O10M20A/GelMA/O10M30A trilayer hydrogel bar in DMEM-LG. Data are presented as mean±SD, N=3.

FIGS. 18(A-E) illustrate (A) schematic diagram for fabricating 4D high cell density model construct based on different swelling ratios between OMA and GelMA. Bilayered high cell density (0.2-1.0±10⁸ cells mL⁻¹) OMA/GelMA constructs spontaneously changed geometry into rolled structures. Scale bars indicate 1 cm. Chemical structures and NMR analysis for B) the OMA with different theoretical oxidation rates of 10% and 15% with fixed theoretical 20% methacrylation rate and C) the GelMA. Magnified OMA NMR spectra from 5.4 to 5.6 ppm is presented in a small box on upper right side of the OMA NMR graph. “M” labels indicate methacrylation peaks of the polymers. Profiles of D) swelling and e) degradation of the individual OMA and GelMA hydrogels over 21 days of culture. “*”, “#”, “†”, and “§” indicate statistical significance compared to 12% OMA10, 12% OMA15, 12% GelMA, and 15% GelMA, respectively (p=0.05).

FIGS. 19(A-D) illustrate A) photographic images and B) angle measurements of the bended or rolled model 4D constructs demonstrating the effect of OMA oxidation rate and macromer percentages of GelMA on 4D geometric changes. Diameter of the wells is 15.6 mm. C) Photographic images and D) angle measurements of the bended or rolled model 4D constructs with varied thickness ratios of OMA and GelMA layers at fixed overall construct thickness of 0.6 mm demonstrating the effect of layer thicknesses on 4D geometric changes. Diameter of the wells is 15.6 mm.

FIGS. 20(A-K) illustrate A) photographic images and B) angle measurements of the bended or rolled model 4D constructs containing NIH3T3 cells in the GelMA layer to determine the effect of cell density and OMA layer thickness on the 4D geometric change. Diameter of the wells is 15.6 mm. “*”, “#”, “†”, and “§” indicate statistical significance compared to 2.0×10⁷, 5.0×10⁷, 1.0×10⁸, and 1.0×10⁸ cells mL⁻¹ with 0.4 mm OMA groups, respectively. C) DNA content of the 4D high cell density constructs with 1.0×10⁸ cells mL⁻¹ and 0.4 mm OMA thickness at different time points. D) Live/dead stained images of the high cell density construct at 1^{st}- and 21^{st}-day of culture. Scale bar indicates 200 μm. E) Photographic images of the bent or rolled model 4D constructs containing ASCs in the GelMA layer at 1.0×10⁸ cells mL⁻¹ density which were cultured in growth, chondrogenic, or osteogenic differentiation media over 21 days to demonstrate differentiation capacity in the system and F) angle measurement of the bent or rolled model ASC 4D constructs. Diameter of the wells is 15.6 mm. G) DNA content of the 4D ASC constructs cultured in the different media for 21 days. H) GAG production/DNA of 4D ASC constructs cultured in chondrogenic and growth media for 21 days. I) ALP activity/DNA of 4D ASC constructs cultured in osteogenic and growth media for 21 days. “*” indicates significant statistical difference (p<0.05). Cross-sectional images of J) toluidine blue 0 stained sections of control group cultured in growth media (left) and chondrogenically differentiated 4D ASC construct demonstrating GAG production (right). K) Alizarin Red S stained sections of control group cultured in growth media (left) and osteogenically differentiated 4D ASC construct demonstrating calcium deposition (right). Scale bars in (J) and (K) indicate 1 mm.

FIGS. 21(A-G) illustrate A) photographic images over 14 days of 4D high cell density constructs composed of 0.2 mm cell-laden GelMA and 0.4 mm cell-laden OMA layers. NIH3T3 cells were incorporated at 1.0×10⁸ cells mL⁻¹ in both layers. The diameter of the wells in the images is 15.6 mm. B) Images exhibiting the presence of NIH3T3 cells in both the GelMA and OMA layers. Left image is a phase contrast image of the construct and right image is a fluorescence image of cells labeled with purple and green dyes in the 12% OMA15 and 12% GelMA layers, respectfully. Scale bars indicate 500 μm. C) Angle measurements of the 4D high density NIH3T3 (1.0×10⁸ cells mL⁻¹) constructs during 14 days of culture and image of the construct at 14th day (scale bar=1 mm). D) Photographic image of a photomask used to generate a pattern of alternating regions of photocrosslinked OMA in the model construct. Scale bar indicates 500 μm. E) Photographic images of the bent constructs over 7 days corresponding to OMA pattern directions perpendicular or parallel to the constructs' longest axis. Diameter of the wells is 15.6 mm. F) CAD image of layered checkerboard pattern. Red and light gray indicate cell-laden OMA and GelMA regions, respectively. G) 4D high cell density layered checkerboard construct fabricated by printing NIH3T3-laden 12% OMA15 (1.0×10⁸ cells mL⁻¹) and NIH3T3-laden 12% GelMA (1.0×10⁷ cells mL⁻¹) inks using a CAD file. The printed construct was flat and did not exhibit curvature or angled shape at day 0, while defined 4D geometric changes occurred over the course of 7 days culture as indicated by white dotted lines. Scale bars in (F) and (G) indicate 1 cm.

FIGS. 22(A-K) illustrate (A) A typical setup for crosslinking gradient hydrogel fabrication; (B) Schematic showing gradient hydrogel cut into specific initial shape and its subsequent deformation after swelling; Curved hydrogel bars of (C) PEGDA, (D) GelMA, and (E) OMA obtained using RhB (0.03% w/v) as UV absorber; (F) Magnified image showing a clear continuous gradient in the OMA hydrogel; Curved hydrogel bars of OMA obtained using (G) FITC (0.03% w/v), (H) AAb (0.05% w/v), and (I) HMAP (0.01% w/v) as UV absorber; bilayer hydrogel bars obtained from (J) OMA/GelMA and (K) OMA(g)/GelMA demonstrating feasibility of multi-material fabrication, where OMA represents non-gradient OMA hydrogel and OMA(g) represents gradient OMA hydrogel. The dotted outlines indicate (G-J) the shape of the hydrogel bars or (J-K) the interface of two layers. Scale bars in C-E and G-K indicate 2 mm.

FIGS. 23(A-E) illustrate bending degree of OMA hydrogels as a function of (A) UV irradiation time (6% w/v polymer, 0.6 mm thickness, 0.03% w/v RhB) in H₂O, *p<0.05 compared with all other groups, (B) RhB concentration (6% w/v polymer, 0.6 mm thickness, 30 s UV) in H₂O, *p<0.05 compared with all other groups, (C) hydrogel thickness (6% w/v polymer, 0.03% w/v RhB, 30 s UV) in H₂O, *p<0.05 compared with all other groups except for “0.4”, #p<0.05 compared with all other groups, (D) polymer concentration (0.6 mm thickness, 0.03% w/v RhB, 30 s UV) in H₂O, *p<0.05 compared with all other groups, and (E) aqueous swelling solution (6% w/v polymer, 0.6 mm thickness, 0.03% w/v RhB, and 30 s UV), *p<0.05 compared with all other groups. All scale bars indicate 2 mm.

FIGS. 24(A-D) illustrate cyclic reversible bending angle of OMA hydrogel bars in water solutions (A) of alternating pH of 1 and 7 and (B) with alternating presence of chemicals EDTA and Ca2+, 0.03% w/v RhB, *p<0.05 compared with all other groups, #p<0.05 compared with “pH 1” and original groups, scale bars indicate 4 mm. (C) Bending degree of cell-free and cell-laden OMA hydrogel bars in GM, 0.02% w/v HMAP, *p<0.05 compared with all other groups, scale bars indicate 2 mm. (D) Viability of encapsulated cells inside non-gradient (without UV absorber) and gradient (with UV absorber) hydrogels in GM after 3 days culture, 0.02% w/v HMAP, *p<0.05 compared to hMSC without UV absorber group. Insets: representative images showing the deformed shapes under respective conditions.

FIGS. 25(A-E) illustrate images of 4D biofabricated cell-laden structures: (A₁) six-petal blossoms and (A₂) four-arm grippers obtained by photomask-aided biofabrication, insets illustrate the deformation process of the microfabricated hydrogels; (B₁) Schematic of a “double-faced” hydrogel bar and its deformation, and (B₂) a typical “S” shape formed by the “double-faced” hydrogel; (C) hydrogel tubes obtained by post-photopatterning of a pre-fabricated gradient hydrogel sheet or disk, (C₁) patterned regions on a hydrogel sheet with dark pink regions denoting the UV-exposed section, (C₂) top-view image obtained using an optical microscope, (C₃) side-view image taken in ambient light; ITP-generated (D₁) hydrogel spiral and (D₂) pseudo-four-petal flower, insets show the ion-printed section on a pre-formed gradient hydrogel bar or hydrogel sheet; and (E) bioprinted multiple-arm grippers and their deformed structures, insets show the as-printed hydrogel construct before photo-gelation (upper left) and the top view of deformed hydrogel constructs (lower left). All hydrogel deformations were performed in GM at 37° C. for at least 2 h to obtain ultimate stable structures, and images were taken after replacing the GM with PBS (pH 7.4) for clarity.

FIGS. 26(A-F) illustrate (A) Bending degree of cell-laden hydrogel bars as a function of culture time in osteogenic medium. Inset: representative images at respective time points showing the shapes of the cell-laden hydrogel bars. (B) Live/dead staining images of encapsulated cells inside a hydrogel bar after 4-week culture in osteogenic medium. Biochemical quantification of (C) DNA content, (D) ALP activity normalized to DNA content, and (E) calcium content normalized to DNA content in the cell-laden hydrogels at varying time points. NC (negative control): cell-laden hydrogel bar obtained in the presence of UV absorber and cultured in GM, EG (experimental group): cell-laden hydrogel bar obtained in the presence of UV absorber and cultured in osteogenic medium, PC (positive control): cell-laden hydrogel bar obtained in the absence of UV absorber and cultured in osteogenic medium. (F) Typical alizarin red stained cell-laden hydrogel bars after 4-week culture. *p<0.05 compared with all other time points within a group, #p<0.05 compared with NC group at the same time point.

FIG. 27 is a schematic illustration of 4D cell-condensate bioprinting, tissue maturation, and tissue release.

FIGS. 28(A-F) illustrate (A) Compressive elastic moduli of various hydrogels, and the change of (B) modulus and (C) viscosity of O5M20A MGs with increasing shear strain and shear rate, respectively. (D) The rapid and reversible phase transition of O5M20A MGs between elastic state and viscous state by alternating the shear strain between 1% and 100%, and the (E) degradation and (F) swelling profiles of various hydrogels. ^{*,#,&}p<0.05 compared to group sharing no or a different symbol.

FIGS. 29(A-F) illustrate (A) Illustration and real sample photographs of a bilayer hydrogel disc (B) before and (C) after shape morphing. Effects of varying UV time applied to (D) the upper layer and (E) the bottom layer and (F) of varying MG layer thickness on the bending behaviors of the hydrogel strips. The hydrogel strip thickness for the UV time variation study was set to 0.8/0.5 (mm/mm) and the UV time for the MG layer thickness variation study was set to 30 s/30 s.

FIG. 30(A-C) illustrate (A) Live/dead cell staining of a printed cell filament inside photocrosslinked MGs after culturing in cell growth media for 4 h. Scale bars indicate 0.5 mm. (B) Photomicrographs of a printed cell strip (18×4×1.2 mm³) from top view (left) and side view (middle) and a cell sheet (13×13×1.2 mm³, right) inside photocrosslinked MG constructs. The cell strip images were taken immediately after UV photocrosslinking, the cell sheet image was taken immediately after placing in cell growth media. Scale bars indicate 5 mm. (C) Representative photomicrographs of cell-laden (left) and cell-free (middle) bilayer strips after culturing in cell growth media for 4 h, and quantified bending angles of the cell-laden and cell-free bilayer strips (right). *p<0.05 and scale bars indicate 5 mm. The UV irradiation times for the gradient layer and the MG layer fabrications were set to 30 s and 20 s (30 s/20 s), respectively.

FIGS. 31(A-D) illustrate large cell-laden bilayer constructs with defined structures of cell filling and the corresponding deformed configurations after culturing in cell growth media for 4 h: (A) sheet, (B) disc, (C) bar grid, and (D) net. Deformed cell-free bilayer counterparts were obtained under the same conditions and included for comparison. HeLa cells were used in this study, and UV irradiation times for the actuation layer (bottom layer) and the cell-laden layer (upper layer) were set to 40 s and 20 s (40 s/20 s), respectively. Scale bars indicate 5 mm.

FIGS. 32(A-D) illustrate (A) Photomicrographs of cell-net infilled bilayer sheet at D3, D6, D9, and D12. The black arrow and blue arrow in the image at D6 show the separated gradient hydrogel layer and the cell condensate-laden layer, respectively. Arrows in images of D9 and D12 show “liberated” cell-net filaments. Scale bars indicate 10 mm. Photomicrographs showing a cell-net filament inside the hydrogel under bright-field channel, green (live) channel, red (dead) channel, and merged channel of green and red at (B) D3, (C) D7, and (D) D12. Scale bars indicate 0.5 mm. UV crosslinking time was set to bottom/top 40 s/20 s.

FIGS. 33(A-K) illustrate (A) Shapes of hMSC cell condensate-laden bilayer strips cultured in chondrogenic media at different times. Exp stands for the experimental group, Ctrl stands for the control group, and scale bar is 10 mm. The UV irradiation times for the gradient layer and the MG layer were set to 50 s and 20 s (50 s/20 s), respectively. (B) 4D engineered letter “C”-shaped cartilage-like tissue after 21 days of culture. Scale bar is 10 mm. (C) The change of bending angle as a function of the culturing time, *p<0.05 compared to control group. (D) Biochemical analysis of GAG production (normalized to DNA content) at D21. (E) Young's moduli of the ex vivo engineered cartilage-like tissues at D21. Histologic characterization of ex vivo 4D engineered cartilage-like tissues: (F) hematoxylin and eosin (H&E) staining, and GAG (G) Safranin O (SafO, pink/red) and (I) toluidine blue O (TBO, blue and purple) staining for GAG. Scale bars indicate 0.2 mm. Helix-shaped cartilage-like tissue obtained at day 21 (I) before and (J) after manual removal of outer hydrogel layer. Scale bars indicate 5 mm. The UV irradiation times for the gradient layer and the MG layer were set to 30 s and 20 s (30 s/20 s), respectively. (K) TBO stained helix-shaped cartilage-like tissue, scale bar is 5 mm.

FIG. 34 is a schematic illustration of the PI and UV absorber incorporated 4D MFH bioprinting: i) printing the jammed cell-laden MFH bioinks into a bioconstruct, ii) UV crosslinking to generate a crosslinking gradient within the 3D printed bioconstruct, iii) culturing in media to drive shape morphing.

FIGS. 35(A-K) illustrate (A) Photomicrograph of safranin O stained MFHs. (B) Schematics showing lower packing density of granular microgels (upper) and more highly packed irregular MFHs (bottom). (C) Storage (G′) and loss (G″) moduli of MFHs as a function of frequency. Material viscosity decreases while continuously increasing (D) shear rate and (E) shear strain over 10% strain. (F) Crossover of G′ and G″ with increasing shear strain indicative of shear yielding. Rapid recovery of MFHs' (G) modulus and (H) viscosity by alternating the applied strain between 1% and 100%. (I) Photomicrograph of a filament printed through a 22-gauge (22G) needle (inner diameter 413 μm). Photographs of 3D printed (J) hydrogel bar (25×4×1 mm³) and (K) hydrogel cuboid (10×8×6 mm³). (1) Fidelity of the as-printed 3D construct in j before UV crosslinking.

FIGS. 36(A-H) illustrate 4D shape-morphing behaviors of hydrogel bars in different incubation solutions or fabricated with different parameters. (A) Hydrogel bending angle kinetics in diH2O, PBS (pH 7.4), and GM at room temperature. (B) Photomicrographs of deformed hydrogel bars in diH2O, PBS (pH 7.4), and GM after swelling for 2 h. Effects of (C) infill density, (D) printing speed, (F) needle gauge, (F) UV irradiation time, (G) hydrogel bar width, and (H) hydrogel bar length on the bending angles of hydrogel bars cultured in PBS (pH 7.4) for 2 h at room temperature. UV absorber: 0.02% 4′-hydroxy-3′-methylacetophenone (HMAP), 0.005% methacryloxyethyl thiocarbamoyl rhodamine B (RhB) was incorporated to impart the hydrogel with red color for better clarity.

FIG. 37 illustrates snapshots showing the shape changes of a hydrogel bar in response to different pH treatments at room temperature.

FIGS. 38(A-I) illustrate (A) Bending behaviors of MFH gradient hydrogel bars with and without embedded cells. Insets show representative photomicrographs of the bent hydrogel bars. (B) Photomicrograph of a NIH3T3-laden MFH-based construct. (C) Representative live/dead image of NIH3T3 fibroblasts in the gradient hydrogel bar. (D) Photomicrograph of a cell-laden “biohelix” structure. (E) Photograph of a cell-laden “bioS” structure. Photographs of (F) a cell-laden “pseudo-four-petal” and (G) a cell-laden “pseudo-six-petal” flower. Inset photographs show the corresponding as-printed structures. Kirigami-based structures and the deformed configurations: hydrogels in bar-grid patterns (H) without and (I) with inner horizontal bars. For cell-laden gradient hydrogel bars (insets in a), only 0.02% HMAP was used as the UV absorber, they were cultured in GM overnight at 37° C., and then imaged and live/dead stained. For complex cell-laden hydrogel configuration formation, a mixture of 0.02% HMAP and 0.005% RhB UV absorbers was used. These bioconstructs were obtained after culturing in GM at 37° C. overnight, and images were immediately taken after replacing the GM with FBS for clarity.

FIG. 39(A-F) illustrate demonstration of 3D-to-3D shape morphing. A pillar gripper (A) before being subject to deformation, after being submerged in media (B) less than 10 s and (C) for 60 s. A shark-fin sheet: (D) as-printed shape, (E) front-view image of deformed shape, and (F) image of deformed shape with construct on its side. Scale bars indicate 5 mm.

FIGS. 40(A-F) illustrate MFH based 4D bioprinting for application in tissue engineering. (A) The bending angles of hydrogel bars in EG and the corresponding photomicrographs depicting the shape changes of 4D bioprinted cell-laden hydrogel bars in CM over time. (B) Photomicrographs depicting cell morphology and distribution and live/dead stained cells within the EG hydrogel bars in CM on D1 and D21. (C) Biochemical quantification of GAG production normalized to DNA, *p<0.05 compared to NC at the same time point, #p<0.05 compared to D7 within a group. TBO stained (D) hydrogel bars and (E) four- and (F) six-petal flower-shaped hydrogels after chondrogenesis for 21 days in CM. NC: negative control, 4D bioprinted cell-laden hydrogel bars cultured in GM; EG: experimental group, 4D bioprinted cell-laden hydrogel bars cultured in CM; PC: positive control, 3D bioprinted (without incorporation of UV absorber) cell-laden hydrogel bars cultured in CM.

FIG. 41 illustrates 1H NMR spectrum of O1M30A (D₂O, 2%).

FIGS. 42(A-D) illustrate photographs of (A) as-prepared MFHs in 70% EtOH (B) and (C, D) reconstituted jammed MFHs (bioink).

FIG. 43 illustrates photomicrographs of safranin O stained MFHs.

FIG. 44 illustrates photograph of a MFH bioink filament extruded through a 22G needle.

FIG. 45 illustrate Young's moduli of as-prepared MFH, UV-crosslinked non-gradient MFH, and crosslinked gradient MFH, *p<0.05 compared to other groups.

FIG. 46 illustrates determination of the bending angle (θ).

FIG. 47 illustrates Swelling ratio of UV crosslinked MFHs in different media and pH, *p<0.05 compared to other groups.

FIGS. 48(A-B) is an illustration of Illustration of the strain distribution on the deformed hydrogel bar. The strain varies along the radical direction (er) but keeps relatively constant along the tangential direction (eθ).

FIG. 49 illustrates reversible bending behavior of hydrogel bars by switching the pH of the PBS incubation solution between 2.0 and 7.4 at room temperature.

FIG. 50 illustrates the viability of hMSCs cultured with 0.02% HMAP (w/v) for different culture times and subsequent culture in fresh GM to reach a total 24 h culture as measured using MTT assay.

FIG. 51 illustrates photomicrographs of printed cell-free MFH hydrogels (i) before and (ii) after UV crosslinking and UV-crosslinked cell-laden MFH hydrogels and live/dead stained images: (iii, v) hMSC and (iv, vi) and HeLa cells.

FIG. 52 is a schematic showing the formation of a discretely patterned gradient hydrogel.

FIG. 53 is a schematic shows the formation of a dual-orientated gradient hydrogel.

FIGS. 54(A-D) illustrate top views of the (A) four- and (B) six-petal flowers, (C) the “rib cage” like structure, and (D) the “net tube”.

FIGS. 55(A-B) illustrate schematics illustrating the gradient creation in two 3D models and their deformations: (A) “pillar bar”, (B) “shark-fin sheet”.

FIGS. 56(A-E) illustrate a demonstration of more complex 3D-to-3D shape morphing: (A) A “double sharkfin” sheet and its (B) morphed structure, (C) a “double pillar gripper” and (D, E) its morphed structure (upside down) with bend pillars pointing horizontally to the left and right, (D) side view and (E) top view. Scale bars indicate 5 mm.

FIG. 57 illustrates the change in DNA levels over 21 days of culture. NC: negative control, 4D bioprinted cell-laden hydrogel bar cultured in GM; EG: experimental group, 4D bioprinted cell-laden hydrogel bar cultured in CM; PC: positive control, 3D bioprinted cell-laden hydrogel bar (without incorporation of UV absorber) cultured in CM.

FIG. 58 illustrates a Change in GAG levels over 21 days of culture. NC: negative control, 4D bioprinted cell-laden hydrogel bar cultured in GM; EG: experimental group, 4D bioprinted cell-laden hydrogel bar cultured in CM; PC: positive control, 3D bioprinted cell-laden hydrogel bar (without incorporation of UV absorber) cultured in CM. *p<0.05 compared to NC at the same time point, #p<0.05 compared to D7 group within a group.

FIGS. 59A-F illustrate rheology of composite OMA/GelMA bioink for 3D printing. (A) Schematic of the overall strategy for CTF-mediated 4D biomaterials. The composite bioink exhibits shear thinning properties, where viscosity decreases as shear rate increases (B). Similarly, the storage modulus is greater than the loss modulus at low shear strains and less than the loss modulus at high shear strains (C). The composite bioink also exhibits self-healing properties, with the storage and loss moduli maintaining their initial values after several oscillations of strain (D). Adding gelatin microspheres at concentrations of 25, 50, and 100 mg/ml increases the bulk modulus (E). After one day of culture, the gelatin microspheres have liquified, causing the G′ to decrease dramatically (F).

FIGS. 60(A-E) illustrate shrinkage of 3D-printed cell-laden constructs. A schematic outlines the 3D printing process (A). OMA microgels are combined with 3% w/v GelMA and varying concentrations of gelatin microspheres. 1 mL of this solution is added to 100 million cells to form the cell-laden bioink. Here, the bioink was used to print 10 mm×10 mm×0.6 mm squares, which were monitored for 14 days to evaluate shrinkage. A selection of images shows the progression from initial to final shape (B). Quantifying the area reveals that increasing gelatin microsphere concentration leads to an increase in shrinkage (C). Scale bar=10 mm. “*”, “†”, “#”, and “$” indicate statistical significance at day 14 compared to 0 mg/ml, 25 mg/ml, 50 mg/ml, and 100 mg/ml, respectively (p<0.05).

FIGS. 61(A-D) illustrate 4D shape morphing in bilayer constructs. (A) Schematic illustration of the printing process. A thin hydrogel layer is printed followed by printing of the cell-laden layer. (B) To demonstrate the role of cellular forces in the 4D process, printed constructs were cultured in media containing 5 μM Cytochalasin D. Constructs were also cultured in media containing 0.1% DMSO as a vehicle control. Constructs in normal growth media and media with DMSO conditions followed similar 4D bending (C), demonstrating that Cytochalasin D is responsible for the lack of bending seen in this condition. The average bending angles in the GM and DMSO conditions are not significantly different from each other at all timepoints (p<0.05). (D) Histological analysis and staining with H&E at day 14 corroborate these observations, with cells in growth medium and DMSO conditions forming fibrous borders while cells in Cyto D are unable to do so. White scale bar: 5 mm. Black scale bar: 200 μm. Red scale bar: 50 μm.

FIGS. 62(A-D) illustrate chondrogenesis in 4D bilayer constructs. hMSCs were printed in bilayer constructs and cultured in normal growth media (GM) or chondrogenic pellet medium (CPM) and cultured for 21 days. (A) Macroscopic images of constructs over 21 days. (B) Quantification of bending angle over time (n=4). (C) Quantification of GAG production relative to DNA content for both GM and CPM conditions. Constructs cultured in CPM produced significantly more GAG (p<0.05). These results are qualitatively confirmed by histological staining at day 21 (D). Samples were stained with H&E to visualize cell condensation. Safranin O and Fast Green were used to visualize GAG production. Since the alginate in these constructs also stains positively with Safranin O, constructs were also stained with Alcian Blue at a pH of 0.2, revealing much greater positive staining in CPM samples. White scale bar=5 mm. Black scale bar=200 μm. “*” indicates statistical significance (p<0.05).

FIGS. 63(A-C) illustrates complex patterning of the cell-laden and hydrogel layers. (A) Complex patterning of the cell-laden layer. Schematics of the printed constructs followed by photomicrographs at days 0, 3, and 14. (B) Complex patterning of the hydrogel layer. Rectangular hydrogel patterns were printed either vertically or horizontally onto a cell-laden rectangle. The direction of the hydrogel pattern influenced the bending direction of the constructs. (C) Complex patterning of both the cell-laden and hydrogel layers. Layers were printed sequentially according to the schematic, resulting in bending around two separate, non-parallel axes.

FIG. 64 illustrates frequency sweeps of all composite bioinks with varying gelatin microsphere concentration. All bioink compositions show dominating solid-like properties at low frequencies. After one day of culture, the moduli of the 50 mg/ml condition have decreased but still maintain the same trend.

FIG. 65 illustrates the viability of printed constructs with varying concentrations of gelatin microspheres at days 0, 7, and 14.

FIGS. 66(A-B) illustrate the viability of GM, DMSO, and Cyto D conditions at day 14. (A) Composite live/dead images reveal that cell morphology appears similar in GM and DMSO conditions, while cells appear more rounded in the Cyto D condition. (B) Live/dead image of a construct in the GM group, centered at an inflection point. Differences in cell morphology can be observed by imaging through the cell-free layer (indicated with *) versus imaging through the cell-laden layer (indicated with #). Scale bar=200 μm.

FIG. 67(A-D) illustrates the description of bending angle measurement. Here, the Day 5 GM photomicrograph from FIG. 66B is used as an example. First, the image is uploaded into PowerPoint (A). Then, a circle with crosshairs is fitted to the contours of the printed construct (B). This overlay is then copied into ImageJ and the angle tool is used to find the angle between the ends of the construct. The middle of the crosshairs is used as the vertex (C). The bending angle is then measured and recorded (D).

FIG. 68 illustrates histological evidence of gelatin microsphere liquefaction. At day 0, gelatin microspheres appear pink under H&E staining. After one day of culture, the gelatin has liquefied and diffused out of the constructs, leaving macroscale pores (black arrows). Scale bar=200 μm.

FIG. 69 illustrates cell-cell interaction generated force mediated 4D system.

FIG. 70 illustrates cell generated force mediated 4D system.

FIG. 71 illustrates cell-matrix interaction force mediated 4D system.

FIG. 72 illustrates cell-generated force mediated 4D systems based on photolithography-produced hydrogel thickness differences/changes.

FIG. 73 illustrates cell-generated force mediated 4D systems based on degradable joints.

FIG. 74 illustrates 4D aptamer hydrogels.

FIG. 75 illustrates 4D cell condensate formation based on a specifically designed bilayer system.

FIG. 76 illustrates fully coat with a thin non-deformable OMA layer and a gradient OMA layer.

FIG. 77 illustrates an alternative strategy.

FIG. 78 illustrates 4D bio-kirigami system.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

The term “bioactive agent” can refer to any agent capable of promoting tissue formation, destruction, and/or targeting a specific disease state. Examples of bioactive agents can include, but are not limited to, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

The term “biomaterial” refers to any naturally occurring, naturally derived, or synthetic material or substance which is compatible with biological systems.

The terms “biodegradable” and “bioresorbable” may be used interchangeably and refer to the ability of a material (e.g., a natural polymer or macromer) to be fully resorbed in vivo. “Full” can mean that no significant extracellular fragments remain. The resorption process can involve elimination of the original implant material(s) through the action of body fluids, enzymes, cells, and the like.

The term “gel” includes gels and hydrogels.

The term “microgel” refers to hydrogels having a diameter less than about 1000 μm, less than about 400 μm, or less than about 300 μm, for example, about 1 μm to about 1000 μm.

The term “function and/or characteristic of a cell” can refer to the modulation, growth, and/or proliferation of at least one cell, such as a progenitor cell and/or differentiated cell, the modulation of the state of differentiation of at least one cell, and/or the induction of a pathway in at least one cell, which directs the cell to grow, proliferate, and/or differentiate along a desired pathway, e.g., leading to a desired cell phenotype, cell migration, angiogenesis, apoptosis, etc.

The term “macromer” can refer to any natural polymer or oligomer or their derivatives.

The term “polynucleotide” can refer to oligonucleotides, nucleotides, or to a fragment of any of these, to DNA or RNA (e.g., mRNA, rRNA, siRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, ribonucleoproteins (e.g., iRNPs). The term can also encompass nucleic acids (i.e., oligonucleotides) containing known analogues of natural nucleotides, as well as nucleic acid-like structures with synthetic backbones.

The term “polypeptide” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term “polypeptide” can also include peptides and polypeptide fragments, motifs and the like, glycosylated polypeptides, and all “mimetic” and “peptidomimetic” polypeptide forms.

The term “cell” can refer to any progenitor cell, such as totipotent stem cells, pluripotent stem cells, and multipotent stem cells, as well as any of their lineage descendant cells, including more differentiated cells. The terms “stem cell” and “progenitor cell” are used interchangeably herein. The cells can derive from embryonic, fetal, or adult tissues. Examples of progenitor cells can include totipotent stem cells, multipotent stem cells, mesenchymal stem cells (MSCs), hematopoietic stem cells, neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells can include de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

The terms “inhibit,” “silencing”, and “attenuating” can refer to a measurable reduction in expression of a target mRNA (or the corresponding polypeptide or protein) as compared with the expression of the target mRNA (or the corresponding polypeptide or protein) in the absence of an interfering RNA molecule of the present invention. The reduction in expression of the target mRNA (or the corresponding polypeptide or protein) is commonly referred to as “knock-down” and is reported relative to levels present following administration or expression of a non-targeting control RNA.

The term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, aves, etc.), which is to be the recipient of a particular treatment. Typically, the terms “patient” and “subject” are used interchangeably herein in reference to a human subject.

The term “tissue” can refer to an aggregate of cells having substantially the same function and/or form in a multicellular organism. “Tissue” is typically an aggregate of cells of the same origin but may be an aggregate of cells of different origins. The cells can have the substantially same or substantially different function and may be of the same or different type. “Tissue” can include, but is not limited to, an organ, a part of an organ, bone, cartilage, skin, neuron, axon, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic, or ascite tissue.

### DETAILED DESCRIPTION

Embodiments described herein relate to shape morphing hydrogel and/or cell condensate actuators or constructs that include a biocompatible and/or cytocompatible polymer-based shape morphing hydrogel and/or cell condensate, which is configured to undergo multiple, reversible, and/or controllable different shape transformations over time via either pre-programmed design or user-controlled environmental condition alterations. Shape-morphing hydrogels bear promising prospects as soft actuators and for robotics. However, they are mostly restricted to applications in the abiotic domain due to the harsh physicochemical conditions typically necessary to induce shape morphing. We developed a biocompatible polymer-based shape morphing hydrogel and cell condensates using biocompatible polymers and cell condensates that permits encapsulation and maintenance of living cells and implements programmed and controlled actuations with multiple shape changes. The biocompatible polymer-based shape morphing hydrogel and cell condensates enable defined self-folding and/or user-regulated, on-demand-folding into specific 3D architectures under physiological conditions, with the capability to partially bioemulate complex developmental processes, such as branching morphogenesis. The biocompatible polymer-based shape morphing hydrogel and cell condensates can be utilized as platforms for promoting new complex tissue formation and regenerative medicine applications.

In some embodiments, a construct includes a biocompatible and/cytocompatible polymer-based shape morphing hydrogel that is configured to undergo one or more multiple, reversible, controllable and/or different shape transformations over time via either pre-programmed design or user-controlled environmental condition alterations, wherein the hydrogel is cytocompatible and, upon degradation, produces substantially non-toxic products.

In some embodiments, the shape morphing hydrogel includes at least one layer, wherein the swelling and/or degradation rate of the at least one layer actuates the shape transformations.

In some embodiments, the construct includes a first layer that includes a first hydrogel forming biocompatible polymer macromer and a second layer that includes a second hydrogel forming biocompatible polymer macromer different than the first hydrogel forming biocompatible polymer macromer.

In other embodiments, the construct includes multiple layers having similar or different swelling ratios.

In some embodiments, at least one of the layers includes hydrogel forming acrylated and/or methacrylated polymer macromers that are optionally oxidized. Acrylated and/or methacrylated natural polymer macromers can include saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose that can be readily oxidized to form free aldehyde units.

In some embodiments, the acrylated and/or methacrylated polymer macromers are acrylated and/or methacrylated polysaccharides that are optionally oxidized.

In some embodiments, at least one of the layers includes an acrylated and/or methacrylated alginate that is optionally oxidized and/or at least one of the layers includes an acrylated and/or methacrylated gelatin.

The acrylated or methacrylated, alginates can be optionally oxidized so that up to about 50% of the saccharide units therein are converted to aldehyde saccharide units. Natural source alginates, for example, from seaweed or bacteria, are useful and can be selected to provide side chains with appropriate M (mannuronate) and G (guluronate) units for the ultimate use of the polymer. Alginate materials can be selected with high guluronate content since the guluronate units, as opposed to the mannuronate units, more readily provide sites for oxidation and crosslinking. Isolation of alginate chains from natural sources can be conducted by conventional methods. See Biomaterials: Novel Materials from Biological Sources, ed. Byrum, Alginates chapter (ed. Sutherland), p. 309-331 (1991). Alternatively, synthetically prepared alginates having a selected M and G unit proportion and distribution prepared by synthetic routes, such as those analogous to methods known in the art, can be used. Further, either natural or synthetic source alginates may be modified to provide M and G units with a modified structure. The M and/or G units may also be modified, for example, with polyalkylene oxide units of varied molecular weight such as shown for modification of polysaccharides in Spaltro (U.S. Pat. No. 5,490,978) with other alcohols such as glycols. Such modification generally will make the polymer more soluble, which generally will result in a less viscous material. Such modifying groups can also enhance the stability of the polymer. Further, modification to provide alkali resistance, for example, as shown by U.S. Pat. No. 2,536,893, can be conducted.

The oxidation of the natural polymer macromers (e.g., alginate material) can be performed using a periodate oxidation agent, such as sodium periodate, to provide at least some of the saccharide units of the natural polymer macromer with aldehyde groups. The degree of oxidation is controllable by the mole equivalent of oxidation agent, e.g., periodate, to saccharide unit. For example, using sodium periodate in an equivalent % of from 2% to 100%, preferably 1% to 50%, a resulting degree of oxidation, i.e., % if saccharide units converted to aldehyde saccharide units, from about 2% to 50% can be obtained. The aldehyde groups provide functional sites for crosslinking and for bonding tissue, cells, prosthetics, grafts, and other material that is desired to be adhered. Further, oxidation of the natural polymer macromer facilitates their degradation in vivo, even if they are not lowered in molecular weight. Thus, high molecular weight alginates, e.g., of up to 300,000 daltons, may be degradable in vivo, when sufficiently oxidized, i.e., preferably at least 5% of the saccharide units are oxidized.

In some embodiments, the natural polymer macromer (e.g., alginate) can be acrylated or methacrylated by reacting an acryl group or methacryl with a natural polymer or oligomer to form the oxidized, acrylated or methacrylated natural polymer macromer (e.g., alginate). For example, oxidized alginate can be dissolved in a solution chemically functionalized with N-hydroxysuccinimide and 1-ethyl-3-(3-dimethylaminopropyl)-carbodiimide hydrochloride to activate the carboxylic acids of alginate and then reacted with 2-aminoethylmethacrylate to provide a plurality of methacrylate groups on the alginate.

The acrylated and/or methacrylated gelatin can be formed by reacting an acryl group and/or methacryl with gelatin. For example, bovine type-B gelatin can be dissolved in a phosphate buffered solution and then reacted with methacrylic anhydride to provide a plurality of methacrylate groups on the gelatin.

The degree of acrylation or methacrylation can be controlled to control the degree of subsequent crosslinking of the acrylate and methacrylates as well as the mechanical properties, and biodegradation rate of the composition. The degree of acrylation or methacrylation can be about 1% to about 50%, although this ratio can vary more or less depending on the end use of the composition.

The acrylated and/or methacrylated polymer macromers can be reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable to adjust the mechanical properties of the hydrogel. The mechanical properties that can be adjusted include, for example, stiffness, Young's modulus, tensile strength, viscosity, resistance to shear or tensile loading and excessive swelling, as well as biodegradation rate.

In some embodiments, at least one of the layers includes a first oxidized and acrylated and/or methacrylated natural polymer macromer and another layer includes a second oxidized and acrylated and/or methacrylated natural polymer macromer, the oxidation and/or acrylation and/or methacrylation of the second natural polymer macromer different from the oxidation and/or acrylation and/or methacrylation of the second polymer macromer.

In some embodiments, the shape morphing hydrogel can exhibit a repeatable and reversible shape change based on exogenous stimulation. The exogenous stimulation can include, for example, at least one of chemical, biochemical, irradiation, magnetic, biological, electric, ultrasound/sound, mechanical or a change in pH or temperature.

In some embodiments, the shape morphing hydrogel is ionically cross-linkable and the shape transformation is actuated by increasing or decreasing the concentration of ionic cross-linker in the shape morphing hydrogel. For example, gluronic acids of different alginates in alginate hydrogel can form ionic crosslinks with Ca²⁺ provide by an aqueous solution of CaCl₂ resulting in a crosslinked hydrogel network. As the concentration of the ionic crosslinker (e.g., Ca²⁺) in the hydrogel increases the secondary crosslinking and elastic moduli increase resulting in an increase in stiffness of the hydrogel. Subsequent decrease in the concentration of the ionic crosslinker by, for example, incubation of the hydrogel in a buffer solution decreases the ionic crosslinking and the elastic moduli of the hydrogel resulting in softening of the hydrogel.

In other embodiments, the shape morphing hydrogel is self-morphing and/or user regulated on-demand morphing into three dimensional architectures under physiological or non-physiological conditions.

In some embodiments, the construct can include a plurality of cells dispersed in the hydrogel. For example, at least a portion of the construct has a cell density up to 1×10¹⁰ cells/ml. The plurality of cells can include progenitor cells, undifferentiated cells, differentiated cells, and/or cancer cells. In one example, the plurality of cells can include mesenchymal stem cells.

The cells provided in the hydrogel can be autologous, xenogeneic, allogeneic, and/or syngeneic. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into or onto the hydrogel. For example, autologous cells can be expanded in this manner if a sufficient number of viable cells cannot be harvested from the host subject. Alternatively or additionally, the cells may be pieces of tissue, including tissue that has some internal structure. The cells may be primary tissue explants and preparations thereof, cell lines (including transformed cells), or host cells.

Generally, cells can be introduced into the hydrogels in vitro, although in vivo seeding approaches can optionally or additionally be employed. Cells may be mixed with the macromers used to form the hydrogels and cultured in an adequate growth (or storage) medium to ensure cell viability. If the hydrogel is to be implanted for use in vivo after in vitro seeding, for example, sufficient growth medium may be supplied to ensure cell viability during in vitro culture prior to in vivo application. Once the hydrogels have been implanted, the nutritional requirements of the cells can be met by the circulating fluids of the host subject.

Any available method may be employed to introduce the cells into the hydrogels. For example, cells may be injected into the hydrogels (e.g., in combination with growth medium) or may be introduced by other means, such as pressure, vacuum, osmosis, or manual mixing. Alternatively or additionally, cells may be layered on the hydrogels, or the hydrogels may be dipped into a cell suspension and allowed to remain there under conditions and for a time sufficient for the cells to incorporate within or attach to the hydrogel. Generally, it is desirable to avoid excessive manual manipulation of the cells in order to minimize cell death during the impregnation procedure. For example, in some situations it may not be desirable to manually mix or knead the cells with the hydrogels; however, such an approach may be useful in those cases in which a sufficient number of cells will survive the procedure. Cells can also be introduced into the hydrogels in vivo simply by placing the hydrogel in the subject adjacent a source of desired cells.

As those of ordinary skill in the art will appreciate, the number of cells to be introduced into the hydrogels will vary based on the intended application of the hydrogel and on the type of cell used. Where dividing autologous cells are being introduced by injection or mixing into the hydrogel, for example, a lower number of cells can be used. Alternatively, where non-dividing cells are being introduced by injection or mixing into the hydrogel, a larger number of cells may be required. It should also be appreciated that the hydrogel can be in either a hydrated or lyophilized state prior to the addition of cells. For example, the hydrogel can be in a lyophilized state before the addition of cells is done to re-hydrate and populate the scaffold with cells.

In other embodiments, the hydrogels can include at least one attachment molecule to facilitate attachment of at least one cell thereto. The attachment molecule can include a polypeptide or small molecule, for example, and may be chemically immobilized onto the hydrogel to facilitate cell attachment. Examples of attachment molecules can include fibronectin or a portion thereof, collagen or a portion thereof, polypeptides or proteins containing a peptide attachment sequence (e.g., arginine-glycine-aspartate sequence) (or other attachment sequence), enzymatically degradable peptide linkages, cell adhesion ligands, growth factors, degradable amino acid sequences, and/or protein-sequestering peptide sequences.

In other embodiments, the construct can include at least one bioactive agent. Advantageously, the release of the bioactive agent from the hydrogel can be controlled by dynamically adjusting the mechanical properties of the hydrogel. The at least one bioactive agent can include any agent capable of modulating a function and/or characteristic of a cell that is dispersed on or within the hydrogel. Alternatively or additionally, the bioactive agent may be capable of modulating a function and/or characteristic of an endogenous cell surrounding the hydrogel implanted in a tissue defect, for example, and guide the cell into the defect.

Examples of bioactive agents include chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., EGF), HGF, VEGF, fibroblast growth factors (e.g., bFGF), PDGF, insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), sonic hedgehog, growth differentiation factors (e.g., GDF5, GDF6, GDF8), recombinant human growth factors (e.g., MP-52 and the MP-52 variant rhGDF-5), cartilage-derived morphogenic proteins (CDMP-1, CDMP-2, CDMP-3), small molecules that affect the upregulation of specific growth factors, polynucleotides, DNA fragments, DNA plasmids, MMPs, TIMPs, interfering RNA molecules, such as siRNAs, DNA encoding for an shRNA of interest, oligonucleotides, proteoglycans, glycoproteins, and glycosaminoglycans.

In some embodiments, the construct includes a plurality of layers of hydrogel forming polymer macromers. At least two of the layers can have different macromer concentration, acrylation and/or methacrylation, oxidation, thickness, and/or cell density.

In some embodiments, at least two layers are covalently linked at adjoining portions.

In some embodiments, the construct can include at least three layers, wherein a middle layer is covalently linked to adjoining portions of two outer layers.

In some embodiments, the construct includes the biocompatible, polymer-derived shape morphing hydrogel and a plurality of cells dispersed in at least a portion of the construct, wherein the plurality of cells has a cell density up to 1×10¹⁰ cells/ml.

In some embodiments, the shape morphing hydrogel is self-morphing and/or user regulated on-demand morphing into three dimensional architectures under physiological conditions.

Other embodiments described herein relates to a method of forming a construct. The method includes adhering a first layer that includes a first hydrogel forming natural polymer macromer to a second layer that includes a second hydrogel forming polymer macromer having a different swelling ratio and/or degradation rate than the first hydrogel forming natural polymer macromer. The different swelling ratio and/or degradation rate allows the hydrogel to undergo multiple, reversible, controllable and/or different shape transformations. The hydrogel is cytocompatible and, upon degradation, produces substantially non-toxic products.

In some embodiments, at least three layers of hydrogel forming natural polymer macromer are adhered. At least two of layers can have different compositions and a different swelling ratio and/or degradation rate.

In some embodiments, the method can further include adhering a third layer to the first and second layer such that the second layer is sandwiched between the first layer and the third layer. The third layer can include a third hydrogel forming polymer macromer.

In some embodiments, the at least one of the first layer, the second layer, and/or third layer can have different swelling ratios.

In some embodiments, at least one of the layers includes a hydrogel forming acrylated and/or methacrylated polymer macromers that are optionally oxidized. The acrylated and/or methacrylated polymer macromers can be reversibly and ionically crosslinkable. The acrylated and/or methacrylated natural polymer macromers can also be photocrosslinkable, ionically crosslinkable, pH crosslinkable, physically crosslinkable, dual crosslinkable, and/or thermally crosslinkable.

In some embodiments, the acrylated and/or methacrylated polymer macromers are acrylated and/or methacrylated polysaccharides that are optionally oxidized.

In some embodiments, at least one of the layers includes an acrylated and/or methacrylated alginate that is optionally oxidized and/or at least one of the layers includes an acrylated and/or methacrylated gelatin.

In other embodiments, at least one layer includes a first oxidized and acrylated and/or methacrylated natural polymer macromer and another layer includes a second oxidized and acrylated and/or methacrylated natural polymer macromer. The oxidation and/or acrylation and/or methacrylation of the second natural polymer macromer can be different from the oxidation and/or acrylation and/or methacrylation of the second polymer macromer.

In some embodiments, the shape morphing hydrogel can exhibit a repeatable and reversible shape change based on exogenous stimulation. The exogenous stimulation can include, for example, at least one of chemical, biochemical, irradiation, magnetic, biological, electric, ultrasound/sound, mechanical or a change in pH or temperature.

In some embodiments, the shape morphing hydrogel is ionically cross-linkable and the shape transformation is actuated by increasing or decreasing the concentration of ionic cross-linker in the shape morphing hydrogel.

In other embodiments, the shape morphing hydrogel is self-morphing and/or user regulated on-demand morphing into three dimensional architectures under physiological or non-physiological conditions.

In some embodiments, the method can include dispersing a plurality of cells in at least a portion of the construct. For example, at least a portion of the construct can have a cell density up to 1×10¹⁰ cells/ml. The plurality of cells can include progenitor cells, undifferentiated cells, differentiated cells, and/or cancer cells. In one example, the plurality of cells can include mesenchymal stem cells.

In some embodiments, the construct includes a plurality of layers of hydrogel forming polymer macromers. At least two of the layers can have different macromer concentration, acrylation and/or methacrylation, oxidation, thickness, and/or cell density.

In some embodiments, at least two layers are covalently linked at adjoining portions.

In some embodiments, the construct can include at least three layers, wherein a middle layer is covalently linked to adjoining portions of two outer layers.

In some embodiments, the construct includes the biocompatible, polymer-derived shape morphing hydrogel and a plurality of cells dispersed in at least a portion of the construct, wherein the plurality of cells has a cell density up to 1×10¹⁰ cells/ml.

In some embodiments, the shape morphing hydrogel is self-morphing and/or user regulated on-demand morphing into three dimensional architectures under physiological conditions.

In some embodiments, the shape morphing hydrogel biomimics tissue developmental processes. For example, the tissue developmental process includes at least one of lung or kidney branching morphogenesis or budding processes.

In some embodiments, the method includes printing the first hydrogel forming polymer macromer into a self-healing, shear thinning, crosslinkable, biocompatible hydrogel support medium. The printed first hydrogel forming polymer macromer can form the first layer having a defined shape. A second hydrogel forming polymer macromer can be printed into the support medium such that the second hydrogel forming polymer macromer forms the second layer with a defined shape. At least a portion of the second layer can adjoin at least a portion of the first layer. The hydrogel support medium can maintain the defined shape of the first layer and the second layer during printing and optionally culturing.

The self-healing, shear thinning, crosslinkable, biocompatible hydrogel support medium can maintain the first hydrogel forming polymer macromer and the second hydrogel forming macromer in a defined shape during printing of the first hydrogel forming polymer macromer and the second hydrogel forming macromer. The hydrogel support medium can be resistant to flow at a first shear stress and behave as a viscous fluid at a second higher shear stress. This allows the hydrogel support medium to behave as a viscous fluid during printing and be resistant to flow before and after printing. For example, initially, the hydrogel support medium is in a flow-resistant or solid-like state before being printed with the first hydrogel forming polymer macromer and the second hydrogel forming macromer. The hydrogel support medium becomes fluidized under the increased shear stress caused by printing the first hydrogel forming polymer macromer and the second hydrogel forming macromer into the hydrogel support medium. Then, after the printing is finished and the increased shear stress is removed, the hydrogel support medium can self-heal and form a flow-resistant or solid-like stable support medium. The hydrogel support medium can be crosslinked after printing to maintain the defined shape of the printed the first hydrogel forming polymer macromer and the second hydrogel forming macromer during culturing in the hydrogel support medium.

In some embodiments, the self-healing, shear thinning, crosslinkable, biocompatible hydrogel support medium can include a plurality of crosslinkable hydrogel particles that are provided in a container. The plurality of crosslinkable hydrogel particles are in contact with each other in the container such that interstitial spaces are provided between individual hydrogel particles. The interstitial spaces between individual particles form pores in the hydrogel support medium in which a culture medium can be provided and/or flow to the printed bioink during culturing of the cells. The sizes of the pores can be dependent on the sizes of the individual hydrogel particles. For example, smaller pores can result from smaller spaces between the smaller hydrogel particles, and, conversely, larger pores can result from larger spaces between the larger hydrogel particles.

In some embodiments, the hydrogel particles can have average diameter of about 10 nm to about 10 mm, for example, about 100 nm to about 1000, about 1 μm to about 500 μm, about 25 μm to about 400 μm, or about 50 μm to 200 μm. The plurality of hydrogel particles can have substantially homogenous diameters or include particles of varying diameters to provide a heterogenous mixture of the hydrogel particles.

The hydrogel particles can be cytocompatible and, upon degradation, produce substantially non-toxic products. In some embodiments, the hydrogel particles can include a plurality of crosslinkable biodegradable natural polymer macromers. The crosslinkable natural polymer macromers can be any crosslinkable natural polymer or oligomer that includes a functional group (e.g., a carboxylic group) that can be further polymerized. Examples of natural polymers or oligomers are saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose.

The natural polymer macromers can optionally be at least partially crosslinked with a first agent and further crosslinkable with the first agent or crosslinkable with a second agent. The crosslinkable natural polymer macromer can include dual crosslinkable natural polymer macromers, such as an acrylated and/or methacrylated natural polymer macromers. Acrylated and/or methacrylated natural polymer macromers can include saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose that can be readily oxidized to form free aldehyde units.

In some embodiments, the acrylated or methacrylated, natural polymer macromers are polysaccharides, which are optionally oxidized so that up to about 50% of the saccharide units therein are converted to aldehyde saccharide units. Control over the degree of oxidation of the natural polymer macromers permits regulation of the gelling time used to form the hydrogel as well as the mechanical properties, which allows for tailoring of these mechanical properties depending on the clinical application.

In other embodiments, acrylated and/or methacrylated, natural polymer macromers can include oxidized, acrylated or methacrylated, alginates, which are optionally oxidized so that, for example, up to about 50% of the saccharide units therein are converted to aldehyde saccharide units. Natural source alginates, for example, from seaweed or bacteria, are useful and can be selected to provide side chains with appropriate M (mannuronate) and G (guluronate) units for the ultimate use of the polymer. Alginate materials can be selected with high guluronate content since the guluronate units, as opposed to the mannuronate units, more readily provide sites for oxidation and crosslinking. Isolation of alginate chains from natural sources can be conducted by conventional methods. See Biomaterials: Novel Materials from Biological Sources, ed. Byrum, Alginates chapter (ed. Sutherland), p. 309-331 (1991). Alternatively, synthetically prepared alginates having a selected M and G unit proportion and distribution prepared by synthetic routes, such as those analogous to methods known in the art, can be used. Further, either natural or synthetic source alginates may be modified to provide M and G units with a modified structure. The M and/or G units may also be modified, for example, with polyalkylene oxide units of varied molecular weight such as shown for modification of polysaccharides in Spaltro (U.S. Pat. No. 5,490,978) with other alcohols such as glycols. Such modification generally will make the polymer more soluble, which generally will result in a less viscous material. Such modifying groups can also enhance the stability of the polymer. Further, modification to provide alkali resistance, for example, as shown by U.S. Pat. No. 2,536,893, can be conducted.

The oxidation of the natural polymer macromers (e.g., alginate material) can be performed using a periodate oxidation agent, such as sodium periodate, to provide at least some of the saccharide units of the natural polymer macromer with aldehyde groups. The degree of oxidation is controllable by the mole equivalent of oxidation agent, e.g., periodate, to saccharide unit. For example, using sodium periodate in an equivalent % of from 2% to 100%, preferably 1% to 50%, a resulting degree of oxidation, i.e., % if saccharide units converted to aldehyde saccharide units, from about 2% to 50% can be obtained. The aldehyde groups provide functional sites for crosslinking and for bonding tissue, cells, prosthetics, grafts, and other material that is desired to be adhered. Further, oxidation of the natural polymer macromer facilitates their degradation in vivo, even if they are not lowered in molecular weight. Thus, high molecular weight alginates, e.g., of up to 300,000 daltons, may be degradable in vivo, when sufficiently oxidized, i.e., preferably at least 5% of the saccharide units are oxidized.

In some embodiments, the natural polymer macromer (e.g., alginate) can be acrylated or methacrylated by reacting an acryl group or methacryl with a natural polymer or oligomer to form the oxidized, acrylated or methacrylated natural polymer macromer (e.g., alginate). For example, oxidized alginate can be dissolved in a solution chemically functionalized with N-hydroxysuccinimide and 1-ethyl-3-(3-dimethylaminopropyl)-carbodiimide hydrochloride to activate the carboxylic acids of alginate and then reacted with 2-amionethylmethacrylate to provide a plurality of methacrylate groups on the alginate.

The degree of acrylation or methacrylation can be controlled to control the degree of subsequent crosslinking of the acrylate and methacrylates as well as the mechanical properties, and biodegradation rate of the composition. The degree of acrylation or methacrylation can be about 1% to about 50%, although this ratio can vary more or less depending on the end use of the composition.

In some embodiments, a solution of natural polymer macromers can be ionically crosslinked and/or chemically crosslinked with a first agent to form a plurality of hydrogel particles. The ionically crosslinked hydrogel can be in the form of a plurality of hydrogel particles. By way of example, a solution of natural polymer macromers can be dispensed as microdroplets into an aqueous solution of CaCl₂) and ionically crosslinked to form the plurality of microgels. The extent of crosslinking can be controlled by the concentration of CaCl₂). The higher concentration can correspond to a higher extent of crosslinking. The extent of crosslinking alters the mechanical properties of the microgel and can be controlled as desired for the particular application. In general, a higher degree of crosslinking results in a stiffer gel.

In some embodiments, the hydrogel particles can be crosslinked with a second agent to form dual crosslinked hydrogel. A plurality of second crosslink networks can be formed by crosslinking acrylate and/or methacrylate groups of the acrylated or methacrylated natural polymer macromer. The second crosslinking networks formed by crosslinking the acrylate groups or methacrylate groups of the acrylated and/or methacrylated natural polymer macromer can provide improved mechanical properties, such as resistance to excessive swelling, as well as delayed biodegradation rate.

In some embodiments, the acrylate or methacrylate groups of the acrylated and/or methacrylated natural polymer macromer of the hydrogel can be crosslinked by photocrosslinking using UV light in the presence of photoinitiators. For example, acrylated and/or methacrylated natural polymer macromers of the hydrogel particles can be photocrosslinked with a photoinitiator that is provided in the hydrogel support medium. The hydrogel particles can be exposed to a light source at a wavelength and for a time to promote crosslinking of the acrylate groups of the polymers and form the photocrosslinked biodegradable hydrogel particles.

A photoinitiator can include any photo-initiator that can initiate or induce polymerization of the acrylate or methacrylate macromer. Examples of the photoinitiator can include camphorquinone, benzoin methyl ether, 2-hydroxy-2-methyl-1-phenyl-1-propanone, diphenyl(2,4,6-trimethylbenzoyl)phosphine oxide, benzoin ethyl ether, benzophenone, 9,10-anthraquinone, ethyl-4-N,N-dimethylaminobenzoate, diphenyliodonium chloride and derivatives thereof.

In other embodiments, the hydrogel support medium can further include at least one bioactive agent that is provided in the hydrogel particles or potentially a culture medium that can be added to the hydrogel support medium during culturing of the printed bioink. The bioactive agent can include polynucleotides and/or polypeptides encoding or comprising, for example, transcription factors, differentiation factors, growth factors, and combinations thereof. The at least one bioactive agent can also include any agent capable of promoting tissue formation (e.g., bone and/or cartilage), destruction, and/or targeting a specific disease state (e.g., cancer). Examples of bioactive agents include chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., EGF), HGF, VEGF, fibroblast growth factors (e.g., bFGF), PDGF, insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), sonic hedgehog, growth differentiation factors (e.g., GDF5, GDF6, GDF8), recombinant human growth factors (e.g., MP-52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparin sulfate, polynucleotides, DNA fragments, DNA plasmids, MMPs, TIMPs, interfering RNA molecules, such as siRNAs, miRNAs, DNA encoding for an shRNA of interest, oligonucleotides, proteoglycans, glycoproteins, and glycosaminoglycans.

In some embodiments, a bioactive agent can comprise an interfering RNA or miRNA molecule incorporated on or within insoluble native collagen fibers or dispersed on or within the cell aggregate. The interfering RNA or miRNA molecule can include any RNA molecule that is capable of silencing an mRNA and thereby reducing or inhibiting expression of a polypeptide encoded by the target mRNA. Alternatively, the interfering RNA molecule can include a DNA molecule encoding for a shRNA of interest. For example, the interfering RNA molecule can comprise a short interfering RNA (siRNA) or microRNA molecule capable of silencing a target mRNA that encodes any one or combination of the polypeptides or proteins described above.

In some embodiments, at least one of the first hydrogel forming natural polymer macromer or the second hydrogel forming natural polymer macromer includes a plurality of cells. The cells provided in the first hydrogel forming natural polymer macromer or the second hydrogel forming natural polymer macromer can be autologous, xenogeneic, allogeneic, and/or syngeneic. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into or onto the hydrogel. For example, autologous cells can be expanded in this manner if a sufficient number of viable cells cannot be harvested from the host subject. Alternatively or additionally, the cells may be pieces of tissue, including tissue that has some internal structure. The cells may be primary tissue explants and preparations thereof, cell lines (including transformed cells), or host cells.

In some embodiments, the method further includes culturing the printed first layer and the printed second layer to form a flow-resistant or free-standing cell condensation structure with a defined shape.

Other embodiments described herein relate to a construct that includes a biocompatible polymer-based shape morphing hydrogel that is configured to undergo multiple, reversible, controllable and/or different shape transformations over time via either pre-programmed design or user-controlled environmental condition alterations. The shape morphing hydrogel includes at least one gradient in polymer concentration, polymer type, polymer swelling, polymer degradation and/or polymer cross-linking density that extends through at least one portion of the shape morphing hydrogel and allows the shape morphing hydrogel to undergo the multiple, reversible, controllable and/or different shape transformations. The hydrogel is cytocompatible and, upon degradation, produces substantially non-toxic product.

In some embodiments, the at least one gradient is provided by layers, regions, or portions of the hydrogel having differing polymer concentration, polymer type, polymer swelling, polymer degradation and/or polymer cross-linking density.

In some embodiments, the hydrogel includes one or more acrylated and/or methacrylated polymer macromers that are optionally oxidized. The acrylated and/or methacrylated polymer macromers are reversibly and ionically crosslinkable.

In some embodiments, the acrylated and/or methacrylated polymer macromers are photocrosslinkable, ionically crosslinkable, physically crosslinkable, pH crosslinkable, dual crosslinkable, and/or thermally crosslinkable. For example, the acrylated and/or methacrylated polymer macromers include acrylated and/or methacrylated polysaccharides that are optionally oxidized.

In some embodiments, the hydrogel includes a mixture of acrylated and/or methacrylated alginate that is optionally oxidized and an acrylated and/or methacrylated gelatin.

In other embodiments, the shape morphing hydrogel exhibits a repeatable and reversible shape change based on exogenous stimulation. For example, the exogenous stimulation can include at least one of chemical, biochemical, irradiation, magnetic, biological, electric, ultrasound/sound, mechanical or a change in pH or temperature.

In some embodiments, the shape morphing hydrogel is self-morphing and/or user regulated on-demand morphing into three dimensional architectures under physiological or non-physiological conditions.

In other embodiments, the construct further includes a plurality of cells dispersed in the hydrogel. At least a portion of the construct can have a cell density up to 1×10¹⁰ cells/ml. The plurality of cells can include progenitor cells, undifferentiated cells, differentiated cells, and/or cancer cells. For example, the plurality cells can include mesenchymal stem cells.

In some embodiments, the shape morphing hydrogel can include a single biocompatible polymer or copolymer.

In some embodiments, the at least one gradient can include a gradient of polymer cross-linking density that extends through at least one portion of the shape morphing hydrogel and allows the shape morphing hydrogel to undergo one or more multiple, reversible, controllable and/or different shape transformations.

In some embodiments, the shape morphing hydrogel includes a photocrosslinkable hydrogel forming polymer and a photo-absorber and a photoinitiator dispersed within the hydrogel.

In other embodiments, the shape morphing hydrogel includes multiple gradients in polymer concentration, polymer type, polymer swelling, polymer degradation and/or polymer cross-linking density that extend through portions of the shape morphing hydrogel and allows the shape morphing hydrogel to undergo multiple, reversible, controllable and/or different shape transformations.

In some embodiments, the construct includes the biocompatible, polymer-derived shape morphing hydrogel and a plurality of cells dispersed in at least a portion of the construct. The plurality of cells can have a cell density up to 1×10¹⁰ cells/ml.

Other embodiments described herein relate to a method of forming a construct as described herein. The method includes providing at least one gradient in polymer concentration, polymer type, polymer swelling, polymer degradation and/or polymer cross-linking density in a biocompatible polymer-based hydrogel. The at least one gradient can extend through at least one portion of hydrogel and allows the hydrogel to undergo multiple, reversible, controllable and/or different shape transformations.

In some embodiments, the at least one gradient is provided by layers, regions, or portions of the hydrogel having differing polymer concentration, polymer type, polymer swelling, polymer degradation and/or polymer cross-linking density.

In some embodiments, the shape morphing hydrogel exhibits a repeatable and reversible shape change based on exogenous stimulation, the exogenous stimulation can include at least one of at least one of chemical, biochemical, irradiation, magnetic, biological, electric, ultrasound/sound, mechanical or a change in pH or temperature.

In some embodiments, the shape morphing hydrogel is self-morphing and/or user regulated on-demand morphing into three dimensional architectures under physiological or non-physiological conditions.

In some embodiments, the shape morphing hydrogel biomimics tissue developmental processes. The tissue developmental process can include at least one of lung or kidney branching morphogenesis or budding processes.

In some embodiments, the hydrogel is cytocompatible and, upon degradation, produces substantially non-toxic products.

In other embodiments, the method further includes providing a plurality of cells in at least one layer of the hydrogel. The cells can be provided in at least a portion of the hydrogel at a cell density of, for example, up to 1×10⁹ cells/ml. The plurality of cells can include progenitor cells, undifferentiated cells, differentiated cells, and/or cancer cells. For example, the plurality cells can include mesenchymal stem cells.

In some embodiments, the hydrogel includes a single biocompatible polymer or copolymer.

In some embodiments, the at least one gradient includes a gradient polymer cross-linking that extends through at least one portion of the shape morphing hydrogel and allows the shape morphing hydrogel to undergo multiple, reversible, controllable and/or different shape transformations.

In other embodiments, the shape morphing hydrogel includes a photocrosslinkable hydrogel forming polymer and a photo-absorber and a photoinitiator dispersed within the hydrogel.

In some embodiments, the method includes forming multiple gradients in polymer concentration, polymer type, polymer swelling, polymer degradation and/or polymer cross-linking that extend through portions of the shape morphing hydrogel and allows the shape morphing hydrogel to undergo multiple and reversible different shape transformations.

Other embodiments relate to a method of forming a construct. The method includes printing a bioink comprising a plurality of cells into a hydrogel support medium. The hydrogel support medium can include at least one gradient in polymer concentration, polymer type, polymer swelling and/or polymer cross-linking, wherein the at least one gradient extends through at least one portion of hydrogel and allows the hydrogel to undergo multiple and reversible different shape transformations.

In some embodiments, the method further includes culturing the printed plurality of cells to form a tissue construct, wherein the support medium maintains the defined shape of the printed bioink during culturing.

Other embodiments described herein relate to a method of forming a construct. The method includes providing a biocompatible polymer-based hydrogel that includes at least one gradient in polymer concentration, polymer type, polymer swelling and/or polymer cross-linking, wherein the at least one gradient extends through at least one portion of hydrogel and allows the hydrogel to undergo multiple and reversible different shape transformations and seeding and culturing a layer of cells on a surface of the hydrogel.

In some embodiments, the hydrogel is firmly adhered on a surface of a glass plate by covalent bonding. The glass plate can include a surface that is modified with at least one molecule that facilitates binding hydrogel to the glass plate.

Still other embodiments relate to a construct that includes shape morphing cell condensate that is configured to undergo one or multiple, reversible, controllable and/or different shape transformations over time via either pre-programmed design or user-controlled environmental condition alterations.

In some embodiments, the cell contractile forces or exogenous stimulation allows the construct to undergo controllable different shape transformations over time.

In some embodiments, the cell to cell interactions, cell to extracellular matrix interactions, cell to aptamer interactions, and/or condensation of the cells of the condensate allow the construct to undergo controllable different shape transformations over time.

In some embodiments, the construct further includes a biocompatible polymer-based shape morphing layer that is conjugated to the cell condensate. The biocompatible polymer-based shape morphing layer including at least one gradient in polymer concentration, polymer type, polymer swelling, polymer degradation and/or polymer cross-linking density that extends through at least one portion of the preformed biocompatible polymer-based shape morphing layer shape.

In some embodiments, the construct includes a preformed biocompatible polymer-based shape morphing layer that includes at least one gradient in polymer concentration, polymer type, polymer swelling, polymer degradation and/or polymer cross-linking density that extends through at least one portion of the preformed biocompatible polymer-based shape morphing layer shape; and a photocurable and degradable cell-supporting microgel (MG) layer that is printed with cells. The MG layer can maintain the shape of the printed cells upon printing. The degradation of the MG layer and/or differential swelling and/or degradation preformed biocompatible polymer-based shape morphing layer during culture in tissue-specific formation conditions allows the construct to undergo controllable different shape transformations over time.

In some embodiments, ach microgel includes a plurality dual crosslinkable biodegradable natural polymer macromers crosslinked with a first agent. The microgels can be capable of being crosslinked with a second agent that is different than the first cross-linking agent. The microgels cross-linked with the second agent can form a free-standing structure, such as a tissue construct, with a defined shape.

The dual cross-linkable natural polymer macromers can be any natural polymer or oligomer that includes a functional group (e.g., a carboxylic group) that can be further polymerized. Examples of natural polymers or oligomers are saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose.

In some embodiments, the dual cross-linkable natural polymer macromer can include an acrylated and/or methacrylated natural polymer macromer. Acrylated and/or methacrylated natural polymer macromers can include saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose that can be readily oxidized to form free aldehyde units.

In some embodiments, the acrylated or methacrylated, natural polymer macromers are polysaccharides, which are optionally oxidized so that up to about 50% of the saccharide units therein are converted to aldehyde saccharide units. Control over the degree of oxidation of the natural polymer macromers permits regulation of the gelling time used to form the hydrogel as well as the mechanical properties, which allows for tailoring of these mechanical properties depending on the clinical application.

In other embodiments, acrylated and/or methacrylated, natural polymer macromers can include oxidized, acrylated or methacrylated, alginates, which are optionally oxidized so that up to about 50% of the saccharide units therein are converted to aldehyde saccharide units.

The natural polymer macromer (e.g., alginate) can be acrylated or methacrylated by reacting an acryl group or methacryl with a natural polymer or oligomer to form the oxidized, acrylated or methacrylated natural polymer macromer (e.g., alginate). For example, oxidized alginate can be dissolved in a solution chemically functionalized with N-hydroxysuccinimide and 1-ethyl-3-(3-dimethylaminopropyl)-carbodiimide hydrochloride to activate the carboxylic acids of alginate and then reacted with 2-amionethylmethacrylate to provide a plurality of methacrylate groups on the alginate.

The degree of acrylation or methacrylation can be controlled to control the degree of subsequent crosslinking of the acrylate and methacrylates as well as the mechanical properties, and biodegradation rate of the composition. The degree of acrylation or methacrylation can be about 1% to about 50%, although this ratio can vary more or less depending on the end use of the composition.

The microgels can have a diameter less than about 500 μm, less than about 400 μm, or less than about 300 μm and include, for example, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 150,000, or 200,000 cells per microgel.

In some embodiments, the preformed hydrogel layer includes a hydrogel forming acrylated and/or methacrylated polymer macromers that are optionally oxidized. The acrylated and/or methacrylated polymer macromers can reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable.

In some embodiments, the acrylated and/or methacrylated polymer macromers include acrylated and/or methacrylated polysaccharides that are optionally oxidized.

In some embodiments, the photocurable and degradable cell-supporting microgel includes an acrylated and/or methacrylated alginate that is optionally oxidized.

In some embodiments, the preformed layer includes a mixture of an acrylated and/or methacrylated alginate that is optionally oxidized and acrylated and/or methacrylated gelatin.

In some embodiments, the printed photocurable and degradable cell-supporting microgel (MG) layer includes a plurality of printed cells. The cells can include any cells, such as, undifferentiated stem cells or progenitor cells with a cell lineage potential that corresponds to the desired tissue being engineered. The cells can be unipotent, oligopotent, multipotent, or pluripotent. In some embodiments, the cells are adult stem cells. The cells can be allogeneic or autologous. In particular embodiments, the cells include mesenchymal stem cells (MSCs).

In some embodiments, the shape morphing hydrogel layer includes a single biocompatible polymer or copolymer.

In other embodiments, the preformed biocompatible polymer-based shape morphing layer includes a gradient of polymer cross-linking density through the thickness of the layer that allows the shape morphing hydrogel to undergo one or more multiple, reversible, and/or controllable different shape transformations.

In some embodiments, preformed biocompatible polymer-based shape morphing layer includes a photocrosslinkable hydrogel forming polymer and a photo-absorber and a photoinitiator dispersed within the hydrogel.

In some embodiments, the preformed biocompatible polymer-based shape morphing layer includes one or multiple gradients in polymer concentration, polymer type, polymer swelling, polymer degradation and/or polymer cross-linking density that extend through portions of the preformed biocompatible polymer-based shape morphing layer.

In some embodiments, the preformed biocompatible polymer-based shape morphing layer includes a plurality of cells, the cells comprising progenitor cells, undifferentiated cells, differentiated cells, and/or cancer cells. For example, the plurality cells can include mesenchymal stem cells.

Other embodiments described herein relate to a method of forming a layered construct. The method includes providing a preformed biocompatible polymer-based shape morphing layer that includes at least one gradient in polymer concentration, polymer type, polymer swelling, polymer degradation and/or polymer cross-linking density that extends through at least one portion of the preformed biocompatible polymer-based shape morphing layer shape. A printed photocurable and degradable cell-supporting microgel (MG) layer that is configured to allow printing of cells inside MG layer and maintains shape initially upon printing is applied over at least a portion of the preformed biocompatible polymer-based shape morphing layer. Cells are then printed within the MG layer. The degradation of the MG layer and differential swelling and/or degradation of the preformed biocompatible polymer-based shape morphing layer during culture in specific tissue-specific formation conditions allows the construct to undergo controllable different shape transformations over time.

In some embodiments, the preformed hydrogel layer includes a hydrogel forming acrylated and/or methacrylated polymer macromers that are optionally oxidized. The acrylated and/or methacrylated polymer macromers can be reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable. For example, the acrylated and/or methacrylated polymer macromers include acrylated and/or methacrylated polysaccharides that are optionally oxidized.

In some embodiments, the photocurable and degradable cell-supporting microgel can include an acrylated and/or methacrylated alginate that is optionally oxidized.

In other embodiments, the preformed layer can include a mixture of an acrylated and/or methacrylated alginate that is optionally oxidized and acrylated and/or methacrylated gelatin.

In some embodiments, the printed cells can include progenitor cells, undifferentiated cells, differentiated cells, and/or cancer cells. For example, the printed cells can include mesenchymal stem cells.

In some embodiments, the preformed biocompatible polymer-based shape morphing layer includes a single biocompatible polymer or copolymer.

In other embodiments, the preformed biocompatible polymer-based shape morphing layer includes gradient of polymer cross-linking density through the thickness of the layer that allows the shape morphing hydrogel to undergo one or more multiple, reversible, controllable and/or different shape transformations.

In some embodiments, the preformed biocompatible polymer-based shape morphing layer includes a photocrosslinkable hydrogel forming polymer and a photo-absorber and a photoinitiator dispersed within the hydrogel.

In other embodiments, the preformed biocompatible polymer-based shape morphing layer includes one or multiple gradients in polymer concentration, polymer type, polymer swelling, polymer degradation and/or polymer cross-linking density that extend through portions of the preformed biocompatible polymer-based shape morphing layer.

In some embodiments, the preformed biocompatible polymer-based shape morphing layer includes a plurality of cells. The cells can include progenitor cells, undifferentiated cells, differentiated cells, and/or cancer cells.

In some embodiments, the method further includes crosslinking the MG layer printed with the cell to enhance the mechanical stability of the MG layer.

In other embodiments, the method includes culturing the layered construct in a culture medium. The culture medium can include a cell differentiation medium.

Other embodiments described herein relate to a composition that includes a plurality of polymer macromer nanoparticle and/or microparticle hydrogels (MGs) and optionally a plurality of cells. The composition is configurable into a stable 3D hydrogel (bio)construct in the absence/presence of cells and is configured to be crosslinkable to form a more robust hydrogel construct.

In some embodiments, the hydrogel construct includes at least one an anisotropic property in crosslinking density, internal strain, and/or micro/macro-pores distribution.

In some embodiments, the MGs comprise jammed heterogenous natural or synthetic polymer macromer hydrogels. The MGs can include a photoinitiator (PI) and UV absorber.

In some embodiments, the composition is printed into 3D hydrogel (bio)constructs that are programmably reshaped into a defined shape.

In other embodiments, the composition is extrudable or printable into a defined shape.

In some embodiments, the composition is capable of being crosslinked to form a flow-resistant structure with the defined shape and with a gradient in crosslinking density that extends through at least one portion of the hydrogel. The gradient in crosslinking density can allowing the 3D hydrogel (bio)construct to undergo one or multiple, reversible, controllable and/or different shape transformations.

In some embodiments, the composition is cytocompatible and, upon degradation, produces a substantially non-toxic product.

In some embodiments, the viscosity of the MGs can decrease with increased shear and/or strain on the MGs and recover after removal of the increased shear and/or strain. The increased shear and/or strain can be associated with extruding or printing the composition, and the viscosity of the composition can recover after extruding or printing the composition to provide the 3D hydrogel (bio)construct with the defined shape.

In some embodiments, the composition can include a plurality of cells. The cells can include progenitor cells, undifferentiated cells and/or differentiated cells. For example, the cells can include mesenchymal stem cells.

In some embodiments, the MGs can have a flake morphology with an average diameter of about 10 μm to about 70 μm.

Other embodiments described herein relate to a method of forming a shape-morphing construct. The method includes providing a plurality of polymer macromer nanoparticle and/or microparticle hydrogels (MGs) and optionally a plurality of cells dispersed with MGs. The MGs and optional cells are then printed into a 3D hydrogel (bio)construct having a defined shape. The 3D hydrogel (bio)construct is crosslinked to further stabilize the 3D hydrogel (bio)construct.

In some embodiments, the 3D hydrogel (bio)construct includes at least one an anisotropic property in crosslinking density, internal strain, and/or micro/macro-pores distribution.

In some embodiments, the MGs can include a photoinitiator (PI) and UV absorber.

In some embodiments, he gradient in crosslinking density allows the 3D hydrogel (bio)construct to undergo one or multiple, reversible, controllable and/or different shape transformations.

In some embodiments, the shape-morphing construct is cytocompatible and, upon degradation, producing substantially non-toxic product.

In some embodiments, the viscosity of the MGs decreases with increased shear and/or strain on the MGs and recovers after removal of the increased shear and/or strain. The increased shear and/or strain can be associated with printing the MFHs and the viscosity of the MGs recovering after printing the MGs to provide the 3D hydrogel (bio)construct with the defined shape.

In some embodiments, the cells can include progenitor cells, undifferentiated cells and/or differentiated cells. For example, the cells can include mesenchymal stem cells.

In some embodiments, the MGs can have a flake morphology with an average diameter of about 10 μm to about 70 μm.

Still other embodiments relate to a composition for forming a shape morphing cell-laden construct. The composition can include a plurality of cells and optionally at least one polymer macromer. The composition can be configurable into a stable 3D bioconstruct having an initial shape, wherein cell contractile forces of the cells of the 3D (bio)construct allows the 3D bioconstruct to undergo one or multiple, reversible, controllable and/or different shape transformations over time.

In some embodiments, the cell contractile forces are associated with at least one of cell to cell interactions, cell to extracellular matrix interactions, cell to aptamer interactions, and/or cell condensation.

In some embodiments, the composition includes a photoinitiator (PT).

In some embodiments, the composition can be printed into 3D hydrogel (bio)constructs that are programmably reshaped into a defined shape.

In other embodiments, the composition is extrudable or printable into a defined shape.

In some embodiments, the composition is capable of being crosslinked to form a flow-resistant structure with the defined shape.

In some embodiments, the shape morphing cell-laden construct can be cytocompatible and, upon degradation, producing substantially non-toxic product.

In some embodiments, the viscosity of the composition decreases with increased shear and/or strain on the composition and recovers after removal of the increased shear and/or strain. The increased shear and/or strain can be associated with extruding or printing the composition and the viscosity of the composition can recover after extruding or printing the composition to provide the 3D hydrogel construct with the defined shape.

In some embodiments, the cells can include progenitor cells, undifferentiated cells and/or differentiated cells. For example, the cells can include mesenchymal stem cells.

Other embodiments described herein relate to a construct that includes at least one degradable cell hydrogel layer or cell condensate layer whose initial shape is maintained by a support, wherein cell contractile forces of the cells of the construct allows the construct to undergo one or multiple, reversible, controllable, and/or different shape transformations over time.

In some embodiments, the cell contractile forces are associated with at least one of cell to cell interactions, cell to extracellular matrix interactions, cell to aptamer interactions, and/or cell condensation.

In some embodiments, the support includes hydrogel and/or microgel in the hydrogel layer.

In some embodiments, the support is external to the cell condensate.

In some embodiments, the at least one degradable cell hydrogel layer or cell condensate layer includes a mixture of oxidized and methacrylated alginate (OMA), methacrylated gelatin, uncrosslinked gelatin microspheres, and plurality of cells as well as optionally a photoinitiator (PI).

In some embodiments, the construct is capable of being crosslinked to form a flow-resistant structure with the defined shape.

In some embodiments, the construct is cytocompatible and, upon degradation, producing substantially non-toxic product.

In some embodiments, the cells can include progenitor cells, undifferentiated cells and/or differentiated cells. For example, the cells can include mesenchymal stem cells.

In some embodiments, the construct can include a hydrogel layer conjugated to the at least one degradable cell hydrogel layer or cell condensate layer. The hydrogel layer can be a non-swelling and/or swelling hydrogel layer.

In some embodiments, condensation of the cells in the cell hydrogel layer or cell condensate layer and optionally degradation of the hydrogel layer during culture allows the construct to undergo one or multiple, reversible, controllable and/or different shape transformations over time.

In some embodiments, the hydrogel layer includes a hydrogel forming acrylated and/or methacrylated polymer macromers that are optionally oxidized. The acrylated and/or methacrylated polymer macromers can be reversibly and ionically crosslinkable. The acrylated and/or methacrylated polymer macromers can also be photocrosslinkable, ionically crosslinkable, physically crosslinkable, pH crosslinkable, dual crosslinkable, and/or thermally crosslinkable.

In some embodiments, the acrylated and/or methacrylated polymer macromers include at least one of an acrylated and/or methacrylated alginate that is optionally oxidized and/or acrylated and/or methacrylated gelatin.

In some embodiments, the construct includes a first degradable cell laden hydrogel layer, and a second degradable cell laden hydrogel layer overlying the first degradable cell laden hydrogel layer. The first degradable cell laden hydrogel layer and second degradable cell laden hydrogel layer can differ in at least one of amount of cells, cell types, or cell adhesive properties.

Other embodiments relate to a construct that includes a biocompatible polymer-based hydrogel. The hydrogel includes a first portion and a second portion separated by an intermediate portion. The first portion and second portion include a plurality of cells encapsulated by hydrogel of the layer and the intermediate portion being devoid of cells. The construct is configured to undergo one or multiple, reversible, controllable, and/or different shape transformations over time via cell interactions between cells in the first portion and the second portion and/or cell to extracellular matrix interaction of cells in the first portion and/or second portion.

Still other embodiments relate to a layered construct that includes a photocurable cell-supporting microgel (MG). The MG includes a first cell condensate layer and second cell condensate layer overlying the first cell condensate layer. The second cell condensate layer is different than the first cell condensate layer. The MG is configured to allow printing of cells inside MG layer and maintains shape initially upon printing. The cell condensation in the first layer and/or second layer during culture allows the construct to undergo one or multiple, reversible, controllable, and/or different shape transformations over time.

Other embodiments relate to a layered construct that includes a first degradable cell laden hydrogel layer, and a second degradable cell laden hydrogel layer overlying the first degradable cell laden hydrogel layer. The first degradable cell laden hydrogel layer and second degradable cell laden hydrogel layer differ in at least one of amount of cells, cell types, or cell adhesive properties, and wherein cell to cell interactions, cell to extracellular matrix interactions, cell to aptamer interactions, and/or condensation of the cells of the construct allows the construct to undergo one or multiple, reversible, controllable, and/or different shape transformations over time.

Other embodiments described herein relate to a construct (FIG. 73) that includes a biocompatible polymer-based hydrogel layer. The hydrogel layer includes a first portion and a second portion separated by an intermediate portion. The first portion and second portion include a plurality cells encapsulated by hydrogel of the layer. The intermediate portion is devoid of cells. The construct is configured to undergo one or multiple, reversible, controllable and/or different shape transformations over time by degradation of the intermediate portion relative to the first portion and second portion and/or cell contractile forces

Still other embodiments relate to a layered construct that includes a first biocompatible and/or cytocompatible hydrogel layer that is non-degradable or slowly degrades, and a second biocompatible and/or cytocompatible aptamer hydrogel layer overlying the first hydrogel layer wherein aptamer interactions result in layer degradation or expansion which allows the construct to undergo one or multiple, reversible, controllable and/or different shape transformations over time.

Other embodiments relate to a bio-kirigami construct (FIG. 78) that includes a preformed hydrogel frame; and a photocurable and degradable cell-supporting hydrogel member that includes a plurality of cell inside a hydrogel of the member. The member maintains shape initially upon fabrication. Swelling of the hydrogel frame and/or cell support hydrogel member, degradation of the hydrogel member and/or condensation of the cells during culture allows the construct to undergo one or multiple, reversible, controllable, and/or different shape transformations over time.

Other embodiments relate to a construct that includes a biocompatible polymer-based hydrogel. The hydrogel includes portions having at least one of differing stiffness, thickness, and/or degradation rates. The hydrogel includes a plurality cells encapsulated by hydrogel. The construct is configured to undergo one or multiple, reversible, controllable and/or different shape transformations over time based on cell mediated contractile forces within the portions of the hydrogel.

The shape morphing hydrogel and/or cell condensate actuator or construct can be used in a variety of biomedical applications, including tissue engineering, drug delivery applications, and regenerative medicine. In one example, the shape morphing hydrogel and/or cell condensate actuator or construct can be used to promote tissue growth in a subject. One step of the method can include identifying a target site. The target site can comprise a tissue defect (e.g., cartilage and/or bone defect) in which promotion of new tissue (e.g., cartilage and/or bone) is desired. The target site can also comprise a diseased location (e.g., tumor). Methods for identifying tissue defects and disease locations are known in the art and can include, for example, various imaging modalities, such as CT, MRI, and X-ray.

The tissue defect can include a defect caused by the destruction of bone or cartilage. For example, one type of cartilage defect can include a joint surface defect. Joint surface defects can be the result of a physical injury to one or more joints or, alternatively, a result of genetic or environmental factors. Most frequently, but not exclusively, such a defect will occur in the knee and will be caused by trauma, ligamentous instability, malalignment of the extremity, meniscectomy, failed aci or mosaicplasty procedures, primary osteochondritis dessecans, osteoarthritis (early osteoarthritis or unicompartimental osteochondral defects), or tissue removal (e.g., due to cancer). Examples of bone defects can include any structural and/or functional skeletal abnormalities. Non-limiting examples of bone defects can include those associated with vertebral body or disc injury/destruction, spinal fusion, injured meniscus, avascular necrosis, cranio-facial repair/reconstruction (including dental repair/reconstruction), osteoarthritis, osteosclerosis, osteoporosis, implant fixation, trauma, and other inheritable or acquired bone disorders and diseases.

Tissue defects can also include cartilage defects. Where a tissue defect comprises a cartilage defect, the cartilage defect may also be referred to as an osteochondral defect when there is damage to articular cartilage and underlying (subchondral) bone. Usually, osteochondral defects appear on specific weight-bearing spots at the ends of the thighbone, shinbone, and the back of the kneecap. Cartilage defects in the context of the present invention should also be understood to comprise those conditions where surgical repair of cartilage is required, such as cosmetic surgery (e.g., nose, ear). Thus, cartilage defects can occur anywhere in the body where cartilage formation is disrupted, where cartilage is damaged or non-existent due to a genetic defect, where cartilage is important for the structure or functioning of an organ (e.g., structures such as menisci, the ear, the nose, the larynx, the trachea, the bronchi, structures of the heart valves, part of the costae, synchondroses, enthuses, etc.), and/or where cartilage is removed due to cancer, for example.

After identifying a target site, such as a cranio-facial cartilage defect of the nose, the shape morphing hydrogel and/or cell condensate actuator or construct can be administered to the target site.

After implanting the shape morphing hydrogel and/or cell condensate actuator or construct into the stiffness of the hydrogel can be repeatably and reversibly adjusted to modulate the growth and/or proliferation of cells provided within the hydrogel or condensate as well as the release of bioactive agents provided in the hydrogel from the hydrogel. Moreover, when hydrogel is used as a tissue, which is exposed to mechanical stresses (i.e., shear and tensile stresses) in in vivo environments, the mechanical properties of the hydrogel can be increased following secondary crosslinking to improve its stability when used in such applications.

The following examples are for the purpose of illustration only and is not intended to limit the scope of the claims, which are appended hereto.

### Example 1

This example describes a multilayer hydrogel actuator systems using biocompatible and photocrosslinkable oxidized, methacrylated alginate and methacrylated gelatin that permits encapsulation and maintenance of living cells within the hydrogel actuators and implements programmed and controlled actuations with multiple shape changes. The hydrogel actuators encapsulating cells enable defined self-folding and/or user-regulated, on-demand-folding into specific 3D architectures under physiological conditions, with the capability to partially bioemulate complex developmental processes such as branching morphogenesis. The hydrogel actuator systems can be utilized as novel platforms for investigating the effect of programmed multiple-step and reversible shape morphing on cellular behaviors in 3D extracellular matrix and the role of recapitulating developmental and healing morphogenic processes on promoting new complex tissue formation.

We describe biocompatible natural polymer-based layered hydrogel systems capable of multiple and reversible different distinct shape changes over time via either pre-programmed design or user-controlled environmental condition alternations. These layered hydrogels feature easy reproducible fabrication, cytocompatibility for cell encapsulation, shape controllability, multiple-shape transformations over time, and tunable durations of different shape phases, and they may be broadly applicable as robust and versatile CHAs.

### Experimental

### Chemicals, Instruments, and General Methods

Unless specified, all solvents and reagents were used without further purification. Sodium alginate (AL, Protanal LF 20/40) was a generous gift from FMC Biopolymer. Bovine skin derived gelatin (type B), photoinitiator (2-Hydroxy-4′-(2-hydroxyethoxy)-2-methylpropiophenone, PI), Dulbecco's Modified Eagle Medium-High Glucose (DMEM-HG), Dulbecco's Modified Eagle Medium-Low Glucose (DMEM-LG), and fetal bovine serum (FBS) were purchased from Sigma. Dexamethasone was purchased from MP Biomedicals (Solon, OH). β-Glycerophosphate was purchased from CalBiochem. ITS⁺ Premix and penicillin/streptomycin (P/S) were purchased from Corning Inc. (Corning, NY). Sodium pyruvate was purchased from HyClone Laboratories. Non-essential amino acid solution was purchased from Lonza Group (Basel, Switzerland). Ascorbic acid and ascorbic acid-2-phosphate were purchased from Wako Chemicals USA Inc. (Richmond, VA). Fibroblast growth factor-2 (FGF-2) was purchased from R&D Systems (Minneapolis, MN), transforming growth factor β1 (TGF-β1) was purchased from PeproTech (Rocky Hill, NJ), and bone morphogenetic protein-2 (BMP-2) was provided by Dr. Walter Sebald from the Department of Developmental Biology, University of Wurzburg, Germany). N-(2-aminoethyl) methacrylate hydrochloride (AEMA) and methacryloxyethyl thiocarbamoyl rhodamine B (RhB) were purchased from Polysciences Inc., and other common chemicals, such as sodium peroxide, methacrylic anhydride, etc., were purchased from Fisher Scientific. ¹H NMR spectra were obtained on a 400 MHz Bruker AVIII HD NMR spectrometer equipped with a 5 mm SmartProbe™ at 25° C. using deuterium oxide (D₂O) as a solvent and calibrated using (trimethylsilyl)propionic acid-d₄ sodium salt (0.05 w/v %) as an internal reference. DMEM-LG containing 0.05% PI (w/w) was used to dissolve the oxidized methacrylate alginate (OMA) and methacrylate gelatin (GelMA). DMEM-LG media was used to culture the hydrogels without cells. Cell growth media (GM) consisting of DMEM-LG (for NIH3T3 cell-laden hydrogels) or DMEM-HG (for stem cell-laden hydrogels) with 10% FBS and 1% P/S was used to culture the hydrogels with encapsulated cells. Hydrogel images were visualized using a Nikon SMZ-10 Trinocular Stereomicroscope equipped with a digital camera. A microplate reader (Molecular Devices iD5) was used to read data from the microplates.

### Synthesis of OMAs and GelMA

OMAs with theoretical 10% oxidation degree and varying theoretical methacrylation degrees (20%, 30% and 45%) and GelMA with a theoretical 100% methacrylation degree were synthesized according to the reported literature. The O10M20A, for example, was synthesized with the following procedure: 10 g of sodium alginate was dissolved in 900 mL of diH₂O overnight, and 1.08 g of sodium periodate (NaIO₄) in 100 mL of diH₂O was rapidly added to the alginate solution under stirring in the dark at room temperature (RT). After reaction for 24 h, 19.52 g of 2-ethanesulfonic acid (MES) and 17.53 g of sodium chloride (NaCl) were added, and the pH was adjusted to 6.5 with 5 N sodium hydroxide (NaOH). Then 1.77 g of N-hydroxysuccinimide (NHS) and 5.84 g of 1-ethyl-3-(3-dimethylaminopropyl)carbodiimide hydrochloride (EDC·HCl) were sequentially added to the mixture. After 10 min, 1.69 g of AEMA was added slowly. The solution was wrapped with aluminum foil to protect it from light and left to react for 24 h at RT. The mixture was then poured into 2 L of chilled acetone to precipitate out the crude OMA solid, which was further purified by dialysis against diH₂O over 3 days (MWCO 3.5 kDa, Spectrum Laboratories Inc.) The dialyzed alginate solution was collected, treated with activated charcoal (0.5 mg/100 mL, 50-200 mesh, Fisher) for 30 min, filtered through a 0.22 μm filter and frozen at −80° C. overnight. The final O10M20A was obtained as white cotton like solid through lyophilization for at least 10 days. Other OMAs were synthesized through a similar procedure by adding different amounts of reactants and reagents, which, together with the actual methacrylation (calculated from ¹H NMR data) and actual oxidation, are shown in Table 1. Methacrylation degree was determined according to the method described in the literature. Briefly, the methacrylate modifications were quantified by comparing the methylene protons of methacrylate (˜6.1 and 5.7 ppm) with the proton integrals of the internal reference from the ¹H NMR spectra. The methacrylation degree (%) was defined as the number of methacrylated units per 100 repeating saccharide units. Oxidation degree was determined using a fluorescamine assay developed by modifying the TNBS (2,4,6-trinitrobenzene sulfonic acid) assay. Briefly, an excess amount of t-butyl carbazate (TBC, 0.1 mM in diH₂O) was used to react each OMA solution (35.2 mg/mL in diH₂O) in diH₂O for 12 h at RT. The unreacted TBC was quantified using a fluorescamine solution (0.1 mM in DMSO). 10 L of each sample was mixed with 10 μL of fluorescamine solution, followed by addition of 80 L of DMSO and allowing for reaction at RT for 2 h. Fluorescence intensity at 470 nm was measured using the microplate reader under an exication of 390 nm. Formaldehyde was used as standard. The degree of oxidation (%) was defined as the number of oxidized units per 100 repeating saccharide units.

The GelMA was synthesized as follows: 20 g of gelatin was dissolved in 200 mL of PBS at 50° C. with stirring. 20 mL of methacrylic anhydride was added dropwise (1 mL/min) while vigorously stirring. The reaction was kept for 1 h at 50° C. with stirring and then at RT overnight. The reaction mixture was precipitated into excess acetone, dried in a fume hood and rehydrated to a 10 w/v % solution in diH₂O. The GelMA was purified by dialysis against diH₂O (MWCO 3.5 kDa) for 7 days at 50° C. to remove salts, unreacted methacrylic anhydride, and byproducts, and then filtered (0.22 μm filter) and lyophilized as described above.

TABLE 1
Feeding ratios of reactants and reagents for synthesis of
OMAs and the actual methacrylation (%) of OMA and GelMA

| | | | | | | Actual | Actual |
|---|---|---|---|---|---|---|---|
| | Alginate | NaIO₄ | MES/NaCl | NHS/EDC | AEMA | methacrylation | oxidation |
| Polymer | (g) | (g) | (g/g) | (g/g) | (g) | (%) | (%) |
| O10M20 | 10 | 1.08 | 19.52/17.5 | 1.18/3.89 | 1.69 | 7.2 | 7.5 |
| O10M30 | 10 | 1.08 | 19.52/17.5 | 1.77/5.84 | 2.53 | 9.8 | 7.6 |
| O10M45 | 10 | 1.08 | 19.52/17.5 | 2.65/8.75 | 3.80 | 12.8 | 6.9 |
| GelMA | | | | | | 80.0 | |

### Swelling and Degradation Tests

Solutions of OMAs (6%) in diH₂O and GelMA (14%) in DMEM-LG containing 0.05% PI were separately placed between two quartz plates with 0.75 mm spacers and UV crosslinked (320-500 nm, EXFO OmnicureR S1000-1B, Lumen Dynamics Group) at ˜12 mW/cm² for 60 s to form the hydrogels. Then hydrogel disks were created using a biopsy punch (d=6 mm). These hydrogel samples were frozen for 4 h at −80° C. and lyophilized for 2 days. The masses of the dried gels were measured as initial weights (Wᵢ). For the swelling test, the dried hydrogels were rehydrated by immersing into 1 mL of DMEM-LG and incubated at 4° C. to minimize the degradation for 10 h. The hydrogels were collected, and the swollen weights (Wₛ) were measured. The swelling ratios were calculated with the following equation: Wₛ/Wᵢ (N=3). For the degradation test, the dried hydrogels were incubated in 1 mL of DMEM-LG at 37° C. with media changes every other day to pre-determined time point. The rehydrated hydrogels were collected and dried by lyophilization to obtain dried mass (W_{d}). Mass loss was quantified as (Wᵢ−W_{d})/Wᵢ×100% (N=3) for each condition per time point.

### Fabrication of Cell-Laden Trilayer Hydrogel Bars

NIH3T3 fibroblast cells were cultured and expanded in NIH3T3 GM at 37° C. and 5% CO₂ with media changes every 2 or 3 days. The cells were harvested for encapsulation when they reached 80% confluence.

To make the trilayer hydrogel bars, OMAs (6%) was dissolved in DMEM-LG and GelMA (14%) were dissolved in DMEM-LG containing NIH₃T₃ cells (5×10⁶ cells/mL) and 0.05% PI. 200 μL of OMA solution was placed between two quartz plates with 0.4 mm spacers and subsequently photocrosslinked with UV light for 30 s to form an OMA hydrogel sheet. Trilayer hydrogel bars were fabricated with a “sandwich” method. Briefly, two single OMA hydrogel sheets (0.4 mm thickness) were made separately and 200 μL of the GelMA solution (14% in DMEM-LG containing 0.05% PI) with cells was placed between them. The “sandwich” was crosslinked by exposure to UV light for a further 60 s to create a trilayer hydrogel sheet, which was cut into trilayer hydrogel bars with dimensions of L×W×H=13×2×1.2 mm (0.4 mm per layer). The trilayer hydrogel bars were immediately immersed in the NIH3T3 GM to record the corresponding shape changes.

To visually distinguish different hydrogel layers, 0.005% RhB was added to either an OMA layer or GelMA layer to impart the corresponding hydrogel with a red color.

### Rheological Properties Test

All single layer hydrogels (cell-free OMA and cell-free/cell-laden GelMA hydrogels) were prepared 0.6 mm thick, and GelMA hydrogels in the tri-layer were fabricated with a thickness of 0.6 mm instead of 0.4 mm, while the outer OMA layer(s) were kept 0.4 mm thick to ensure the same degradation times for the outer OMA layers as those in triple layers described above (section 1.4). Hydrogels, (1) cell-free single layers (OMA hydrogels and GelMA hydrogels) for UV crosslinking time screening, (2) cell-laden GelMA single layers cultured in the GM for 2 h at 37° C. to represent the GelMA layers in the beginning phase (phase I), (3) triple layers for providing the cell-laden GelMA layer at final phase (phase V) after complete degradation of the two outer OMA layers, and (4) as-prepared single cell-free GelMA layers (GelMA w/o cells) for comparison, were made into 2 cm disks with a biopsy punch and punched again into small hydrogel disks with another biopsy punch (d=8 mm) to test the rheological properties on a Kinexus Ultra+ rheometer (Malvern Panalytical). In oscillatory mode, a parallel plate (8 mm diameter) geometry measuring system was employed, and the gap was set to 0.6 mm. After each hydrogel disk was placed between the plates, all the tests were carried out at 25° C. Oscillatory frequency sweep (0.1-100 Hz at 1% strain) tests were performed to measure storage moduli (G′) and loss moduli (G″). N=3.

### Mechanical Testing

The tensile testing was performed according to the reported literature using a mechanical testing machine (225 lbs Actuator, TestResources, MN, USA) equipped with a 25 N load cell to evaluate the interfacial adhesive strength of the OMA and GelMA hydrogels. Briefly, the hydrogel samples with an interfacial cross-sectional area of 5×1 mm² were attached to two hard paper backings using cyanoacrylate glue (Krazy Glue®, Elmer's Products Inc., Columbus, OH). The hard paper backings were then attached firmly with common commercial transparent tape to a “plastic loading platen”, which was attached to the “load cell”, and to a “sample cup”, which was fixed on the bottom platform of the mechanical testing machine with a 4 mm gap. The adhesion strength was determined by performing constant strain rate (1.25%/sec) tensile tests at room temperature (RT). The tests were performed no less than 3 times per group (N≥3) and all the samples ruptured in the same relative location (i.e., O10M20A/GelMA and O10M45A/GelMA samples ruptured on the OMA sides, while O10M30A/GelMA ruptured at the interface).

For the compressive modulus tests, the cell-laden GelMA samples were prepared as described above (section 1.6, rheological test). The compressive moduli of the cell-laden GelMA layer (as-prepared single layer or obtained from the degradation of a trilayer) in the beginning and final phases were determined by performing uniaxial, unconfined constant strain rate (0.8%/sec) compression tests at RT on the mechanical testing machine. Note that the samples were compressed gently with a spatula to make a full contact with a common flat glass plate, and then the glassplate with the sample was loaded into the testing machine for the test. The Young's modulus of each sample was determined using the first non-zero slope of the linear region of the stress-strain curve within 0-10% strain (N=3).

### The Bending Angle Measurement

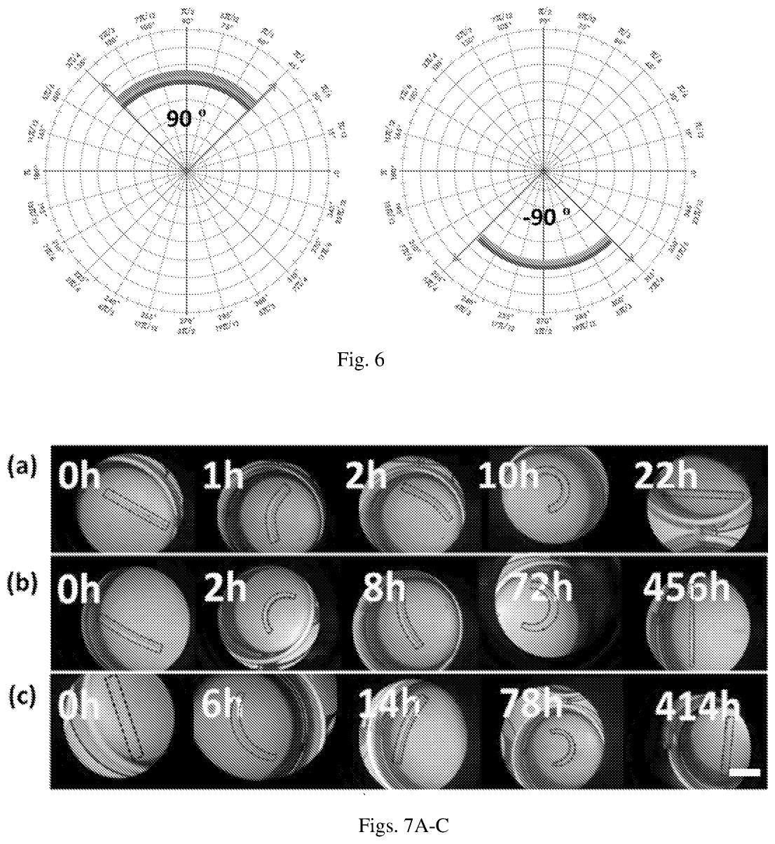

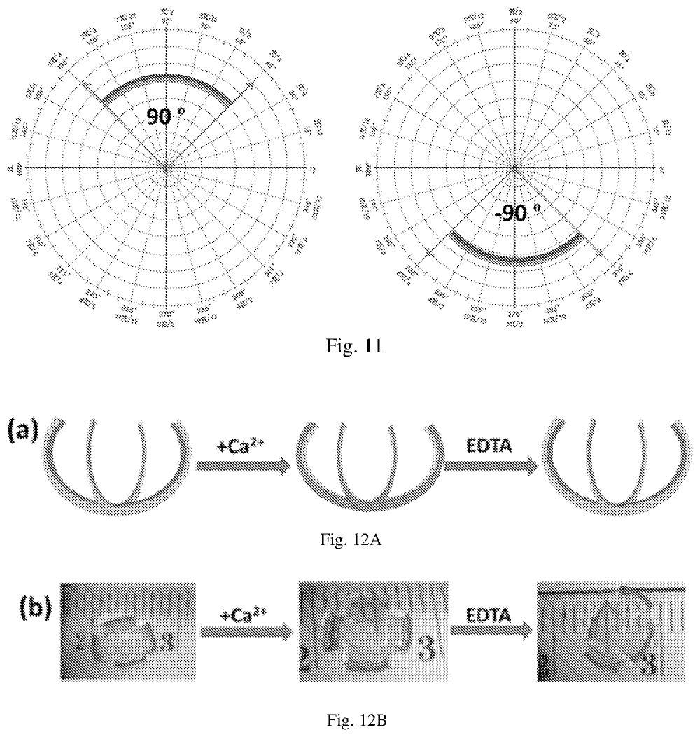

The bending angle in this manuscript was defined according to previously reported literature. Briefly, the hydrogel curve (bended hydrogel bar) is extended to a circle that matches well with the hydrogel curvature using Image J software (FIGS. 6 and 11). The bending angle is determined by measuring the central angle generated by drawing two lines between the endpoints of the hydrogel curve and the circle center, respectively. The determination of bending angles of programmable trilayer hydrogel bars, where the angle is positive when the trilayer bends toward the slower-swelling OMA layer side (purple layer), and it turns negative when the bending direction changes after the degradation of the outer faster-swelling OMA layer (red layer). The determination of “on-demand” bending angles of bilayer hydrogel bars is shown in FIG. 11. The angle is positive when the bilayer bends toward the GelMA layer side, and it turns into negative when it bends toward the OMA layer side. N=3.

### Surface Patterning of the Cell-Laden GelMA Hydrogel

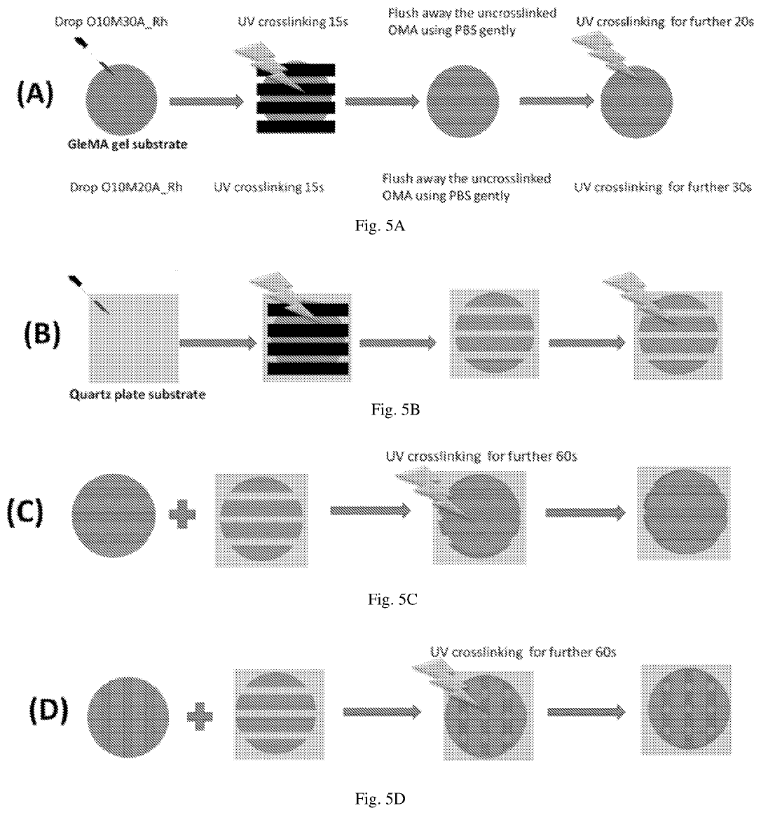

A photomask-based photolithography technique was adopted to pattern the cell-laden GelMA hydrogel surface with OMA hydrogel strips. To pattern OMAs onto the dual surfaces of a cell-laden GelMA hydrogel, a single surface patterned GelMA hydrogel (FIG. 5A) and a pre-patterned OMA layer (FIG. 5B) were fabricated separately. Briefly, to pattern OMA onto the single surface of a cell-laden GelMA hydrogel, 200 μL of GelMA solution (14% in DMEM-LG containing 0.05% PI) with NIH3T3 cells (5×10⁶ cells/mL) was placed between two quartz plates with a 0.4 mm spacer and UV crosslinked for 30 s. Then 200 μL of OMA solution (6% in DMEM-LG containing 0.05% PT) was placed onto the surface of the cell-laden GelMA hydrogel, which was covered immediately with another quartz plate. Then a photomask with a parallel strip pattern was attached onto the surface of the top quartz plate. After UV irradiation for 30 s, the uncrosslinked OMA solution was gently flushed using PBS (pH 7.4). To pattern OMA alone onto the quartz plate, 200 μL of OMA solution (6% in DMEM-LG containing 0.05% PI) was placed between two quartz plates with 0.4 mm spacers. Then a photomask with a strip pattern was attached onto the surface of the top quartz plate. After UV irradiation for 30 s, the top quartz plate was removed carefully and the uncrosslinked OMA solution remaining on the bottom quartz plate was gently flushed using PBS (pH 7.4). The two parts resulting from 5A and 5B were then aligned manually and then further crosslinked to form the final dual-surface patterned GelMA hydrogel (FIGS. 5C and 5D). The dual-surface patterned hydrogels were then cropped into hydrogel disks using a biopsy punch (d=15 mm) and immediately immersed into NIH3T3 GM to observe the shape changes.

Encapsulation of NIH3T3 and Human Mesenchymal Stem Cells (hMSCs) in GelMA Hydrogel Layer for Cell-Related Experiments: Live/Dead Staining, Chondrogenesis and Osteogenesis

In these studies, 0.005% RhB was incorporated into the OMA hydrogels to visually distinguish different hydrogel layers.

### Cell Expansion, Encapsulation and Cell-Laden Hydrogel Incubation

NIH3T3 fibroblasts were expanded as described earlier in section 1.4 and encapsulated in the GelMA hydrogel layer to examine cell viability based on the live/dead staining assay at each predetermined time point during and/or after the hydrogel deformation. GelMA was dissolved in DMEM-LG (14%) containing NIH3T3 cells (5×10⁶ cells/mL) and 0.05% PI. Hydrogel bars or other constructs were produced as described earlier and cultured in NTH3T3 GM to investigate shape morphing and cell viability.

hMSCs were encapsulated in GelMA hydrogels (5×10⁶ cells/mL) for both the chondrogenesis and osteogenesis studies. hMSCs from two different donors were used for the cell differentiation study. The hMSCs from donor 1 were isolated as described previously for the osteogenesis study. The hMSCs from donor 2 were isolated in the same manner but expanded in FGF-2 as described previously for the chondrogenesis study. hMSCs were expanded from passage 2 (P2) to passage 3 (P3) in the hMSC GM for osteogenesis or in hMSC GM containing 10 ng/mL FGF-2 for chondrogenesis in an incubator at 37° C. and 5% CO₂ with media changes every 2 or 3 days. The cells were harvested for encapsulation when they reached 80% confluence. The cell-laden hydrogel bars or other constructs were fabricated as above. Hydrogels for chondrogenesis were cultured in basal pellet media (BPM) consisting of DMEM-HG with 1% ITS' Premix, 100 nM dexamethasone, 1 mM sodium pyruvate, 100 μM non-essential amino acids, 34.7 μg/mL ascorbic acid-2-phosphate and 1% P/S supplemented with 10 ng/mL TGF-β1. Hydrogels for osteogenesis were cultured in osteogenic media (OM) consisting of DMEM-HG with 10% FBS, 1% P/S, 10 mM β-glycerophosphate, 50 μM ascorbic acid, and 100 nM dexamethasone supplemented with 100 ng/mL BMP-2. All these hydrogels were cultured in 12-well tissue culture plates filled with 2 mL of culturing media and placed in a humidified incubator at 37° C. with 5% CO₂ for 3 (chondrogenesis) or 4 (osteogenesis) weeks with media changes every 2 days.

### Live/Dead Staining

A live/dead staining assay was carried out to examine the viability of encapsulated cells at each designated time point using fluorescein diacetate (FDA, Sigma), which stains the cytoplasm of viable cells green, and propidium iodide (PI, Sigma), which stains the nuclei of dead cells red. 1 mL of live/dead staining solution, which was freshly prepared by mixing 8 μL of FDA solution (5 mg/mL in DMSO) and 8 μL of PI solution (2 mg/mL in PBS, pH 7.4) with 5 mL of PBS (pH 8.0), was added to each well containing the cell-hydrogel constructs. After 5 min incubation at RT in the dark and washing with 1 mL of PBS two times, z-stacked fluorescence images of the samples were taken using an ImageXpress Pico Automated Cell Imaging System equipped with a 5 mega Pixel CMOS digital camera (Molecular Devices). The individual z-stacked images were assembled using CellReporterXpress software (Molecular Devices). Quantification of the cell viability was based on the live/dead staining images, in which the green staining and red staining represented live cells and dead cells, respectively. Cell counts were carried out using Image J software (NIH). Cell viability was calculated as follows: (number of green (live) stained cells)/(number of green+red stained cells)×100%. Ten random fields (1.8 cm×2.3 cm) from 3 images of 3 samples were selected for each group.

### Biochemical Quantification of Chondrogenesis and Osteogenesis

To investigate the osteogenic and chondrogenic differentiations of hMSCs within the GelMA layer of the trilayer hydrogel bars, hydrogel actuators were cultured as described in section 1.91, removed from the plates at predetermined time points (d1, d2, d14 and d21 for the chondrogenic differentiation of the trilayer hydrogel bars, and d1, d2, d12 and d28 for the osteogenic differentiation of the trilayer hydrogel bars) and stored at −20° C. until all samples were collected. The chondrogenic hydrogels were put in 0.6 mL of papain buffer (Sigma) and the osteogenic hydrogels were put in 0.5 mL of CelLytic™ buffer (Sigma), and these hydrogels were then homogenized at 35,000 rpm for 2 min using a TH homogenizer (Omni International) on ice. The samples for chondrogenic studies were digested at 65° C. for 24 hours and centrifuged for 10 min at 15,000 rpm, and then the supernatants were collected for DNA and glycosaminoglycan (GAG) quantifications (N=3). The samples for the osteogenic study were centrifuged for 5 min at 500 g and 4° C., and the samples were collected for DNA, alkaline phosphatase (ALP), and calcium analysis (N=3).

Per manufacturer's instructions, a Picogreen assay kit (Invitrogen) was used to quantify the DNA content in the supernatant. Fluorescence intensity of the dye-conjugated DNA solution was measured using a microplate reader with an excitation of 480 nm and emission of 520 nm.

The GAG content was quantified using a DMMB (1,9-dimethylmethylene blue) assay. Briefly, DMMB dye solution was prepared by dissolving 21 mg of DMMB and 2 g of sodium formate in 5 mL of absolute ethanol, and then 795 mL of diH₂O was added to the solution to reach a total volume of 800 mL. The pH of the solution was adjusted to 2 using formic acid. Then diH₂O was added to the solution again to bring the solution to a total volume of 1000 mL. For GAG quantification, 40 μL of supernatant from the digested samples was transferred into 96-well plate, to which 125 μL of DMMB solution was then added. Absorbance at 595 nm was recorded on a microplate reader. GAG content was normalized to DNA content.

For ALP quantification, 100 μL of supernatant was treated with p-nitrophenylphosphate ALP substrate (pNPP, 100 μL, Sigma) at 37° C. for 30 min, and then 0.1 N NaOH (50 μL) was added to stop the reaction. The absorbance was measured at 405 nm using a microplate reader.

To quantify the calcium content, an equal volume of 1.2 N HCl was added into each remaining lysate solution and pellet, and then the mixed solutions were centrifuged at 500 g for 5 min at 4° C. A calcium assay was then performed using a kit (Pointe Scientific) per the manufacturer's instructions. Briefly, supernatant (4 μL) was mixed with a color and buffer reagent mixture (250 μL), and the absorbance was recorded at 570 nm on a microplate reader. All ALP and calcium content measurements were normalized to DNA content.

### Statistical Analysis

Statistical analysis was performed with one-way analysis of variance (ANOVA) with Tukey honestly significant difference post hoc tests using Origin software (OriginLab Corporation). A value of p<0.05 was considered statistically significant. The sample sizes for data quantification are indicated in the corresponding experimental sections and figure legends. All quantitative data was expressed as mean±standard deviation (±SD).

### Results

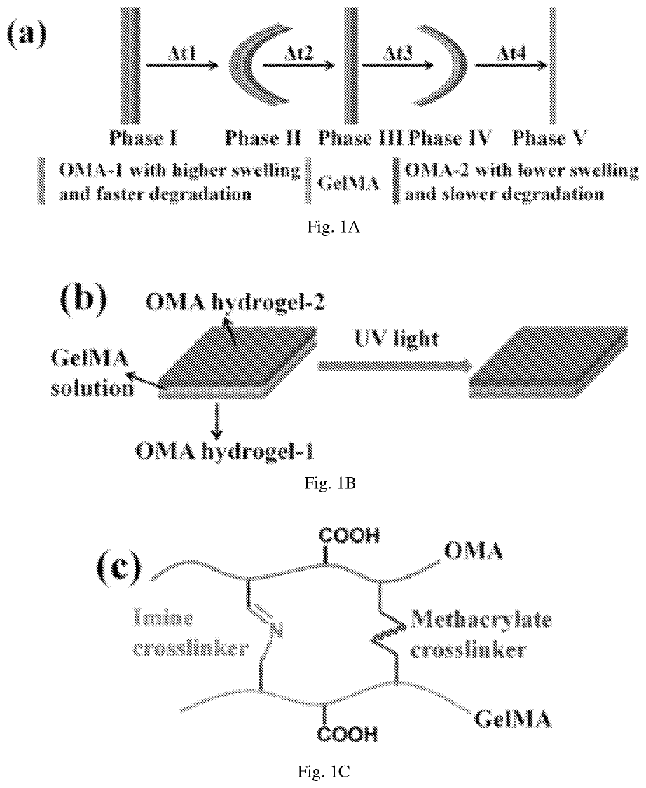

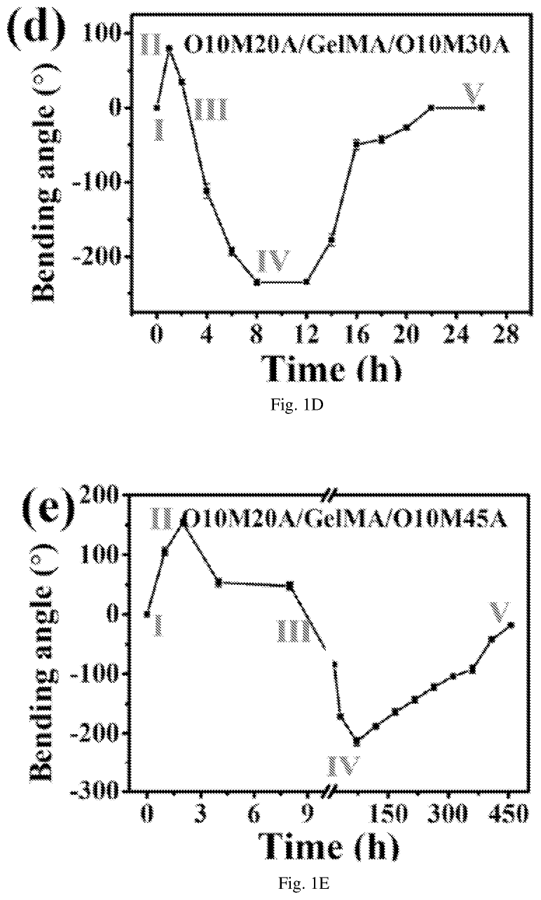

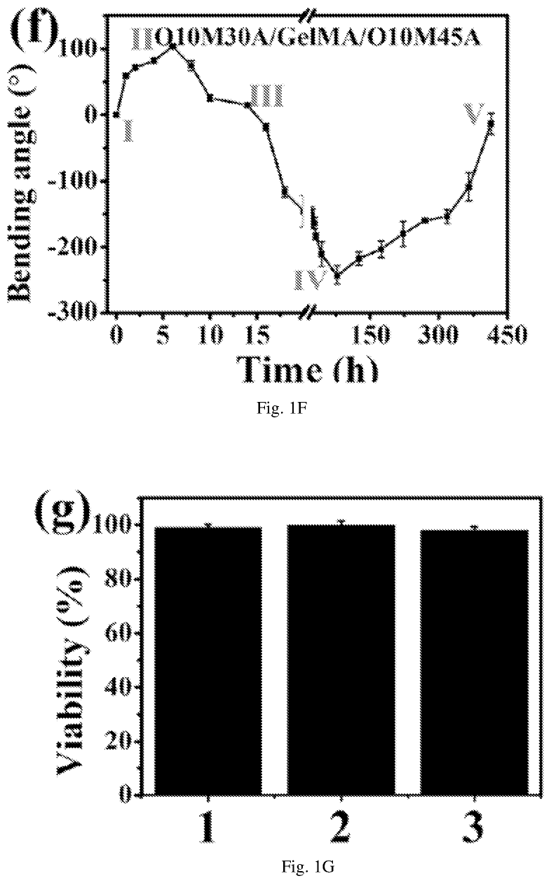

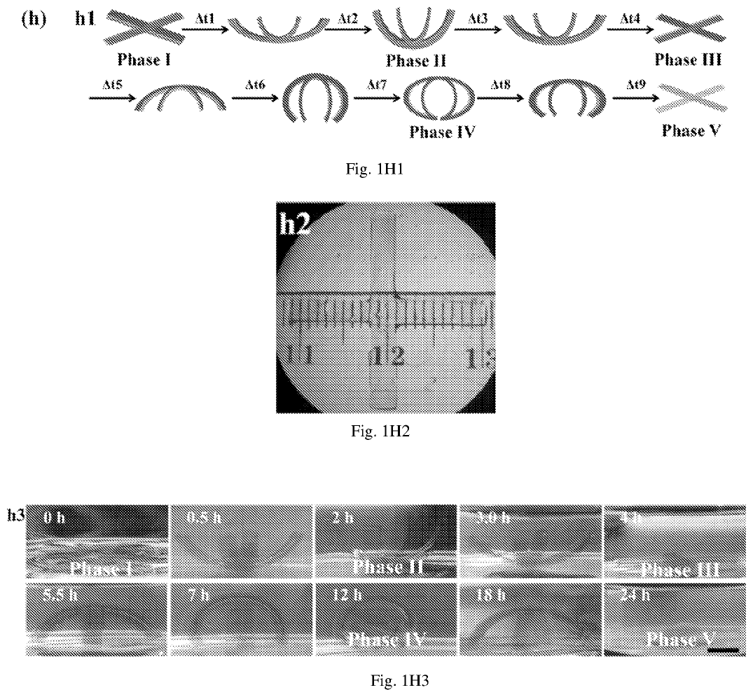

The overall strategy for a pre-programmed multiple-shape morphing CHA is based on a trilayer approach depicted in FIG. 1A. The trilayer consists of two outer OMA (oxidized methacrylated alginate) layers with different swelling ratios and degradation rates and a GelMA (methacrylated gelatin) layer. This unique trilayer design is expected to undergo multiple-shape (five-phase) transformations during culture in media due to the swelling and degradation discrepancies of these layers. OMAs (O10M20A, O10M30A, and O10M45A) were synthesized by functionalizing alginate through both oxidation (10% oxidation) and methacrylation (20%, 30%, and 45%), and GelMA was synthesized by the reaction of type-B gelatin with methacrylic anhydride (Table 1). To fabricate the multiple-step, pre-programmed shape morphing trilayer hydrogels, a “sandwich” method was used: GelMA solution containing live cells was placed between two pre-fabricated individual OMA layers and then crosslinked under UV light (FIG. 1B). For the OMA layers fabrication, 30 s UV irradiation was applied to crosslink the OMA hydrogel precursor to form a stable hydrogel, while at the same time preserving some unreacted methacrylate groups for subsequent adhesion to the GelMA layer. Then, 60 s UV irradiation was applied to crosslink the GelMA solution between the prefabricated OMA layers to fully crosslink the methacrylates in the GelMA and OMA layers to form a stable triple-layered hydrogel. The working principle for the hydrogel layer interface adhesion lies in the formation of the crosslinks (FIG. 1C, blue bond) through the photopolymerization of the remaining methacrylates in OMAs with the methacrylates in GelMA. In addition, the aldehyde groups on the OMA hydrogel surface react slowly with the amine groups on the GelMA hydrogel surface to form imine bonds, generating a second covalent bond, which further reinforces the interface adhesion (FIG. 1C, red bond). As a result, the adhesion strength at the interface was similar to or even stronger than the ultimate tensile strength of the OMA and GelMA hydrogels alone. This simple hydrogel coupling method makes it more adaptable and flexible compared to other routine methods, such as adhesion by addition of supramolecular glue, surface crosslinking by post-surface modification, and self-curing of two-independent layers,^{[39]} which typically require additional steps and longer time, making them time-consuming and lower efficiency protocols.

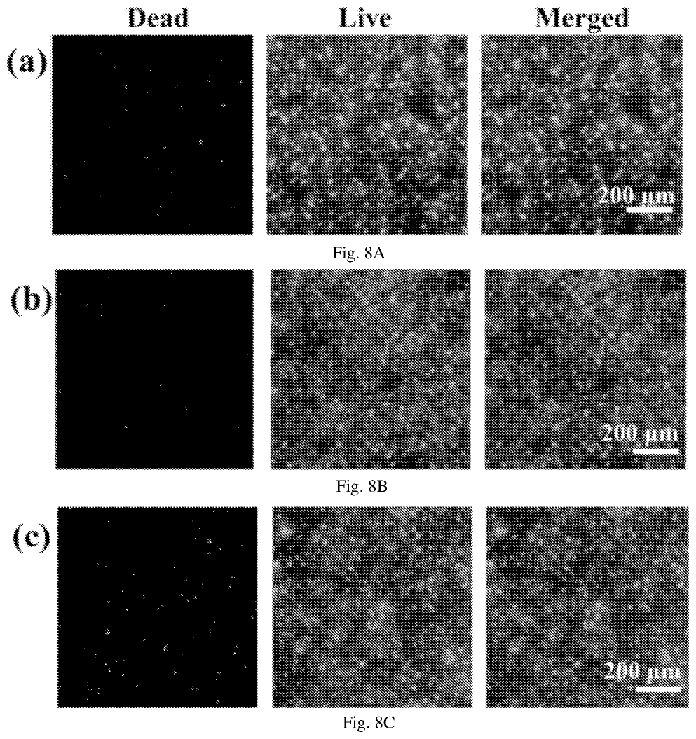

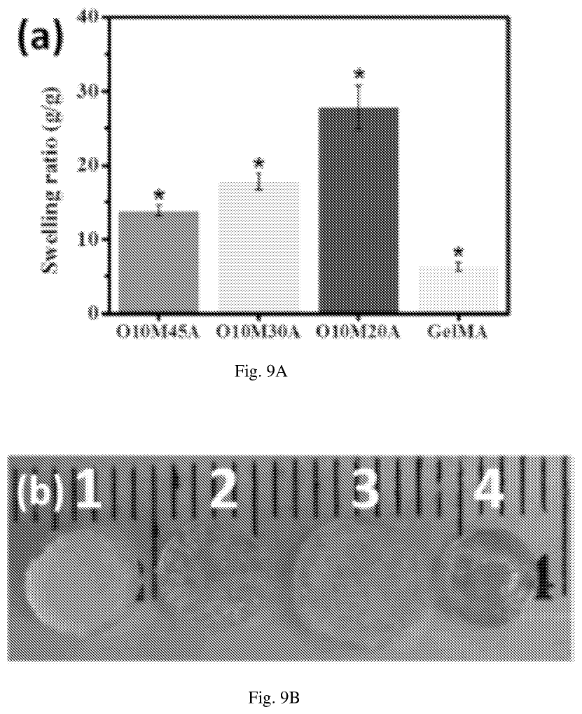

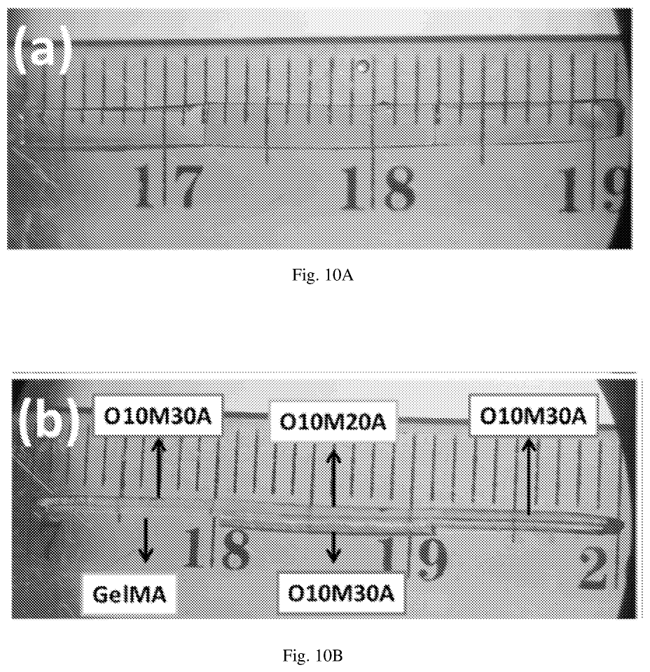

To characterize the programmable actuation behaviors of the trilayer CHA, three trilayer hydrogel bars (O10M20A/GelMA/O10M30A, O10M20A/GelMA/O10M45A, and O10M30A/GelMA/O10M45A) encapsulating NIH3T3 fibroblasts in the GelMA layer were prepared and then cultured in growth media (GM) at 37° C. to investigate their shape changes over time. All three trilayer CHAs exhibited five programmable phase transitions (bidirectional bending) with distinct bending angles (the bending angle measurement calculation is described in FIG. 6), phase durations (FIGS. 1D-F and 7), and high cell viability (FIGS. 1G and 8). Since the bending of the trilayer constructs originates from the anisotropic swelling of the three hydrogel layers, the swelling properties of these hydrogels were examined to gain insight to the bending behaviors. It was found that the swelling of OMA hydrogels decreased with increased methacrylation (S_{O10M20A}>S_{O10M30A}>S_{O10M45A}), and these OMA hydrogels exhibited much higher swelling ratios compared to the GelMA hydrogel (FIG. 9A). The dimensions of equilibrated hydrogels were consistent with the corresponding swelling ratios (FIG. 9b). This result indicates, that within the trilayer system, the OMA hydrogel with the highest swelling ratio acted as an actuation layer while the GelMA and/or OMA hydrogel with a lower swelling ratio(s) acted as constraint layer(s). The transition of Phase I to Phase II was driven by the competitive swelling of the two OMA layers, and the degree of bending was determined by the swelling difference. Consequently, O10M20A/GelMA/O10M45A, which presented the largest swelling difference between two OMA hydrogels, generated the largest curvature in Phase II (FIG. 1E). The competitive swelling and the unceasing but asynchronous degradation of the two OMA layers dominated the ensuing three steps and determined the entire timespan of the 5 phases. Since the OMA hydrogels degrade more rapidly with decreased methacrylation (O10M20A>O10M30A>O10M45A), O10M20A/GelMA/O10M30A exhibited the shortest time period through all the phases. Since the degradation of the OMA layers over time simultaneously resulted in swelling and weakened the hydrogel mechanics, the ultimate bending arose from the net outcomes of the competition between these two factors. In comparison with the actuation OMA layers, the cell-laden GelMA layer was very stable and showed minimal degradation during 4 weeks of culture. In addition, the Young's modulus and storage modulus of the cell-laden layer minimally changed after the five-phase transition. Thus, an initially stable microenvironment was provided to the residing cells. With cell-laden trilayered hydrogel actuators, we further fabricated more complicated 4-arm gripper structures which also showed five distinct phase transitions in a programmed manner (FIG. 1H).

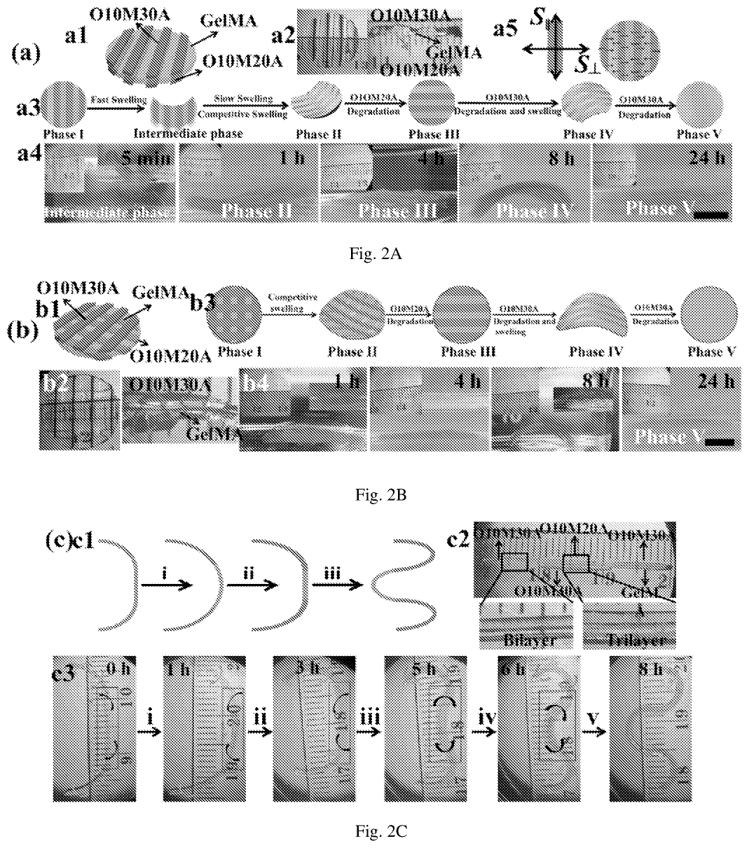

Photolithography techniques offer powerful tools to incorporate antistrophic structures within a hydrogel with high precision, enabling complex shape transformation in a pre-designed way. Mask-based photolithography allows facile patterning of OMA the GelMA hydrogel surface, and the design of the pattern enables unique control over pre-programmed CHA shape deformations. To demonstrate the feasibility of more sophisticated structure transformations with programmability, OMA-patterned GelMA hydrogel disks showing parallel OMA strips on both surfaces [overlapping patterns (FIGS. 2A1 and 2A2) and perpendicular patterns (FIGS. 2B1 and 2B2)] were fabricated. Unexpectedly, the overlapping patterned disk plunged into an intermediate phase where the disk bent perpendicularly with the long axes of the parallel strips instead of going directly to the expected Phase II (FIG. 2A4, 5 min). This was likely due to the swelling in the transverse direction of the strips (S⊥) exhibiting much greater strain than that from the longitudinal swelling (S_{|}) at the initial stage (FIG. 2A5). Meanwhile, the gradually increasing S_{|} along the long axis of the strip overcame the deformation in the intermediate phase, and thus the hydrogel sheet transformed to Phase II at 0.5 h. Then the subsequent phases occurred successively in a similar manner to that of the hydrogel bars and grippers. Interestingly, perpendicularly patterned hydrogel disks went through a five-phase transition in a different manner (FIG. 2B). The 3D structure in Phase II also originated from the competitive swelling of two OMA layers, and the disk bent along the longitudinal directions of the O10M20A strips, while the O10M30A strips stayed perpendicularly to the bending orientation. Once the bending force from the O10M20A strips was relieved by their degradation, the swelling of the O10M30A strips on the other side of the construct started reversing the bending from concave to convex and with the bending orientation perpendicular to the long axes of O10M30 strips (Phases III-IV). The construct then again resumed a flattened shape after degradation of O10M30A strips (Phase V). These results establish that by deliberately engineering the trilayer, complex structures may be obtained with multiple stages of deformation, which is beyond the capability of conventional shape-morphing systems that only show unidirectional deformation.

After having demonstrated this system can be designed to undergo multiple distinct different shape changes over time, we then wanted to explore its capacity to biomimic an actual developmental process such as branching morphogenesis. During development, branching morphogenesis is a pivotal process that occurs during the formation of many important organs/tissues, including the lung, kidney, salivary gland and mammary gland. Some of the shape changes that take place in these organs/tissues are relatively similar. For example, branching morphogenesis of the lung occurs through the repeated formation of nascent buds and subsequent cleft creation and bifurcation (FIG. 2C1). To mimic this process using our understanding of the programmable multi-phase change behavior of the CHAs, cell-laden discrete hydrogel bars were fabricated (FIGS. 2C2 and 10), which consisted of two bilayers on both sides (O10M30A/GelMA) and a trilayer in the middle (O10M20A/GelMA/O10M30A). The morphogenetic process was then investigated in GM at 37° C. (FIG. 2C3). The deformation started by the evagination of the middle segment (step i), which resembles the budding process. Upon the degradation of the O10M20A layer, the effect of the antagonistic strain of the O10M30A layer in the middle segment slowly emerged (step ii) and then dominated at 5 h (steps iii-v) to create a cleft-like structure. The evolution of the tissue-mimic continued by proceeding to form an invaginated basin accompanied with formation of two arcs on both sides after 8 h (step iv and v), which resemble two new “buds”. The increasing bending of the bilayers on both sides synchronized with the evagination and invagination, which further augmented the formation of the final bifurcated structure. The timing of each of these stages could be further controlled by changing the composition of the layer components to regulate their relative swelling and degradation rates. These findings demonstrate that this discrete multilayer hydrogel self-deformed in a spatiotemporally programmed manner that enabled biomimicry of the important individual stages of branching morphogenesis.

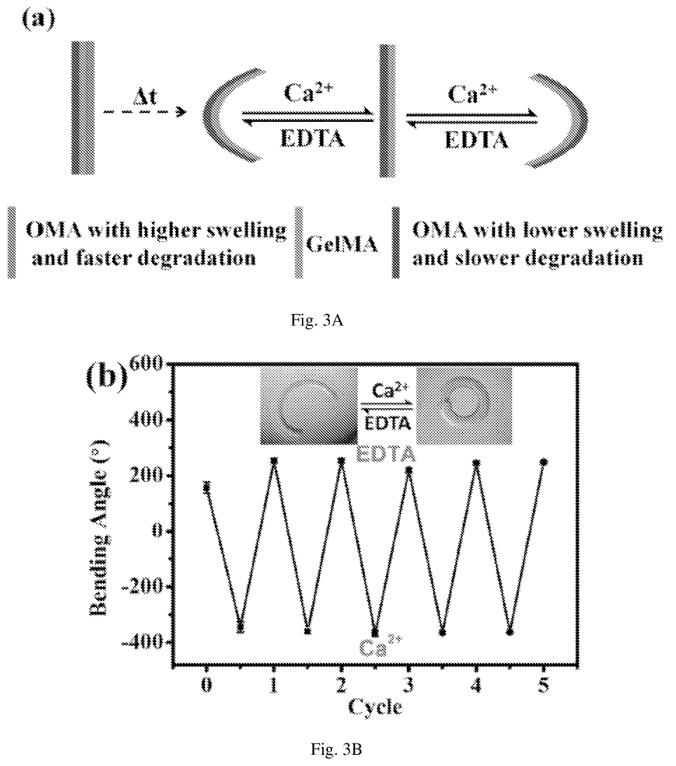

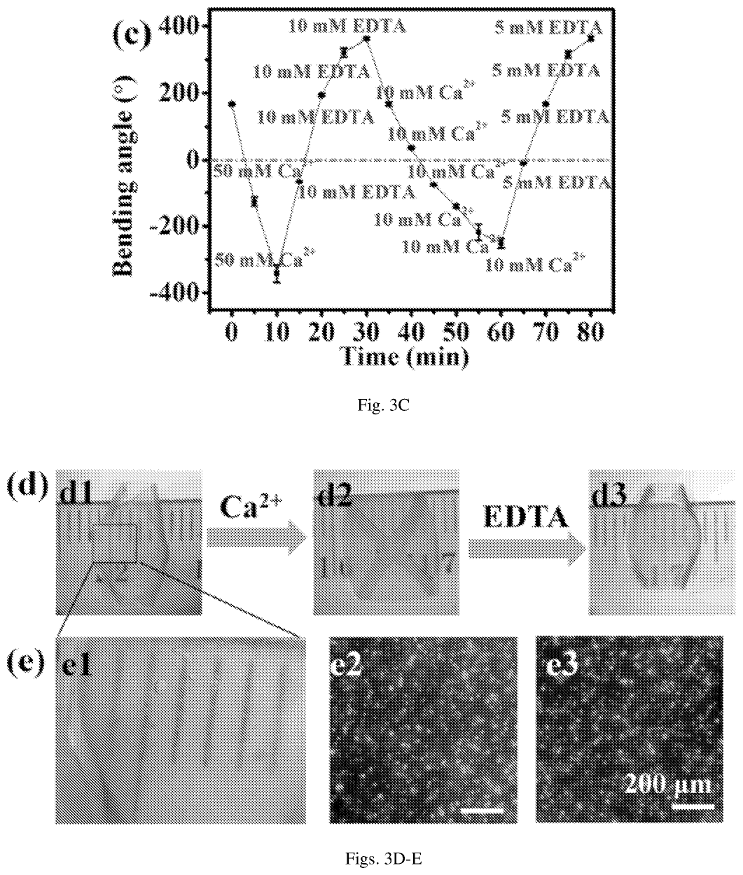

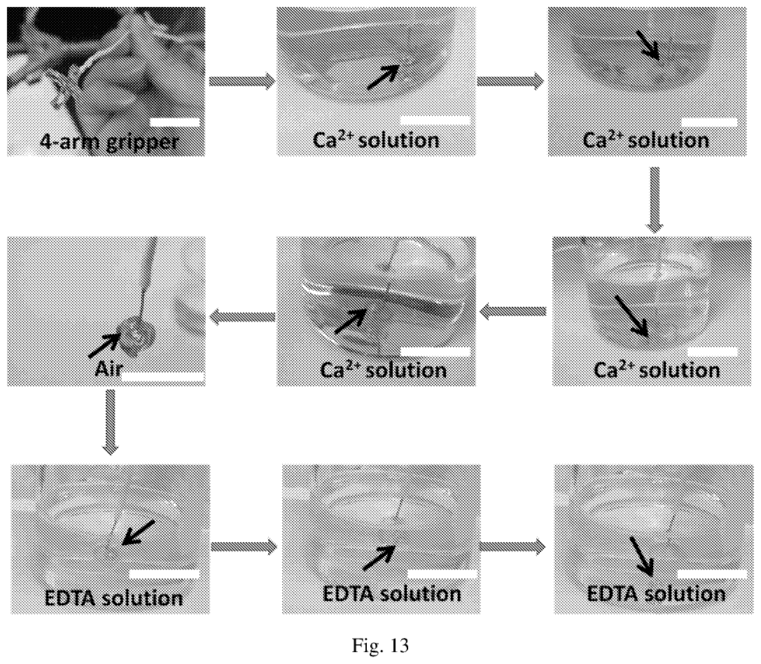

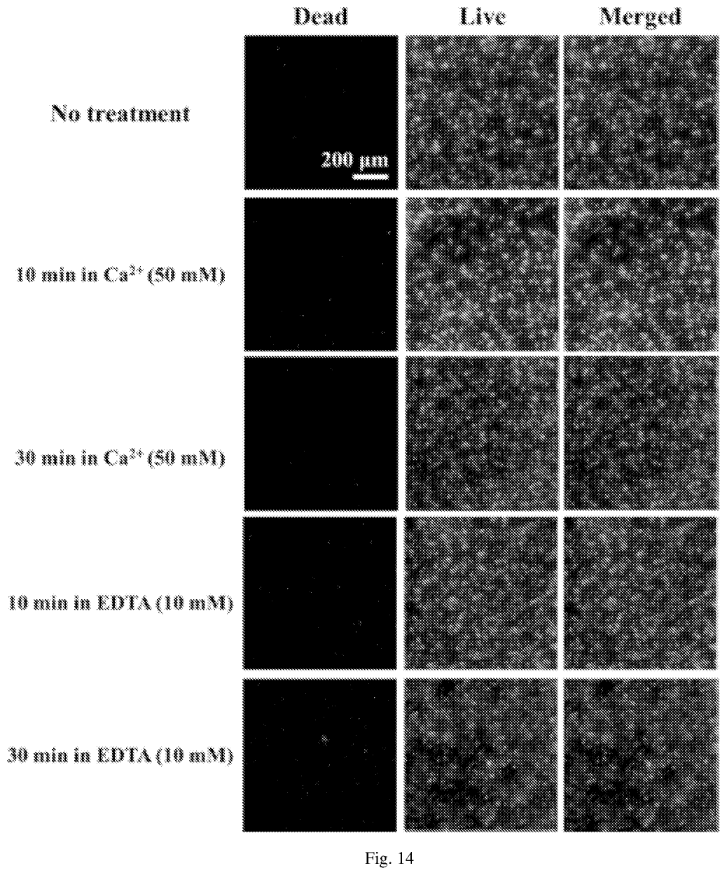

In addition to programmability, external “on-demand” control over construct shape change may also be highly desirable for a cytocompatible hydrogel actuator as this would allow precisely defined and robust user-regulated shape manipulation during the tissue formation process. Alginate and its derivatives form ionically crosslinked hydrogels with divalent cations such as calcium ions (Ca²⁺), and these crosslinks can be reversibly removed in the presence of chelating agents such as EDTA (ethylene diamine tetraacetic acid). The ionic crosslinking induces hydrogel network contraction while the removal of the cations results in hydrogel swelling relaxation. User controlled application of either ionic crosslinking or chelator solution permits regulation of construct environmental conditions and reversible deformation of layered hydrogel composites. Therefore, the bilayer obtained from the trilayer after the degradation of the fast-degradation OMA layer offers a second opportunity to regulate the shapes of hydrogel actuators on demand (FIG. 3A). Taking this into consideration, the GelMA/O10M30A bilayer resulting from the quick degradation of the O10M20A layer in the O10M20A/GelMA/O10M30A trilayer CHA was utilized to verify the shape responses upon external environmental stimulation. By soaking the bilayer bar in solutions containing Ca²⁺ or EDTA, they completely changed bending directions (FIG. 3B, inset). When the immersion solution was repeatedly switched, the hydrogels almost entirely recovered their previous shape for 5 cycles (FIGS. 3B and 11). Based on this property, an intelligent 4-arm hydrogel that reversibly switched its shape with Ca²⁺ or EDTA alternations (FIG. 12) was fabricated to act as an environmentally controlled gripper for cargo transportation, as demonstrated by its ability to transfer an aluminum ball (0.2 g) from the Ca²⁺ solution to the EDTA solution (FIG. 13). Notably, the curvature of the cell-laden bilayer could be readily tuned by varying the incubation time and/or the concentration of Ca²⁺/EDTA (FIG. 3C). The bending rate highly depended on the concentration of Ca²⁺ (−55° and −25 min⁻¹ with 50 and 10 mM Ca²⁺, respectively), whereas the concentration of EDTA exerted no obvious influence on the bending rate (−30° min⁻¹ with both 10 and 5 mM EDTA). Importantly, the cells inside the hydrogel remained highly viable after treating with both Ca²⁺ and EDTA (FIGS. 3E and 14). Furthermore, a cell-laden 3D bilayer construct, designed to fold into the shape of “quasi-four-petal flower” via OMA surface-patterning on a cell-encapsulated GelMA hydrogel sheet (FIG. 15), bent reversely after treating with Ca²⁺ and reverted to the original shape after treating with EDTA (FIG. 3d) while maintaining high cell viability (FIG. 3E). These results establish that the shape of cytocompatible CHAs can be modulated reversibly and repeatedly by external stimuli.

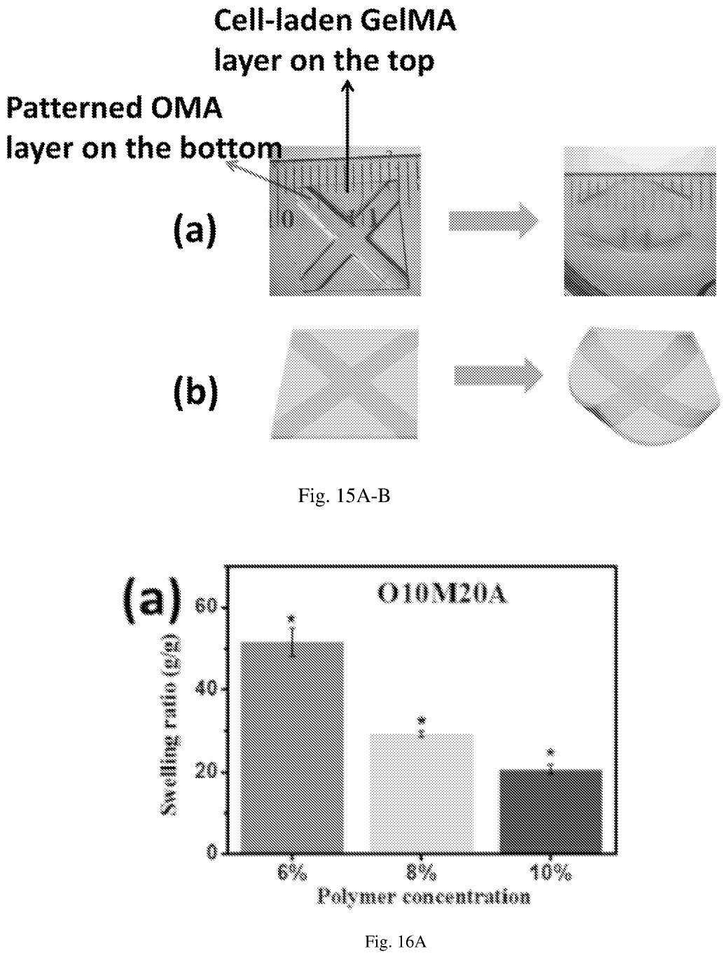

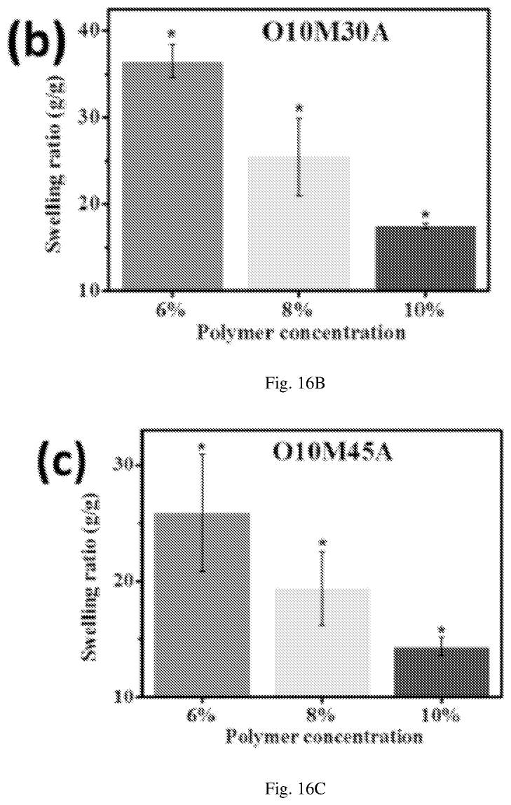

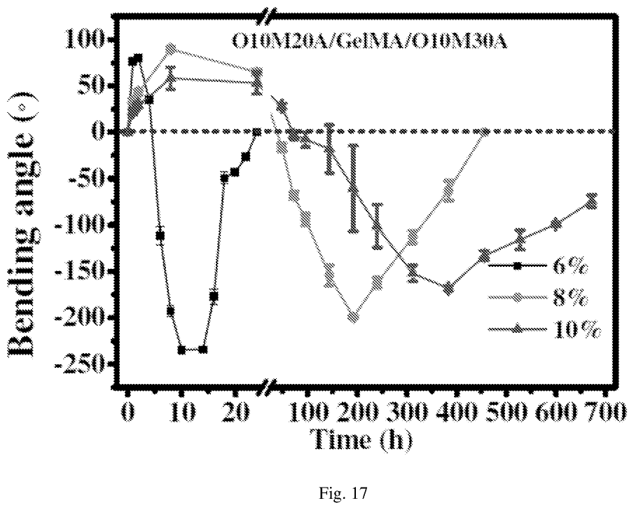

Currently, the majority of cell-laden scaffold-based tissue engineering strategies involve preparing, culturing and/or implanting a construct that does not change in shape over time, and providing instructive signals to the cells from the scaffold itself and/or the defect site to guide their differentiation and function. These geometrically static systems cannot directly contribute to the morphogenesis of complex tissue architectures. In addition to the drawbacks described earlier, most reported shape morphing hydrogel systems generally utilize differential swelling to evoke deformations in a short time frame. Therefore, they may be unsuitable for biomimicking native tissue developmental shape changes, where the shape evolution processes of tissue morphogenesis can occur over slow and/or fast time scales. Since our hydrogel actuators enable temporally controllable shape evolution in the presence of encapsulated cells, this system is well suited for morphodynamic tissue engineering applications. To slow down the shape morphing progress to a course of 3 or 4 weeks, the concentration of the OMAs was increased from 6% to 8%. The increased concentration diminished the hydrogel swelling (FIG. 16) and decelerated the degradation rate at the same time, resulting in protracted bending durations (FIG. 17).

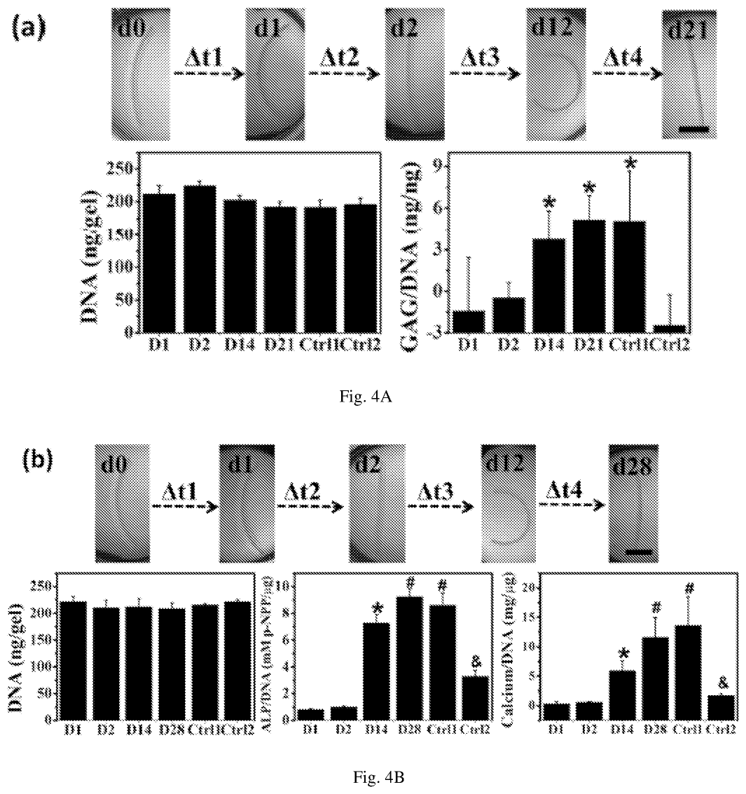

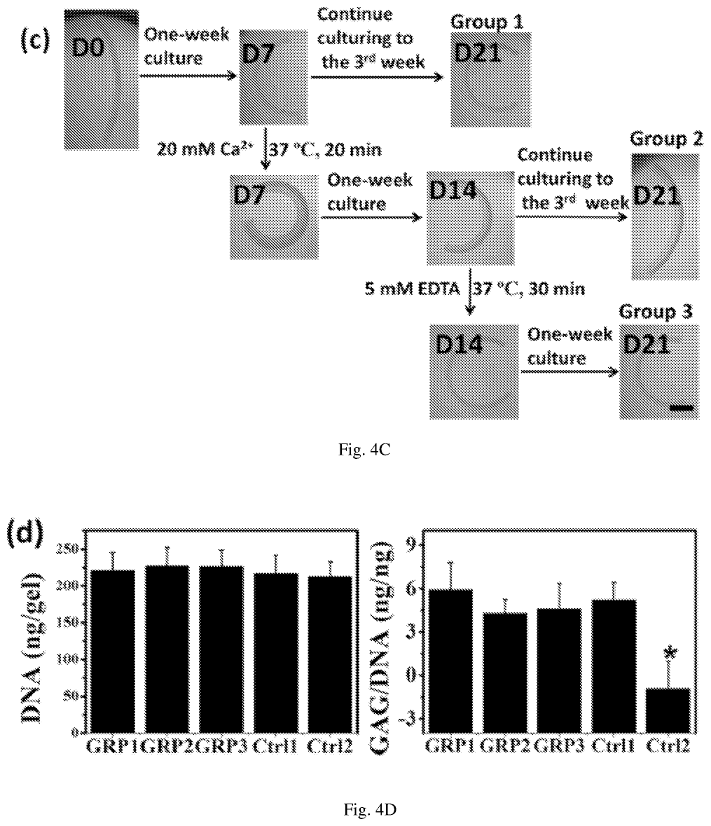

To demonstrate the capacity to control and change the shape of an engineered tissue during cell differentiation and new tissue formation, human mesenchymal stem cells (hMSCs) were seeded in the GelMA layer of the trilayer hydrogel bars (O10M20A/GelMA/O10M45A) and the constructs were cultured in chondrogenic and osteogenic media to induce chondrogenic and osteogenic differentiation of the cells, respectively (FIGS. 4A and 4B). In both conditions, the trilayer CHAs programmably exhibited the five-phase shape transitions during the culture period described previously (FIG. 1A). The live/dead staining results indicated that the encapsulated cells maintained high viability. Moreover, the production of a primary cartilage extracellular matrix component, glycosaminoglycan (GAG), during chondrogenesis and early and late osteogenic markers alkaline phosphatase (ALP) and calcium (Ca), respectively, during osteogenesis, all gradually increased over time and were significantly higher in comparison with the negative controls (trilayer hydrogel bars cultured in growth media at D21 or D28, Ctrl2). There was no statistical difference in DNA content between any of the groups or in differentiation marker expression between these experimental groups and positive controls (GelMA only hydrogel bar cultured in chondrogenic media at D21 or osteogenic media at D28, Ctrl1). These results indicate that the CHAs serving as a cell scaffold can change their shape in a programmable manner while not preventing encapsulated cell differentiation, and conversely cell differentiation did not prevent the hydrogel actuation.

To realize “on-demand” shape controllability of the CHA system during cell differentiation and tissue regeneration, hMSC-laden O10M45A/GelMA bilayer hydrogel bars derived from the O10M20A/GelMA/O10M45A trilayers after degradation of the O10M20A layer were cultured in chondrogenic media over 3 weeks. During culture in the differentiation media, shape changes with and without external stimulation over 3 weeks (21 days) were investigated (FIG. 4C). The O10M20A layer in the O10M20A/GelMA/O10M45A trilayer completely disappeared after two-day culture and the remaining bilayer further curled up to the GelMA side with continuously increasing curvature (group 1). The shape of the remaining bilayer could be tuned at any time point throughout the chondrogenesis process. For example, when the bilayer was treated with Ca²⁺ at week 1 (D7), this stimulus served to invert the orientation of the construct to curve toward the OMA side (group 2). This inversion could be reversed by EDTA stimulation at D14 (group 3). After treatment(s), the bilayers continued being cultured to D21. The inverted hydrogel (group 2) sustained its orientation despite some loss of the bending extent, and the recovered shape construct (group 3) stayed almost unchanged in its bending extent. Regardless of the external stimulation treatment, the cells remained highly viable (FIG. 4D) and similar DNA levels were detected in all groups at D21 (FIG. 4E). The production of the GAG was quantified to further assess the impact of the shape manipulation on chondrogenesis. The three experimental groups and the positive control group (GelMA only hydrogel bars cultured in chondrogenic media at D21, Ctrl1) exhibited similar amounts of GAG production to each other, and significantly more compared to the negative control group (GelMA/O10M45A bilayer hydrogel bars cultured in growth media at D21, Ctrl2). These results demonstrated that cells can be incorporated into the hydrogels and induced to differentiate, while at the same time external stimuli may be applied to provoke multi-stage shape morphing of the constructs in an “on-demand” manner. Alternative stimuli may be explored for a tissue such as bone, where Ca²⁺ production by cells might interfere with shape change and EDTA could impede osteogenesis.

In conclusion, this example demonstrated a potential strategy for “on-demand”, multiple, and reversible shape morphing hydrogels using multilayered OMA and GelMA hydrogels. The simplicity, convenience, and strong adaptability of the fabrication methods make it simple to manufacture hydrogel actuators with various complexities. The CHAs transform into specific 3D architectures and undergo diverse alterations in either pre-programmed and/or user-controlled manners with tunable phase durations. These CHAs can be designed to biomimic developmental and healing processes, such as branching morphogenesis, which may have great potential for constructing models of these processes and in tissue engineering applications. Such goals would be difficult to achieve with traditional systems due to limited deformation capacity, non-biocompatible and/or cytotoxic materials and fabrication techniques, and harsh shape changing activation environments. This system provides a powerful tool and broadens the bio-applications of shape morphing hydrogels to investigate the role of multiple and reversible pre-programmed and “on-demand” shape changes of extracellular matrix on cell behavior and tissue formation.

### Example 2

To address the aforementioned challenges with current 4D systems, we aimed to engineer a new 4D biomaterial platform endowed with the capacity to undergo tailored geometric changes over time while simultaneously permitting the incorporation of high densities of cells with maintained viability and functionality. To accomplish this, oxidized and meth-acrylated alginates (OMAs) with different degrees of oxidation and GelMA were synthesized to form the basis of bilayered hydrogels by attaching OMA and GelMA hydrogels via simple sequential crosslinking mediated by ultraviolet irradiation. We hypothesized that temporal differences in swelling rates between the two materials would drive structural deformations. Specifically, it was examined whether higher oxidation of OMA hydrogels would lead to greater swelling and shape change over time compared to the adjacent GelMA hydrogels, which possess relatively lower swelling properties. The roles of layer thickness, controlled by varying the thickness of spacers between glass plate molds, and the density of incorporated cells (i.e., NIH3T3 fibroblasts and human adipose tissue-derived stromal cells [ASCs]) on the system's 4D characteristics were also evaluated. NIH3T3 and ASCs were then incorporated into the 4D constructs at high densities of ≈1.0×10⁸ cells mL⁻¹ to determine the capacity for the system to maintain high cell viability. Organization and function of the high cell density constructs were assessed by investigating cell-cell and cell-ECM interactions and differentiation capabilities. Last, the feasibility of using the two cell-laden polymer components as bioink materials for bioprinting 4D high cell density constructs with complex initial and preprogrammed final geometries was explored. This example describes a new, versatile 4D high cell density tissue condensation system based on biodegradable and cytocompatible materials enabling precise control of geometric change over time and development of functional 4D tissue constructs.

### Methods

### Experimental Section

### Synthesis of OMA and GelMA

To synthesize OMA, 1% sodium alginate (10 g, Protanal LF 20/40, FMC Biopolymer) solution was dissolved in ultrapure deionized water (diH₂O, 900 ml) by stirring overnight at room temperature (RT). Either 1 or 1.5 g of sodium periodate were respectively dissolved in 100 ml of diH₂O, mixed with the alginate solution to achieve 10% and 15% theoretical alginate oxidation and reacted in the dark at RT for 24 hrs under stirring.

2-morpholinoethanesulfonic acid (MES, 19.52 g, Sigma) and NaCl (17.53 g) were then dissolved in the oxidized alginate solution and the pH was adjusted to 6.5 using 4 N NaOH.

N-hydroxysuccinimide (NHS, 1.176 g, Sigma) and 1-ethyl-3-(3-dimethylaminopropyl)-carbodiimide hydrochloride (EDC, 3.888 g, Sigma) were dissolved into the mixture. AEMA (1.688 g, Polysciences) (molar ratio of NHS:EDC:AEMA=1:2:1) was then slowly added to the solution to achieve a theoretical methacrylation level of 20%. The reaction was conducted at RT for 24 hrs in the dark. The reacted OMA solution then was poured into excess acetone to precipitate the OMA. The precipitate was dried in a fume hood and subsequently dissolved in diH₂O at a 1% w/v concentration. The OMA solution was dialyzed for purification using a dialysis membrane (MWCO 3500, Spectrum Laboratories Inc.) for 3 days. The dialyzed OMA solution was collected and treated with activated charcoal (5 g/L, 50-200 mesh, Fisher) for 30 min. The solution was further purified and sterilized by filtering through a 0.22 μm pore membrane and then lyophilized.

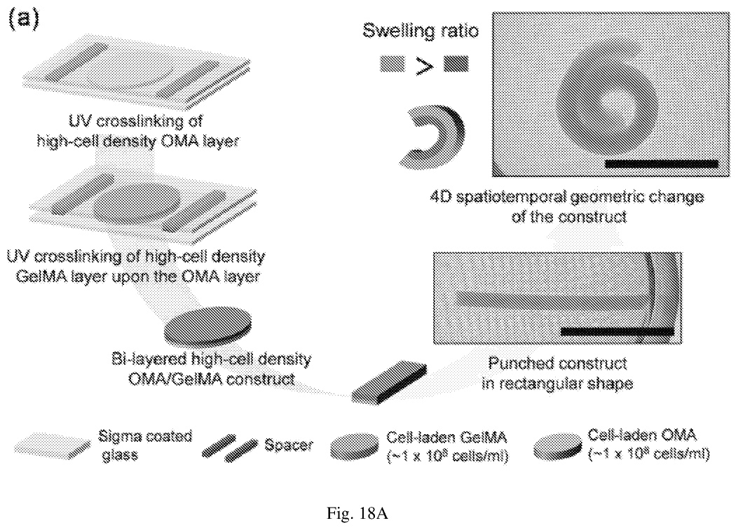

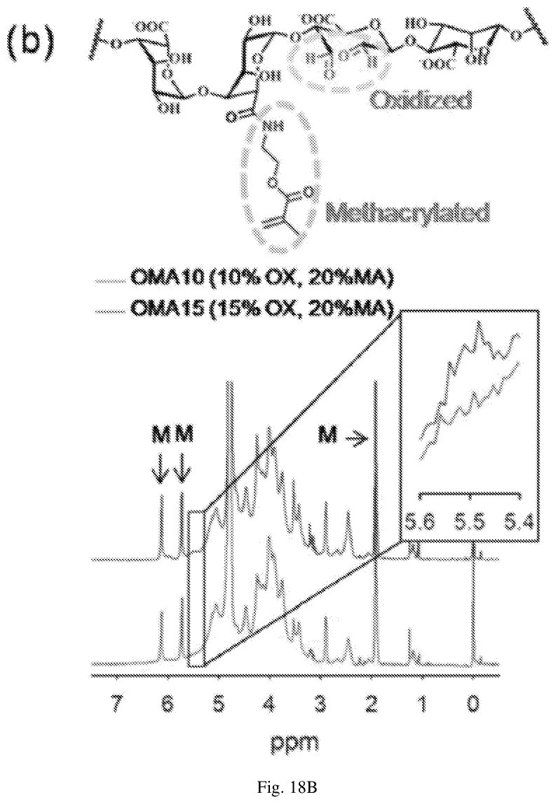

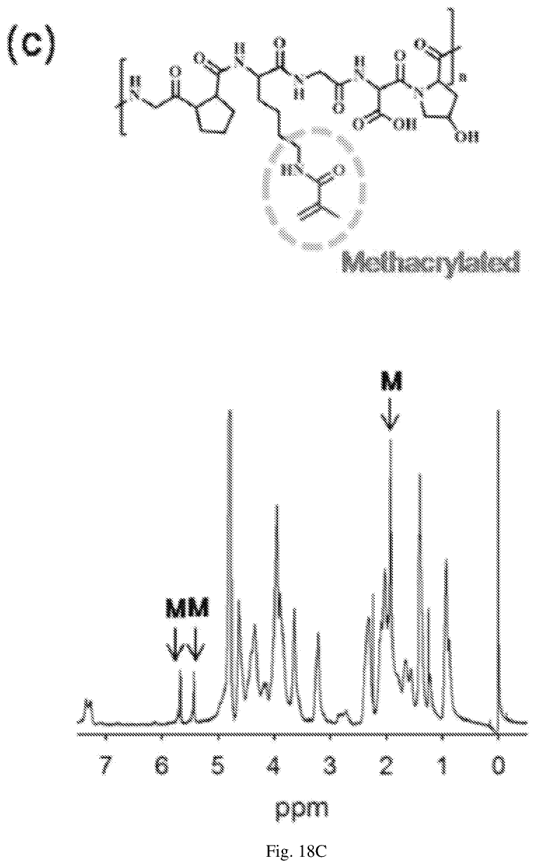

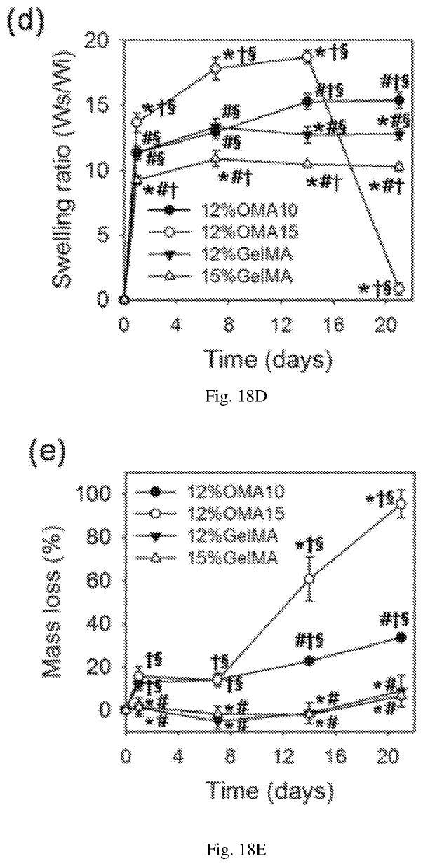

To synthesize GelMA, 10 g of gelatin (type A, Sigma Aldrich) was dissolved in 100 ml of PBS (pH 7.4) and heated to 50° C. Then 10 ml of methacrylic anhydride was added into the 10% gelatin solution and reacted for 1 hr at 50° C. and then stirred overnight at RT. GelMA was precipitated with acetone, purified via dialysis at 50° C. for 7 days with a MWCO 12-14 k membrane (Spectrum Laboratories Inc.), sterilized via a 0.22 mm pore filter, and then lyophilized. To obtain ¹H-NMR spectra, the OMA and GelMA were separately dissolved in deuterium oxide (D₂O) at 2 w/v % and the samples were analyzed via ¹H-NMR spectrometer (Varian Unity-300 (300 MHz) NMR spectrometer (Varian Inc.)). 3-(trimethylsilyl)propionic acid-d₄ sodium salt (0.05 w/v %) was used as an internal standard. The actual methacrylation of the OMA and GelMA was determined from ¹H NMR spectra based on the ratio of the integrals for the internal standard protons to the methyl and methylene protons of methacrylate (indicated as M, FIGS. 18B and C).

### Cell Isolation and Expansion Culture

NIH3T3s were cultured and in NIH3T3 growth medium (10% FBS, 1% PS in HG-DMEM). ASCs were obtained from the adipose tissue using a previously reported method. Briefly, lipoaspirates were treated with 200 U/mg collagenase type I (Worthington Biochemical Products, Lakewood, NJ) digestion for 40 min at 37° C. The stromal fraction was then isolated though centrifugation and plated and cultured on tissue culture plastic (TCP) in DMEM/nutrient mixture F12 (DMEM/F12, BioWhittaker, Suwance, GA) with 10% defined fetal bovine serum (FBS, HyClone, Logan, UT), 100 U/ml penicillin and 100 mg/ml streptomycin (1% P/S, BioWhittaker). Cell culture was conducted under standard conditions (95% humidity, 5% CO₂, 37° C.). The medium was changed every 2 days. Cells were detached from TCP using 0.05% of trypsin/EDTA for passage and experimentation. Passage 3 ASCs were used in this study.

### Fabrication of Bi-Layered OMA/GelMA Hydrogel as a Model Construct

OMA and GelMA were separately dissolved at multiple different concentrations in HG-DMEM containing 0.05% photoinitiator (PI, Irgacure-2959). For cell incorporation, NIH3T3s or ASCs were collected via standard trypsinization, and pre-determined numbers of cells were collected after cell counting and centrifugation. Cell pellets were dissociated into OMA or GelMA solutions at designated densities of 2.0×10⁷, 5.0×10⁷ and/or 1.0×10⁸ cells/ml. The OMA solution (with or without cells) was first cured under UV light (320-500 nm, EXFO OmniCure S1000-1B, Lumen Dynamics Group, Mississauga, Ontario, Canada) at 20 mW/cm² for 45 sec between hydrophobic glass plates treated with Sigmacote (Sigma Aldrich) separated with 0.2 or 0.4 mm thick spacers. GelMA solution was then cured under UV at 20 mW/cm² for 45 sec on top of the OMA hydrogel between glass plates with 0.6 or 0.8 mm gaps controlled by assembling 0.2 mm spacers. The dual-layered hydrogel was punched into rectangular shapes (width=2.8 mm, length=12.7 mm). To generate OMA patterns in the 4D constructs, GelMA was cured first by UV irradiation then OMA was selectively cured later using a photomask.

### Culture of Cell-Laden 4D Constructs

4D constructs containing NIH3T3s were cultured in NIH3T3 growth media. 4D constructs containing ASCs were cultured in ASC growth media or differentiation media. Chondrogenic differentiation of 4D ASC constructs was conducted in a media composed of 1% ITS+ Premix (Corning), 100 nM dexamethasone (MP Biomedicals), 37.5 μg/ml L-ascorbic acid-2-phosphate (Wako USA), 1 mM sodium pyruvate (Hyclone), 100 μM nonessential amino acids (Hyclone), and 10 ng/ml TGF-β1 (Peprotech) in DMEM-high glucose. For osteogenic differentiation, 10 mM β-glycerophosphate (CalBiochem), 50 mM ascorbic acid (Wako USA), 100 nM dexamethasone (MP Biomedicals), and 100 ng/ml BMP-2 (Perkin-Elmer) in DMEM-high glucose containing 10% FBS and 1% PS was used.

### Angle Measurement of 4D Model Constructs

Photographic images (Galaxy Note 10, Samsung, South Korea) of the model constructs were obtained at different time points. Using Image J software, the center and two end points were determined from a bird's-eye view and two lines starting from the center extending to each end were drawn. The angle between the two lines was measured to determine degree of the geometric change.

For example, 0° indicates no shape change, 180° denotes a closed circle, and negative values imply a rolled structure beyond a complete circle.

### Swelling Ratio and Degradation Test of the OMA and GelMA Hydrogels

Hydrogels were fabricated into the shape of 0.8 mm diameter, 0.75 mm thick discs by placing polymer solution between a quartz and regular glass plate separated by spaces and crosslinking through the quartz plate via UV light at 20 mW/cm² for 45 sec and using a biopsy punch. The samples were lyophilized to obtain their initial dry weights (Wi). The lyophilized samples were then immersed in HG-DMEM containing 10% FBS and 1% PS and incubated at 37° C. The media was replaced every other day. Weight of the swollen hydrogels was measured (Ws) and used to calculate the swelling ratio Ws/Wi (n=4) for each condition per time point. After the measurement, the samples were lyophilized to obtain dry weight (Wd). Mass loss was quantified as (Wi−Wd)/Wi×100 (n=3) for each condition per time point.

### Live/Dead Staining

Cell-laden 4D constructs were treated with Live/Dead staining solution (0.1% Calcein AM and 0.2% Propidium Iodide in PBS) for 15 min. Fluorescence images were obtained via an ImageXpress PICO (Molecular Devices, CA, USA) with 40× magnification.

### Biochemical Assays

4D high cell density constructs were digested in 1 ml papain buffer solution (25 mg/ml papain (Sigma); 2 mM L-cysteine (Sigma); 50 mM sodium phosphate (Sigma); 2 mM ethylenediaminetetraaceticacid (EDTA) (Fisher); pH 6.5 in nuclease-free water (Ambion, Austin, TX) at 65° C. overnight. The samples were then centrifuged at 16200 g for 10 min. 100 μl of supernatant was collected in a 96 well plate then treated with 100 μl of PicoGreen® reagent (Invitrogen, Carlsbad, CA). Fluorescence intensity was then measured on a microplate reader (SpectraMax®, Molecular Devices, CA, USA) at 520 nm (excitation at 480 nm). For GAG quantification, 100 μl of dimethylmethylene blue dye solution was added to 40 μl of the digested supernatant. Absorbance values were measured on the microplate reader at 595 nm. ALP assay was conducted by adding 100 μl of ALP yellow substrate (Sigma) to 100 μl of the digested supernatant. The mixture was incubated at 37° C. for 30 min and then a reaction stop solution (50 μl of 0.1N NaOH) was added. Absorbance at 405 nm was measured on the microplate reader.

### Histology

The 4D high cell density constructs were fixed in 4% neutral buffered formalin and embedded in paraffin. 5 μm thickness sections were loaded on slide glasses. After deparaffinization, sections were stained with Toluidine Blue O or Alizarin Red S for 5 min to visualize GAG and calcium, respectively. For hematoxylin and eosin (H&E) staining, sections were treated with these stains for 10 and 2 min, respectively.

### Live Cell Labeling

Pre-labeling of NIH3T3 was conducted by treating the cells with 0.5% Vybrant™ DiD (purple fluorescence dye) (Invitrogen) or 0.5% Vybrant™ DiO (green fluorescence dye) (Invitrogen) in culture medium for 1 hr.

### 3D Bioprinting

The gelatin slurry for supporting bath was prepared as described previously. NIH3T3 laden 12% OMA15 (at 1.0×10⁸ cells/ml) and 12% GelMA (at 1.0×10⁷ cells/ml) were loaded on separate extruders on a Biobot™ Basic 3D printer (Advanced Solutions Life Sciences, KY, USA) with ½ inch stainless metal 25G needles (McMaster-Carr). A gelatin slurry bath filled petri dish was placed on the printer platform, and then a CAD file was used to print a 2×2 bilayered checkerboard construct with total dimensions of 2 cm×2 cm×0.4 cm. The printed 4D construct was immersed in HG-DMEM media then moved to a 37° C. incubator to dissolve out the gelatin slurry and further culture in NIH3T3 growth media. After 30 min and 7 days of culture, optical images were obtained using digital camera (Galaxy Note 10, Samsung).

### Statistical Analysis

All quantitative data is presented as mean±standard deviation. Statistical analysis was conducted via one-way analysis of variance (ANOVA) with Tukey significant difference post hoc test using Graphpad Prism 5 software (GraphPad Software, San Diego, CA). A value of p<0.05 indicates statistical significance.

### Results

4D tissue engineering may be a promising technology to partially recapitulate the controlled, programmed geometric reorganization of developing and healing tissues while synchronizing with growth and shape changes of surrounding tissues. By recreating the spatiotemporal changes during native development and repair processes, it also may find utility in the development of artificial tissue models for drug screening. In this study, we exploited the differential swelling of OMA and GelMA, which are widely used biocompatible, biodegradable hydrogel materials, to form constructs that can change their geometry over time when placed in aqueous solutions. Importantly, 4D high cell density constructs were fabricated by incorporating cells into the photocrosslinkable OMA/GelMA hydrogels at high densities of up to 1.0±10⁸ cells mL⁻¹. Bilayered OMA/GelMA hydrogel constructs were prepared via sequential photocrosslinking (FIG. 18A). Rectangular shaped punches of the hydrogels were used as model constructs to assess 4D spatiotemporal geometric changes over time. We hypothesized that the OMA layer could be designed to exhibit greater swelling than the GelMA layer, and thus drive construct shape changes, due to its greater expansion by water absorption and easily tunable degradation rate. The degree of expansion of the OMA layer was controlled by extent of theoretical oxidation. Additionally, the effects of hydrogel layer thickness, macromer concentration, and density of incorporated cells on the degree of shape change of the model constructs were assessed, and ultimately fabrication of 4D high cell density tissues with defined spatiotemporal geometric change properties were evaluated.

¹H-NMR analysis was performed to characterize the OMA and GelMA macromer materials. It was determined that the 15% oxidized OMA (OMA15) exhibited greater intensity at 5.5 ppm compared to the 10% oxidized OMA (OMA10), indicating control over alginate oxidation rate (FIG. 18B). Efficient methacrylation of the OMA and GelMA (FIG. 18C) was demonstrated by recognizable peaks labeled as M (OMA at 6.2, 5.7, and 1.9 ppm and GelMA at 5.6, 5.4, and 1.9 ppm), with actual MA rates of OMA10, OMA15, and GelMA being 10.1%, 12.5%, and 97.4%, respectively. The swelling ratios of OMA10, OMA15, and GelMA hydrogels were then measured over time (FIG. 18D). At the same macromer concentration of 12%, OMA15 hydrogels presented the highest swelling ratio, 18.7±0.5, at the 14th day of incubation and then dramatically decreased at the 21st day due to rapid degradation (FIG. 18E). 12% OMA10 and 12% GelMA presented similar values through 7 days, but 12% OMA10 exhibited greater values at 14th- and 21st-day (15.2±0.6 and 15.4±0.6) than those of 12% GelMA. The 15% GelMA hydrogel presented the lowest values for all time points except at the 21st day. To measure degradability in serum containing high glucose Dulbecco's Modified Eagle's Medium (HG-DMEM) over time, the mass loss of the hydrogels was measured. OMA hydrogels displayed significantly faster degradation than the GelMA hydrogels. OMA15 presented similar mass loss as OMA10 after 7 days, and then underwent more rapid degradation by days 14 to 21 with values of 60.6±10.2 and 95.3±6.5%, respectively. This finding sup-ports the more rapid and extensive swelling observed with the OMA15 compared to the OMA10. However, repeated lyophilization and manipulation of the hydrogels while measuring weight may have played a role in accelerating the mass loss of the OMA15 hydrogels. The GelMA hydrogels exhibited minimal degradation after 14 days, and then gradually lost mass by 21 days. Oxidation cleaves C—C bonds of the cis-diol groups of the alginate uronate residues and converts them to dialdehyde groups, making the alginate more susceptible to degradation via hydrolysis. Ester bonds generated by photocrosslinking of the methacrylated polymers are hydrolysable as well. Although GelMA exhibits little degradation in DMEM, its degradation can be accelerated by incorporation of cells due to proteolysis mediated by cell-secreted MMPs. Since it was anticipated that increased shape change would occur in hydrogel bilayers with greater differences in swelling ratios between the OMA and GelMA, bilayers of 12% OMA15 and 15% GelMA were expected to exhibit the most extensive change in shape of the conditions examined.

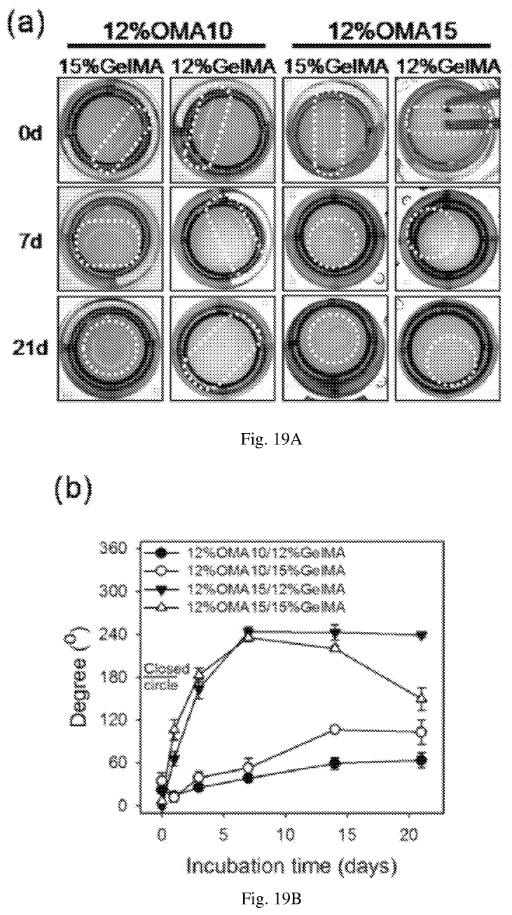

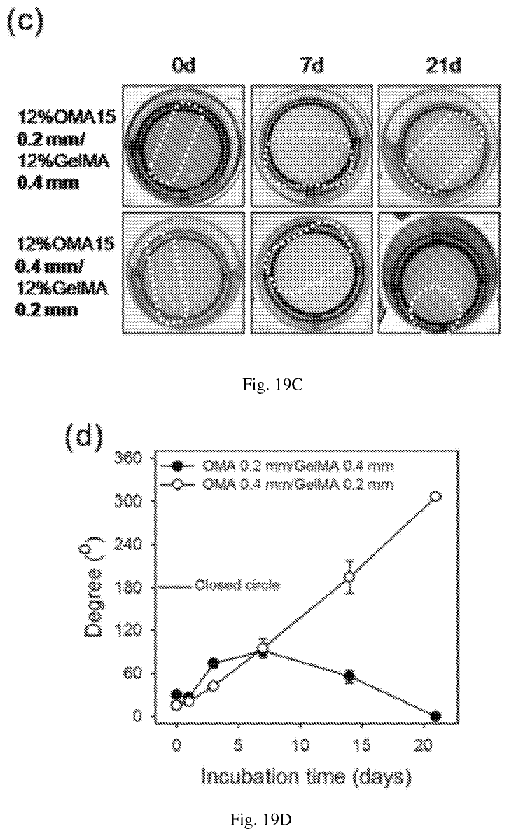

Bilayered OMA/GelMA hydrogels were fabricated by sequential photocrosslinking to determine the effect of swelling ratio on degree of shape change by observing rectangular hydrogels (width: 2.8 mm, length: 12.7 mm, and thickness: 0.4 mm [OMA 0.2 mm+GelMA 0.2 mm]) incubated in NIH3T3 fibroblast culture media for 21 days at 37° C. Shape change was quantified by measuring the angle between two lines respectively drawn from bird's-eye view by connecting the center point of the longest dimension of the construct and each end point of the construct. Briefly, a higher value indicates greater shape change with a flat construct in its original shape having an angle of 0°, a construct forming a closed circle measuring at 180°, and a rolled structure where the end points pass each other having a >180° value. All the hydrogels were flat at day 0 and curved into a “C” shape or rolled structure over time (FIG. 19A, B). As hypothesized, hydrogels composed of an OMA15 layer exhibited greater degrees of shape change when compared to those containing OMA10. Interestingly, the 12% OMA15/12% GelMA and 12% OMA15/15% GelMA constructs both rapidly attained a rolled structure by 7 days. However, only the 12% OMA15/12% GelMA maintained the rolled structure through 21 days (239±3°). 8% OMA15/12% and 15% GelMA constructs were also examined, but the 8% OMA15 constructs were unable to undergo strong shape changes, possibly due to decreased water absorption as compared to the 12% OMA15. The effects of increasing the thickness of either the OMA or GelMA layer in the 12% OMA15/12% GelMA constructs was then investigated. Total thickness of bilayered constructs was set to 0.6 mm, while thicknesses of OMA/GelMA were varied as 0.2/0.4 and 0.4/0.2 mm, respectively, through the use of spacers. It was observed that a 0.4 mm OMA layer induced greater shape change as compared to a 0.2 mm OMA layer (FIG. 19C). Remarkably, an increase in OMA thickness from 0.2 to 0.4 mm resulted in continuously increasing shape change over 21 days, reaching a curvature of 306±1° (FIG. 19D). Conversely, decreasing OMA thickness to 0.2 mm while increasing the GelMA thickness to 0.4 mm induced minimal geometric change after 7 days, which was not maintained beyond this time point. Therefore, by varying the design parameters (i.e., macromer concentration, degree of alginate oxidation, and layer thickness) of degradable bilayered OMA/GelMA constructs with potential for high cell density encapsulation and condensation, we have gained an under-standing of the underlying principles that regulate their 4D geometric change. Exquisite control of these shape changes over a wide range of deformations may enable mimicry of varied tissue structures from the gentle curvature of the cornea to the coiled cochlea.

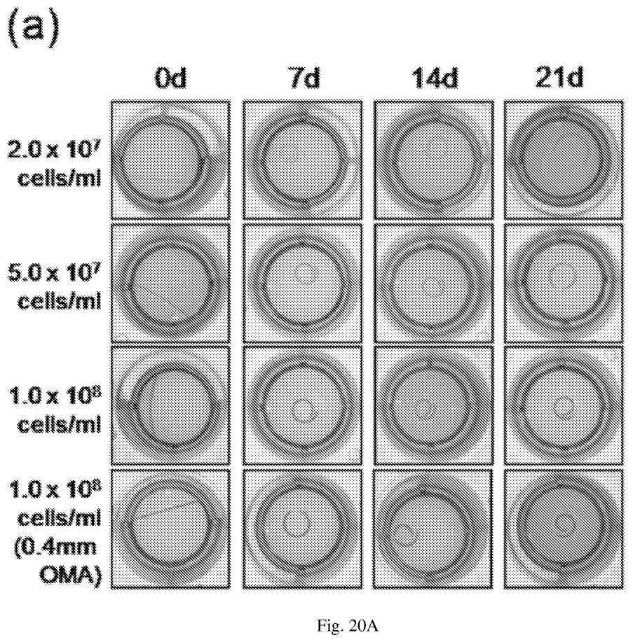

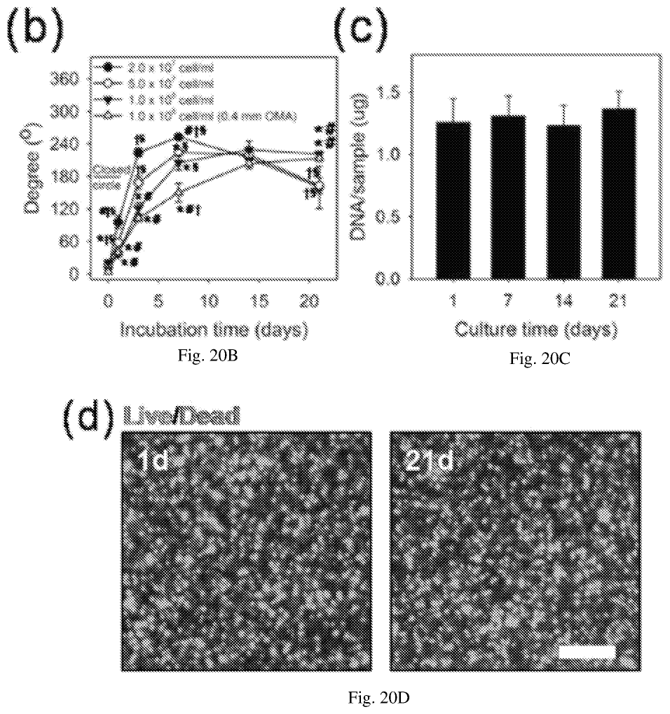

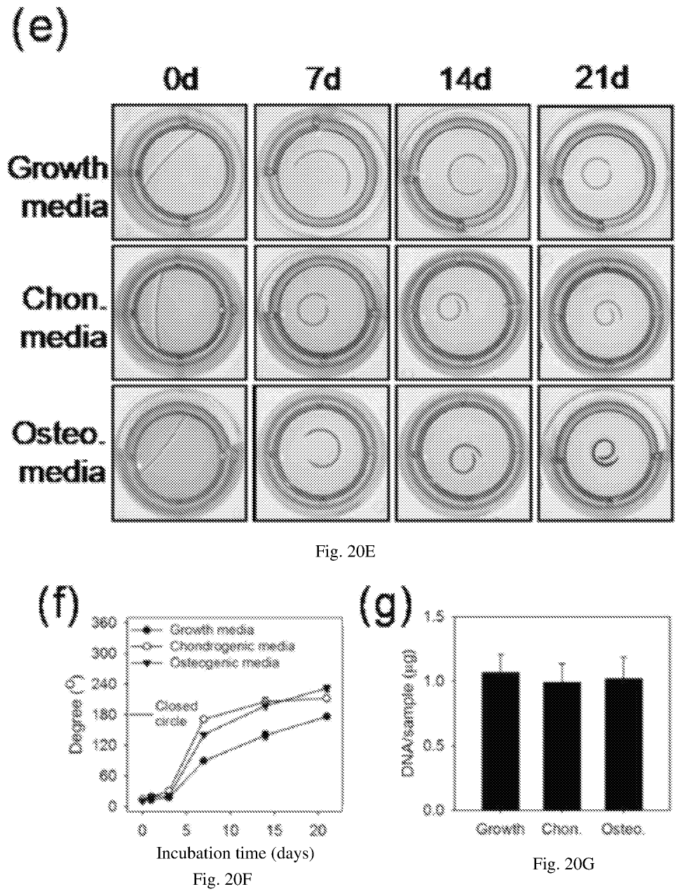

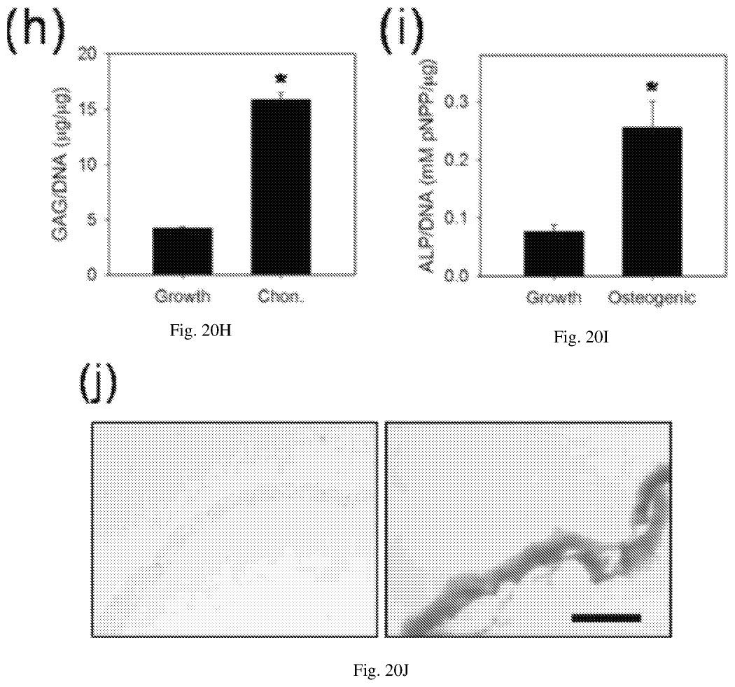

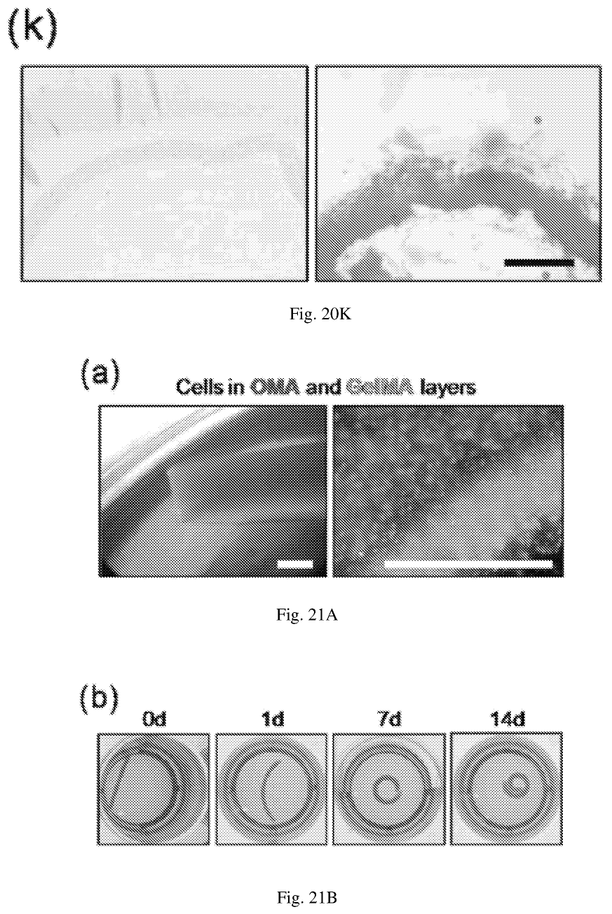

Although studies regarding 4D biomaterials accompanied with cell encapsulation have reported time dependent shape change, they have been limited in immediate or very short term geometric changes. For example, in the case of alginate and hyaluronic acid hydrogels with gradient crosslinking and bilayered PEG hydrogels, focus was only on the time scale of seconds and minutes without precise temporal control over 4D shape changes on the time scale of days and weeks that is required for healing and development processes of tissue and that can be achieved with the OMA/GelMA system. In addition, there has not previously been a system that permits encapsulation of a high density of viable cells in biocompatible and biodegradable materials with predesigned geometric changes over time. It was expected that the bilayered constructs composed of cell friendly, biodegradable OMA/GelMA would enable cells to be incorporated at a high density. The shape-changing feature of the cell containing 4D biodegradable constructs was not anticipated to be affected by the high cell density encapsulation. The GelMA, synthesized from gelatin that is denatured collagen and has important biochemical functionality, was anticipated to provide a favorable environment to the incorporated cells by presenting cell adhesive moieties that are not present in non-degradable synthetic polymers. To assess the ability to incorporate high cell density concentrations without affecting subsequent 4D geometric changes, NIH3T3 cells were incorporated in a 12% GelMA layer with varied densities of 2.0×10⁷, 5.0×10⁷, and 1.0×10⁸ cells mL⁻¹. A 0.2 mm layer of 12% OMA15 hydrogel was used to induce geometric change. Increase in the cell density could be visualized using phase contrast microscopy from a side view. For the 1.0×10⁸ cells mL⁻¹ condition, a 0.4 mm OMA15 layer was additionally applied in efforts to induce slow but longer lasting shape change for long term culture periods (FIG. 20A). As quantified in FIG. 20B, the 2.0×10⁷ and 5.0×10⁷ cells mL⁻¹ conditions showed more extensive geometric changes than 1.0×10⁸ cells mL⁻¹ condition at 3rd- and 7th-day with statistical significance, but lower cell concentration conditions were unable to maintain their maximal rolled structures at 21st day. Interestingly, the 1.0×10⁸ cells mL⁻¹ condition with a thinner OMA layer exhibited a slower shape change profile but steadily increased and maintained the rolled structure until 21 days (221±6°) (FIG. 20B). The highest cell density with a thicker OMA layer displayed slower shape changes, but also successfully induced a rolled structure similar to that of its thinner construct counterpart during 21 days of culture (213±5°). To the best of our knowledge, 1.0×10⁸ cells mL⁻¹ is the highest density to ever be reported in a shape-changing material, which is 20 times greater than the highest previous reported concentration, 5.0×10⁶ cells mL⁻¹. It may not be possible to incorporate higher concentrations of cells in systems that rely on gradient crosslinking through light irradiation as the increase in cell density in the macromer solutions can inhibit light penetration and hinder gradient crosslinking generation. The ability to use a wide range of cell densities shown in this study is beneficial for engineering different target tissues that have varied cell concentrations. A DNA assay conducted on samples from the 1.0×10⁸ cells mL⁻¹ and 0.4 mm OMA layer condition revealed maintenance of DNA content, about 1.2 g/sample over 21 days, indicating viable cells within the constructs (FIG. 20C). Live/dead stained images of the constructs obtained at the 1^{st}- and 21^{st}-day showed predominantly live (green) fluorescence evidence of high cell viability in the 4D constructs (FIG. 20D).

ASCs were then incorporated into the constructs at a density of 1.0×10⁸ cells mL⁻¹ into a 0.2 mm 12% GelMA layer and 0.4 mm 12% OMA15 was additionally layered. To assess potential to drive cellular differentiation simultaneously during the induction of 4D material shape change, the samples were incubated in three different media: growth, chondrogenic, and osteogenic (FIG. 20E). Similar to the NIH3T3 cells, it was observed that every group presented steady increases in degree of geometric change after 3 days of culture (FIG. 20F). Samples cultured in control growth media formed a closed circle, while the experimental groups formed even more fully rolled structures beyond a complete circle by 21 days (232±7° and 212±5° for osteogenic and chondrogenic differentiation conditions, respectively). The incorporated ASCs presented high viability regardless of culture condition or time period. DNA content of the constructs presented similar levels without significant differences, near 1.0 g/sample (FIG. 20G). However, significant increases in GAG/DNA and ALP activity/DNA were observed between the growth media condition and chondrogenic and osteogenic differentiation media conditions, respectively (FIGS. 20H, I). Positive histologic staining for GAG production and calcium deposition in the samples cultured in differentiation media corroborated the biochemical findings, demonstrating the capacity to differentiate encapsulated stem cells down specific connective tissue lineages in the 4D material while also achieving controlled changes in construct shape (FIGS. 20J, K). Previous reports with encapsulated cells in bio-degradable 4D materials have mainly investigated geometric changes without focusing on cellular behaviors. In this study, a range of cellular activities and functions have been investigated, including viability, proliferation, and differentiation.

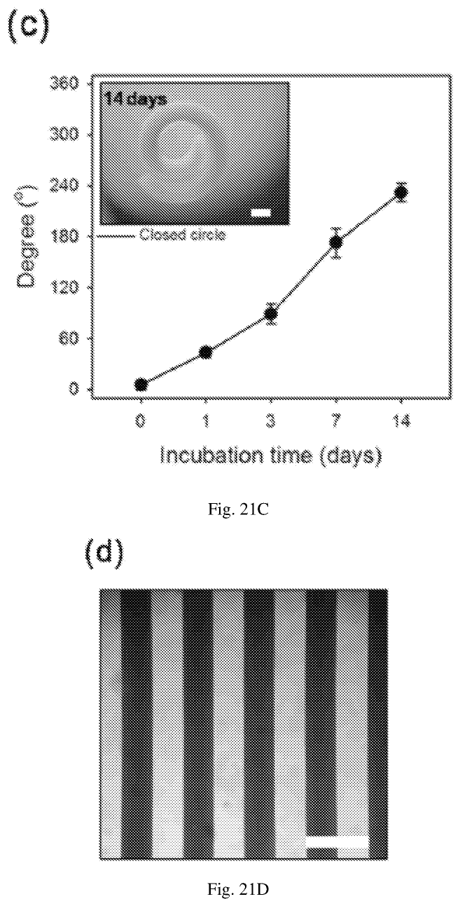

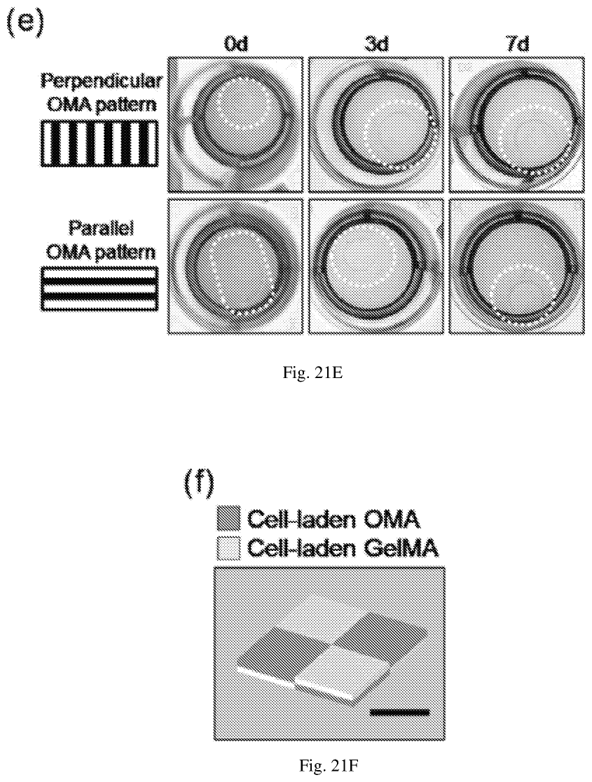

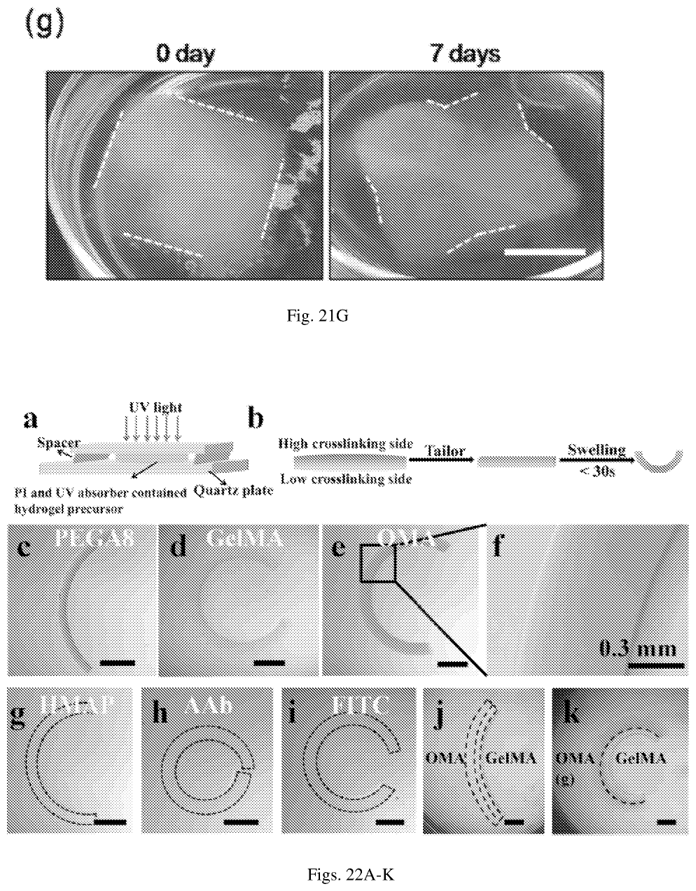

To demonstrate the potential to generate high cell density 4D tissue constructs comprised of two physically separated cell populations, NIH3T3 cells were further incorporated into both the GelMA and OMA15 layers (cell density=1×10⁸ cells mL⁻¹); 12% OMA15 with 0.4 mm thickness and 12% GelMA with 0.2 mm thickness. It was clearly apparent that the cells were incorporated into both layers of the high cell density 4D constructs via phase contrast and fluorescence imaging (FIG. 21A), and H&E staining. NIH3T3 cells labeled with violet and green dyes and incorporated in GelMA and OMA layers, respectively, can be seen in their respective layers in fluorescence photomicrographs. Incorporation of at least two different cell types in separate distinct compartments is also possible via this sequential bilayer crosslinking system, which is not achievable in platforms relying on gradient crosslinking of a single component. This capability may be advantageous in efforts toward building 4D high cell density constructs recapitulating complex native tissues composed of multiple cell types. The dual cell incorporated samples presented a similar degree of geometric change (232±10°) at the 14th day (FIG. 21B,C) compared to 4D materials containing cells only in the GelMA layer at a density of 1×10⁸ cells mL⁻¹ (FIG. 20A, B).

To investigate additional capacity to further control 4D construct shape change via photolithography, an OMA layer comprised of evenly spaced 250 μm wide strips (FIG. 21D) “parallel” or “perpendicular” to the longest dimension of the model construct was selectively added to the GelMA layer using a photomask. Generation of shape-morphing 12% OMA15/12% GelMA (0.2 mm/0.2 mm in thickness) bilayered regions with different directionality of the OMA strips was expected to control degree of the self-rolling. The perpendicular pattern group showed rapid rolling immediately after the fabrication (at 0th day) and formed and maintained a “C” shape over 7 days of culture (FIG. 21E). The parallel pattern group exhibited less shape change immediately after fabrication (at 0th day) and presented a different time course of curved structure formation from that of perpendicular pattern group, but also underwent geometric change into a “C” shape. These results highlight additional system capabilities for guided geometric shape change by simple application of photomasks with varied patterns and directions.

Last, the capabilities of the system performance were investigated in conjunction with bioprinting constructs possessing more complex structures using a 3D bioprinter (FIG. 21F, G). To demonstrate capacity to simultaneously modulate multiple bio-printing system parameters such as type of ink material and cell density, cells were incorporated at densities of 1.0×10⁸ and 1.0×10⁷ cells mL⁻¹ in the 12% OMA15 and 12% GelMA solutions, respectively. The inks were printed by adapting the free form reversible embedding of suspended hydrogels (FRESH) technique using a multinozzle printer with assistance of a gelatin microgel slurry bath. This printing system was used to produce a 2×2 bilayered checkerboard construct with total dimensions of 2 cm×2 cm×0.4 cm. Each small cube of the cell-laden OMA and cell-laden GelMA, indicated with red and white, respectively, in FIG. 21F, has a dimension of 1 cm×1 cm×0.2 cm. After removing the gelatin slurry by incubating at 37° C. for 1 h, the printed 4D construct reflected the original CAD file (FIG. 21G). Although the cube did not show any curved or angular structures at the borders of the OMA and GelMA regions in the checkerboard pattern at 0th day, after 7 days the cultured construct exhibited slight curvature in these areas as indicated with white dashed lines. Patterning cell-laden inks with different swelling ratio in this system has great potential for creating 4D high cell density constructs with complicated structural hierarchy. To our best knowledge, this is the first study demonstrating successful printing of cytocompatible and biodegradable 4D constructs containing cells in high density to achieve controlled geometric shape changes by thoughtful design and selection of the system parameters.

In summary, we have developed novel 4D high cell density constructs which undergo controlled temporal changes in geometry that are regulated by tailored spatial patterning of biodegradable OMA and GelMA materials with differential swelling ratios. By exploiting the simple model construct of bilayered OMA/GelMA hydrogels, it was demonstrated that higher oxidation extent of the OMA, which increases its degradation rate and swelling, facilitates greater geometric shape changes. Multiple construct parameters such as macromer percentage, layer thickness, cell density, and patterns generated by photolithography were varied to precisely program the final geometry of the 4D construct. In addition, high cell density encapsulation in biomaterial-based 4D constructs (1.0×10⁸ cells mL⁻¹) was achieved to mimic cellular condensations in native tissues. This technique supported normal cellular activities such as differentiation down toward osteogenic and chondrogenic lineages with minimal apparent adverse effects on viability. The photocrosslinking-based system permits building of complex tissues with multiple cellular components via specific spatial placement of different cell types. Bioprinting the cell-laden OMA and GelMA inks confirmed the ability to print 4D responsive high cell density constructs with complex geometries. Importantly, this strategy may be more broadly applied using other commonly used biodegradable materials with differential swelling ratios. This study presents a paradigm changing platform technology that has the potential to significantly impact 4D tissue-engineered therapeutics for treatment of damaged tissues, investigation of questions in developmental biology, and formation of tissue models for drug testing.

### Example 3

In this example, we demonstrate a simple and versatile approach to generate a tunable and robust gradient in a single-component biocompatible hydrogel for 4D biofabrication through one-step photocrosslinking of a biopolymer solution containing a photoinitiator (PI) and a cytocompatible UV absorber. The instant generation of graded scaffolds was demonstrated using various biocompatible hydrogels, such as those fabricated from 8-arm PEG-acrylate (PEGA8), oxidized and methacrylated alginate (OMA), and methacrylated gelatin (GelMA). The tunable crosslinking gradient was easily attained by adjusting fabrication parameters such as polymer concentration, UV absorber concentration, UV irradiation time, and hydrogel thickness, enabling pre-programmable hydrogel deformation. Importantly, multiple cell types (i.e., fibroblasts, stem cells, and cancer cells) were encapsulated into the graded scaffolds with high viability. This simple and cytocompatible strategy permits easy and fast fabrication of cell-laden hydrogel scaffolds with complex structures, which was demonstrated by harnessing several representative techniques, including photomask-aided microfabrication, photomask-based photolithography, ion transfer printing (ITP), and 3D bioprinting. For the proof-of-concept, 4D bone tissue engineering was ultimately explored using this platform system.

### Materials and Methods

### Hydrogel Fabrication

A mixed solution of polymer (OMA 6% w/v, GelMA 14% w/v, or PEGA8 20% w/v), P1 (0.05% w/v), and UV absorber [methacryloxyethyl thiocarbamoyl rhodamine B (RhB), 4-aminoazobenzene (AAb), fluorescein isothiocyanate derivatives (FITC), and/or 4′-hydroxy-3′-meth-ylacetophenone (HMAP)] in Dulbecco's modified eagle medium-low glucose (DMEM-LG) in the absence/presence of cells (hMSC, NIH3T3, or HeLa, 4×10⁶ cells/mL) was placed between two quartz plates with a 0.6 mm spacer and subsequently photocrosslinked with UV light (EXFO OmnicureR S1000, Lumen Dynamics Group) at ˜20 mW/cm² for varied time to form the hydrogel sheets (OMA 30 s, GelMA 180 s, PEGA8 30 s), which were segmented into hydrogel bars with a dimensions of 13×2×0.6 (mm mm mm). The hydrogel bars were immediately immersed in aqueous solution (specifically defined throughout the Results and Discussion section) to record the corresponding shape changes.

For the bilayer fabrication, OMA hydrogel precursors with or without UV absorber were placed underneath a pre-gelled GelMA layer (formed by 30 s UV crosslinking) and subsequently UV crosslinked for 30 s to form bilayer hydrogels (0.6 mm per layer), which were cut into bilayer bars (L×W×H=13×2×1.2, mm mm mm) and immersed in water for 30 min at room temperature (RT) to reach maximal bending.

Note that to minimize the potential impact of HMAP on the cell behaviors, culture medium for cell-laden constructs was changed 4 times during the first 2 h (every 30 min) to remove as much of the UV absorber as possible. The details about the definition of bending angle and the angle measurements can be found in the literature.

### Reversible Shape Change Study

Hydrogel strips (13 2 0.6, mm mm mm) fabricated as above (0.03% w/v RhB, UV 30 s) were cultured in aqueous solutions with varying pH under agitation (Bellco Glass 7744-01010 orbital shaker, Bellco Biotechnology, NJ, USA) at RT to record the shape changes. The agitation speed was set to 2.37 rev/s and incubation solutions were changed every 15 min to record the shapes for each cycle.

### Photomask-Aided Microfabrication, Photo-Patterning, Ion-Transfer Printing, and 3D Bioprinting

Unless otherwise stated, OMA hydrogel precursor solution was freshly made by dissolving OMA (6% w/v), PI (0.05% w/v) and UV absorber (0.02% w/v HMAP and 0.01% w/v RhB) in DMEM containing hMSCs (4×10⁶ cells/mL) for the experiments described below, and all the resulting hydrogels were cultured in cell growth medium (GM) consisting of DMEM, 10% v/v fetal bovine serum (FBS), and 1% v/v penicillin-streptomycin (P/S) in an incubator at 37° C. and 5% CO₂ for 2 h to allow them to fully deform into their final state. The medium was then replaced with PBS (pH 7.4) to take the pictures.

### Photomask-Aided Microfabrication

Hydrogel precursor solution was placed between two quartz plates with spacers (h 0.4 mm) and covered with a patterned photomask. The solution was exposed to UV light (˜20 mW/cm²) for 30 s. The photomask was removed and the quartz plates were gently separated, the microfabricated hydrogels attached on the surface of both plates were gently flushed into wells of a 6-well plate (Corning, NY, USA) using GM, and a total volume of 5 mL medium was used for hydrogel culture in each well.

### Photo-Patterning

Hydrogel precursor solution was placed between two quartz plates separated with spacers (h 0.6 mm) and then exposed to UV light for 30 s to form the hydrogels. Subsequently, a stripe-patterned photomask (stripe width 0.2 mm) was placed over the top plate, and UV light was further applied to crosslink for 60 s. The hydrogels were tailored using a punch into squares (sheet, 19×19, mm×mm) or circles (disk, d=20 mm), which were immersed in 5 mL of GM in 6-well plate wells for culture. Non-patterned hydrogel sheets (19×19, mm×mm) and disks (d=20 mm) were fabricated under UV irradiation for different times (40, 60, and 80 s) without patterning and then cultured to afford 3D cell-laden constructs with varying curvatures for comparison.

### Ion-Transfer Printing

Hydrogels formed as above (UV 30 s) were cut into hydrogel bars (14×20×0.6, mm×mm×mm) or hydrogel squares (20×20×0.6, mm×mm×mm). Then these tailored hydrogel constructs were covered by a filter paper with a specific pattern for 30 s. Note that the filter paper was pre-soaked in calcium chloride (1 M) solution for 5 min. Then, the post-treated hydrogels were immersed in 5 mL of GM medium in wells of a 6-well plate for culture. 4D bioprinting

Hydrogel precursor solution was loaded into a 1 mL syringe with a 0.5-inch 30G stainless steel needle (McMaster-Carr) and printed using a 3D printer (PrintrBot SimpleMetal 3D Printer, Printrbot). More details about this printer can be found in the literature. Digital models for 3D printing were generated from www.tinkercad.com. An empty Petri dish was placed on the building platform. The tip of the needle was positioned at the center and near the bottom of the dish, and the print instructions were sent to the printer using the host software (Cura Software, Ultimaker), which is an open source 3D printer host software. The printed hydrogel precursor solution was imaged and immediately subjected to photocrosslinking (UV 30 s at ˜20 mW/cm² intensity). Then, the hydrogel construct was gently transferred into a well of a 6-well plate filled with 5 mL of GM, cultured under the same conditions as above for 30 min, and imaged.

### Results

A typical setup and the process for the fast fabrication of graded hydrogel scaffolds is schematically illustrated in FIGS. 22A and B. Photocurable polymer was first dissolved in DMEM containing both PI and UV absorber (DMEM was used as solvent to promote subsequent viability of encapsulated cells) to form the hydrogel precursor solution, which was then placed between two quartz plates located at an adjustable distance from a UV light source. Since photo-curing efficiency relies on the irradiation intensity that the PI receives, and the irradiation intensity decays more rapidly with distance along the light travelling pathway with the co-existence of UV absorber, a gradient in crosslinking density can be readily generated with the top portion of the hydrogel closest to the light source showing high crosslinking density while the bottom portion is crosslinked to the lowest extent. This hydrogel bearing the crosslinking gradient can be further tailored into specifically shaped hydrogels and directly cultured in a medium to initiate deformation (FIG. 22B). It is noteworthy that it is the subsequent anisotropic swelling induced internal strain that causes the deformation towards the high-crosslinking side herein, which is different from deformations resulting from the generated anisotropic internal strain during the desolvation step that causes deformation towards the low-crosslinking side in previously reported gradient hydrogels.

To examine the versatility of this approach, synthetic polymer PEGA8, natural polysaccharide-derivatived OMA, and natural protein-derivatived GelMA were employed as biocompatible polymers, and a fluorescence dye RhB with an effective overlap in the UV absorption spectrum with the PI was selected as a UV absorber. Hydrogels were prepared and incubated in water for 4 h to ensure equilibrated swelling (see details in Supporting Information). As shown in FIG. 22C-E, hydrogels that were fabricated in the presence of RhB exhibited evident bending, while those hydrogels formed without RhB incorporation in the precursor solution exhibited almost no or only slight bending. It should be noted that although photocrosslinked PEGDA hydrogels typically do not swell much in medium and no deformation was shown in previous PEGDA hydrogels during rinsing, a clearly discernible bending was observed in the present case, which can likely be ascribed to the large difference in crosslinking density across the height of the construct.

There are other common compounds in addition to RhB that possess some overlap in UV absorbance spectrum with that of PI, such as colorless HMAP, the non-fluorescent dye AAb, and FITC, and can be used as UV absorbers. Similarly, rapid hydrogel bending was also clearly observed with these three additional examples (FIG. 22G-I), indicating the effectiveness of crosslinking gradient formation caused by the attenuated UV absorption from the hydrogel surface that first receives light to the surface furthest from the source due to use of these UV absorbers. Importantly, the UV absorber plays a critical role in the attenuation of UV light absorption in this system. In OMA hydrogels, for example, RhB or HMAP incorporation preserved substantially more methacrylate groups compared with the hydrogel lacking a UV absorber, with the amount of methacrylate groups remaining after UV crosslinking reflecting the extent of the photo-induced reaction. The continuous nature of the formed gradient was directly visualized in RhB incorporated hydrogels at higher magnification (FIG. 22F).

Although single material fabrication is straightforward, some application may benefit from or even require a multi-material system. In addition to single material fabrication, this strategy also applies to multi-material fabrication in a single step. To demonstrate this, bilayer hydrogels comprised of one gradient OMA [OMA(g)] layer and one GelMA layer, referred to as OMA(g)/GelMA, were fabricated. The key difference for the bilayer fabrication lies in the use of a hydrogel layer containing crosslinkable methacrylates on the surface that directly covers the OMA hydrogel precursor solution so that a strong interfacial adhesion can be formed after UV crosslinking. For comparison, bilayers that consist of non-gradient OMA and GelMA hydrogels (OMA/GelMA) were also fabricated. After reaching equilibrium swelling in water, OMA (g)/GelMA bent to a much larger extent than OMA/GelMA, due to the remarkably enhanced deformation ability of the gradient layer (FIG. 22J, K), implying that the integration of a gradient layer into a multi-material system can be an effective way to alter or improve the deformability.

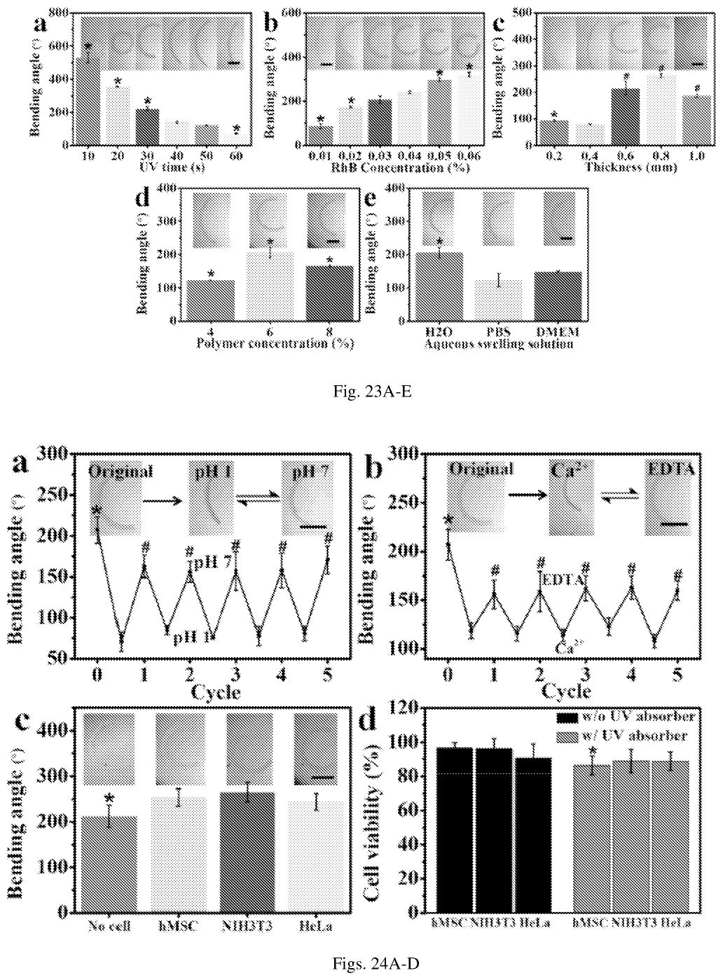

The effects of important parameters including UV irradiation time, UV absorber concentration, hydrogel thickness, polymer concentration, and swelling medium on construct morphing behavior were comprehensively investigated using OMA hydrogel bars as a prototype. Since the extent of crosslinking gradient differential across the hydrogel bar thickness strongly affects the bending angle, those parameters with the greatest capacity to augment the crosslinking gradient such as decreased UV exposure time (FIG. 23A), increased RhB concentration (FIG. 23B), and increased hydrogel thickness (<0.8 mm, FIG. 23C) resulted in a larger bending. Interestingly, while it is usually observed that increasing hydrogel bar thickness decreases its bending degree, the opposite phenomenon was observed in our case. However, the bending extent decreased again when the thickness reached 1.0 mm. It should be noted that, in the above hydrogels, in addition to the augmented gradient range, increased volumetric swelling caused by lowered overall crosslinking density also contributed to the increased bending. Moreover, polymer concentration and choice of aqueous swelling solution also exerted clear influence on the hydrogel bending. Hydrogels prepared at a concentration of 6% (w/v) and immersed in the H₂O displayed maximum bending (FIGS. 23D and E). The increase in the polymer concentration not only augments the gradient range but also diminishes hydrogel swelling at the same time. The final bending result stems from the competition of these two factors. The hydrogel swelling findings in different aqueous solutions, in contrast, is consistent with those of hydrogel deformation, in that higher swelling gave rise to increased bending. Thus, it can be seen that the bending degree can be finely tuned by altering any of these parameters, including the formulation of the hydrogel precursor solution, hydrogel dimensions, as well as culture medium. The parameters were set at 6% polymer concentration, 30 s UV irradiation, and 0.6 mm thickness for the following experiments unless stated otherwise.

The implementation of tunability of the deformation extent after hydrogel fabrication by treatment post-fabrication offers a second powerful opportunity to modulate the hydrogel geometry. Given that swelling of alginate-derived hydrogels can be modulated by both the incubation solution pH and divalent cation chelators such as ethylenediaminetetraacetic acid (EDTA), a strong Ca2⁺ chelating agent with the ability to snatch Ca2⁺ from the alginate network, the shape controllability of hydrogels post-fabrication was then demonstrated using the stimuli-responsive OMA hydrogels as an example. By soaking the hydrogel bars in water solutions of pH 1 or 7 or water solutions containing Ca2⁺ or EDTA, they showed reversible shrinkage and swelling (insets of FIGS. 24A and B), which in turn brought about changes in the hydrogel bending. When repeatedly switching the solutions (24A and B), the hydrogels repeatedly recovered their previous shape after the first deformation cycle. These findings demonstrate the capacity to adjust the shape of these constructs on demand.

Owing to biocompatibility of the materials and friendliness of the fabrication process towards the cells, encapsulation of cells within the hydrogels was examined. Cell-laden hydrogel bars were fabricated under the same conditions as described earlier and then cultured in the GM to investigate both the role of cells in construct shape change and cell viability in the system. Specifically, three cell types were examined: human mesenchymal stem cell (hMSC), a fibroblast cell line (NIH3T3), and a cervical cancer cell line (HeLa). In addition, HMAP (0.02%) was used as UV absorber due to its high efficiency for gradient generation, noninterference with the live/dead cell staining assay, and high cyto-compatibility (>90% cell viability in all the tested concentrations). Cell-laden hydrogel bars exhibited larger extent of bending compared to those without cells (FIG. 24C). This may be a result of the cells weakening light penetration by absorption, reflection, and scattering in the hydrogels. Nonetheless, non-gradient cell-laden hydrogel bars with cell density up to 1×10⁸ cells/mL hydrogel precursor solution displayed no obvious bending, suggesting the essential contribution of the effective gradient formation in driving the shape changes of the cell-laden constructs. All the three types of embedded cells maintained predominantly round morphology and high average viability after 3 days culture (˜86%-89%, FIG. 24D), albeit slightly lower than the control group (UV absorber-free group, ˜90%-96% viability, 24D). These results indicate that the dilemma of live-cell encapsulation in the shape-morphing hydrogels, which is deemed as a current challenge in the 4D biofabrication field, can be easily overcome using this one-step gradient-generation strategy.

Taking advantage of this platform system's simplicity and biocompatibility, the utility of applying additional hydrogel-engineering techniques, such as photo-aided microfabrication, photo-patterning, ITP, and 3D bioprinting, to fabricate cell-laden OMA hydrogel scaffolds with more sophisticated structures was investigated. To aid in visualization of the constructs, a combination of 0.02% RhB and 0.01% HMAP was used as a mixed UV absorber.

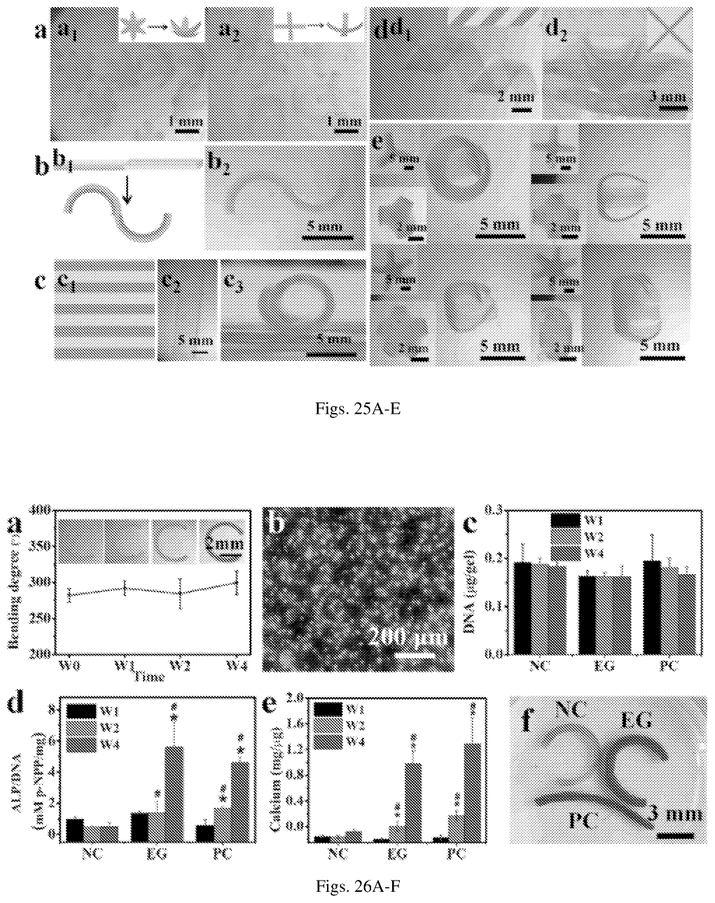

First, a photomask-aided 4D bio-microfabrication process was developed for rapid manufacture of large-scale bio-microstructures. For example, multiple cell-laden six-petal micro-blossoms (FIG. 25A1) and four-arm micro-grippers (FIG. 25A2) can be produced from a single batch. Samples with larger sizes using the same method were also made. Compared with the fabrication of shape-morphing micro-multilayers using photomask-based multistep photocrosslinking or micro-molding approaches, which either needs accurate alignment or long-time preparation, regardless of biocompatibility, this one-step 4D biofabrication strategy on a whole is much simpler, faster and more economical. These bio-microstructures might in the future be expanded to be applied as bio-microactuators if a stimuli-responsiveness is integrated. Based on the gradient crosslinking principle, multiple-gradients can be intentionally produced with more than one direction in a single hydrogel construct. For example, a single hydrogel bar with a “double-faced” gradient (FIG. 25B1) was fabricated by simply controlling the photomask and UV irradiation direction. This bi-gradient hydrogel bar deformed into an “S” shape after swelling (FIG. 25B2). Therefore, using this technique, it is easy to generate more complex structures with dissymmetric geometries in a single-layer hydrogel.

Next, photo-patterning of prefabricated gradient hydrogels was conducted using a mask-based photolithography technique to yield cell-laden macroscopic 3D structures. The post-patterning of the hydrogels enables patterning multiple user-defined regions to induce specific 4D architectural shape conversions. Pre-fabricated cell-laden gradient hydrogel sheets or disks were covered with a patterned photomask and then transferred to the GM for saturating at least 2 h at 37° C. Then clear images of deformed hydrogels were taken after replacing the GM with PBS. It was observed that the hydrogel sheets formed under photomasks with parallel strips (FIG. 25C1) curled up into well-shaped hydrogel tubes (FIGS. 25C2 and 25C3). Similarly, disk-shape-based hydrogel tubes were also obtained. Interestingly, by changing the patterned parallel strips into inclined strips, hydrogel sheets curled up into misaligned hydrogel tubes through diagonal rolling. Even though large cell-laden constructs with well-defined 3D structures, such as curled hydrogels with varying curvatures, can be easily obtained by rolling of those hydrogel sheets and disks fabricated with different UV irradiation times, the photo-patterning technique could endow hydrogels with larger deformations and greater curvature [e.g., patterned hydrogels with 60 s UV irradiation (FIG. 24C) and with more abundant structures [e.g., misaligned tube formation than those formed from non-patterned hydrogel sheets with the same or even lower UV irradiation time.

In addition to the above photolithographic techniques, an ITP strategy was introduced to locally treat the pre-formed gradient hydrogel with Ca2+ to locally induce secondary ionic crosslinking to the OMA hydrogel, causing constrained local swelling. By printing these divalent cations on specific regions of cell-laden hydrogel bars or hydrogel sheets (insets in FIG. 25D, dark pink section denotes cation-printed section), cell-laden spirals (FIG. 25D1) and cell-laden pseudo-four-petal followers (FIG. 25D2) were obtained.

Finally, this platform strategy was integrated into the realm of 4D bioprinting, a state of the art biofabrication technology that incorporates time-dependence into 3D bioprinting to confer conformational changes to printed biosamples, which may enable recapitulation of dynamic tissue and organ geometric evolution that occurs during development and regeneration. A cell-laden hydrogel bar was first printed and photocrosslinked to verify the feasibility of 4D bioprinting. As expected, this hydrogel bar folded into a hydrogel ring after being immersed in GM. Next, various cell-laden multiple-arm grippers were printed (insets on the upper left of FIG. 25E), photocrosslinked, and cultured to permit respective structural deformation to occur (FIG. 25E). Importantly, all these techniques do not compromise cell viability. To exclude the influence of RhB on live/dead cell staining visualization, parallel experiments under the same conditions were performed with HMAP (0.02% w/v) as the sole UV absorber to assess cytocompatibility. hMSCs within these 4D fabricated hydrogels were highly viable after 3 days culture, as indicated by the live/dead staining results.

Thus, this 4D biofabrication approach is compatible with other live-cell encapsulation techniques, making it useful for biomedical engineering applications.

4D biofabricated scaffolds capable of undergoing dynamic shape transformations may enable the engineering of tissues with complex geometries and replication of critical morphodynamic evolutions that occur during native tissue development. Design of conventional inductive hydrogel scaffolds for tissue engineering that are geometrically static often primarily focuses on controlling microenvironmental physiochemical niches for guiding cell behavior and new tissue formation and generally neglects important macroscopic morphing. The potential to differentiate stem cells within this 4D biofabrication platform for 4D tissue engineering applications was thus explored. A 4D osteogenesis study was conducted as a proof-of-concept by culturing an hMSC-laden hydrogel bar in the osteogenic medium over a course of four weeks, during which time its shape was continuously monitored. Cell viability, DNA content, and osteogenic markers such as alkaline phosphates (ALP) activity and calcium deposition were examined to evaluate the extent of osteogenic differentiation. Similar to findings after culturing in the GM, these hydrogel bars rapidly curled into an opened ring (<3 min) and then remained almost unchanged in shape during the 4-week culture (FIG. 26A), demonstrating robust geometrical stability. The live/dead staining results also revealed high cell viability after 4-week culture (FIG. 26B). For the osteogenesis study, hydrogel bars serving as the negative control (NC) were fabricated the same as those of the experimental group (EG), but they were cultured in the GM. Hydrogel bars serving as the positive control (PC) were prepared without incorporation of a UV absorber and cultured in osteogenic medium. The DNA levels were relatively constant over the entire culturing period (FIG. 26C), whereas the ALP activity and calcium content (both normalized to DNA) in the EG and PC significantly increased over time (except for ALP/DNA within the EG group from W1 to W2) and were significantly higher than those of NC. There was no significant difference found between the EG and PC at any time point (FIGS. 26D and E). The calcium content, a critical component of hydroxyapatite formation during osteogenesis, in EG and PC hydrogel bars was confirmed by dark alizarin red staining throughout the constructs (FIG. 26F). These results support the conclusion that the hydrogel deformation and cell differentiation within the hydrogel are generally independent of each other in this system, and thus they can be designed separately.

Morphological change is a common phenomenon that occurs during tissue maturation. Compared with 3D scaffolds, 4D scaffolds with the ability to reconfigure their shapes during culture show huge potential for morphodynamic tissue engineering. Hydrogels that harness non-uniform swelling, post-programmed anisotropic internal strains, or cell contractile forces can accomplish this task. However, in addition to the stringent requirements regarding material cytocompatibility, the fabrication process, and imposed stimulation, complexity in fabrication and lack of controllability present a significant impedance to 4D tissue engineering. 4D biofabrication using a simple, cost-effective, and robust one-step photocrosslinking method to generate instantly applicable scaffolds may help to realize the potential of 4D strategies in tissue engineering, as clearly demonstrated by our 4D bone-like tissue engineering study. It is interesting to note we did not observe a difference between the PC and EG groups (FIG. 26). One plausible reason is that there was negligible difference in the response of the encapsulated cells to the internal strain within the straight hydrogel slabs compared to the anisotropic internal strain distribution within the curved hydrogel slabs. In the future, using different mechanically sensitive cells (e.g., cardiomyocytes) or further increasing the structural anisotropy and internal strain represent possible ways to examine the impact of the 4D process on cell behaviors. Nevertheless, additional engineering of the materials, such as modification with cell adhesive ligands (e.g., RGD peptide motif), tuning matrix physical properties, facilitation of cell-cell interactions and/or controlled delivery of bioactive factors, may also be further explored to promote survival of diverse encapsulated cell population and enhance cell functioning.

4D biofabrication for biomedical applications requires not only effective anisotropy formation but also often necessitates conditions that facilitate cell encapsulation and long-term viability. A simple and efficient strategy comprised of only one-step photocrosslinking of cell-containing hydrogel precursor solution was engineered to achieve this goal. This example demonstrates a versatile technology in which the incorporation of UV absorber directly into the photocrosslinkable biopolymer solution creates an adjustable gradient, allowing for on-demand, controllable and cytocompatible hydrogel shape remodeling. This approach also exhibited enormous tolerance and adaptability, where not only diverse polymers and UV absorbers can be used as needed but also the strategy itself can be effectively integrated with other hydrogel-engineering techniques or platforms. In this manner, a facile and straightforward path has been established for concurrently enabling controllable, time-dependent shape changes into biomaterials along with encapsulated living cells, which has great potential in 4D biofabrication for biomedical applications such as biosensing and bio-actuation in living systems and tissue engineering.

### Example 4

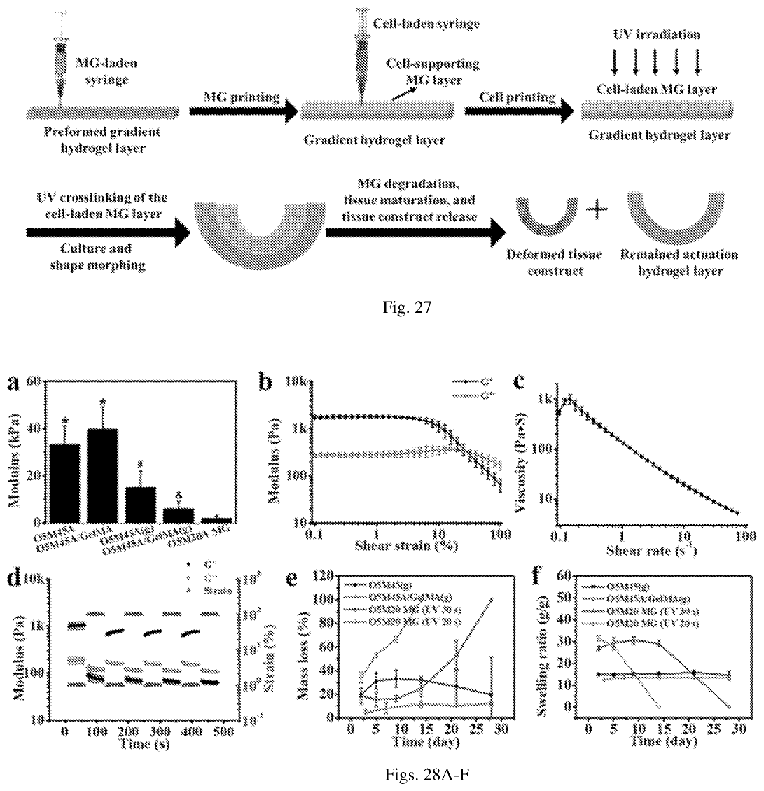

This example describes a 4D cell-condensate bioprinting strategy using a unique bilayered system has been developed in this work to impart a shape-morphing feature to a 3D printed cell construct (FIG. 27). The proposed bilayer consists of a preformed gradient-crosslinking hydrogel layer as the actuation layer that drives the shape morphing and a printed photocurable and degradable cell-supporting microgel (MG) layer that allows printing a cell-only bioink inside and maintains the shape of the printed cells as they form a condensate in the initial stage. Upon the gradual and controllable degradation of the photocrosslinked MG layer during culture in tissue specific differentiation media, 4D transformation into a mature tissue with a well-defined configuration can be obtained on demand.

### Experimental

### Chemicals, Instruments, and General Methods

Unless specified, all solvents and reagents were used without further purification. Sodium alginate (AL, Protanal LF120M, 157 Pa·s and 251 Pa·s) was a generous gift from FMC Biopolymer. Bovine skin derived gelatin (type B), photoinitiator (2-Hydroxy-4′-(2-hydroxyethoxy)-2-methylpropiophenone, PI), 4′-hydroxy-3′-methylacetophenone (HMAP), fluorescein diacetate (FDA), ethidium bromide (EB), Dulbecco's Modified Eagle Medium-Low Glucose (DMEM-LG), and fetal bovine serum (FBS) were purchased from Sigma. ITS⁺ Premix and penicillin/streptomycin (P/S) were purchased from Corning Inc. (Corning, NY). Sodium pyruvate was purchased from HyClone Laboratories. Non-essential amino acid solution was purchased from Lonza Group (Basel, Switzerland). Ascorbic acid-2-phosphate was purchased from Wako Chemicals USA Inc. (Richmond, VA). Fibroblast growth factor-2 (FGF-2) was purchased from R&D Systems (Minneapolis, MN). Transforming growth factor β1 (TGF-β1) was purchased from PeproTech (Rocky Hill, NJ). N-(2-aminoethyl) methacrylate hydrochloride (AEMA) and methacryloxyethyl thiocarbamoyl rhodamine B (RhB) were purchased from Polysciences Inc., and other common chemicals, such as sodium peroxide, methacrylic anhydride, etc., were purchased from Fisher Scientific. ¹H NMR spectra were obtained on a 400 MHz Bruker AVIII HD NMR spectrometer equipped with a 5 mm SmartProbe™ at 25° C. using deuterium oxide (D₂O) as a solvent and calibrated using (trimethylsilyl)propionic acid-d₄ sodium salt (0.05 w/v %) as an internal reference. DMEM-LG containing 0.05% PI (w/w) was used to dissolve the oxidized methacrylate alginate (OMA) and methacrylate gelatin (GelMA). Cell growth media (GM) consisted of DMEM-LG with 10% FBS and 1% P/S, and chondrogenic media consisted of DMEM-LG with 1% ITS⁺ Premix, 100 nM dexamethasone, 1 mM sodium pyruvate, 100 μM non-essential amino acids, 37.5 μg/mL ascorbic acid-2-phosphate and 1% P/S supplemented with 10 ng/mL TGF-β1. Hydrogel images were visualized using a Nikon SMZ-10 Trinocular Stereomicroscope equipped with a digital camera. A microplate reader (Molecular Devices iD5) was used to read data from the microplates. A UV device (EXFO OmnicureR S1000-1B, Lumen Dynamics Group) with an intensity of 12 mW/cm² was used for photocrosslinking. All quantitative data was expressed as mean±standard deviation. Statistical analysis was performed with one-way analysis of variance (ANOVA) with Tukey honestly significant difference post hoc tests using Origin software (OriginLab Corporation). A value of p<0.05 was considered statistically significant.

### Synthesis of OMAs and GelMA

OMAs with a theoretical 5% oxidation degree and varying theoretical methacrylation degrees (20%, and 45%) were synthesized according to a similar method as described in the literature. The O5M20A (5% theoretical oxidation and 20% theoretical methacrylation) was synthesized with the following procedure: 10 g of sodium alginate (251 Pa·s) was dissolved in 900 mL of diH₂O overnight, and 0.54 g of sodium periodate (NaIO₄) in 100 mL of diH₂O was rapidly added to the alginate solution under stirring in the dark at room temperature (RT). After reaction for 24 h, 19.52 g of 2-ethanesulfonic acid (MES) and 17.53 g of sodium chloride (NaCl) were added, and the pH was adjusted to 6.5 with 5 N sodium hydroxide (NaOH). Then 1.18 g of N-hydroxysuccinimide (NHS) and 3.89 g of 1-ethyl-3-(3-dimethylaminopropyl)carbodiimide hydrochloride (EDC-HCl) were sequentially added to the mixture. After 10 min, 1.69 g of AEMA was added slowly. The solution was wrapped with aluminum foil to protect it from light and left to react for 24 h at RT. The mixture was then poured into 2 L of chilled acetone to precipitate out the crude OMA solid, which was further purified by dialysis against diH₂O over 3 days (MWCO 3.5 kDa, Spectrum Laboratories Inc.). The dialyzed alginate solution was collected, treated with activated charcoal (0.5 mg/100 mL, 50-200 mesh, Fisher) for 30 min, filtered through a 0.22 μm filter and frozen at −80° C. overnight. The final O5M20A was obtained as white cotton-like solid through lyophilization for at least 10 days. O5M45A (5% theoretical oxidation and 45% theoretical methacrylation) was synthesized through the same procedure according to the reported literature. The actual methacrylation of O5M20A and O5M45A was determined to be 5.7% and 16.2% from ¹H NMR data according to the method described in the literature. Note that the actual oxidations were not provided due to the overlap of the proton peak assigned to the CHO group (˜5.4 ppm) with the polymer proton peak (broad peak located at ˜5.1 ppm). The GelMA was the same material that was used in our previous work. The ¹H NMR spectra for newly synthesized OMAs.

### Microgel (MG) Preparation

MGs was prepared using a modified procedure from the reported literature. To make the stock MGs, O5M20A (1.2 g) was dissolved in deionized water (diH₂O, 60 mL) and then was slowly dispensed into a gelling bath containing an aqueous solution of CaCl₂ (600 mL, 0.2 M) under fast stirring with a magnetic stir bar. After fully ionically crosslinking overnight, the resultant hydrogel beads were collected, washed once with 40 mL of 70% ethanol (EtOH)/water (H₂O), and then blended twice using a household blender (Osterizer MFG, at “pulse” speed) for 2 min with 120 mL of 70% EtOH/H₂O. Then, the MGs were obtained and loaded into 50 mL conical tubes and centrifuged at 4200 rpm for 5 min and stored at 4° C. for future use.

To recover the MGs for use, the as-prepared MGs (5 mL) in 70% EtOH/H₂O were washed 3 times by replacing the previous media with 25 mL of 0.05% (w/w) PI-contained diH₂O and then vortexed (Fisher STD Vortex Mixer, Fisher Scientific, 10× speed) for 2 min every time and subsequently washed 1 time by replacing the previous media with 25 mL of 0.05% (w/w) PI-containing DMEM-LG and vortexed (10× speed) for 10 s. This recovered MGs were used for further experiments.

### Gradient and Non-Gradient Hydrogel Preparation

Gradient hydrogels were used as the actuation layer to drive shape morphing. The preparation was according to a similar procedure described in the reported literature. Briefly, a mixed solution of polymer [OMA 6% w/w, OMA (6% w/w)/GelMA (6% w/w)], PI (0.05% w/w), and UV absorber [RhB (0.01% w/v)/HMAP (0.02%)] in DMEM-LG was placed between two quartz plates with 0.6 mm spacers and subsequently photocrosslinked with UV light (EXFO OmnicureR S1000, Lumen Dynamics Group) at 12 mW/cm² for a certain time (20 s˜40 s) to form a O5M45A hydrogel disc with a gradient in crosslinking density throughout the thickness, which was denoted as O5M45A(g) or O5M45A/GelMA(g). Non-gradient hydrogels were used for comparison and were fabricated similarly without using a UV absorber and were denoted as O5M45A or O5M45A/GelMA. The gradient/non-gradient hydrogels discs were cut into hydrogel sheets with dimensions of 25 mm×25 mm as a substrate for the MG printing (described later).

### Microgel (MG) Printing

The MG printing was performed using a 3D printer (PrintrBot Simple Metal 3D Printer, Printrbot) modified with a syringe-based extruder. More information about this printer can be found in the literature. The STL files for the bioink printing were generated from www.tinkercad.com.

The MGs were loaded into a 1 mL glass syringe (Hamilton, Reno, NV), which was connected to a 22-gauge (22G) stainless-steel needle (McMaster-Carr) and mounted into the syringe pump extruder on the 3D printer. The above O5M45A(g) hydrogel sheet was flipped and placed on a quartz plate with low-crosslinking side attaching the quartz plate surface. The tip of the needle was positioned at the center and near the surface of the O5M45A or O5M45A(g) hydrogel sheet, and the print instructions were sent to the printer using the host software (Cura Software, Ultimaker), which is an open-source 3D printer host software. The MGs were printed under 4 mm/s printing speed with 100% infilling density. After printing, the obtained constructs were used for further cell-only printing (described later) or immediately photocured under UV irradiation. Then, the cell-free constructs were cut into specific shapes (e.g., strip, sheet, and disc). These hydrogels were then carefully transferred into a tissue culture plate for culturing to monitor the shape change or/and allow cell differentiation. The hydrogels were imaged, and the bending angles were quantified according to the previous literature.

For printing MGs for swelling and degradation studies, Young's modulus testing, rheological testing, and the cell printability study, MGs were printed according to a similar method described above. The MGs were directly printed on the quartz plate instead of on the surface of a gradient hydrogel.

### Bilayer Hydrogel Fabrication

Gradient/non-gradient hydrogels as substrates were fabricated as above and MGs were printed on the surface of the hydrogel substrates as above. The bilayer hydrogels were then photocrosslinked for a certain time. To distinguish the UV time for the bilayer fabrication, the total UV time was denoted as gradient layer time/MG layer time. For example, if the UV time for the gradient hydrogel layer and the MG layer is set to 30 s and 20 s, respectively, the overall UV time for the bilayer fabrication is denoted as 30 s/20 s. The bilayer is denoted as bottom hydrogel_upper hydrogel. For example, O5M45A/GelMA(g)_O5M20A MG is a bilayer consisting of O5M45A/GelMA(g) (bottom layer) and O5M20A MG (upper layer).

### Swelling and Degradation Tests

Gradient/non-gradient hydrogels and UV crosslinked MGs were used for swelling and degradation tests. As described above, gradient/non-gradient hydrogels were fabricated with a specific UV crosslinking time of 60 s, and MGs were printed into cuboids with dimensions of 8×6×3 mm³ and then UV crosslinked for 20 s or 30 s. The hydrogel samples were frozen for 4 h at −80° C. and lyophilized for 2 days. The masses of the dried gels were measured as initial weights (Wi). The dried hydrogels were rehydrated by culturing in 5 mL of GM at 37° C., and the media was changed every 3 days. At predetermined time point, the hydrogels were collected, and the swollen weights (Ws) were measured. The swelling ratio was calculated with the following equation: Ws/Wi (N=3). For the degradation test, the rehydrated hydrogels were collected at predetermined time points over 28 days and dried by lyophilization to obtain dried mass (W_{d}). Mass loss was quantified as (Wᵢ−W_{d})/Wᵢ×100% (N=3) for each condition per time point.

### Young's Modulus Measurement

The elastic moduli of the gradient/non-gradient hydrogels and UV crosslinked MGs were determined by performing uniaxial, unconfined constant strain rate compression testing at RT using a constant crosshead speed of 0.8%/sec on a mechanical testing machine (225 lbs Actuator, TestResources, MN, USA) equipped with a 5 N load cell. To obtain the Young's modulus of the engineered cartilage-like tissue (described later), the tissues were punched into a cylinder (d=2 mm, h=1.2 mm) using a biopsy punch. The compression tests were performed with the same protocol as above except a 0.5%/sec crosshead speed was used. The Young's modulus of each sample was determined using the first non-zero slope of the linear region of the stress-strain curve within 0˜10% strain (N=3).

### Rheological Testing

Dynamic rheological examination of the uncrosslinked and photocrosslinked MGs was performed to measure the hydrogel storage and loss moduli and viscosity and evaluate the shear-thinning, shear yielding, and self-healing properties with a Kinexus ultra+ Highest specification rheometer (Malvern Panalytical). In oscillatory mode, a parallel plate geometry (8 mm diameter) measuring system was employed, and the gap was set to 1 mm. After each hydrogel was placed between the plates, all the tests were carried out at RT. Oscillatory frequency sweep (0.1˜100 Hz at 1% strain) tests were performed to measure storage moduli (G′), loss moduli (G″), and viscosity. Oscillatory strain sweep (0.01˜100% strain at 1 Hz) tests were performed to examine the shear-thinning characteristics of the MGs and to determine the shear-yielding points at which the MGs behave fluid-like. To investigate self-healing properties, cyclic deformation tests were performed at 100% strain with recovery at 1% strain, each for 1 min at 1 Hz.

### Interfacial Adhesion Testing

The tensile testing was performed according to the reported literature using a mechanical testing machine (225 lbs Actuator, TestResources, MN, USA) equipped with a 25 N load cell to evaluate the interfacial adhesive strength of the bilayer hydrogels (O5M45A/GelMA(g)_O5M20A). Briefly, the hydrogel samples with an interfacial cross-sectional area of 5×1 mm² were attached to two hard paper backings using cyanoacrylate glue (Krazy Glue®, Elmer's Products Inc., Columbus, OH). The hard paper backings were then attached firmly with common commercial transparent tape to a “plastic loading platen”, which was attached to the “load cell”, and to a “sample cup”, which was fixed on the bottom platform of the mechanical testing machine with a 5 mm gap. The adhesion strength was determined by performing constant strain rate (0.6%/sec) tensile tests at RT.

### Cell Expansion

Human mesenchymal stem cells (hMSC) were isolated according to the literature. hMSC cells were expanded in GM supplemented with 10 ng/mL FGF-2 and HeLa cells were expanded in GM. The incubation was performed in a humidified incubator at 37° C. and 5% CO₂ with media changes every 2 or 3 days. The cells were harvested as cell-only bioinks according to the protocol described in the literature for printing when they reached ˜80% confluence.

### Cell-Only Bioprinting

HeLa and hMSC cells as bioinks were loaded into 3 mL of luer lock syringes (Becton, Dickinson and Company, NJ), connected to a 25G stainless steel needles (McMaster-Carr) and mounted into the BIO X 3D printer (CELLINK, MA). Pre-printed MGs described above were used as the supporting batch for 3D cell-only printing. The printing parameters were set to 95% infilling density, 2 mm/s printing speed, and 0.8 μL/s (HeLa cells) or 1.0 μL/s (hMSC cells) extrusion rate. After 3D printing of the cell-only bioinks, cell-laden O5M20A MGs were stabilized by photocrosslinking under UV for a specified time. The photocrosslinked cell-laden bioconstructs were transferred into a 4-well tissue culture plate for culturing the strip-shaped hydrogels with 4 mL of GM or a 6-well tissue culture plate for culturing bioconstructs with other shapes with 10 mL of GM to record shape change and assess cell viability through a live/dead staining assay (described later). The media was changed every day.

### Live/Dead Staining

The viability of cells was assessed using live/dead staining comprised of FDA and EB. The staining solution was freshly prepared by mixing 1 mL of FDA solution (1.5 mg/mL in DMSO) and 0.5 mL of EB solution (1 mg/mL in PBS) with 0.3 mL PBS (pH 8). At predetermined time points, 20 μL of staining solution per 1 mL of culture media was added into each well and incubated for 5 min at RT. Fluorescence images of the samples were taken using a Nikon Eclipse TE300 fluorescence microscope (Nikon, Japan) equipped with a 14 MP Aptina Color CMOS digital camera (AmScope, CA).

### Ex Vivo Cartilage-Like Tissue Engineering, Biochemical Quantification, and Histological Staining

The hMSCs at passage 4 (P4) were used for the chondrogenesis study. The cells were harvested for cell-only bioprinting when they reached ˜80% confluence. The cell-laden bilayer hydrogel strips were fabricated as above and cultured in chondrogenic media. The UV application time for the letter “C”- and helix-shaped bioconstruct formation was set to 50 s/20 s and 30 s/20 s, respectively. For cartilage-like tissue formation, the hMSC-laden hydrogels were cultured in 4 mL of chondrogenic media in a humidified incubator at 37° C. with 5% CO₂ over a course of 21 days, and 2 mL of media was changed every day. The cartilage-like tissues were obtained at day 21 and cut into small pieces for biochemical analysis, Young's modulus testing, and histological staining.

For biochemical analysis, the small tissue pieces were digested in 0.5 mL of papain solution (Sigma) at 65° C. for 24 hours and centrifuged for 10 min at 15,000 rpm, and then the supernatants were collected for DNA and glycosaminoglycan (GAG) quantifications (N=3).

Per the manufacturer's instructions, a Picogreen assay kit (Invitrogen) was used to quantify the DNA content in the supernatant. Fluorescence intensity of the dye-conjugated DNA solution was measured using a microplate reader with an excitation of 480 nm and emission of 520 nm.

The GAG content was quantified using a DMMB (1,9-dimethylmethylene blue) assay. 40 μL of supernatant from the digested samples was transferred into 96-well plate, to which 125 μL of DMMB solution was then added. Absorbance at 595 nm was recorded on a microplate reader. GAG content was normalized to DNA content.

The small cartilage-like tissues were fixed with 10% neutral buffered formalin (NBF) overnight at 4° C., dehydrated, and embedded in paraffin. Briefly, tissue samples were cut into 5 μm thick sections using a Leica RM2255 rotary microtome (Leica Microsystem Ltd., Milton Keynes, UK). Slide sections were then deparaffinized and stained with hematoxylin and eosin (H&E) stains to observe gross cell and tissue morphology, and safranin 0 (SafO) with a Fast Green counterstain and toluidine blue 0 (TBO) for glycosaminoglycan (GAG) indication. Stained samples were imaged using a fluorescence microscope (Nikon Eclipse TE300, Japan) under bright field.

TBO staining was also used to stain the entire helix-shaped cartilage-like tissue. The whole tissue was collected and stained with Toluidine blue 0 for 30 min and washed with PBS 3 times to remove the unbound stain.

### Results

### Cell-Free Bilayer Fabrication and Shape-Morphing Behaviors

We have previously demonstrated the feasibility of incorporation of a UV absorber into a single-component photocrosslinkable biopolymer, such as an oxidized and methacrylated alginate (OMA), to effectively generate a crosslinking gradient throughout the hydrogel thickness by a one-step UV photocrosslinking, which is attributed to UV absorber-based light attenuation along the light pathway. Consequently, an internal anisotropic strain was generated after swelling in media, leading to hydrogel deformation. Herein, a bicomponent photocrosslinkable biopolymer composite of O5M45A (OMA with 5% theoretical oxidation and 45% theoretical methacrylation) and GelMA (methacrylated gelatin) was employed to form a gradient hydrogel [O5M45A/GelMA(g)]. While the bicomponent hydrogel without gradient formation (O5M45A/GelMA) exhibited a similar modulus with the single-component non-gradient hydrogel (O5M45A), the elastic modulus of O5M45A/GelMA(g) was smaller than that of O5M45A(g) (FIG. 28A), which implies that O5M45A/GelMA(g) had a larger gradient range in comparison with the single-component gradient hydrogel [O5M45A(g)]. As a result, this bicomponent gradient hydrogel exhibited greater deformability.

The printability of O5M20A microgels (MGs), which are calcium ion (Ca²⁺)-crosslinked hydrogels fabricated according to our previously described method (supporting information), was then studied prior to the bilayer hydrogel fabrication. The prepared MGs behaved as a stable bulk hydrogel under low shear strains, (FIG. 28B) but yielded at a shear strain over 25% (FIG. 28B). Shear-thinning behavior was also identified by increasing the shear rate (FIG. 28C), shear strain, and shear stress. Moreover, the MGs displayed a rapid and reversible phase transition between the solid-like (elastic) state and the liquid-like (viscous) state by alternating the shear strain applied between 1% and 100% (FIG. 28D), suggesting favorable extrudability and rapid self-healing after deposition. Consequently, MG structures with high resolution were readily printed and further stabilized by subsequent UV-crosslinking, resulting in dual-crosslinked constructs. As expected, due to the low methacrylation degree, the MGs exhibited a rapid and tunable degradation profile in cell growth media. For example, the dual-crosslinked MG completely degraded in 14 days when UV-crosslinked for 20 s, while it took approximately 28 days for complete degradation if the MGs were UV irradiated for 30 s (FIG. 28E). This result provides evidence that the liberation of a cell condensation construct at a predetermined time point may be accomplished by simply adjusting the UV-crosslinking time. In addition, the UV-crosslinked MGs also exhibited a higher swelling ratio (FIG. 28F) but much weaker mechanics (FIG. 28A) than the non-MG gradient hydrogels at the initial stage, and the swelling and mechanics decreased along with the degradation over time (FIG. 28F). In contrast, the O5M45A/GelMA(g) was relatively stable in the media during the course of a 28-day culture. The higher stability of the proposed O5M45A/GelMA(g) actuation layer can provide a stable shape-morphing force to maintain the shape of the deformed cell construct for long-term culture.

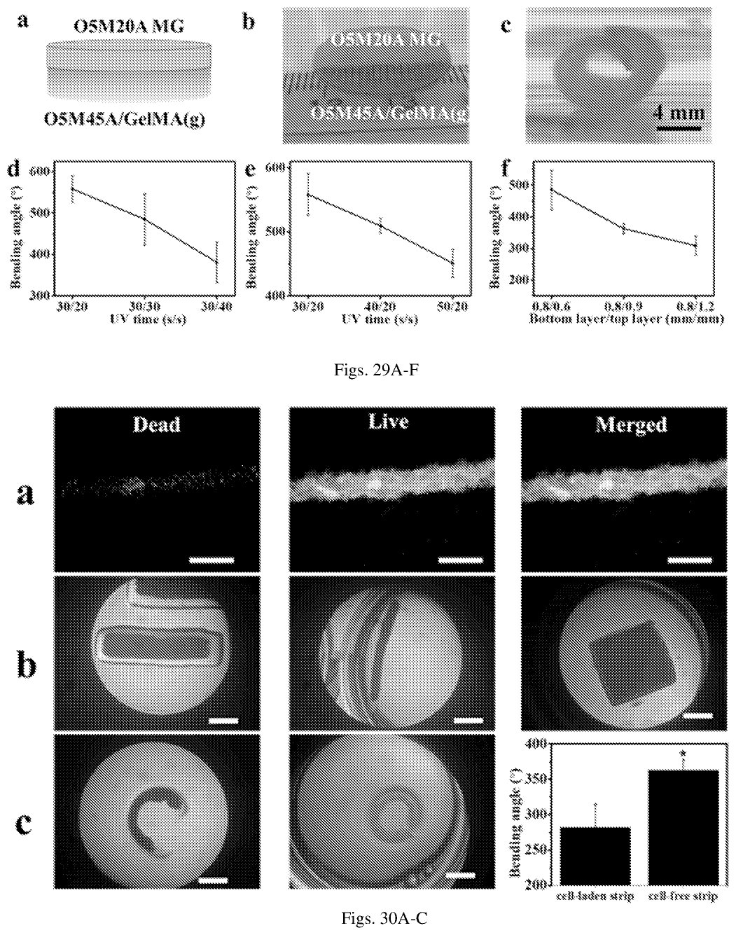

Owing to its printability and photocrosslinkability, the O5M20A MGs can be finely printed using a 22-gauge needle onto the surface of a pre-fabricated O5M45A/GelMA(g) substrate. The printed MG layer was subsequently UV-crosslinked, forming a stable bilayer system with robust interfacial adhesion between the two layers (FIGS. 29A, B) through the covalent crosslinking of the remaining methacrylate groups on the preformed O5M45A/GelMA(g) surface with the methacrylate groups on the O5M20A MG. Although the as-crosslinked MGs exhibited a higher swelling ratio than the O5M45A/GelMA(g) hydrogel (FIG. 28F), this bilayer hydrogel (i.e., a disc) rapidly rolled into a tubular structure towards the MG side when immersed in the phosphate buffered saline (PBS, pH 7.4) media (FIG. 29C), due to a synergistic contribution of (i) the large gradient within the actuation layer that generates a substantial actuation force large enough to overcome the opposite layer strain brought by the swelling discrepancy and (ii) the extremely weak stiffness of the MG layer that allows easy deformation. The role of the actuation layer in driving the shape-morphing process was further verified by a comparison experiment. Among hydrogel strips tested, including single-layer non-gradient O5M45A/GelMA, gradient O5M45A/GelMA and MG hydrogels and bilayer hydrogels with a non-gradient or gradient O5M45A/GelMA layer, only the hydrogels with a gradient layer showed remarkable rolling, while the shapes of the hydrogels involving no gradient remained unchanged. Interestingly, the bilayer that consisted of an MG layer and a non-gradient O5M45A/GelMA did not show any shape change regardless of the swelling difference between the two layers. This is because the MG layer is too soft to serve as either an actuation layer or a shape-constraint layer. For this reason, the bilayer composed of an MG layer and a gradient O5M45A/GelMA layer shared a comparable bending angle with the single-layer gradient hydrogel. This exquisite design offers a unique bilayer system in which the two layers have specific, independent functions.

The prototype of the bilayer hydrogel strip was then used to investigate the impact of UV crosslinking time exposed to each layer (the gradient layer and the MG layer) and the thickness of the MG layer on shape-morphing behavior. Results of the quantified bending angles revealed a clear influence of these parameters on the hydrogel deformation (FIGS. 29D-E). Increasing UV exposure time and MG layer thickness both exerted negative effects on the hydrogel bending. As far as the UV time, the receiving time for each layer can be selectively tuned to control the hydrogel bending. For example, the UV time for the upper layer (MG layer) can be adjust after being deposited onto the preformed gradient layer (FIG. 29D) or the UV time can be adjusted when fabricating the bottom layer (gradient layer) while fixing the UV time for the upper layer (FIG. 29E). The lowered bending arose from the combined outcome of reduced gradient range and lowered volumetric swelling by increasing UV time. Differing from the UV time, the lowered bending achieved by the increasing MG thickness (FIG. 29F) can be mainly attributed to increased resistance of the MG layer to the morphing. This controllable shape-morphing property will benefit applications where the capacity to tailor the extent of geometric change is desirable.

Cell Condensate-Laden Bilayer Fabrication and their Shape-Morphing Behaviors

The printability of the live cells within the MG layer was examined before fabricating the cell condensate-laden bilayer system. Live cells themselves can serve as a cell-only bioink to be printed into an O5M20A supporting MG bath, which was first printed as described above. In this cell-only printing process, the supporting material is typically shear-thinning and rapidly self-healing, permitting free embedding and deposition of live cells by replacing the MGs along the needle moving pathway and concurrently maintaining the printed cell construct with high fidelity. After printing, the dual-crosslinkable property of the MGs enables UV crosslinking to further stabilize the printed cell-only bioink and permits cell condensation formation without an intervening scaffold material. HeLa cells were first printed into a cell filament with a 25-gauge needle inside an as-printed MG strip (22×5×1 mm³). The cell filament-laden MG strip was subsequently UV crosslinked and imaged under a microscope to examine the printing resolution and then cultured in cell-growth media for 4 h to examine the cell viability. Results showed that the cells were printed into a filament with high resolution, confirming reliable cell printability, and remained highly viable (FIG. 30A), suggesting no obvious adverse effects of the bioprinting process and UV crosslinking on cell survival. Next, larger cell constructs, e.g., a cell strip (18×4×1.2 mm³, FIG. 30B, left and middle images), a cell sheet (13×13×1.2 mm³, FIG. 35B, right image), and a cell cuboid (5×5×4 mm³), with high resolution were then printed into the supporting MG bath, further confirming the cell printability.

Cell condensate-laden bilayer hydrogel strips were then fabricated by a sequential printing process (30 s/20 s UV crosslinking time) and the shape-morphing behaviors were recorded. The O5M20A MG strip (22×5×1.2 mm³) was first printed onto the surface of a preformed O5M45A/GelMA(g) hydrogel disc and cells were subsequently printed within the MG layer to form a cell strip-laden bilayer construct, which was then UV crosslinked. Cell condensate-laden bilayer constructs were then obtained by cutting from the large construct and cultured in cell growth media for 4 h until no further morphological changes could be observed. As expected, the cell-laden bilayer strip underwent a bending process into a letter “C” shape towards the cell-laden layer side (FIG. 30C, left) with, however, a smaller bending angle in comparison to the cell-free bilayer strip (FIG. 30C, middle and right), most likely due to enhanced morphing resistance by the infilled cell condensate. Noteworthily, the shape-morphing process did not compromise the integrity of the printed cell construct because of the firm support by the surrounding dual-crosslinked MGs.

Printing of Large Cell Constructs with Complex Structures and their Shape-Morphing Behaviors

The successful 4D bioprinting of the bilayer cell condensate-laden hydrogel strips inspired printing of larger cell condensate-laden constructs with more complex structures and investigation of their shape-morphing behaviors. Four different bilayer bioconstructs, including sheet-based bilayers with a cell filling in the shape of a sheet, bar grid, and net, and a disc-based bilayer with a disc-shaped cell infilling, were fabricated with a similar sequential printing process described above (FIG. 31, 40 s/20 s UV crosslinking time). HeLa cells were printed into specific geometries within the MG layer with high resolution, and these self-transformable cell condensate-laden and cell-free constructs morphed into concave structures by curling up to the cell layer side after culturing in cell growth media for 4 h. In addition, these bioconstructs appeared to be less curled compared to their cell-free counterparts, in agreement with the results obtained from the bilayer hydrogel strips. The above results collectively demonstrated the feasibility and effectiveness of this strategy to fabricate cell condensates with prescribed configurations by the 4D bioprinting technique.

To assess the long-term cell viability and stability of the bioconstructs, the cell-net infilled bilayer sheet (FIG. 31D) was cultured in cell growth media, cell viability was examined using a live/dead staining assay and shape changes were monitored over a course of 14 days (FIG. 32). The curling of the bioconstruct progressively increased during the first few days of culture (before day 5) due to MG layer degradation-induced softening of the cell-laden layer. The overall structure remained in a cylindrical shape, and the printed cells went through condensation to reinforce the “cell-net filament” during this period. It is interesting to note that the two layers (i.e., the actuation layer and cell condensate-laden layers) were observed to have visibly separation after a media change on day 6 while the structures of the individual layers were maintained (FIG. 32A D6). The layer separation stemmed from the degradation of the interfacial covalent crosslinks between the two layers. However, the shape of the cell condensate-laden MG layer was still stable because most crosslinked MGs were still retained at this time point. The gradually increased degradation of the MG layer with increasing culture time ultimately resulted in the disintegration of the MG layer on day 9 and some of the cell-net filaments were “liberated” from the MG layer (FIG. 32A, D9). After continued culture of the bioconstruct to day 12, the MG layer was found to have further disintegrated, (FIG. 32A, D12) and it was difficult for the bioconstruct to withstand the media change-mediated disturbance. With further culturing, the dual-crosslinked MGs almost completely degraded and were unable to support the cell-net structure, which eventually crumbled on day 14. During the entire culturing period, the cell-net filaments maintained integrity, regardless of the MG degradation (FIGS. 32C-D, bright-field images), suggesting that strong physical cell-cell cadherin interactions were present within the cell condensate, and the cells were highly viable (FIGS. 32C-D, live/dead stained images), indicative of good cytocompatibility of this system. As the MG degradation can be tuned by controlling UV crosslinking time, in another independent experiment, a cell-net infilled bilayer construct was fabricated by increasing the UV exposure time for the MG layer from 20 s to 30 s, and the obtained bioconstruct (40 s/30 s UV crosslinking time) went through a similar but slower 4D process to form a tubular structure and maintained stable configuration over 21 days.

Ex Vivo Engineering Cartilage-Like Tissue with Pre-Programmed Shape

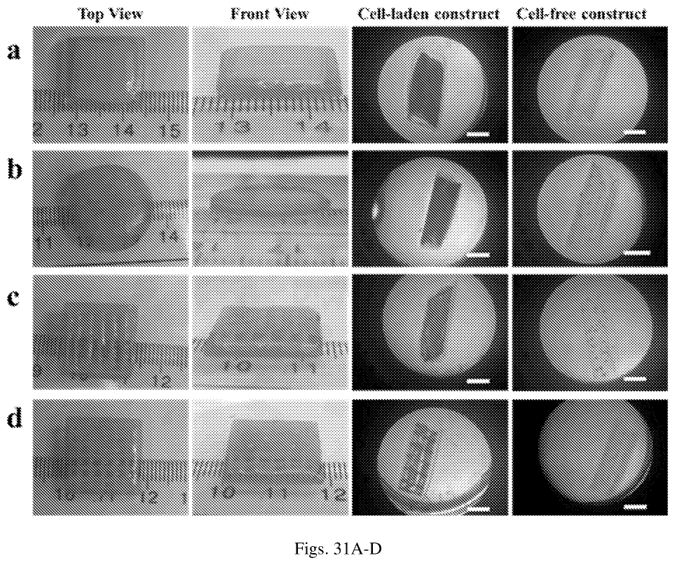

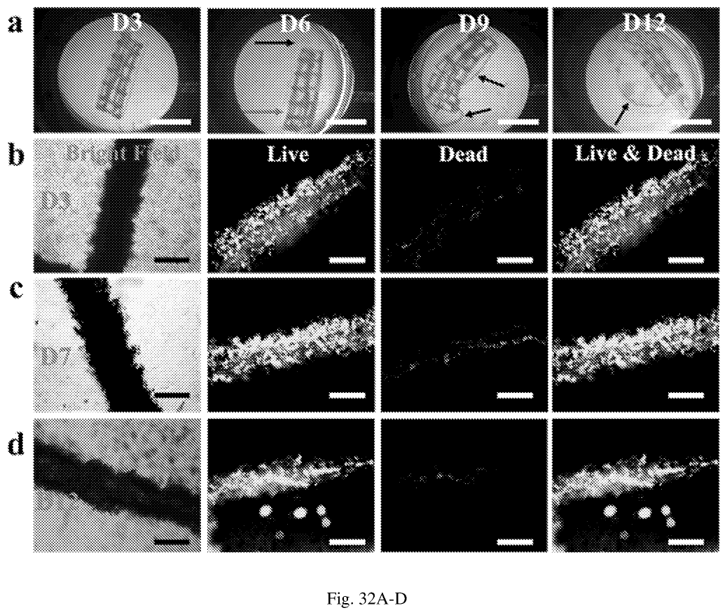

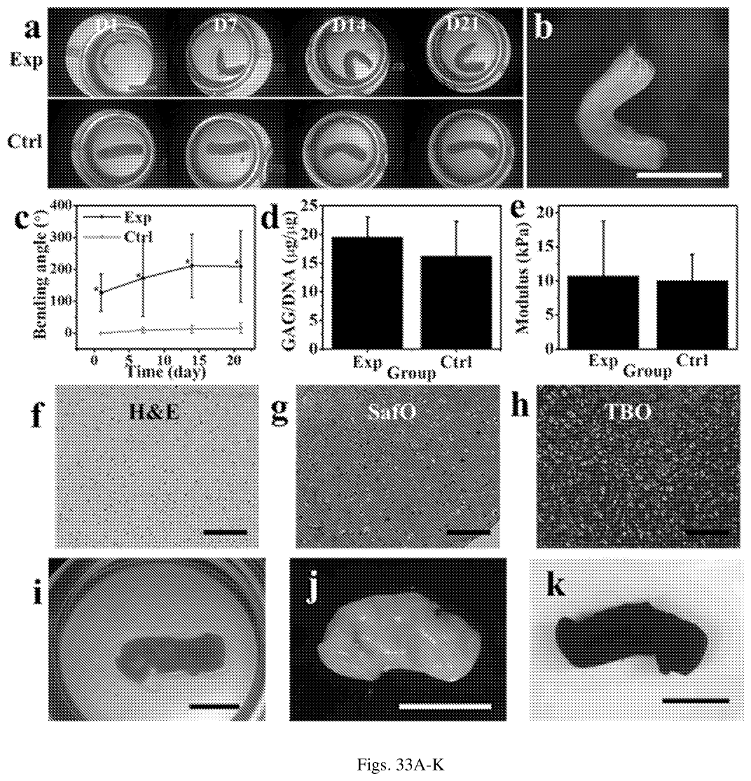

With this system, specific tissues with well-defined configurations through a reshaping process can be obtained after culturing the cell-condensate construct in a cell-differentiation environment for an appropriate time course. To explore the application potential of this 4D-condensate bioprinting system in tissue engineering, a proof-of-concept study was performed by fabricating hMSC cell condensate-laden bilayer strips (50 s/20 s UV crosslinking time) with this 4D bioprinting strategy and then culturing the bilayer strips in chondrogenic media for 21 days to generate hydrogel-free 4D tissues. Like the progressive shape-morphing behaviors observed in the aforementioned sheet-shape-based systems, the bilayer strips in the experimental groups (Exp) deformed into a letter “C” shape in the first 24 h culture and the bending slowly increased until 14 days. Then the structures remained stable from 14 to 21 days (FIG. 33A, C). Since the two layers detached at D7, we speculate the limited increased bending after D7 may have resulted from cell cytoskeleton-based contractile forces within the cell condensates. This similar phenomenon was also observed in the controls (cell condensate-laden single-layer MG strips, Ctrl), although minimal bending occurred in this group (FIGS. 33A, C). Cartilage-like tissues in the shapes of the Letter “C” (FIG. 38B) and a nearly straight line were finally obtained after full degradation of the supporting MGs in the Exp and Ctrl groups, respectively, after culture for 21 days. The 4D engineered cartilage-like tissues presented a similar level of glycosaminoglycan (GAG) production (FIG. 33D), a key cartilage extracellular matrix component, and similar Young's modulus compared to the cartilage-like tissue obtained from the controls (FIG. 33E). Hematoxylin and eosin (H&E) staining showed that the engineered constructs exhibited a homogeneous pattern of tissue comprised of uniformly distributed chondrocytes. Strong safranin O (SafO) and toluidine blue O (TBO) staining in the 4D constructs also revealed substantial GAG production (FIGS. 33F-H) and appeared similar to the staining in the control.

As indicated earlier (FIGS. 32C and D), the shape of the bioconstruct can be tuned by adjusting the UV time for either the bottom layer or the upper layer. We demonstrated that increasing the UV time for the upper layer can also prolong the retention duration of the cell condensate layer before being released (FIG. 32). To keep the same MG degradation profile and increase the deformability of the bilayer structure, we reduced the UV irradiation time from 50 s to 30 s to fabricate the bottom layer while maintaining a 20 s UV irradiation time for the upper layer, offering cell-strip laden bilayers (30 s/20 s UV crosslinking time) with greater curling, and yielding helix-shaped cartilage-like tissues after culturing for 21 days in chondrogenic media (FIG. 33I). Unlike the “C”-shaped cartilage-like tissues, the helix cartilage-like tissues were wrapped tightly with the gradient hydrogel and did not separate spontaneously despite complete MG degradation. Since the formed cartilage-like tissues were very robust, they could be separated manually while maintaining their helix structures (FIG. 33J). The whole construct also strongly stained with TBO due to the presence of a large amount of GAG (FIG. 33K). The tunability illustrated herein highlights the versatility of this system to engineer cell condensation-based tissues with defined configurations on demand. Our results sufficiently demonstrated this 4D cell-condensate bioprinting strategy possesses high potential in the field of tissue engineering and may be expanded to other regenerative medicine related areas.

The 4D bioprinting technique opens a new avenue to engineer bioconstructs through a user-defined shape-morphing process, enabling dynamic 4D biofabrication at physiologically relevant timescales. Currently, in the 4D bioprinting field, research is still focused on the development of intelligent materials that undergo geometrical transformations under physiological conditions. Several reports to date have shown the feasibility of using cytocompatible polymers as bioinks to print high-resolution hydrogel constructs. In these studies, cells were either seeded on the hydrogel surface or encapsulated inside the formed hydrogels. However, the seeding of cells on the hydrogel surface fails to replicate the 3D cellular microenvironment, while encapsulation of cells inside the hydrogels interferes with critical cell-to-cell interactions. Here we have proposed a novel 4D bioprinting strategy to address the challenges in the formation of geometrically complex scaffold-free 3D cell condensate constructs with defined configurations capable of undergoing temporally controllable and predefined architectural changes. Through a rational bilayer design using a gradient hydrogel as an actuation layer and a photocurable and biodegradable MG layer as a temporally controlled cell condensate-supporting layer, stable cell condensates with diverse and tunable conformational changes over time were obtained through programmable deformations. The cell condensate-derived tissues or other bioconstructs can be liberated after being cultured to a predetermined time point. As a proof-of-concept study, we demonstrated the fabrication of letter “C”-shaped and helix-shaped cartilage-like tissues. The unique features of this system on the whole lie in the (i) adjustable shape morphability, (ii) smooth and high-resolution MG printing and high-fidelity cell-only printability within the pre-printed MG layer, (iii) cytocompatible materials and processing, including 4D printing and shape transformations, and importantly (iv) controllable cell condensate formation, maturation, and release. This is the first time, to the authors' best knowledge, that a 4D system has been implemented to achieve deformable cell condensations. We also observed very limited shape change caused by cell-contractile forces in the ex vivo 4D tissue engineering study. Given that cell-contractile forces can occur strongly within cell condensates and as observed in this study, can be a natural trigger for impelling the 4D process, it might be of value to design a 4D cell-condensate bioprinting system that involves the cell-contractile force as a collaborative, primary or even the sole stimulus.

### Example 5

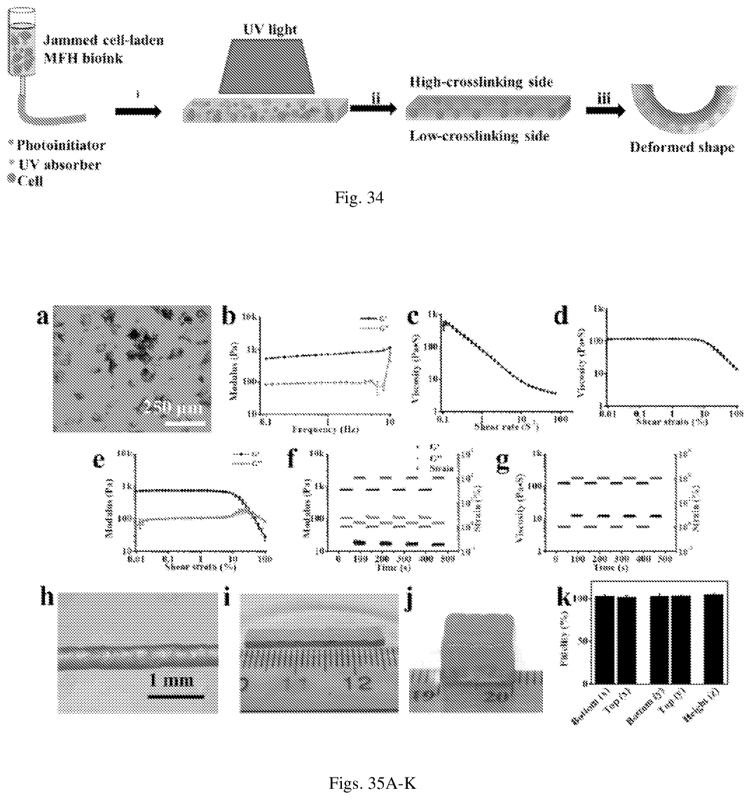

This example describes a jammed heterogeneous single-component micro-flake hydrogel (MFH) system consisting of only ionically crosslinked oxidized and methacrylate alginate (OMA) hydrogels as a cell-laden bioink for 4D living cell bioprinting. This MFH can be easily printed into a stable 3D (bio)construct and can be further crosslinked to form a more robust hydrogel construct with a crosslinking gradient within the hydrogel when a photoinitiator (PI) and a UV absorber are incorporated. The crosslinking gradient of the MFHs in the printed 3D (bio)constructs permits controlled morphing into defined geometries after being cultured in cell culture media (FIG. 34). With this system, bioconstructs with complex structures and high cell viability were obtained through a shape transformation of the 3D printed counterparts. Finally, 4D cartilage-like tissue, as a proof-of-concept, was engineered to demonstrate the potential of the MFH in the field of tissue engineering and regenerative medicine. To the best of our knowledge, this is the first example of a jammed microgel system as the cell-laden bioink for 4D living cell bioprinting.

### Experimental

### Chemicals, Instruments, and General Methods

Unless specified, all solvents and reagents were used without further purification. Sodium alginate (Protanal® LF120M, 251 mPa-S) was a generous gift from FMC Biopolymer (Philadelphia, PA). Photoinitiator (PI) 2-Hydroxy-4′-(2-hydroxyethoxy)-2-methylpropiophenone,3-(4,5-dimethylthiazol-2-yl)-2,5-diphenyl tetrazolium bromide (MTT), fluorescein diacetate (FDA), Dulbecco's Modified Eagle Medium-Low Glucose (DMEM-LG), Dulbecco's Modified Eagle Medium-High Glucose (DMEM-HG), and fetal bovine serum (FBS) were purchased from Sigma (St. Louis, MO). Dexamethasone was purchased from MP Biomedicals (Solon, OH). B-Glycerophosphate was purchased from CalBiochem. ITS+ Premix and penicillin/streptomycin (P/S) were purchased from Corning Inc. (Corning, NY). Sodium pyruvate was purchased from HyClone Laboratories. Non-essential amino acid solution was purchased from Lonza Group (Basel, Switzerland). Fibroblast growth factor-2 (FGF-2) was purchased from R&D Systems (Minneapolis, MN), and transforming growth factor 31 (TGF-β1) was purchased from PeproTech (Rocky Hill, NJ). N-(2-aminoethyl) methacrylate hydrochloride (AEMA) and methacryloxyethyl thiocarbamoyl rhodamine B (RhB) were purchased from Polysciences. Ethidium bromide (EB), 2-(N-morpholino)ethanesulfonic acid (MES), sodium peroxide, sodium bicarbonate, sodium hydrate (NaOH), sodium chloride (NaCl), and calcium chloride dihydrate were purchased from Fisher Scientific (Waltham, MA). N-hydroxysuccinimide (NHS) was purchased form Acros Organic (Fair Lawn, NJ). 1-Ethyl-3-(3-dimethylaminopropyl)carbodiimide hydrochloride (EDC·HCl) was purchased from Proto Chemical (Mumbai, India). 1H NMR spectra were obtained on a 600 MHz Bruker AVIII HD NMR spectrometer equipped with a 5 mm SmartProbe™ at 25° C. using deuterium oxide (D₂O) as a solvent and calibrated using (trimethylsilyl)propionic acid-d4 sodium salt (0.05 w/v %) as an internal reference. Cell growth media (GM) consisting of DMEM-LG with 10% FBS and 1% P/S was used to culture the cell-free and cell-laden hydrogels. Images of hydrogel deformation extent were obtained using a Nikon SMZ-10 Trinocular Stereomicroscope equipped with a cellphone camera. A microplate reader (Molecular Devices iD5, San Jose, CA) was used to read data from the microplates. Bright-field images of stained hydrogels and hydrogels with/without cells were captured on a Nikon Eclipse TE300 inverted fluorescence microscope (Tokyo, Japan) equipped with a 14 MP Aptina Color CMOS digital camera (AmScope, Irvine, CA).

### Synthesis of Oxidized and Methacrylated Alginate (OMA)

OMA with 1% theoretical oxidation and 30% theoretical methacrylation (O1M30A) was synthesized according to the reported literature. Briefly, 10 g of sodium alginate was dissolved in 900 mL of deionized water (diH₂O) overnight, and 0.108 g of sodium periodate (NaIO₄) in 100 mL of diH₂O was rapidly added to the alginate solution under stirring in the dark at room temperature (RT). After reaction for 24 h, 19.52 g of MES and 17.53 g of NaCl were added, and the pH was adjusted to 6.5 with 5 N NaOH. Then 1.77 g of NHS and 5.84 g of EDC-HCl were sequentially added to the mixture. After stirring at RT for 10 min, 2.54 g of AEMA was slowly added. The solution was wrapped with aluminum foil to protect it from light and left to react for 24 h at RT. The mixture was then poured into 2 L of chilled acetone to precipitate out the crude OMA solid, which was further purified by dialysis against diH₂O over 3 days (MWCO 3.5 kDa, Spectrum Laboratories Inc., Rancho Dominguez, CA). The dialyzed OMA solution was collected, treated with activated charcoal (0.5 mg/100 mL, ˜100 mesh, Oakwood Chemical, Estill, SC) for 30 min, filtered through a 0.22 μm filter, and frozen at −80° C. overnight. Following lyophilization for at least 10 days, the final O1M30A product obtained was a white cotton-like solid. The actual methacrylation degree was determined to be 5.7% from 1H NMR data according to the method described in the literature. Note that the actual oxidation was not provided due to the overlap of the proton peak assigned to the CHO group (˜5.4 ppm) with the polymer proton peak (broad peak located at ˜5.1 ppm).

### Jammed Micro-Flake Hydrogel (MFH) Preparation

To make the stock MFHs, O1M30A (1.2 g) was dissolved in diH2O (60 mL) and then slowly dispensed (approximately 20-30 mL/min) into a gelling bath containing an aqueous solution of CaCl₂ (600 mL, 0.2 M) under fast stirring with a magnetic stir bar. After being fully ionically crosslinked overnight, the resultant O1M30A beads were collected, washed with 40 mL of 70% ethanol (EtOH)/water (H₂O) once, and then blended using a household blender (Osterizer MFG, at “pulse” speed) for 2 min with 120 mL of 70% EtOH/H₂O. Then, the OMA microgels were loaded into 50 mL conical tubes and centrifuged at 2000×g (Sorvall ST40R centrifuge, ThermoScientific, Waltham, MA) for 5 min and stored in 70% EtOH at 4° C. for future use.

To make the jammed MFHs, the as-prepared microgels above (5 mL) were washed 3 times by replacing the previous media with 25 mL of diH₂O containing PI (0.05% w/v) and UV absorber (0.02% HMAP or 0.02% HMAP/0.005% RhB w/v), while vortexing (Fisher Scientific, 10× speed) for 2 min between washes, and then washed 2 times with 25 mL of DMEM-LG containing PI and UV absorber while vortexing (10× speed) for 1 min each time between washes.

To evaluate the morphology and measure the size of the jammed MFHs, 100 μL of MFHs were stained with 1 mL of 0.1% safranin 0 solution for 2 h. After staining, the MFH mixture was vortexed for 10 s to disperse homogeneously in the media, and then 200 μL of the stained samples were added into 3 mL of PBS (pH 7.4) and imaged using a fluorescence microscope equipped with a 14 MP APTNA Color CMOS Microscope Camera (AmScope, Irvine, CA) under a bright field. The average diameter of the MFHs was determined by measuring 86 microgels in a representative image (FIG. 35A) using ImageJ.

### Rheological Properties of the MFHs

Dynamic rheological examination of the MFHs was performed to evaluate shear-thinning, shear yielding, and self-healing properties with a Kinexus ultra+ rheometer (Malvern Panalytical). In oscillatory mode, a parallel plate (8 mm diameter) geometry measuring system was employed, and the gap was set to 1 mm. MFHs were placed between the plates. All the tests were carried out at 25° C. Oscillatory frequency sweep (0.1˜100 Hz at 1% strain) tests were performed to measure storage moduli (G′), loss moduli (G″), and viscosity. Oscillatory strain sweep (0.01˜100% strain at 1 Hz) tests were performed to show the shear-thinning characteristics of the MFHs and to determine the shear-yielding points at which the jammed MFHs behave fluid-like. To demonstrate the self-healing properties, cyclic deformation tests were performed at 100% strain with recovery at 1% strain, each for 1 min at 1 Hz.

### Cell Expansion

Human mesenchymal stem cells (hMSCs) were isolated according to previous literature. NIH3T3 fibroblasts, HeLa cells, and hMSCs (P2 to P4) were cultured and expanded in the GM (for NIH3T3 and HeLa cells) or the GM supplemented with 10 ng/mL FGF-2 (for hMSCs) at 37° C. and 5% CO₂ with media changes every 2 or 3 days. The cells were harvested for use when they reached approximately 80% confluence.

### 4D Bioprinting

The printing of the cell-free and cell-laden bioinks was performed using a 3D printer (PrintrBot Simple Metal 3D Printer, Printrbot) modified with a syringe-based extruder. More information about this printer can be found in the literature. The STL files for printing were generated from www.tinkercad.com under open license.

To load the cells into the bioink, MFHs and cells (5×10⁶ cells/mL bioink) were separately loaded into two 3 mL syringes. After the two syringes were connected with a female-female luer lock coupler (Value Plastics), the MFHs and cells were thoroughly mixed, and this cell-laden bioink was ready to use.

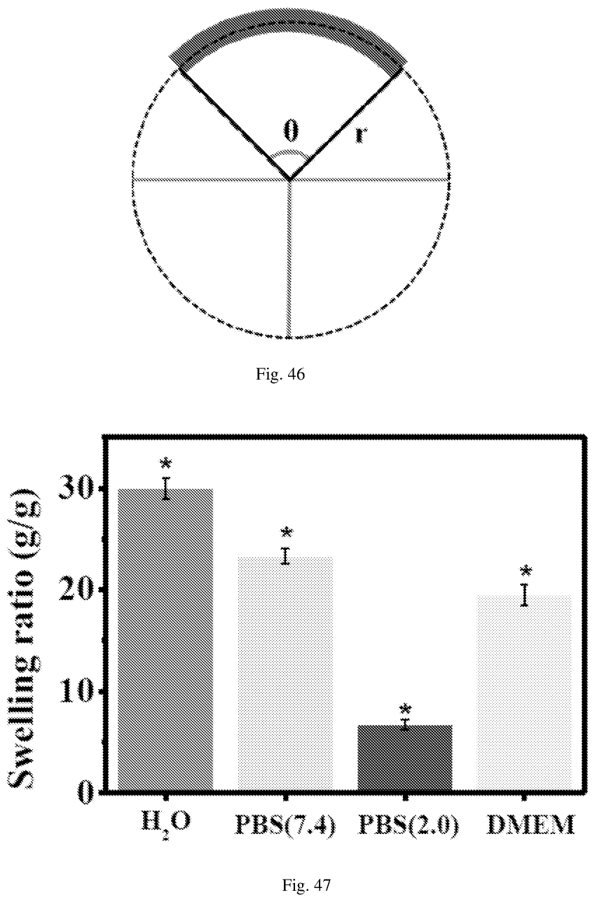

The cell-free and cell-laden bioinks were separately loaded into 1 mL glass syringes (Hamilton, Reno, NV), which were connected to a stainless-steel needle (McMaster-Carr, Elmhurst, IL) and mounted into the syringe pump extruder on the 3D printer. A petri dish was placed on the building platform. The tip of the needle was positioned at the center and near the bottom of the dish, and the print instructions were sent to the printer using the host software (Cura Software, Ultimaker, Geldermalsen, the Netherlands), which is an open-source 3D printer host software. After 3D printing of the bioinks, the resulting constructs were immediately photocured under UV (EXFO OmnicureR S1000-1B, Lumen Dynamics Group, Ontario, Canada) at 12 mW/cm². Then the cell-free or cell-laden constructs were carefully transferred into the wells of 6-well tissue culture plates with 8 mL of media and further cultured to record shape changes. The hydrogels were imaged, and the bending angles were quantified according to the previous literature. Briefly, as shown in FIG. 46, a circle was drawn to match well with the shape of the bent hydrogel curve. The bending angle (0) is defined as the central angle generated by drawing two lines between the endpoints of the hydrogel curve and the circle center, respectively. Unless otherwise specified, MFH bioinks were printed using a 22G needle under 4 mm/s printing speed and 80% infill density and subsequently photocured for 40 s.

### Young's Moduli Measurements

The elastic moduli of the as-printed MFHs and photocured MFHs were determined by performing uniaxial, unconfined constant strain rate compression tests at RT using a constant crosshead speed of 0.8%/sec on a mechanical testing machine (225 lbs Actuator, TestResources, Shakopee, MN) equipped with a 25 N load cell. Elastic moduli of the hydrogels were determined from the first non-zero linear slope of the stress versus strain plots within 10% strain (N=3).

### Determination of Printing Fidelity

The printing fidelity was determined by comparing the dimensions of printed 3D objects with the original CAD cuboid. The designed dimensions were 24 mm (x)×4 mm (y)×0.8 mm (z). The measured dimensions included the bottom (x), bottom (y), top (x), top (y), and height (z), and were compared with the designed structure. The fidelity (%) was calculated as Length_{measured}/Length_{designed}×100% (N=3).

### Swelling Test

The cell-free bioink was printed into cuboids with a dimension of 10 mm×4 mm×3 mm and photocured. The samples were frozen for 4 h at −80° C. and lyophilized for 2 days. The masses of the dried gels were measured as initial weights (Wi). The dried hydrogels were rehydrated by immersing into 1 mL of diH₂O, PBS (pH 7.4 or 2.0), or DMEM-LG and incubated at 37° C. for 10 h. The hydrogels were collected, and the swollen weights (Ws) were measured. The swelling ratios were calculated with the following equation: Ws/Wi (N=3).

### MTT Assay

The cytotoxicity of the HMAP (0.02%, w/v) on monolayer hMSC cells (P4) was assessed by a standard MTT assay. hMSCs were seeded in wells of a 96-well plate (10,000 cells/well) and cultured in 200 μL of GM for 3 days. The GM was then replaced with freshly prepared HMAP-contained DMEM-LG and cultured for different times (0.5-6 h). After the treatment, the media was replaced with GM, and the cells were further cultured to reach a total of 24 hrs of culture time (from the start of treatment to the end of culture). 20 μL of MTT (5 mg/mL in pH 7.4 PBS) was added to each well and further incubated at 37° C. for 1 h. The media was gently aspirated and 100 μL of dimethylsulfide (DMSO) was filled to each well. After 30 min additional incubation, the absorbance at 590 nm was measured using a microplate reader. The control is the group wherein cells were cultured in DMEM-LG only. The cytotoxicity was calculated with the following equation: cell viability (%)=(ODₛₐₘₚₗₑ−OD_{blank})/(OD_{control}−OD_{blank})×100%. N=6.

### Live/Dead Staining

The viability of cells in hydrogels was visualized using Live/Dead staining comprised of FDA and EB. The staining solution was freshly prepared by mixing 1 mL of FDA solution (1.5 mg/mL in DMSO) and 0.5 mL of EB solution (1 mg/mL in PBS) with 0.3 mL PBS (pH 8). At predetermined time points, 20 μL of staining solution per 1 mL of media was added into each well and incubated for 5 min at RT, and z-stacked (stepwise 50 m, totally 1.0 mm) fluorescence images of the samples were taken using an ImageXpress Pico Automated Cell Imaging System equipped with a 5 megapixel CMOS digital camera (Molecular Devices, San Jose, CA). The individual z-stacked images were assembled using CellReporterXpress software (Molecular Devices, San Jose, CA).

### Biochemical Quantification of Chondrogenesis and Toluidine Blue O (TBO) Staining

The hMSC cell (P4)-laden hydrogel bars or other constructs were fabricated as described above. 4D bioprinted hydrogel bioconstructs (hydrogel bar: 24×4×0.6 mm3; four/six-petal flowers: arm length 28 mm, arm width 4 mm, thickness 0.6 mm) for chondrogenesis were cultured in chondrogenic media (CM), which is basal pellet media (BPM) consisting of DMEM-HG with 1% ITS+ Premix, 100 nM dexamethasone, 1 mM sodium pyruvate (CM), 100 μM non-essential amino acids, 37.5 g/mL ascorbic acid-2-phosphate and 1% P/S supplemented with 10 ng/mL TGF-β1. Hydrogel bars were cultured in 6-well tissue culture plates filled with 5 mL of GM (negative control, NC) or CM (experimental group, EG; and positive control, PC), hydrogel four/six-petal flowers were cultured in 6-well tissue culture plates filled with 10 mL of CM, and all were placed in a humidified incubator at 37° C. with 5% C02 for 21 days (3 weeks). Half of the volume of the media was changed every 3 days. Samples for biochemical quantification were collected at predetermined time points (D1, D14 and D21) and stored at −20° C. The harvested bioconstructs were placed in 0.8 mL of papain solution (Sigma) and then homogenized at 35,000 rpm for 2 min using a TH homogenizer (Omni International) on ice. The samples were digested at 65° C. for 24 hours and centrifuged for 10 min at 15,000 rpm, and then the supernatants were collected for DNA and glycosaminoglycan (GAG) quantification (N=3).

Per the manufacturer's instructions, a Picogreen assay kit (Invitrogen) was used to quantify the DNA content in the supernatant. Fluorescence intensity of the dye-conjugated DNA solution was measured using the microplate reader with an excitation of 480 nm and emission of 520 nm.

The GAG content was quantified using a DMMB (1,9-dimethylmethylene blue) assay according to the method described in the literature. Briefly, 40 μL of supernatant from the digested samples was transferred into a 96-well plate, to which 100 μL of DMMB solution was then added. Absorbance at 595 nm was recorded on the microplate reader. GAG content was normalized to DNA content.

A TBO staining assay was used to stain the cartilage-like tissues. Samples for staining were collected and fixed immediately in 10% neutral buffered formalin (NBF) overnight at 4° C., stained with TBO for 30 min, washed with PBS (pH 7.4) 3 times and then imaged. N=2 for strip samples, N=1 sample for flower samples.

### Data Presentation and Statistical Analysis

All quantitative data was expressed as mean±standard deviation. Statistical analysis was performed with one-way analysis of variance (ANOVA) with Tukey honestly significant difference post hoc tests using Origin software (OriginLab Corporation, Northampton, MA). A value of p<0.05 was considered statistically significant.

### Results

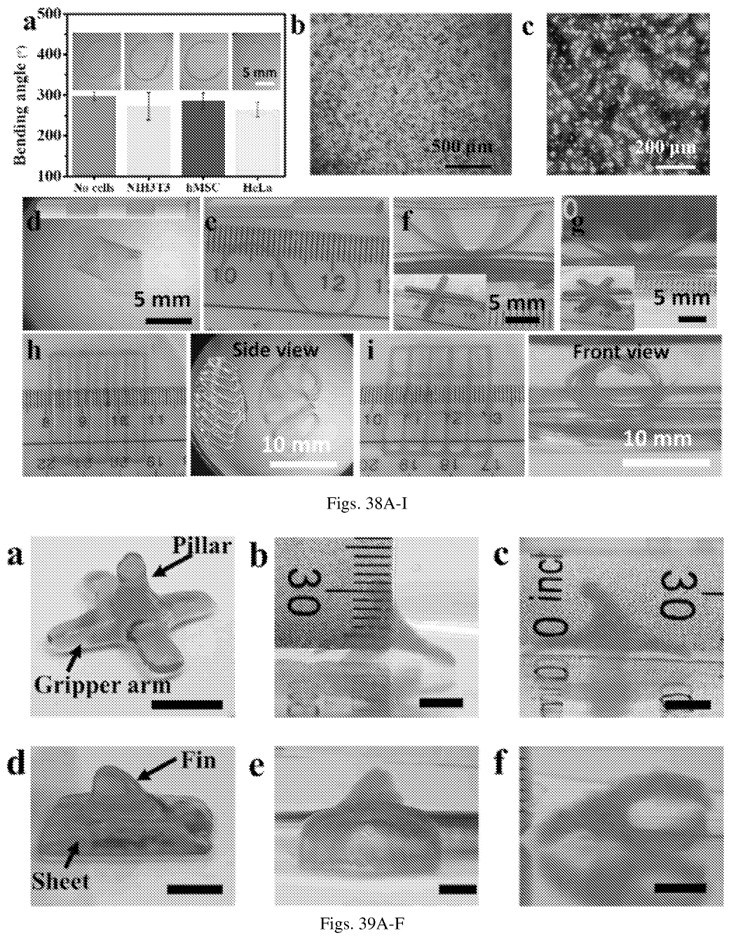

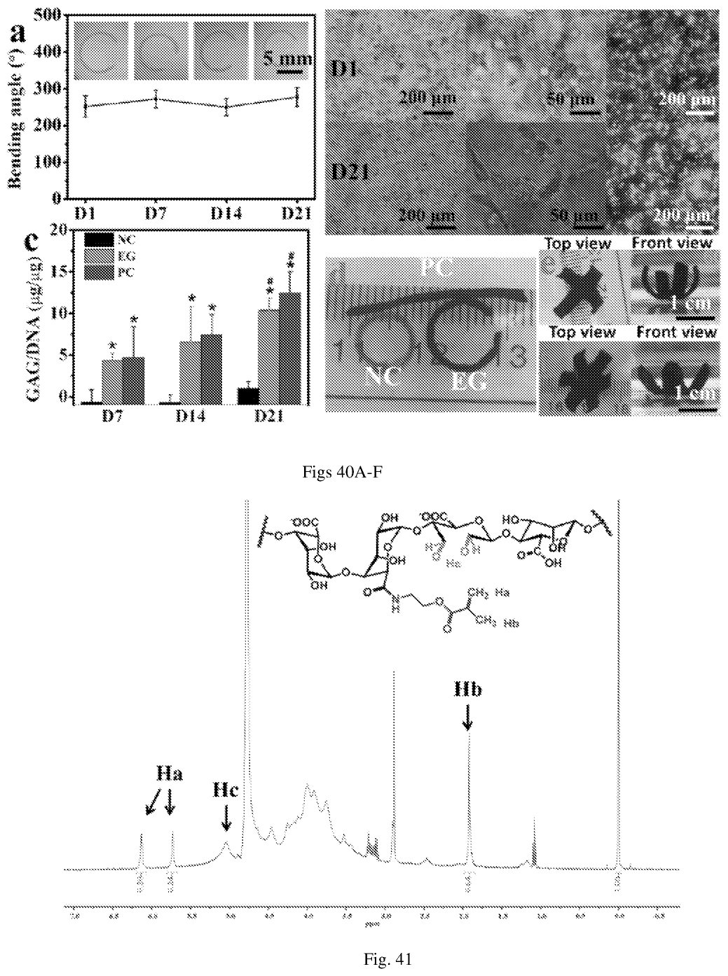

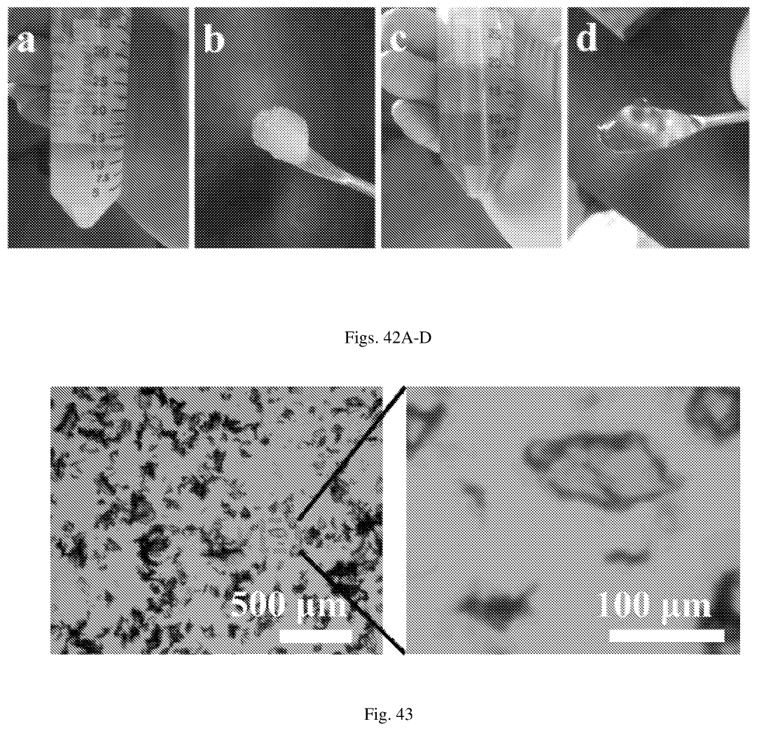

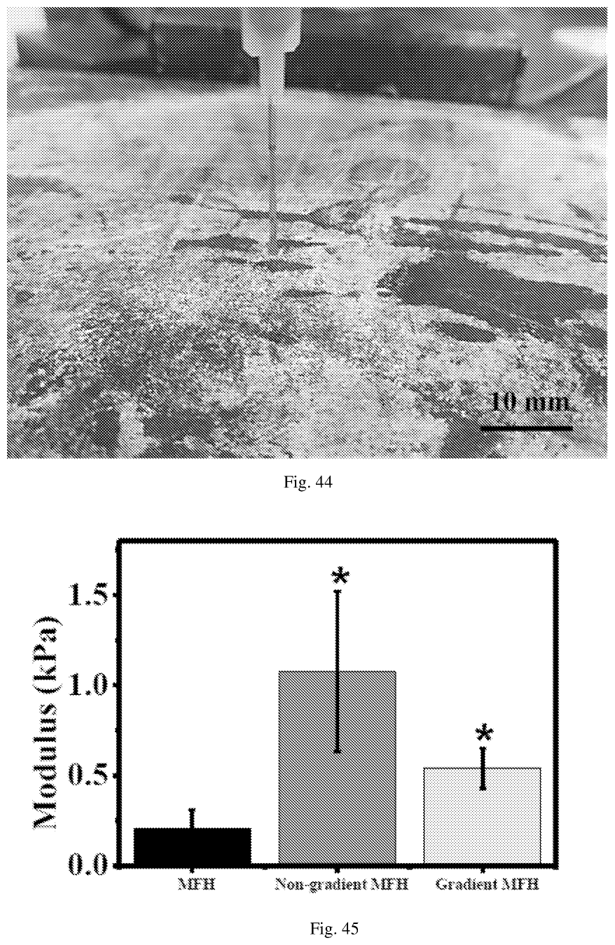

OMA with theoretical oxidation and methacrylation of 1% and 30%, respectively, were synthesized and fully crosslinked with calcium ion (Ca2⁺) to form ionically crosslinked hydrogels by dropping the OMA solution (2%) into a Ca2⁺ solution (0.2 M), and then MFH precursors were fabricated by simple blending using a household blender. The as-prepared MFH precursors could be stably stored in 70% ethanol at −20° C. for few months. The MFH precursors turned into the jammed state with a flake morphology (41.7±19.8 μm, FIGS. 35A and 43) after reconstitution by washing with the PI- and UV absorber-containing media, most likely due to the volume expansion during reconstitution (FIG. 42). These jammed microgels exhibited solid-like behavior at low shear strain (FIG. 35B). Once the MFHs receive an increasing shear rate (FIG. 35C) or a shear strain>10% (FIGS. 35D and 40E), they displayed typical shear-thinning and shear-yielding behaviors. Importantly, the MFHs underwent rapid and repeatable phase transitions upon receiving alternating shear strains between 1% and 100%, demonstrating its capacity for rapid self-healing (FIGS. 35F and 35G). The rheological behaviors of this newly developed MFH agree with our previous findings with different OMA microgel systems. Possessing these properties, the MFHs could be readily extruded evenly through a needle (FIG. 44), form a uniform filament with high printing accuracy (FIG. 35H, 117% of the inner diameter of the needle), and be printed into stable freeform 3D constructs with various shapes (FIG. 35I, J) with high fidelity (FIG. 35K) and stability.

Due to the presence of the methacrylate groups, the microgels can be further stabilized by photocrosslinking under UV light in the presence of a PI (FIG. 45). The incorporation of a UV absorber results in the generation of a light attenuation pathway within the hydrogel and a subsequent a gradient in the crosslinking density (structural anisotropy) (FIG. 34). As a result, the crosslinked gradient MFH showed significantly lower elastic modulus than the crosslinked non-gradient MFH (FIG. 45). This novel post-printing anisotropization approach to generate structural heterogeneity within a 3D printed construct differs from the widely adopted synchronous-programming approach in the current 4D printing field, by which the structural heterogeneity is generated during printing, thus making it a more facile and more flexible approach to design a 3D printable (bio)ink for formation of 4D constructs. The printed construct is then able to morph into a predefined shape after culturing in media.

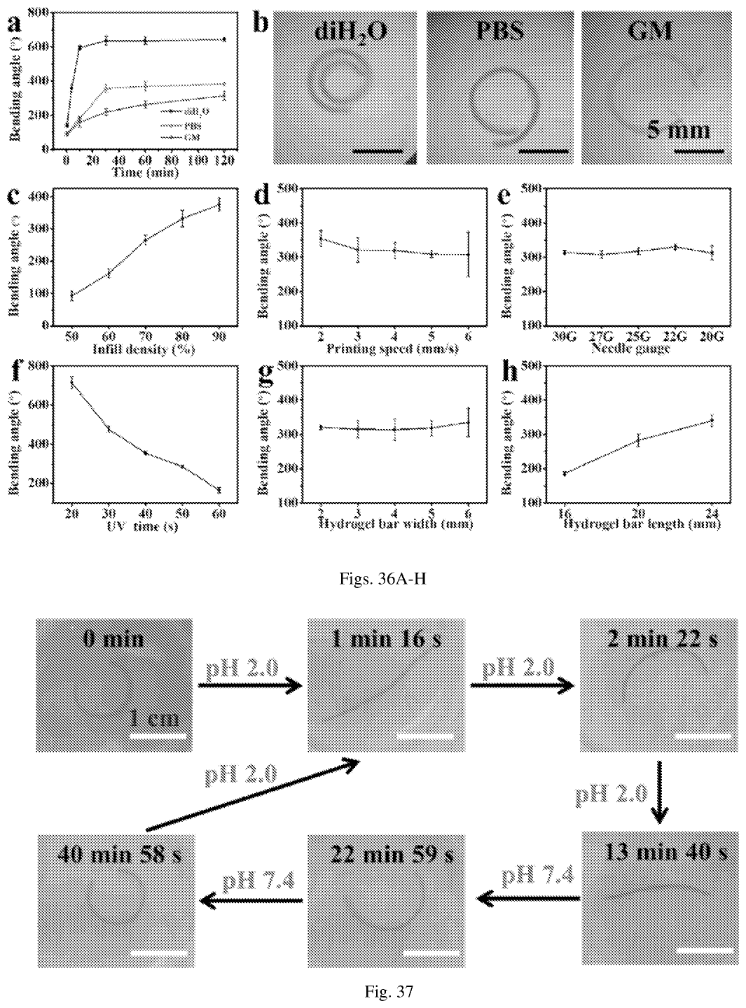

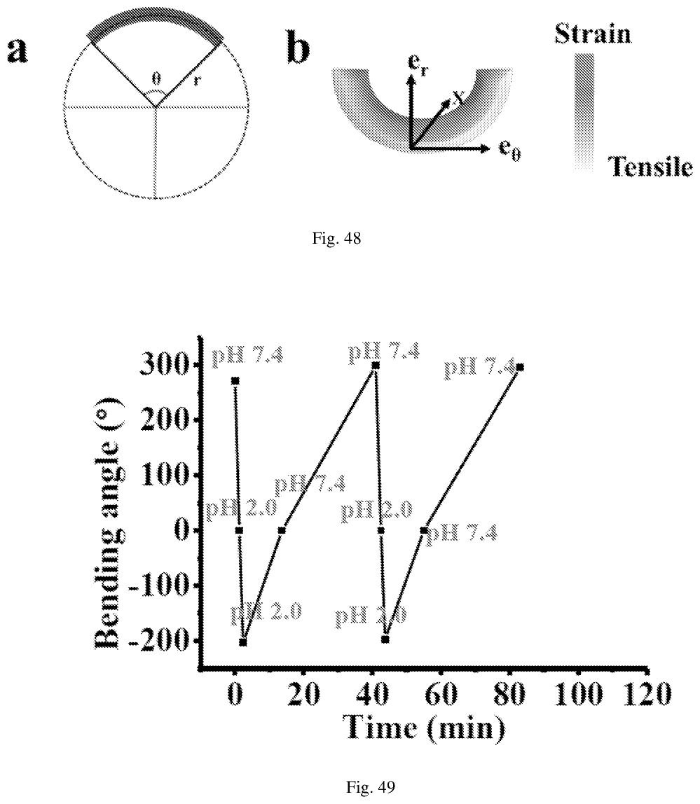

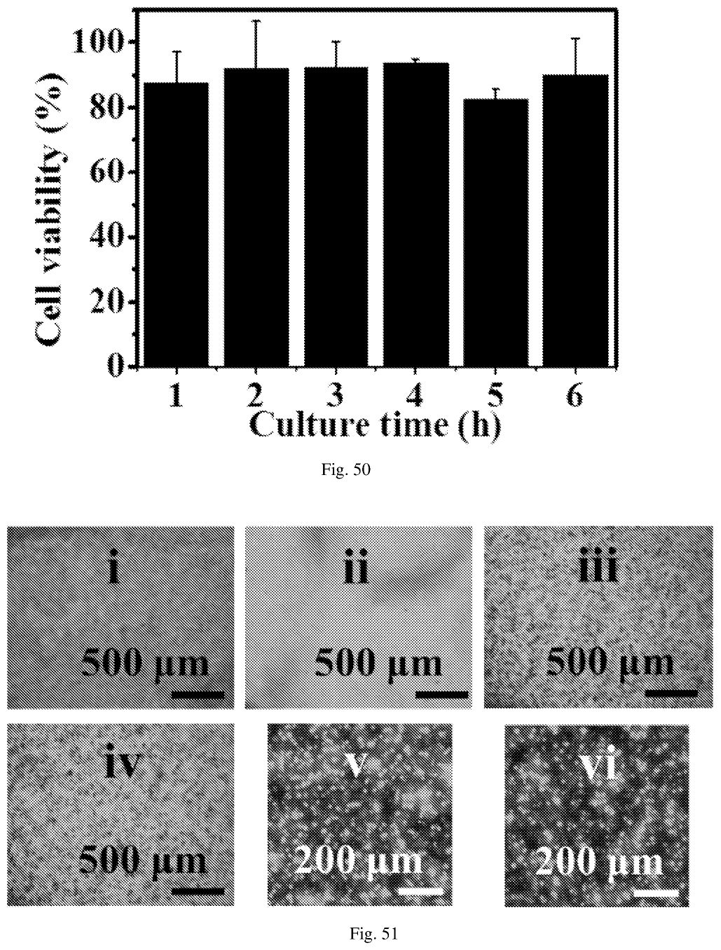

To study the shape-morphing behaviors of the 4D constructs, hydrogel bars with a gradient crosslinking density throughout their thickness were used as prototypes. Unless specified, hydrogel bars with dimensions of 24×4×0.6 mm3 were printed at 80% infill density and 4 mm/s printing speed using a 22G needle. The resulting deformations, which were quantified by bending angles as described in the supporting information (FIG. 46), depend on the structure dimensions, printing parameters, UV crosslinking time, as well as the incubation media. As expected, the hydrogel bars bent to the high-crosslinked side, forming a closed or open hydrogel ring, in the three types of media (i.e., deionized water (diH₂O,), PBS (pH 7.4), and cell growth media (GM)) (FIG. 36A, B). The hydrogel bars in diH₂O exhibited much faster bending kinetics and much larger bending angles than those in PBS and GM, and hydrogel bars in PBS showed slightly higher bending kinetics and angles compared to GM. The distinct variations in bending angles are caused by the swelling differences of the hydrogel bars in the respective medias; that is, a higher swelling ratio, S, led to a larger bending angle (SdiH₂O>SPBS>SGM, FIG. 47). For those printing parameters determined herein (FIG. 41C, D, E), only the infill density obviously influenced the bending angles, while the printing speed and needle gauge imposed negligible effects. Higher filling density brought about larger crosslinking-gradient range due to increased light attenuation, resulting in greater bending. Varying UV irradiation time resulted in a dramatic impact on the bending of the hydrogel bars (FIG. 36F). The bending angle decreased along with the increase of the irradiation time as a higher light dose reduces the crosslinking gradient range throughout the thickness of the hydrogel bar. Additionally, the bending angle increased with the length of the hydrogel bar but did not change with the width (FIG. 36G, H), which aligned with our previous investigations. The bending behaviors of these crosslinked printed hydrogel bars under different conditions can be explained by Timoshenko's theory, a thermal expansion bilayer beam model describing the bending of a bilayer based on mismatched strain in the two layers, which is also widely employed as an empirical theory to interpret the bending behaviors of bilayer hydrogel systems. According to equation (1) following FIG. 47 presented in the supporting information, if we approximately view the gradient hydrogel as a bilayer with a high crosslinking layer and a low crosslinking layer, the resultant bending angle (θ) is proportional to the mismatch of expansion strain (Δε), which is attributable to differential hydrogel swelling resulting from the differences in microgel crosslinking density (Dc). Thus, it is understood that those parameters contributing to a larger Δε, such as enhanced swelling media, increased infill density, and lowered overall crosslinking, could effectively increase the bending angle. However, Timoshenko's theory cannot be used to explain the impact of the length on the bending angle because it assumes bending curvature is independent to beam length. Interestingly, it was observed that increasing in aspect ratio (length/width) of samples with fixed width could result in larger bending (FIG. 36H), which is consistent with the results in reported literature, while the change in aspect ratio in samples with varying width but fixed length did not give rise to observable changes in the bending angle (FIG. 36G). The length of the hydrogel bar is much longer than the width, thereby those hydrogel bars tend to bend perpendicularly with the longitudinal axis to reach a thermodynamically stable state. Since the strain only varies in the radical direction (er) but keeps relatively constant in the tangential direction (eθ) (FIG. 47), the bending curvature κ does not depend on the aspect ratio. According to equation (2) following FIG. 48 in supporting information, the bending angle only correlates with hydrogel length (L) and curvature (κ). That is the reason why length change rather than width change influences the bending angle in these systems. These results indicate that deformation extent can be readily adjusted by tuning the printing parameters and/or hydrogel dimensions. Thus, by varying these parameters, we may obtain a pre-programmable tailored deformation.

In addition to programmable deformation, external stimulation can be applied to further manipulated the shape of a printed construct on demand. To demonstrate the capacity to control shape changes of the MFH-based constructs by external stimuli, a gradient hydrogel bar was first incubated in PBS (pH 7.4) to form a curve (FIG. 37, 0 min) and then subsequently transferred to another PBS solution with a different pH to record the shape changes over time (FIG. 37). The hydrogel bar rapidly stretched after incubation at a low pH of 2.0 for 1 min 16 s and bent to form a backward-facing curve upon further treatment to 2 min 22 s. Interestingly, this curve re-stretched and contracted at a much slower rate until reaching an equilibrium state at 13 min 40 s. After switching the pH back to 7.4, the hydrogel bar reverted to the initial state in a much slower manner (from 13 min 40 s to 40 min 58 s). This may be explained by the differential swelling properties of alginate hydrogel in solutions of different pH. Alginate is a polyelectrolyte that contains both weak acidic and weak basic groups on the polymer chains and these groups can respond to the environmental pH via protonation or deprotonation, leading to a volume change in the way of swelling/shrinkage. For instance, the MFH hydrogels exhibited much smaller swelling ratios at pH 2.0 than at pH 7.4 (FIG. 47). In the initial state (0 min, FIG. 37), the outer side (low-crosslinking side) of the “unclosed” ring is the high-swelling side and has larger pore sizes than the inner side. Therefore, the protons in the solution surrounding the hydrogels diffuse into this side at a faster speed. Thus, the outer side shrank faster than the inner side (high-crosslinking side) due to the better access to the carboxyl groups on the outer side. As a result, the hydrogel bar rapidly stretched and bent to the opposite direction in the first 2 min 22 s. However, since the available carboxyl groups, the reactive moiety receiving the protons, are homogeneously distributed inside the hydrogel, the inverted hydrogel curve at 2 min 22 s re-stretched over time, and by 13 min 40 s was stabilized as a straightened hydrogel at pH 2.0, showing no further shape change. After altering the pH back to 7.4, the straight hydrogel bar in the shrunken phase releases the bound protons to the surrounding solution in a much slower manner due to the smaller pores compared to the hydrogel bar in the fully swelled state at pH 7.4, thereby exhibiting a much slower shape recovery process. This is the first example of a single-component and single-layer hydrogel showing multiple shape transitions with only pH stimulation, and this multiple-shape transition can be realized in multiple cycles without showing signs of fatigue (FIG. 49). Although the low pH (2.0) is not applicable for live cell culture, the reversible shape conversion suggests the potential to use this 4D printed hydrogel as an environment-controlled actuator/robot in some specific conditions, such as the gastric environment (pH 1.5˜3.5).

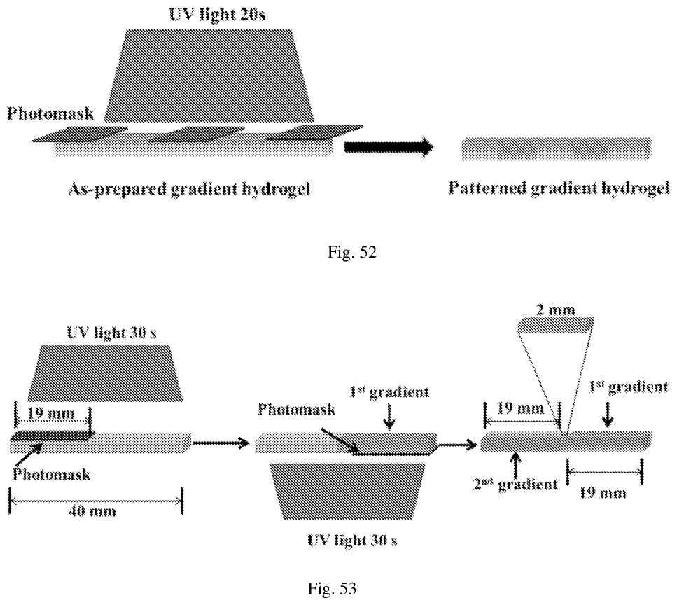

Hydrogels fabricated with covalently crosslinked and/or ionically crosslinked OMA have been extensively used as cell scaffolding materials for tissue engineering. The HMAP is a highly efficient and cytocompatible UV absorber for crosslinking gradient generation. To demonstrate the feasibility to use the jammed MFHs as cell-laden bioinks for 4D living cell bioprinting, three types of cells were examined: a fibroblast cell line (NIH3T3), a cancer cell line (HeLa), and primary stem cells (human bone marrow-derived mesenchymal stem cells, hMSCs). These cells were individually mixed with the PI- and UV absorber-containing MFHs (5×10⁶ cells/mL MFH) and printed into hydrogel bars as described earlier, which were then cultured in GM to investigate the resulting shape changes. The printed cell-embedded hydrogel bars showed comparable bending with the cell-free counterparts in all cases (FIG. 38A), and the encapsulated cells (FIGS. 38B, 51i and 51ii) remained highly viable after 24 h culture (FIG. 38C, 51iii-51vi). The results indicate this 4D system is highly compatible for inclusion of live cells and may be useful for fabricating other bioconstructs with more sophisticated geometries. Inspired by this, we sought to fabricate various bioconstructs with more complex geometries by integrating gradient formation with a mask-based photolithography or intricate bioprinted geometric designs. With masked-based photolithography, we locally photocrosslinked the cell-laden hydrogel bar using photolithography and cultured the hydrogel bar in GM at 37° C. to elicit more complex shape transformations beyond unidirectional bending. For example, a pre-formed gradient hydrogel bar with a further discrete local photocrosslinking (schematic in FIG. 38D, FIG. 52) or an as-printed hydrogel bar subsequently photocrosslinked with two separate gradients in opposite directions (schematic in FIG. 38E, FIG. 53) turned into a “biohelix” (FIG. 38D) or a “bioS” structure (FIG. 38E), respectively, after deformation. Alternatively, by printing MFH bioinks into specific geometries, cell-laden hydrogels with more complex structures can be obtained. Printed multi-arm gradient hydrogels morphed into “pseudo-four petal” and “pseudo-six petal” flowers (FIGS. 38F, 53A, 38G, and 54). When the cell-laden MFH bioinks were printed into specific “kirigami-based” structures displaying bar-grid patterns, the bioconstruct with no inner horizontal bars self-curled into a curved cage that crudely resembles the human rib cage (FIGS. 38H and 54C), and the construct with inner horizontal bars self-curled into a “net tube” (FIGS. 38I and 54D). It is worth mentioning that these 4D-engineered bioconstructs are very robust and can maintain their geometry even when exposed to strong agitation, with manipulation of the “net tube” presented as an example.

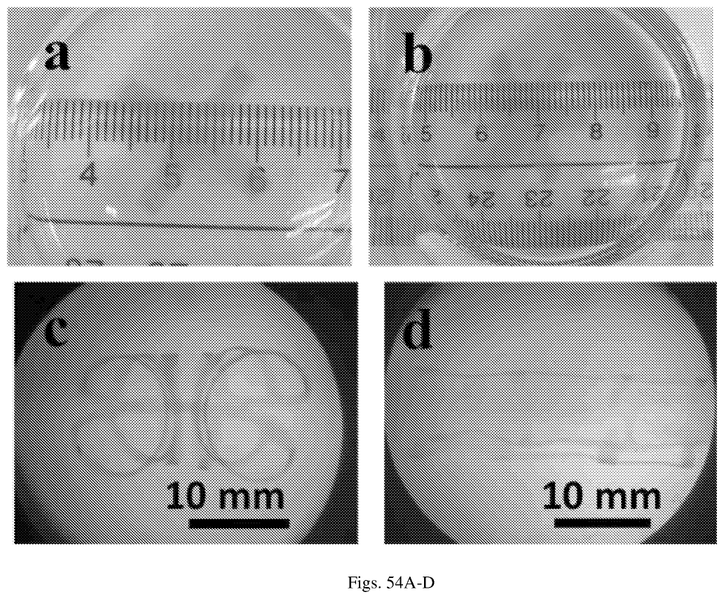

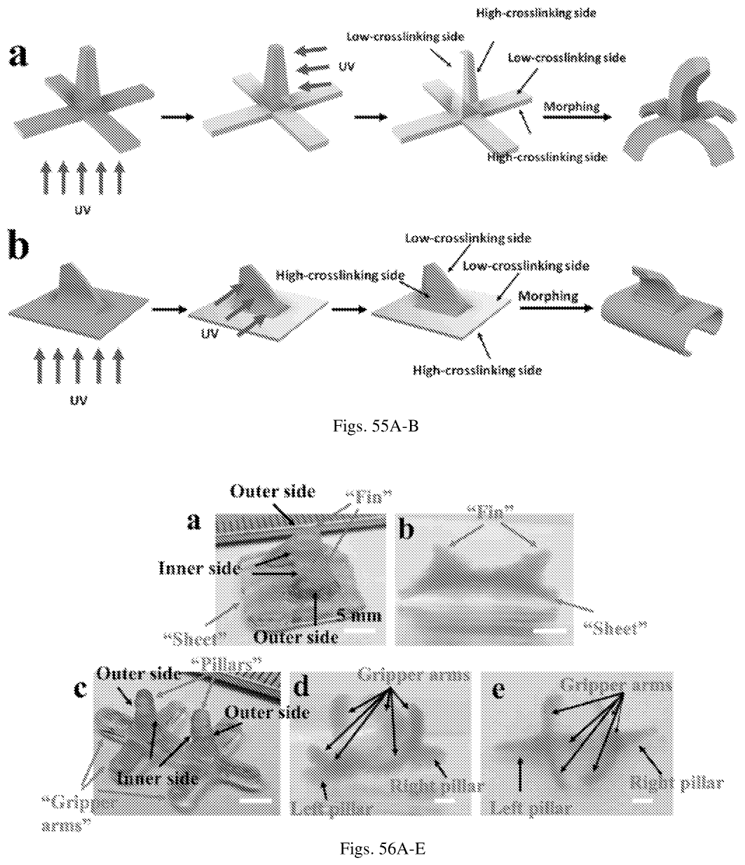

Currently, all previous 4D bioprinting work only presents shape transformation from 2D and/or 2.5D (2D structure with a certain addition in the z-direction) to 3D. To the best of our knowledge, 3D-to-3D shape morphing of cytocompatible biomaterials with encapsulated cells, enabled by 4D bioprinting or any other means, has not been reported. 3D-to-3D morphing is particularly challenging for hydrogel materials due to the difficulty in obtaining a stable printed 3D structure with effective structural anisotropy incorporation. Since our system allows 3D printing and independent anisotropy generation, it is possible to achieve 3D-to-3D transformations of constructs fabricated in a single print in a controllable manner. 3D architectures such as a “pillar gripper” (FIG. 49A) and a “shark-fin sheet” (FIG. 39D) were readily printed. Multiple location-specific crosslinking gradients in the two representative 3D constructs were then created. For example, gradients in the bases of the gripper and the sheet were created by applying UV irradiation from the bottom of the constructs, while the gradients in the pillar and shark-fin were created by applying UV irradiation from the side of the pillar and shark-fin (FIG. 54). With this unique structural anisotropy, complex 3D-to-3D shape morphing with controlled location-specific deformations was then achieved (FIG. 39B, C, E, F). With advanced designs, 3D constructs with more sophisticated structures, such as a “double shark-fin” sheet (FIG. 55A) and a double “double pillar gripper” (FIG. 55C) both presenting a crosslinking gradients from the inner sides (low-crosslinking sides) to the outer sides (high-crosslinking sides) within the “fins” or “pillars” and a separate different crosslinking gradient from bottom to top within the “sheet” or “gripper”, demonstrate the capacity for more complex shape morphing (FIG. 55B, D, E). These 3D-to-3D shape transformations suggest the feasibility and reliability of this system for developing more transformable 3D structures due to excellent printability and ease of anisotropy incorporation.

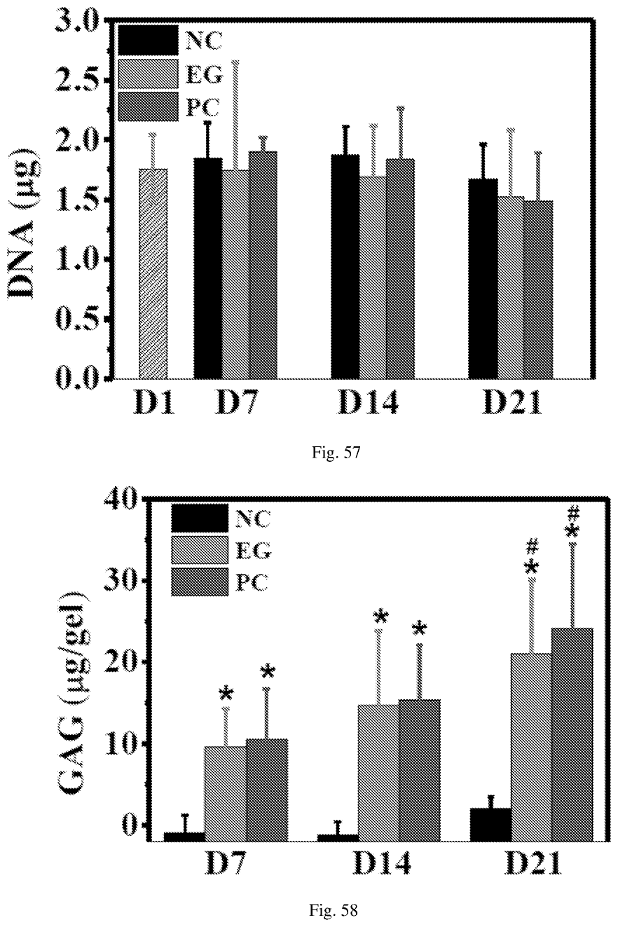

To utilize this type of shape morphing strategy for tissue engineering applications, it is important that the 4D cell-laden constructs enable and/or drive encapsulated cell differentiation and formation and maturation of new tissue. The 4D bioprinting system reported here enables fabrication of architecturally complex bioconstructs while at the same time facilitating the engineering of functional tissues. Since the hMSC is a multipotent stem cell with the capacity to differentiate down multiple connective tissue lineages when provided with appropriate environmental cues, it is a promising cell source for engineering tissues such as cartilage, bone and fat. Hence, we cultured 4D bioprinted hMSC-incorporated MFHs in chondrogenic media (CM) to induce the formation of cartilage-like tissue with relatively predefined final configurations. The chondrogenesis of the 4D hMSC-laden hydrogel bars along with their shape changes was continuously monitored over a course of 21 days. The initially straight gradient hydrogel bars bent into “C” shapes in CM within 2 h and the shapes of the bent hydrogel bars changed very little during the course of chondrogenesis (FIG. 39A), suggesting good stability of the 4D bioconstructs. Meanwhile, the morphologically round cells on day 1 (D1) maintained a predominantly round morphology and high cell viability after 21 days of culture (FIG. 39B, left and right panels). However, elongation of some of the cells that resided near the hydrogel surface was observed on D21 (FIG. 40B, middle panel). To quantify the chondrogenesis, levels of DNA and the primary cartilage extracellular matrix component, glycosaminoglycan (GAG), were analyzed. The DNA content manifested a relatively constant level over time, with no significant difference found when comparing the experimental groups (EG, 4D bioprinted hydrogel bars cultured in CM) to the negative control (NC, 4D bioprinted hydrogel bars cultured in GM) and positive control (PC, 3D bioprinted hydrogel bar photocrosslinked in the absence of UV absorber cultured in CM) (FIG. 56). In contrast, GAG production steadily increased during the 21 days of culture and was similar to the PC group but significantly higher than the NC group (FIGS. 40C and 57). This difference in GAG production was corroborated by the intense positive toluidine blue O (TBO) staining only in EG and PC samples (FIG. 40D). The results imply that the structural self-remodeling is independent of cell differentiation and tissue maturation, and vice versa. Therefore, this decoupling of shape morphing with tissue maturation enables flexible 4D design for 4D tissue engineering. With respect to the formation of 4D cartilage-like tissues with more complex shapes, four- and six-petal flower-shaped cartilage-like tissues were also fabricated (FIGS. 40E and F). Results from this study demonstrate that this jammed MFH system satisfies two critically important criteria for its use in 4D bioprinting for tissue engineering: i) controlled shape morphing capacity and ii) support of new tissue formation by incorporated cells.

4D bioprinting opens new avenues to fabricate cell-laden constructs with dynamic shape morphing capabilities and complex configurations, which are beyond the capacity of conventional 3D bioprinting. The bioprinted constructs can be further induced to form specific tissues when culturing in an appropriate environment. Thus, this newly emerging technology also enables morphodynamical tissue engineering, bearing the potential to biomimic the conformational evolutions occurring during tissue development and healing. So far, a few reports have prepared shape-morphing constructs through 3D printing to fabricate scaffolds with sophisticated structures and investigate and/or modulate cell behaviors, such as proliferation, alignment, and differentiation. However, the cells involved in those studies were only seeded on the post-printed scaffolds rather than encapsulated within the constructs due to the lack of or limited cytocompatibility of materials, harsh printing process, and/or extreme stimulation conditions applied to induce shape change. Hence, those studies could not meet the critical requirement of loading and maintaining viable cells within 4D bioprinted constructs. Although live-cell 4D bioprinting was realized using alginate-, silk-, and gelatin-derived cytocompatible materials, limitations were still present in terms of either shape morphing or capacity to use the materials as cell-laden bioinks for direct ink writing (DIW). For example, the cell-free parts rather than the cell-laden parts in the constructs were the active parts driving the morphing of the constructs, or the inks were just simple macromer solutions lacking appropriate rheological properties for deposition to form stable freestanding 3D objects. Here, we show the first 4D DIW bioprinting system enabling live cell encapsulation using microgels and cells as bioinks. The single-component jammed MFHs by themselves, without the need of other filler components or a slurry support bath, are rheologically favorable for excellent printability (FIG. 36). Specifically, anisotropy (gradient crosslinking) was generated using a post-printing anistropization approach, which liberates the anisotropy generation from printing. With this approach, the anisotropy formation is tunable, enabling facile user-adjustable shape morphing (FIG. 36). In addition, anisotropy can be incorporated in multiple ways within a single construct to produce complex 3D geometries (FIGS. 38D and E). With this system, 4D tissue maturation was also demonstrated using hMSCs and MFHs in a proof-of-concept 4D cartilage regeneration study (FIG. 39). Cartilage-like tissues with complex geometries, such as “C” shape and four-/six-petal flowers were engineered. It is noteworthy that curved, bent, folded, and rolled structures often emerge in tissue morphogenesis during processes such as development of gut villi and mammary epithelial acini. Our results suggest important progress for 4D live-cell bioprinting, which would benefit morphodynamic tissue engineering.

In this Example, a new single-component jammed MFH system with heterogeneous size distribution has been developed as a cell-laden bioink for 4D living cell bioprinting. This new bioink showed desirable shear-thinning, shear-yielding, and rapid self-healing properties, and was directly deposited into various 3D bioconstructs with high resolution and high fidelity in the absence of a support bath. 4D shape changes were achieved under physiological conditions and high cell viability was maintained after an effective generation of a crosslinking gradient within the hydrogels using a specific post-printing anisotropization method. In addition, it was also demonstrated that multiple-shape transformations (multiple bending and stretching cycles) could be elicited by a singular, although non-biocompatible, stimulation (low pH) in this single-component and single-layer system, which has not been reported in other existing systems. By utilizing this bioink, shape morphing cell-laden bioconstructs with well-defined configurations were fabricated by combining photomask based photolithography and/or intricate geometric designs. Ultimately, proof-of-concept 4D cartilage-like tissue formation was demonstrated in curved hydrogel bars and folded four- and six-petal flowers. We anticipate this unique 4D bioprinting system will have promising applications in 4D tissue and organ engineering and potentially aid in the study of developmental processes.

### Example 6

In this example, cell-laden hydrogels with predetermined temporal changes in geometry due to cell contraction forces are generated using extrusion bioprinting. Oxidized and methacrylated alginate (OMA), gelatin methacrylate (GelMA), and gelatin microspheres are combined to form both an extrudable bioink and a microenvironment that can be deformed by cellular forces. Hydrogel bilayers with one cell-laden and one cell-free layer are generated, and the rate, extent, and final shape of the constructs is precisely controlled by patterning either the cell-laden or cell-free layer. Finally, hMSC-laden constructs are generated to illustrate the ability to simultaneously induce temporal shape change and differentiation of hMSCs into chondrocytes to produce cartilaginous constructs with complex geometries.

### Methods

### Cell Culture

NIH 3T3 (ATCC) were cultured in low-glucose Dulbecco's modified eagle medium (DMEM) supplemented with 10% fetal bovine serum (FBS) and 1% penicillin/streptomycin (P/S). Upon reaching 90% confluence, cells were trypsinized, counted, and pelleted at 100 million cells per vial to be combined with 1 mL of composite bioink. After printing, constructs were cultured in high-glucose DMEM supplemented with 10% FBS and 1% P/S.

### OMA Microgel Synthesis

Alginate was modified with 2% oxidation and 30% methacrylation according to previously published protocols. Briefly, 1% sodium alginate (10 g, Protanal LF 20/40, FMC Biopolymer) solution was dissolved in ultrapure deionized water (diH₂O, 900 ml) by stirring overnight at room temperature (RT). 216 mg of sodium periodate was dissolved in 100 ml of diH2O, mixed with the alginate solution to achieve 2% theoretical alginate oxidation and reacted in the dark at RT for 24 hrs under stirring. 2-morpholinoethanesulfonic acid (MES, 19.52 g, Sigma) and NaCl (17.53 g) were then dissolved in the oxidized alginate solution and the pH was adjusted to 6.5 using 4 N NaOH. N-hydroxysuccinimide (NHS, 1.77 g, Sigma) and 1-ethyl-3-(3-dimethylaminopropyl)-carbodiimide hydrochloride (EDC, 5.84 g, Sigma) were dissolved into the mixture. AEMA (2.54 g, Polysciences) was then slowly added to the solution to achieve a theoretical methacrylation level of 30%. The reaction was conducted at RT for 24 hrs in the dark. The reacted OMA solution then was poured into excess acetone to precipitate the OMA. The precipitate was dried in a fume hood and subsequently dissolved in diH₂O at a 1% w/v concentration. The OMA solution was dialyzed for purification using a dialysis membrane (MWCO 3500, Spectrum Laboratories Inc.) for 3 days. The dialyzed OMA solution was collected and treated with activated charcoal (5 g/L, 50-200 mesh, Fisher) for 30 min. The solution was further purified and sterilized by filtering through a 0.22 μm pore membrane and then lyophilized. After lyophilization, OMA was dissolved in MilliQ water to form a 2% w/v solution. OMA was then added dropwise to a beaker of 0.2 M calcium chloride under vigorous stirring and allowed to ionically crosslink for four hours. Crosslinked OMA was collected and placed in a blender (Oster) with 100 mL of 70% ethanol. OMA was blended for two minutes before adding 50 mL of 70% ethanol and blending for two more minutes. OMA microgels and ethanol were collected into 50 mL conical tubes, centrifuged at 4200 rpm for 5 minutes, and stored at 4° C.

### GelMA Synthesis

GelMA was synthesized according to previously established protocols. Briefly, 10 g of gelatin (type A, Sigma Aldrich) was dissolved in 100 ml of PBS (pH 7.4) and heated to 50° C. Then 10 ml of methacrylic anhydride was added into the 10% gelatin solution and reacted for 1 hr at 50° C. and then stirred overnight at RT. GelMA was precipitated with acetone, purified via dialysis at 50° C. for 7 days with a MWCO 12-14 k membrane (Spectrum Laboratories Inc.), sterilized via a 0.22 mm pore filter, and then lyophilized.

### NMR Analysis

To obtain ¹H-NMR spectra, the OMA and GelMA were separately dissolved in deuterium oxide (D2O) at 2 w/v % and the samples were analyzed via ¹H-NMR spectrometer (Varian Unity-300 (300 MHz) NMR spectrometer (Varian Inc.)). 3-(trimethylsilyl)propionic acid-d4 sodium salt (0.05 w/v %) was used as an internal standard. The actual methacrylation of the OMA and GelMA was determined from ¹H NMR spectra based on the ratio of the integrals for the internal standard protons to the methyl and methylene protons of methacrylate (FIG. 63).

### Gelatin Microsphere Synthesis

Gelatin microspheres were synthesized using previously described methods. Briefly, 11.1 wt % acidic gelatin (Sigma-Aldrich) was added dropwise into 250 ml of olive oil (Gia Russa, Coitsville, OH) and stirred for 10 minutes at 45° C. The temperature of the stirring solution was lowered to 4° C. for 30 minutes, and 100 ml of chilled acetone was added. After 1 hour of constant stirring, another 100 ml of chilled acetone was added and stirring was continued for 5 minutes. Microspheres were then collected via filtration, washed with acetone to remove residual oil, and air dried.

### Composite Cell-Laden Bioink Preparation

On the day of an experiment, OMA microgels were reconstituted through three washes of 0.05% photoinitiator (PI)—containing MilliQ water and two washes of low-glucose DMEM with 0.05% PI and without sodium bicarbonate. Lyophilized GelMA was weighed and dissolved directly in the reconstituted OMA microgels. Lyophilized gelatin microspheres were weighed and rehydrated for 15 minutes in low-glucose DMEM with 0.05% PI at a rate of 15 L/mg. The desired volume of OMA/GelMA mixture was measured and added to the rehydrated gelatin microspheres. One mL of this solution was added to a pellet of 100 million cells to form the cell-laden bioink.

### Single Layer Printing

A 3 mL syringe with a 22 gauge needle was loaded with cell-laden bioink and placed in the Cellink BIOX 3D printer. Square constructs measuring 10 mm×10 mm×0.6 mm were printed from a custom-made STL file and using a print speed of 4 mm/s, and extrusion rate of 1.2 μl/s, and an infill density of 60%. Printed constructs were crosslinked with ultraviolet (UV) light at an intensity of 12 mW/cm² and subsequently transferred to 6 well plates containing 8 mL of media. To minimize disturbance to the constructs during culture, half the media was removed and replenished every day. Constructs were imaged daily using a dissection microscope and a Samsung Galaxy S9.

### Bilayer Printing

To accomplish bilayer printing, two syringes were loaded into the 3D printer: the first contained only reconstituted OMA microgels (cell-free bioink); the second contained the cell-laden bioink. For bilayer rectangles, a 25 mm×25 mm×0.2 mm square was printed with the cell-free bioink. Three cell-laden rectangles measuring 18 mm×4 mm×0.6 mm were then printed directly on top of the cell-free square, spaced 4 mm apart. After crosslinking constructs with UV light, the cell-free layer was cut with a razor blade to match the geometry of the cell-laden rectangles, forming bilayer rectangles. Dead controls were generated by incubating cells in acetone for 30 minutes before forming the cell-laden bioink.

To print constructs with complex cell-laden geometries, similar protocols were followed. A large cell-free layer was printed, followed by a patterned cell-laden layer. If necessary, the cell-free layer was cut to match the cell-laden layer. Constructs with complex cell-free geometries were created by first printing a 12 mm×9 mm×0.6 mm rectangle and subsequently printing the patterned cell-free layer directly on top.

### Cytochalasin D Treatment

To further illustrate the role of cell-generated forces in the observed spatiotemporal changes, the effect of Cytochalasin D, a known inhibitor of actin polymerization, was studied. Cytochalasin D was weighed and dissolved in sterile dimethyl sulfoxide (DMSO) at a concentration of 5 mM and stored at 4° C. Bilayer rectangles were printed and cultured in culture media supplemented with 0.1% v/v Cytochalasin D, resulting in a final Cytochalasin D concentration of 5 μM. Half of the media was replaced every day, with fresh Cytochalasin D added at 0.1% v/v each time. To ensure that any change in behavior is being caused by Cytochalasin D and not DMSO, four bilayer rectangles were cultured in culture media with 0.1% v/v DMSO. These media conditions were also compared to bilayer rectangles cultured in normal culture media.

### Chondrogenesis in 4D Printed Constructs

Human mesenchymal stem cells (hMSC) were cultured in low-glucose DMEM supplemented with 10% FBS, 1% P/S, and 10 ng/mL fibroblast growth factor-2 (R&D). Upon reaching 80% confluence, cells were trypsinized, counted, and pelleted at 100 million cells per vial to be combined with 1 mL of composite bioink. After printing, constructs were cultured in high-glucose DMEM supplemented with ITS+, NEAA, P/S, sodium pyruvate, dexamethosone, and 10 ng/mL TGF-β1.

### Histology

After culture duration was complete, samples were fixed overnight in 10% neutral buffered formalin at room temperature. Samples were then dehydrated through one-hour incubations in 70% ethanol, 95% ethanol, 100% ethanol, 1:1 mixture of ethanol and xylenes, and two separate 100% xylenes. Samples were then submerged in molten paraffin for at least 24 hours before being embedded in paraffin blocks. 5-μm sections were obtained from blocks using a microtome and mounted on microscope slides. Hematoxilyn and eosin (H&E) sections were stained with hematoxilyn for 2 minutes followed by counterstaining with eosin for 30 seconds. Safrananin O and fast green sections were stained with 0.1% Safranin O for 5 minutes followed by counterstaining with 0.05% fast green for 1 minute. Alcian blue and nuclear fast red sections were stained with alcian blue (pH=0.2) for 30 minutes followed by counterstaining with nuclear fast red for 5 minutes.

### Biochemical Assays

DNA and GAG values were quantified according to previously described methods. Briefly, GAG values were quantified by measuring the absorbance of DMMB-bound samples at 595 nm using a plate reader. DNA values were similarly quantified by measuring fluorescence intensity of PicoGreen-bound samples at an excitation of 480 nm and emission of 520 nm. GAG values were normalized to DNA values to produce a quantitative measure of GAG production per cell.

### Bending Angle Quantification

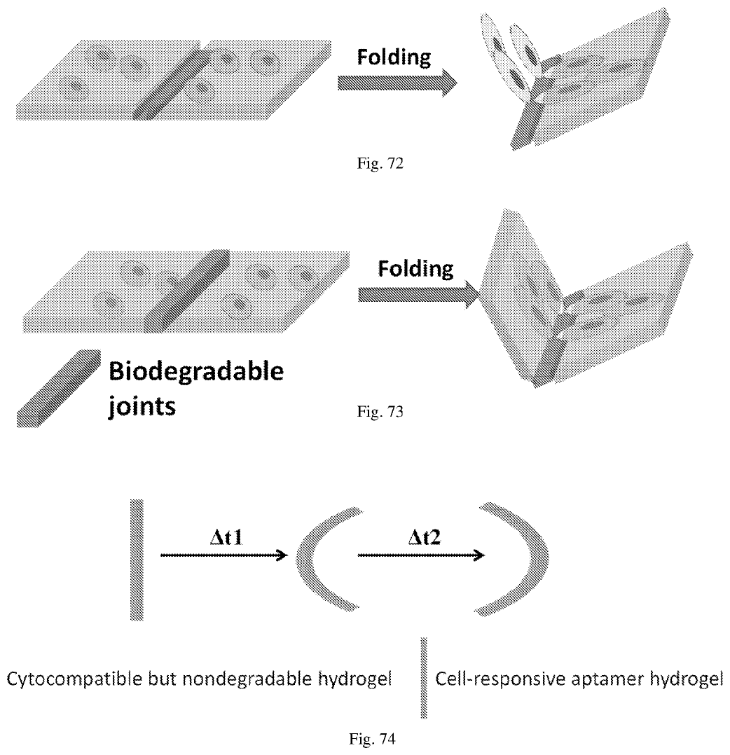

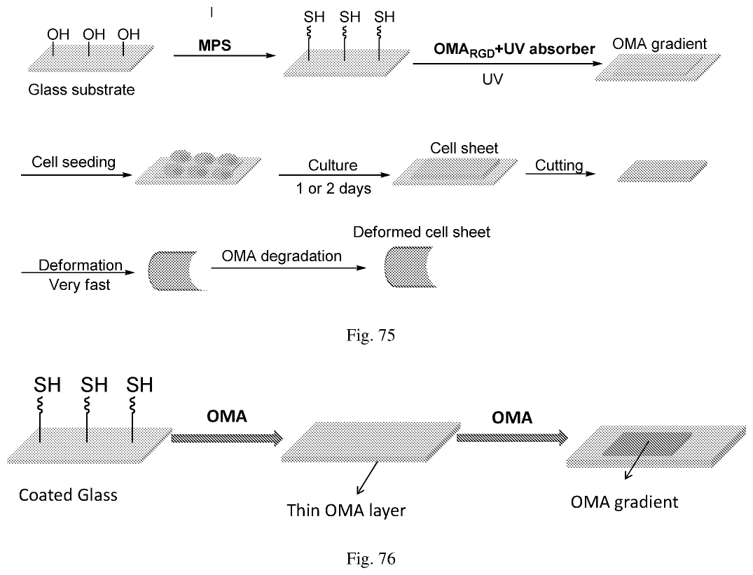

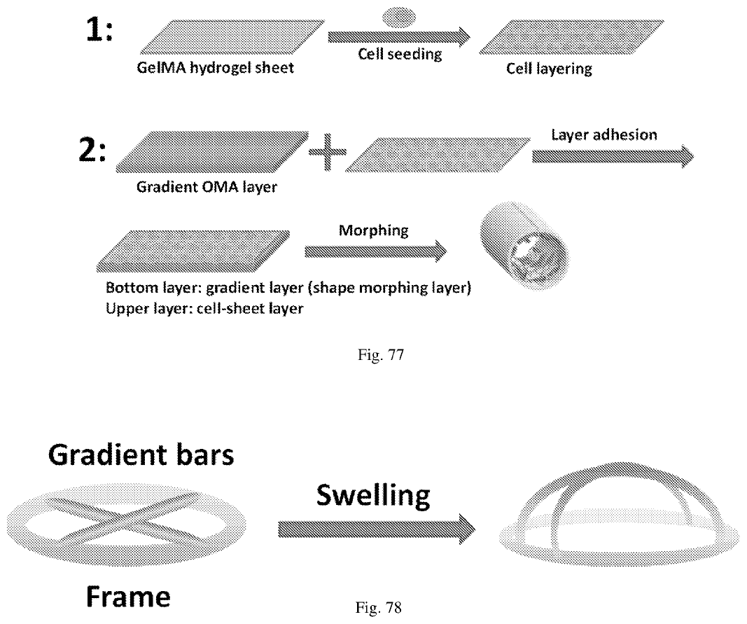

Dissection microscope images were used to measure bending angle as described in FIG. 72. Briefly, a circle with crosshairs was superimposed on each image using Microsoft Powerpoint. The dimensions of the circle were modified to fit the arc of the construct in the image. The image and circle were then copied into ImageJ and the angle tool was used to measure the angle between one end of the construct, the intersection of the crosshairs, and the other end of the construct. Using this method, a construct that has bent into a half circle is measured as 180 degrees, while a construct whose ends are touching is measured as 360 degrees.

### Live/Dead Staining

Printed constructs were stained with fluorescein diacetate and propidium iodide to visualize live and dead cells, respectively. These stains were incubated with printed constructs for 5 minutes, after which all media was removed and samples were immediately imaged.

### Statistics

All graphs are reported as mean±standard deviation. For data with more than two groups, significance was determined using one-way ANOVA with a post-hoc Tukey HSD test. For data with only two groups, significance was determined using the student's t-test. The level of significance is α=0.05 unless otherwise specified.

### Results and Discussion

A composite bioink was carefully tuned to satisfy two opposing mechanical needs: (1) the bioink must be strong enough to ensure stability of the construct after printing, and (2) the bioink must be weak enough for cell-generated forces to be sufficient to drive shape changes. To accomplish this, OMA was processed into a jammed microgel state as previously described, creating the foundation of a bioink with known printability. Since OMA does not include cell-binding sites, GelMA was added to enhance cell-ECM interactions. Finally, uncrosslinked gelatin microspheres, which liquefy when transferred to an incubator, were added to form pores within the printed constructs. The presence of these pores weakens the scaffold and enhances the effect of cell-generated forces by allowing cell proliferation, stretching, and migration.

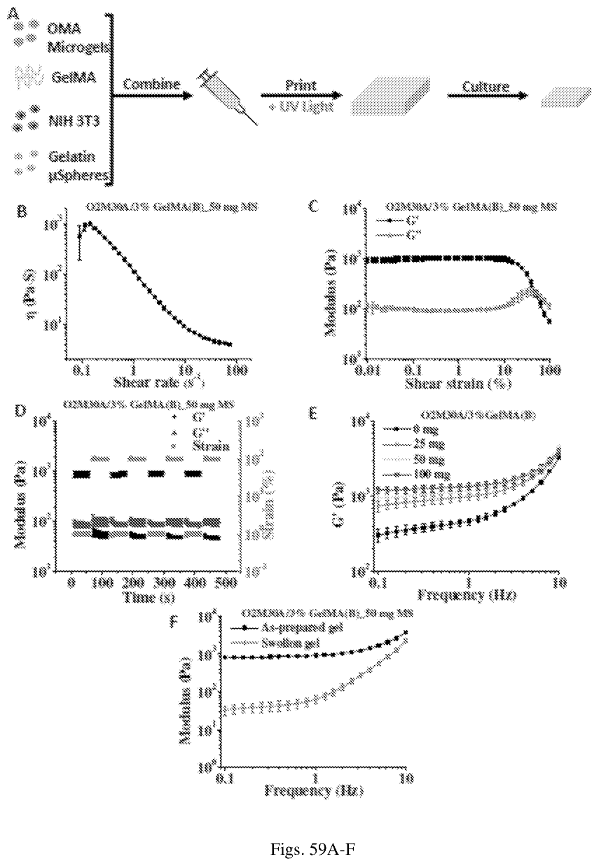

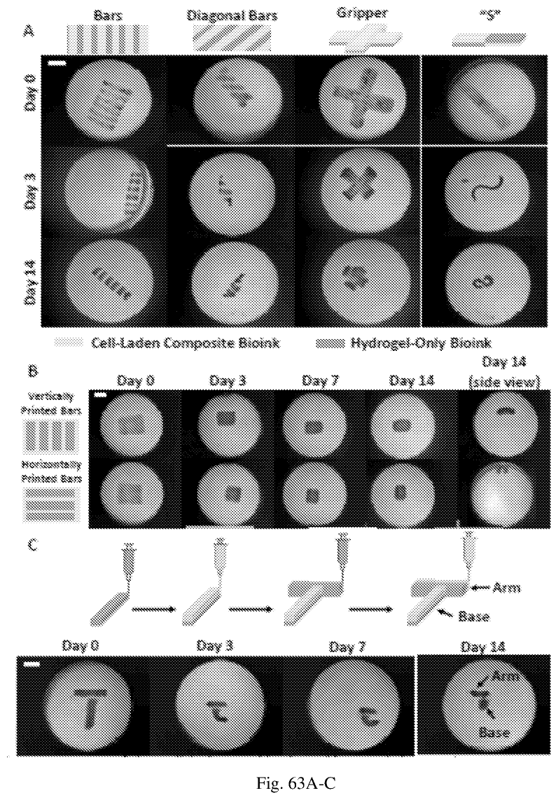

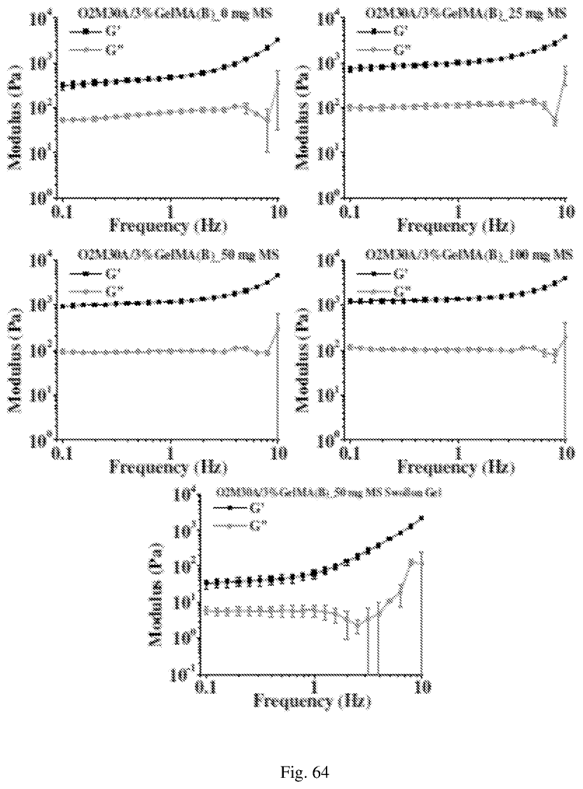

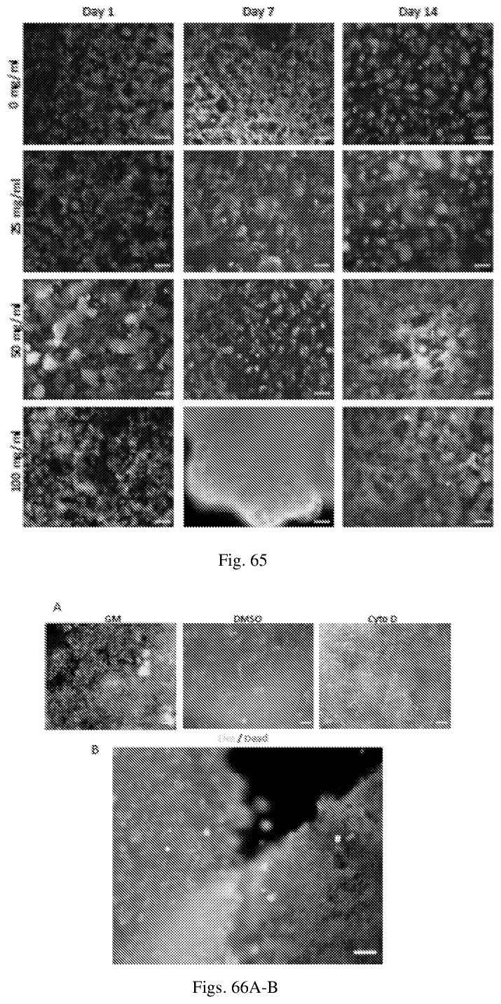

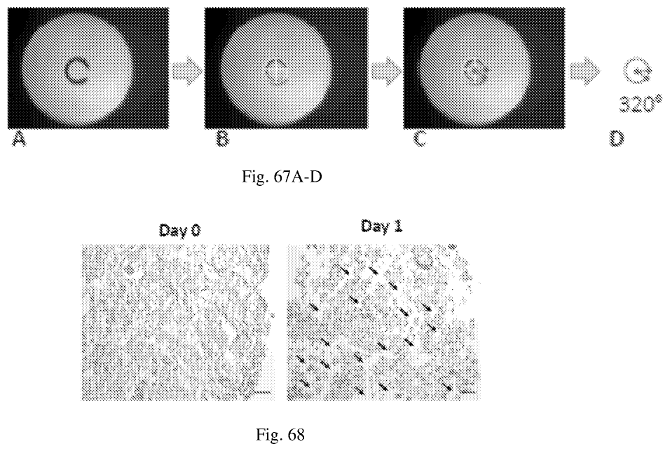

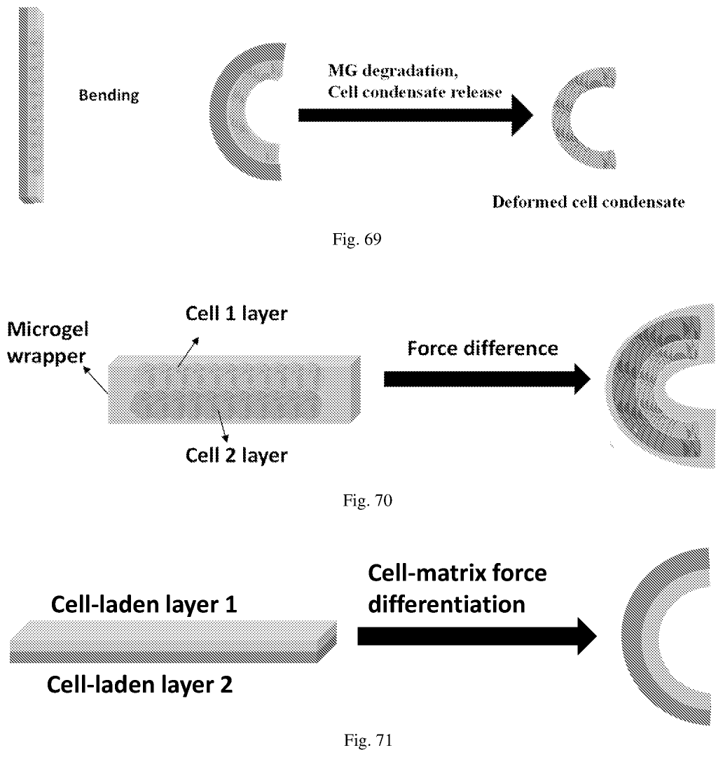

In order to be used for free-standing 3D printing (i.e., not printing in a support bath), bioinks must exhibit shear-thinning and rapid self-healing properties. Shown in FIG. 65B, the viscosity of the composite microenvironment decreases dramatically as shear rate increases, confirming shear-thinning behavior. Additionally, the storage (G′) and loss (G″) moduli of the bioink cross over each other as shear strain increases (FIG. 65C). At low shear strains (i.e., when the bioink is at rest), G′ is greater than G″, indicating that the bioink behavior is mainly solid-like. However, at high shear strains (i.e., when the bioink is flowing through the needle), G′ is less than G″, indicating that the bioink behavior is mainly liquid-like. Along with these shear-thinning properties, an oscillatory strain test reveals the self-healing quality of the bioink (FIG. 65D). As strain oscillates between 1% and 100%, the moduli oscillate between the same values, indicating that the bioink is able to self-heal and consistently respond to strains even after multiple exposures. Taken together, these shear-thinning and self-healing behaviors impart the composite bioink with exceptional printability. However, since the purpose of this bioink is to aid in CTF-mediated changes in shape, these rheological properties are rendered moot unless the crosslinked bioink is also soft enough to be deformed by cellular forces (G′<200 Pa). To determine this, G′ was measured at frequencies less than 10 Hz for formulations of the bioink containing different concentrations of gelatin microspheres. G′ was observed to increase with increasing gelatin microsphere concentration (FIG. 59E). G″ was less than G′ for all bioink formulations at frequencies less than 10 Hz, indicating that all formulations exhibit solid-like behavior at rest (FIG. 64). Since the gelatin microspheres are uncrosslinked and liquefy when cultured at 37° C., the G′ of the crosslinked composite hydrogel after one day of culture is significantly reduced, enabling cellular forces to deform their microenvironment (FIG. 59F).

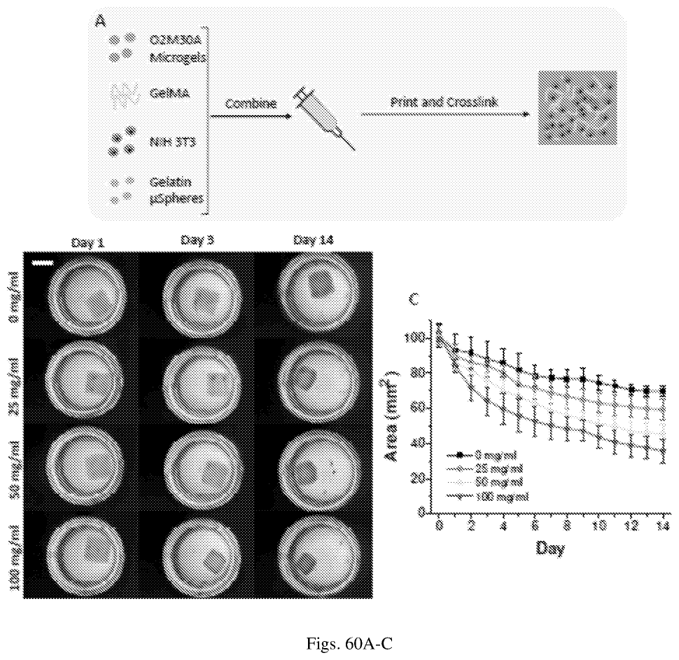

To investigate the effect of porosity on the ability of cellular forces to deform the composite matrix, cell-laden OMA/GelMA bioinks with various concentrations of gelatin microspheres were printed into 10 mm×10 mm×0.6 mm squares, crosslinked with UV light, and cultured for 14 days (FIG. 60A). Photomicrographs of each sample (n=4) were obtained each day using a dissection microscope. Representative photomicrographs for each condition at the day 1, 3, and 14 timepoints are presented in FIG. 59B. The shrinkage of each cell-laden hydrogel was quantified by measuring the area of each square, revealing the trend that increased gelatin microsphere concentration results in reduced area at day 14 (FIG. 60C). These results are intuitive, given that the gelatin microspheres liquefy at the beginning of culture, leaving microscale pores within the printed constructs (FIG. 68). These pores reduce the resistance of the microenvironment to deformation by cellular forces, resulting in a noticeable increase in contraction. Constructs in all conditions exhibited >80% viability at days 1, 7, and 14 (FIG. 65), indicating that all bioink formulations demonstrate high cytocompatibility. While the 100 mg mL⁻¹ condition conferred the greatest amount of contraction, it also introduced some difficulties in hydrogel stability. Since large portions of the constructs in this condition liquefy, four out of eight constructs dissociated within 24 hours of culture. Therefore, we determined that 50 mg mL⁻¹ was the optimal concentration of gelatin microspheres to ensure macroscopic changes in shape while maintaining proper hydrogel stability after liquefaction of the gelatin microspheres.

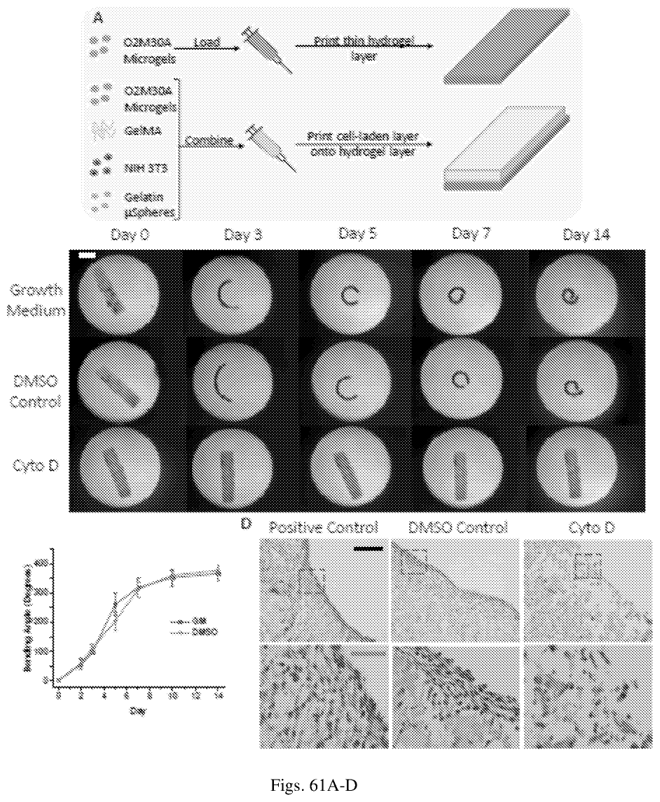

4D biomaterials may be useful in modelling the complex changes in shape observed during embryonic development. To investigate the ability to control the direction of bending in our 4D system, we pursued a bilayer approach. Here, the bottom layer consists only of OMA microgels while the top layer consists of the cell-laden composite microenvironment. The top layer was printed directly onto the bottom layer and the entire construct was crosslinked with UV light to obtain the desired bilayer structure (FIG. 61A). Cells within the top layer made physical connections with each other and their matrix via cell adhesions. This enabled cytoskeleton-generated cellular contraction forces in the cell-laden layer to be propagated into the hydrogel layer, resulting in macroscopic shrinkage in the cell-laden layer. The hydrogel layer resisted this contraction, causing both layers to bend, as shown in the growth medium condition in FIG. 61B. To validate the role of cellular forces in stimulating the observed geometric changes, bilayer constructs were cultured in growth medium containing Cytochalasin D, a known inhibitor of actin polymerization. Since cellular forces are generated by the activation of the actin-myosin complex, inhibiting actin polymerization greatly diminishes a cell's ability to generate forces. Here, Cytochalasin D (CytoD) was supplemented in growth medium at a concentration of 5 μM according to established literature. Constructs cultured in this media displayed no macroscopic changes in shape over the duration of culture with no sign of cell death at day 14 (FIG. 66). Since CytoD is soluble in DMSO, the supplemented growth media also contained 0.1% v/v DMSO. To corroborate that this concentration of DMSO has no effect on bending, a vehicle control condition was established in which constructs were cultured in growth medium with 0.1% DMSO. The normal growth medium and DMSO conditions showed no significant differences in bending angle over 14 days (FIG. 61C). Histological analysis was performed to investigate the microscale effects of CytoD treatment (FIG. 61D). H&E staining of samples in each media condition illustrate that cells in the growth medium and DMSO groups were able to form a fibrous border with cell bodies aligned along the outer surfaces of the constructs, consistent with normal behavior of fibroblasts. Cells in the CytoD group were unable to form this fibrous border and were unable to align their bodies, consistent with the inhibition mechanism of this drug. These results suggest that deformation is ascribed to the cell-mediated matrix contraction of the upper layer. Accordingly, this is the first report of a prolonged and controllable scaffold shape change observed in a homogeneously cell-loaded construct relying on cell generated forces.

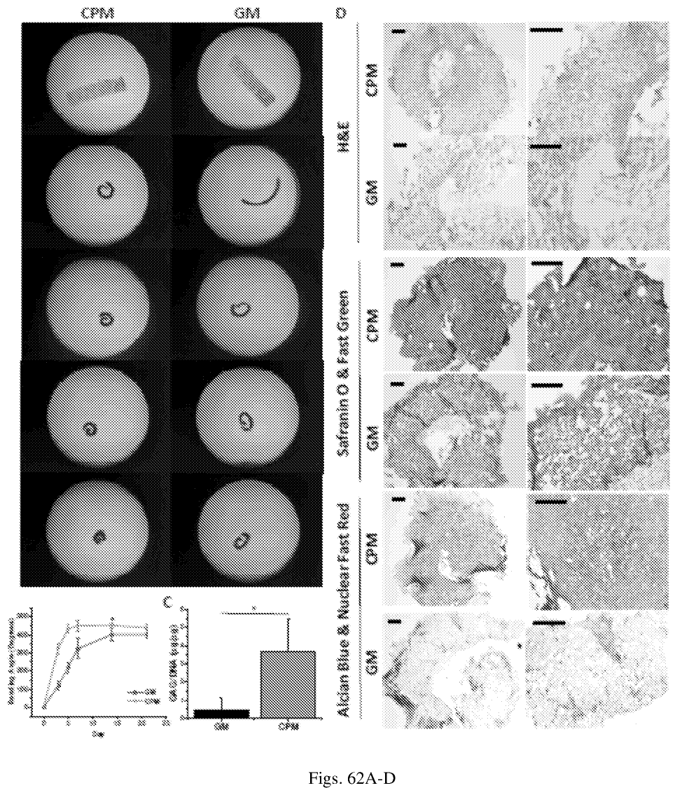

Previous reports have shown that the condensation of human mesenchymal stem cells (hMSCs) is accelerated when exposed to chondrogenic growth factors such as transforming growth factor beta 1 (TGFβ1). Therefore, we investigated the effects of chondrogenic differentiation of primary hMSCs on the rate and extent of 4D geometric changes in the bilayered constructs. To accomplish this, 1×10⁸ hMSCs were suspended in the composite bioink and bilayer constructs were printed. Constructs were cultured in one of two media: (1) normal growth medium (GM), or (2) chondrogenic pellet medium (CPM), a serum-free medium which includes TGFβ1. FIG. 62A shows representative photomicrographs of constructs in each condition over 21 days of culture. The bending angles of constructs in CPM were significantly greater (p<0.01) than those of constructs in GM at days 3, 5, and 7 (FIG. 62B), indicating that exposure to chondrogenic factors induces a greater rate of bending in hMSC-laden constructs but does not increase the maximum possible bending angle.

During chondrogenic differentiation, hMSCs excrete a matrix rich in negatively charged polysaccharides known as glycosaminoglycans (GAGs), which are responsible for the unique mechanical properties of cartilage. Therefore, the presence of GAGs is a useful measure to evaluate the extent of chondrogenesis of bilayer constructs in the GM and CPM media conditions. Biochemical analysis revealed that constructs cultured in CPM had significantly higher (p<0.05) GAG content normalized to DNA content, consistent with an increase in chondrogenesis (FIG. 62C). Additionally, histological analysis was performed to visually corroborate the biochemical results (FIG. 62D). H&E staining reveals that cells cultured in CPM condensed more than cells cultured in GM, consistent with the macroscopic photomicrographs. Staining for Safranin O and Fast Green reveals the presence of negatively charged proteoglycans and Collagen I, respectively. In both CPM- and GM-cultured constructs, much of the collagen I staining is localized on the inner surface of the constructs, consistent with our previous results that the cells build a fibrous border around the outside of the constructs. CPM-cultured constructs stained more intensely for Safranin O, reinforcing the increase in normalized GAG measured in the biochemical analysis. However, the OMA in the composite bioink also stains positively with Safranin O. Therefore, constructs were also stained with Alcian blue at a pH of 0.2, which only stains strongly sulphated proteoglycans, such as cell-produced GAG. The lack of blue staining in the GM-cultured constructs shows that the majority of Safranin O staining in these constructs is due to the presence of OMA and not cell-produced GAG. Additionally, the intense blue staining of the CPM-cultured constructs reveals that much of the Safranin O staining is indeed cell-produced GAG. These results indicate that chondrogenesis can not only be induced simultaneously with shape morphing, but can also be used to accelerate the rate of shape morphing. Using cell forces to control changes in shape during tissue maturation enables new avenues to mimic natural tissue development, opening new possibilities for tissue engineering and developmental modeling.

Next, we investigated whether the direction of contraction can be controlled by patterning the cell-laden layer to induce complex 4D events (FIG. 63A). For example, printing parallel lines of cell-laden hydrogel on a rectangular hydrogel layer resulted in the formation of a cylindrical tube-like structure. Similarly, printing parallel cell-laden lines diagonally across a rectangular hydrogel layer resulted in the formation of a helical structure. Using 3D printing to create these structures also allows for the generation of 4D constructs that change shape along multiple axes simultaneously. To illustrate this, a 4-armed bilayer “gripper” shape was printed. Each arm was observed to bend upward and inward, similar to how one's fingers bend to grip an object in one's palm. Additionally, a bilayer rectangle was printed and adjoined to a second bilayer rectangle printed cell-laden layer first. Accordingly, bending was observed around two separate axes, resulting in an “S”-shaped structure. These results indicate that cellular forces are sufficient to drive spatiotemporal changes within this biopolymer microenvironment, and that these changes continue to occur over 14 days of culture. Importantly, the complex final structures can be generated and controlled by the precise patterning of the printed hydrogel and cell-laden layers.

Up to this point, directional bending has been controlled solely by patterning the cell-laden layer. Therefore, we investigated whether the direction of 4D bending could be influenced by patterning the cell-laden layer. To accomplish this, the cell-laden composite bioink was printed into 9×12×0.6 mm rectangles. A patterned hydrogel layer consisting of either horizontal or vertical bars was then printed onto the cell-laden layer. The hydrogel bars were observed to induce bending of the constructs around an axis perpendicular to the direction of the bars. FIG. 63B shows how the differences in initial geometry contributed to differences in shape change over the duration of culture. Rotating these constructs 90 degrees along the horizontal axis clearly shows the differences in final geometry at day 14.

In addition to controlling the direction of contraction by separately patterning the cell-free and cell-laden layers, multiple bending axes can be incorporated into a single construct by patterning both layers simultaneously. To demonstrate this, T-shaped bilayer structures were printed in a step-by-step protocol. FIG. 63C presents the schematic of printing, where the base of the T is first formed by printing a rectangular hydrogel layer, followed by a matching cell-laden layer directly on top. Next, the arm of the T was formed by printing a hydrogel layer directly adjacent to the base and subsequently printing a cell-laden layer adjacent to the hydrogel layer. As observed, this initial geometry caused the base of the T to exhibit an out-of-plane bending while the arm of the T exhibited an in-plane bending. Such multi-axial bending around two non-parallel axes has not previously been demonstrated using cell-laden biomaterials.

In this study, we exploited cells' inherent ability to contract and deform their matrix to generate constructs that change their geometry over time. Using our carefully designed bioink, complex 3D printed structures can be generated, enabling the patterning of cell-laden and cell-free layers to maximize the possibilities of spatiotemporal transformations. The ability to precisely and temporally control the shape of 3D tissue constructs using cell-generated forces may be useful to study the processes of development and healing, as well as contribute to tissue engineering and personalized medicine approaches.

In summary, we have developed a novel bioink for the 3D printing of 4D biomaterials that undergo controlled spatiotemporal geometric changes mediated by cell-generated forces. The combination of OMA, GelMA, and gelatin microspheres forms a cell-friendly bioink that enables deformation by cellular forces. By forming bilayered constructs, it was demonstrated that the direction of 4D bending can be controlled, and the role of cell-generated forces was confirmed by supplementing media with CytoD. hMSCs can be encapsulated in bilayered constructs at a density of 1.0×10⁸ cells mL⁻¹, and the inclusion of chondrogenic signals leads to an increased rate of 4D bending while simultaneously leading cells down the chondrogenic lineage. Additionally, each layer of the bilayered constructs can be patterned with 3D printing to generate complex initial and final structures. Importantly, complex patterning enables the production of 4D biomaterials where different portions of a construct bend around different axes simultaneously. To the best of our knowledge, this is the first report of CTF-mediated 4D biomaterials that can be generated using free-standing 3D bioprinting. This study represents a great increase in biomimicry of 4D technologies and has the potential to significantly impact 4D tissue engineering for modelling embryonic development and creating personalized treatments for damaged tissues.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A construct comprising:
a biocompatible and/or cytocompatible polymer-based shape morphing hydrogel that is configured to undergo one or more reversible, controllable and/or different shape transformations over time via either pre-programmed design or user-controlled environmental condition alterations, wherein the hydrogel is upon degradation produces substantially non-toxic products.

2. The construct of claim 1, the shape morphing hydrogel including at least one layer, wherein the swelling and/or degradation rate of the at least one layer actuates the shape transformations.

3. The construct of claim 1, including a first layer that includes a first hydrogel forming biocompatible polymer macromer and a second layer that includes a second hydrogel forming biocompatible polymer macromer different than the first hydrogel forming biocompatible polymer macromer.

4. The construct of claim 1, including multiple layers having similar or different swelling ratios.

5. The construct of claim 3, wherein the at least one of the layers includes hydrogel forming acrylated and/or methacrylated polymer macromers that are optionally oxidized.

6. (canceled)

7. The construct of claim 5, wherein the acrylated and/or methacrylated polymer macromers are photocrosslinkable, ionically crosslinkable, physically crosslinkable, pH crosslinkable, dual crosslinkable, and/or thermally crosslinkable.

8. (canceled)

9. The construct of claim 3, wherein at least one of the layers includes an acrylated and/or methacrylated alginate that is optionally oxidized and/or at least one of the layers includes an acrylated and/or methacrylated gelatin.

10. The construct of claim 3, wherein at least one of the layers includes a first oxidized and acrylated and/or methacrylated natural polymer macromer and another layer includes a second oxidized and acrylated and/or methacrylated natural polymer macromer, and wherein the oxidation and/or acrylation and/or methacrylation of the second natural polymer macromer is different from the oxidation and/or acrylation and/or methacrylation of the second polymer macromer.

11. The construct of claim 1, wherein the shape morphing hydrogel exhibits a repeatable and reversible shape change based on exogenous stimulation.

12. The construct of claim 10, wherein the exogenous stimulation includes at least one of chemical, biochemical, irradiation, magnetic, biological, electric, ultrasound/sound, mechanical or a change in pH or temperature.

13. The construct of claim 1, wherein the shape morphing hydrogel is ionically cross-linkable and the shape transformation is actuated by increasing or decreasing the concentration of ionic cross-linker in the shape morphing hydrogel.

14. The construct of claim 1, wherein the shape morphing hydrogel is self-morphing and/or user regulated on-demand morphing into three dimensional architectures under physiological or non-physiological conditions.

15. The construct of claim 1, further comprising a plurality of cells dispersed in the hydrogel and wherein at least a portion of the construct has a cell density up to 1×10¹⁰ cells/ml.

16. (canceled)

17. The construct of claim 15, wherein the plurality of cells comprises progenitor cells, undifferentiated cells, differentiated cells, and/or cancer cells.

18. (canceled)

19. The construct of claim 1, including a plurality of layers of hydrogel forming polymer macromers, wherein at least two of the layers have different macromer concentration, acrylation and/or methacrylation, oxidation, thickness, and/or cell density.

20. The construct of claim 19, wherein at least two layers are covalently linked at adjoining portions.

21. The construct of claim 19, comprising at least three layers, wherein a middle layer is covalently linked to adjoining portions of two outer layers.

22. A method of forming a construct of claim 1, the method comprising:
adhering a first layer that includes a first hydrogel forming natural polymer macromer to a second layer that includes a second hydrogel forming polymer macromer having a different swelling ratio and/or degradation rate than the first hydrogel forming natural polymer macromer, wherein the different swelling ratio and/or degradation rate allows the hydrogel to undergo multiple, reversible, controllable and/or different shape transformations, and wherein the hydrogel is cytocompatible and, upon degradation, produces substantially non-toxic products.

23. The method of claim 22, further comprising adhering at least three layers of hydrogel forming natural polymer macromer, wherein at least two of layers have different compositions and a different swelling ratio and/or degradation rate.

24. The method of claim 22, further comprising adhering a third layer to the first second layer such that the second layer is sandwiched between the first layer and the third layer, the third layer including a third hydrogel forming polymer macromer.

25-179. (canceled)
